(19) United States
(12) Patent Application Publication
(10) Pub. No.: US 2014/0243356 A1
(43) Pub. Date: Aug. 28, 2014

(54) USE OF TRANSLATIONAL PROFILING TO IDENTIFY TARGET MOLECULES FOR THERAPEUTIC TREATMENT

(71) Applicant: The Regents of the University of California, Oakland, CA (US)

(72) Inventors: Davide Ruggero, San Francisco, CA (US); Andrew Hsieh, San Franicisco, CA (US); Merritt Edlind, San Francisco, CA (US)

(73) Assignee: The Regents of the University of California, Oakland, CA (US)

(21) Appl. No.: 14/175,736

(22) Filed: Feb. 7, 2014

Related U.S. Application Data
(60) Provisional application No. 61/762,115, filed on Feb. 7, 2013.

(51) Int. Cl. C12Q 1/68

(52) U.S. Cl. CPC C12Q 1/6874
USPC 514/262.1; 506/9; 514/291

(57) ABSTRACT

The present invention provides methods of identifying an agent that modulates an oncogenic signaling pathway in a biological sample by generating a translational profile of gene translational levels in the biological sample. The present invention also provides diagnostic and therapeutic methods using the translational profiling methods described herein.

### CROSS-REFERENCE TO RELATED APPLICATIONS

The present application claims priority to U.S. Provisional Application No. 61/762,115, filed Feb. 7, 2013, the entire content of which is incorporated by reference herein for all purposes.

### STATEMENT AS TO RIGHTS TO INVENTIONS MADE UNDER FEDERALLY SPONSORED RESEARCH AND DEVELOPMENT

This invention was made with government support under Grant No. RO1 CA154916 awarded by the National Institutes of Health. The government has certain rights in the invention.

### BACKGROUND OF THE INVENTION

Gene expression studies have been used to examine mRNA in cell populations under different conditions, e.g., for comparing gene expression under different drug treatments or in different cell types. For example, Cheok et al. (Nat Genet. 34:85-90 (2003)) demonstrated that lymphoid leukemia cells of different molecular subtypes share common pathways of genomic response to the same treatment, and that changes in gene expression are treatment-specific and that gene expression can illuminate differences in cellular response to drug combinations versus single agents. However, these types of gene expression studies have many drawbacks. For example, genome-scale predictions of synthesis rates of mRNAs and proteins have been used to demonstrate that cellular abundance of proteins is predominantly controlled at the level of translation. Schwanhausser et al. (Nature 473:337-342 (2011)).

The mammalian target of rapamycin (mTOR) kinase is a master regulator of protein synthesis that couples nutrient sensing to cell growth and cancer. However, the downstream translationally regulated nodes of gene expression that may direct cancer development have not been well characterized. Thus, there remains a need for methods of characterizing the translational control of mRNAs in oncogenic mTOR signaling and in cell populations generally. The present invention addresses this need and others.

### BRIEF SUMMARY OF THE INVENTION

In one aspect, the present invention relates to methods for identifying an agent that modulates an oncogenic signaling pathway (e.g., an agent that inhibits an oncogenic signaling pathway) in a biological sample. In some embodiments, the method comprises:
(a) contacting the biological sample with an agent;
(b) determining a first translational profile for the contacted biological sample, wherein the translational profile comprises translational levels for one or more genes having a 5′ terminal oligopyrimidine tract (5′ TOP) and/or a pyrimidine-rich translational element (PRTE); and
(c) comparing the first translational profile to a second translational profile comprising translational levels for the one or more genes in a control sample that has not been contacted with the agent;
   wherein a difference in the translational levels of the one or more genes in the first translation profile as compared to the second translation profile identifies the agent as a modulator of the oncogenic signaling pathway.

In some embodiments, the method comprises:
(a) contacting the biological sample with an agent;
(b) determining a first translational profile for the contacted biological sample,
   wherein the translational profile comprises translational levels for one or more genes selected from the group consisting of SEQ ID NOs:1-144; and
(c) comparing the first translational profile to a second translational profile comprising translational levels for the one or more genes in a control sample that has not been contacted with the agent;
   wherein a difference in the translational levels of the one or more genes in the first translation profile as compared to the second translation profile identifies the agent as a modulator of the oncogenic signaling pathway.

In some embodiments, the method comprises:
(a) contacting the biological sample with an agent;
(b) determining a first translational profile for the contacted biological sample,
   wherein the translational profile comprises a measurement of gene translational levels for a substantial portion of the genome;
(c) comparing the first translational profile to a second translational profile comprising a measurement of gene translational levels for the substantial portion of the genome translational levels for the one or more genes in a control sample that has not been contacted with the agent;
(d) identifying in the first translational profile a plurality of genes having decreased translational levels as compared to the translational levels of the plurality of genes in the second translational profile; and
(e) determining whether, for the plurality of genes identified in step (d), there is a common consensus sequence and/or regulatory element in the untranslated regions (UTRs) of the genes that is shared by at least 10% of the plurality of genes identified in step (d);
   wherein a decrease in the translational levels of at least 10% of the genes sharing the common consensus sequence and/or UTR regulatory element in the first translational profile as compared to the second translational profile identifies the agent as an inhibitor of an oncogenic signaling pathway.

In some embodiments, the one or more genes are selected from the genes listed in Table 1, Table 2, and/or Table 3. In some embodiments, the one or more genes are cell invasion and/or metastasis genes. In some embodiments, the one or more genes are selected from Y-box binding protein 1 (YB1), vimentin, metastasis associated 1 (MTA1), and CD44.

In some embodiments, the oncogenic signaling pathway is the mammalian target of rapamycin (mTOR) pathway, the PI3K pathway, the AKT pathway, the Ras pathway, the Myc pathway, the Wnt pathway, or the BRAF pathway. In some embodiments, the oncogenic signaling pathway is the mTOR pathway.

In some embodiments, the translational level for the one or more genes is decreased for the first translational profile as compared to the second translational profile, thereby identifying the agent as an inhibitor of the oncogenic signaling pathway. In some embodiments, the translational level of the one or more genes in the first translational profile is decreased by at least three-fold as compared to the second translational profile. In some embodiments, the translational level for the one or more genes is increased for the first translational profile as compared to the second translational profile, thereby identifying the agent as a potentiator of the oncogenic signaling pathway. In some embodiments, the translational level of the one or more genes in the first translational profile is increased by at least three-fold as compared to the second translational profile.

In some embodiments, the first and/or second translational profiles are generated using ribosomal profiling. In some embodiments, the first and/or second translational profiles are generated using polysome microarray. In some embodiments, the first and/or second translational profiles are generated using immunoassay. In some embodiments, the first and/or second translational profiles are generated using mass spectrometry analysis.

In some embodiments, the first and/or second translation profile comprises measuring the translational levels of at least 500 genes in the sample (e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more). In some embodiments, the first and/or second translational profile comprises a genome-wide measurement of gene translational levels.

In some embodiments, the biological sample comprises a cell. In some embodiments, the cell is a human cell. In some embodiments, the cell is a cancer cell. In some embodiments, the cancer is prostate cancer, breast cancer, bladder cancer, lung cancer, renal cell carcinoma, endometrial cancer, melanoma, ovarian cancer, thyroid cancer, or brain cancer.

In some embodiments, the identified agent binds to a 5′ TOP or PRTE sequence in the one or more genes having a different translational level in the first translational profile as compared to the second translational profile. In some embodiments, the identified agent inhibits the activity of a downstream effector of the oncogenic signaling pathway, wherein the effector is 4EBP1, p70S6K1/2, or AKT.

In some embodiments, the method further comprises chemically synthesizing a structurally related agent derived from the identified agent. In some embodiments, the method further comprises administering the structurally related agent to an animal and determining the oral bioavailability of the structurally related agent. In some embodiments, the method further comprises administering the structurally related agent to an animal and determining the potency of the structurally related agent.

In another aspect, the present invention relates to a structurally related agent to an agent identified as described herein.

In still another aspect, the present invention relates to methods of validating a target for therapeutic intervention. In some embodiments, the method comprises:
(a) contacting a biological sample with an agent that modulates the target;
(b) determining a first translational profile for the contacted biological sample, wherein the first translational profile comprises translational levels for a plurality of genes; and
(c) comparing the first translational profile to a second translational profile comprising translational levels for the plurality of genes in a control sample that has not been contacted with the agent;
   wherein identifying one or more genes of a biological pathway as differentially translated in the first translational profile as compared to the second translational profile validates the target for therapeutic intervention, wherein said biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway.

In some embodiments, the one or more genes have a 5′ terminal oligopyrimidine tract (5′ TOP) and/or a pyrimidine-rich translational element (PRTE). In some embodiments, the one or more genes are selected from the group consisting of SEQ ID NOs:1-144.

In some embodiments, the target for therapeutic intervention is part of an oncogenic signaling pathway. In some embodiments, the oncogenic signaling pathway is the mammalian target of rapamycin (mTOR) pathway. In some embodiments, the target for therapeutic intervention is a protein. In some embodiments, the target for therapeutic intervention is a nucleic acid.

In some embodiments, one or more genes from each of at least two of the biological pathways is differentially translated in the first translational profile as compared to the second translational profile. In some embodiments, one or more genes from each of at least three of the biological pathways is differentially translated in the first translational profile as compared to the second translational profile. In some embodiments, there is at least a two-fold difference in translational level for the one or more genes in the first translational profile as compared to the second translational profile.

In some embodiments, the first and/or second translational profile comprises a genome-wide measurement of gene translational levels. In some embodiments, less than 20% of the genes in the genome are differentially translated by at least two-fold in the first translational profile as compared to the second translational profile. In some embodiments, less than 5% of the genes in the genome are differentially translated by at least two-fold in the first translational profile as compared to the second translational profile. In some embodiments, less than 1% of the genes in the genome are differentially translated by at least two-fold in the first translational profile as compared to the second translational profile.

In some embodiments, the first and/or second translational profiles are generated using ribosomal profiling. In some embodiments, the first and/or second translational profiles are generated using polysome microarray. In some embodiments, the first and/or second translational profiles are generated using immunoassay. In some embodiments, the first and/or second translational profiles are generated using mass spectrometry analysis.

In some embodiments, the biological sample comprises a cell. In some embodiments, the cell is a human cell. In some embodiments, the cell is a cancer cell. In some embodiments, the cancer is prostate cancer, breast cancer, bladder cancer, lung cancer, renal cell carcinoma, endometrial cancer, melanoma, ovarian cancer, thyroid cancer, or brain cancer.

In some embodiments, the therapeutic intervention is an anti-cancer therapy.

In some embodiments, the agent is a peptide, protein, RNA, or small organic molecule. In some embodiments, the agent is an inhibitory RNA.

In yet another aspect, the present invention relates to methods of identifying a drug candidate molecule. In some embodiments, the method comprises:
(a) contacting a biological sample with the drug candidate molecule;
(b) determining a translational profile for the contacted biological sample, wherein the translational profile comprises translational levels for a plurality of genes; and
(c) comparing the first translational profile to a second translational profile comprising translational levels for the plurality of genes in a control sample that has not been contacted with the drug candidate molecule,
   wherein the drug candidate molecule is identified as suitable for use in a therapeutic intervention when one or more genes of a biological pathway is differentially translated in the first translational profile as compared to the second translational profile, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and DNA methylation pathway.

In some embodiments, the one or more genes have a 5′ terminal oligopyrimidine tract (5′ TOP) and/or a pyrimidine-rich translational element (PRTE). In some embodiments, the one or more genes are selected from the group consisting of SEQ ID NOs:1-144.

In some embodiments, one or more genes from each of at least two of the biological pathways is differentially translated in the first translational profile as compared to the second translational profile. In some embodiments, one or more genes from each of at least three of the biological pathways is differentially translated in the first translational profile as compared to the second translational profile. In some embodiments, there is at least a two-fold difference in translational level for the one or more genes in the first translational profile as compared to the second translational profile.

In some embodiments, the first and/or second translational profile comprises a genome-wide measurement of gene translational levels. In some embodiments, less than 20% of the genes in the genome are differentially translated by at least two-fold in the first translational profile as compared to the second translational profile. In some embodiments, less than 5% of the genes in the genome are differentially translated by at least two-fold in the first translational profile as compared to the second translational profile. In some embodiments, less than 1% of the genes in the genome are differentially translated by at least two-fold in the first translational profile as compared to the second translational profile.

In some embodiments, the first and/or second translational profiles are generated using ribosomal profiling. In some embodiments, the first and/or second translational profiles are generated using polysome microarray. In some embodiments, the first and/or second translational profiles are generated using immunoassay. In some embodiments, the first and/or second translational profiles are generated using mass spectrometry analysis.

In some embodiments, the method further comprises comparing the translational profile for the contacted biological sample with a control translational profile for a second biological sample that has been contacted with a known therapeutic agent. In some embodiments, the known therapeutic agent is a known inhibitor of an oncogenic signaling pathway. In some embodiments, the known therapeutic agent is a known inhibitor of the mammalian target of rapamycin (mTOR) pathway.

In still another aspect, the present invention relates to methods of identifying a subject as a candidate for treatment with an mTOR inhibitor. In some embodiments, the method comprises:
(a) determining a first translational profile in a sample from the subject, wherein the first translational profile comprises translational levels for one or more genes having a 5′ terminal oligopyrimidine tract (5′ TOP) and/or a pyrimidine-rich translational element (PRTE); and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder; wherein a translational level of the one or more genes in the first translational profile that is at least as high as the translational level of the one or more genes in the second translational profile identifies the subject as a candidate for treatment with the mTOR inhibitor.

In some embodiments, the method comprises:
(a) determining a first translational profile in a sample from the subject, wherein the first translational profile comprises translational levels for one or more genes selected from the group consisting of SEQ ID NOs:1-144; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
   wherein a translational level of the one or more genes in the first translational profile that is at least as high as the translational level of the one or more genes in the second translational profile identifies the subject as a candidate for treatment with the mTOR inhibitor.

In some embodiments, the one or more genes are selected from the genes listed in Table 1, Table 2, and/or Table 3. In some embodiments, the one or more genes are cell invasion and/or metastasis genes. In some embodiments, the one or more genes are selected from Y-box binding protein 1 (YB1), vimentin, metastasis associated 1 (MTA1), and CD44.

In some embodiments, the method comprises:
(a) determining a first translational profile in a sample from the subject, wherein the first translational profile comprises translational levels for one or more genes of a biological pathway, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
   wherein a translational level of the one or more genes in the first translational profile that is at least as high as the translational level of the one or more genes in the second translational profile identifies the subject as a candidate for treatment with the mTOR inhibitor.

In some embodiments, the translational level of one or more genes from each of at least two of the biological pathways is at least as high in the first translational profile as in the second translational profile. In some embodiments, the translational level of one or more genes from each of at least three of the biological pathways is at least as high in the first translational profile as in the second translational profile.

In some embodiments, there is at least a two-fold difference in translational level for the one or more genes in the first translational profile as compared to the second translational profile.

In some embodiments, the first and/or second translation profile comprises measuring the translational levels of at least 500 genes in the sample (e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more). In some embodiments, the first and/or second translational profile comprises a genome-wide measurement of gene translational levels. In some embodiments, the first and second translational profiles are differential profiles from before and after administration of the mTOR inhibitor.

In some embodiments, the subject has a cancer. In some embodiments, the cancer is prostate cancer, breast cancer, bladder cancer, lung cancer, renal cell carcinoma, endometrial cancer, melanoma, ovarian cancer, thyroid cancer, or brain cancer.

In some embodiments, the method further comprises administering an mTOR inhibitor to the subject.

In still another aspect, the present invention relates to methods of identifying a subject as a candidate for treatment with a therapeutic agent. In some embodiments, the method comprises:
(a) determining a first translational profile in a sample from the subject, wherein the translational profile comprises translational levels for one or more genes of a biological pathway, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the therapeutic agent prior to administration of the therapeutic agent to the known responder;
   wherein a translational level of the one or more genes that is at least as high as the translational level of the one or more genes in the second translational profile identifies the subject as a candidate for treatment with the therapeutic agent.

In some embodiments, the translational level of one or more genes from each of at least two of the biological pathways is at least as high in the first translational profile as in the second translational profile. In some embodiments, the translational level of one or more genes from each of at least three of the biological pathways is at least as high in the first translational profile as in the second translational profile.

In some embodiments, the first and second translational profiles are differential profiles from before and after administration of the therapeutic agent.

In some embodiments, the subject has a disease. In some embodiments, the disease is cancer. In some embodiments, the cancer is prostate cancer, breast cancer, bladder cancer, lung cancer, renal cell carcinoma, endometrial cancer, melanoma, ovarian cancer, thyroid cancer, or brain cancer. In some embodiments, the biological sample comprises diseased cells.

In yet another aspect, the present invention relates to methods of treating a subject having a cancer. In some embodiments, the method comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes having a 5′ terminal oligopyrimidine tract (5′ TOP) and/or a pyrimidine-rich translational element (PRTE); and wherein the control sample is from a known responder to the mTOR inhibitor prior to administration of the mTOR inhibitor to the known responder;
thereby treating the cancer in the subject.

In some embodiments, the method of treating a subject having a cancer comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes selected from the group consisting of SEQ ID NOs:1-144; and wherein the control sample is from a known responder to the mTOR inhibitor prior to administration of the mTOR inhibitor to the known responder;
thereby treating the cancer in the subject.

In some embodiments, the one or more genes are selected from the genes listed in Table 1, Table 2, and/or Table 3. In some embodiments, the one or more genes are cell invasion and/or metastasis genes. In some embodiments, the one or more genes are selected from Y-box binding protein 1 (YB1), vimentin, metastasis associated 1 (MTA1), and CD44.

In some embodiments, the method of treating a subject having a cancer comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes of a biological pathway selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the mTOR inhibitor prior to administration of the mTOR inhibitor to the known responder;
thereby treating the cancer in the subject.

In some embodiments, the translational level of one or more genes from each of at least two of the biological pathways is at least as high in the first translational profile as in the second translational profile. In some embodiments, the translational level of one or more genes from each of at least three of the biological pathways is at least as high in the first translational profile as in the second translational profile.

In some embodiments, the first and/or second translation profile comprises measuring the translational levels of at least 500 genes in the sample (e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more). In some embodiments, the first and/or second translational profile comprises a genome-wide measurement of gene translational levels. In some embodiments, the first and second translational profiles are differential profiles from before and after administration of the mTOR inhibitor.

In some embodiments, the subject has a cancer. In some embodiments, the cancer is prostate cancer, breast cancer, bladder cancer, lung cancer, renal cell carcinoma, endometrial cancer, melanoma, ovarian cancer, thyroid cancer, or brain cancer. In some embodiments, the cancer is an invasive cancer.

In some embodiments, the method further comprises monitoring the translational levels of the one or more genes in the subject subsequent to administering the mTOR inhibitor.

In still another aspect, the present invention relates to methods of treating a subject in need thereof. In some embodiments, the method comprises:
administering a therapeutic agent to a subject that has been selected as having a first translational profile comprising a translational level of one or more genes that is at least as high as the translational level of the one or more genes in a second translational profile;
wherein the first and second translational profiles comprise translational levels for one or more genes of a biological pathway selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the therapeutic agent prior to administration of the therapeutic agent to the known responder;
thereby treating the subject.

In some embodiments, the translational level of one or more genes from each of at least two of the biological pathways is at least as high in the first translational profile as in the second translational profile. In some embodiments, the translational level of one or more genes from each of at least three of the biological pathways is at least as high in the first translational profile as in the second translational profile.

In some embodiments, the first and second translational profiles are differential profiles from before and after administration of the therapeutic agent.

In some embodiments, the subject in need of treatment has a disease. In some embodiments, the disease is cancer. In some embodiments, the cancer is prostate cancer, breast cancer, bladder cancer, lung cancer, renal cell carcinoma, endometrial cancer, melanoma, ovarian cancer, thyroid cancer, or brain cancer. In some embodiments, the cancer is an invasive cancer. In some embodiments, the biological sample comprises diseased cells.

In still another aspect, the present invention relates to methods of identifying an agent for normalizing a translational profile in a subject in need thereof. In some embodiments, the method comprises:
(a) determining a first translational profile for a first biological sample from the subject, wherein the first translational profile comprises translational levels for a plurality of genes;
(b) comparing the first translational profile to a second translational profile comprising translational levels for the plurality of genes, wherein the second translational profile is from a control sample, wherein the control sample is from a non-diseased subject;
(c) identifying one or more genes of a biological pathway as differentially translated in the first translational profile as compared to the second translational profile, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway;
(d) contacting a second biological sample from the subject with the agent;
(e) determining a third translational profile for the second biological sample, wherein the third translational profile comprises translational levels for the one or more genes identified as differentially translated in the first translational profile as compared to the second translational profile; and
(f) comparing the translational levels for the one or more genes in the third translational profile to the translational levels for the one or more genes in the first and second translational profiles;
wherein a translational level for the one or more genes in the third translational profile that is closer to the translational level for the one or more genes in the second translational profile than to the translational level for the one or more genes in the first translational profile identifies the agent as an agent for normalizing the translational profile in the subject.

In yet another aspect, the present invention relates to methods of normalizing a translational profile in a subject in need thereof. In some embodiments, the method comprises:
administering to the subject an agent that has been selected as an agent that normalizes the translational profile in the subject, wherein the agent is selected by:
   (a) determining a first translational profile for a first biological sample from the subject, wherein the first translational profile comprises translational levels for a plurality of genes;
   (b) comparing the first translational profile to a second translational profile comprising translational levels for the plurality of genes, wherein the second translational profile is from a control sample, wherein the control sample is from a non-diseased subject;
   (c) identifying one or more genes of a biological pathway as differentially translated in the first translational profile as compared to the second translational profile, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway;
   (d) contacting a second biological sample form the subject with the agent;
   (e) determining a third translational profile for the second biological sample, wherein the third translational profile comprises translational levels for the one or more genes identified as differentially translated in the first translational profile as compared to the second translational profile; and
   (f) comparing the translational levels for the one or more genes in the third translational profile to the translational levels for the one or more genes in the first and second translational profiles; wherein a translational level for the one or more genes in the third translational profile that is closer to the translational level for the one or more genes in the second translational profile than to the translational level for the one or more genes in the first translational profile identifies the agent as an agent for normalizing the translational profile in the subject;
thereby normalizing the translational profile in the subject.

In some embodiments, one or more genes from each of at least two of the biological pathways is differentially translated in the first translational profile as compared to the second translational profile. In some embodiments, one or more genes from each of at least three of the biological pathways is differentially translated in the first translational profile as compared to the second translational profile. In some embodiments, there is at least a two-fold difference in translational level for the one or more genes in the first translational profile as compared to the second translational profile.

In some embodiments, the first and/or second translation profile comprises measuring the translational levels of at least 500 genes in the sample (e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more). In some embodiments, the first, second, and/or third translational profiles comprise a genome-wide measurement of gene translational levels.

In some embodiments, the agent is a peptide, protein, inhibitory RNA, or small organic molecule.

### BRIEF DESCRIPTION OF THE DRAWINGS

FIG. 1. Ribosome profiling reveals mTOR-dependent specialized translational control of the prostate cancer genome. (a) Representative comparison of mRNA abundance and translational efficiency after a 3 hr treatment with an ATP site inhibitor (PP242) versus an allosteric inhibitor (rapamycin). (b-d) Free energy, length and percentage G+C content of the 5′ UTRs of mTOR target versus non-target mRNAs (error bars indicate range, non-target n=5,022, target n=144, two-sided Wilcoxon). (e) Functional classification of translationally regulated mTOR-responsive mRNAs. (f) Representative Western blot from three independent experiments of mTOR-sensitive invasion genes in PC3 cells after a 48-hr drug treatment. Rapa: rapamycin.

FIG. 2. mTOR promotes prostate cancer cell migration and invasion through a translationally regulated gene signature. (a) Matrigel invasion assay in PC3 cells: 6-hr pre-treatment followed by 6 hr of cell invasion (n=6, ANOVA). (b, c) Migration patterns and average distance traveled by GFP-labeled PC3 cells during hours 3-4 and 6-7 of drug treatment (n=34 cells per condition, ANOVA). (d) Matrigel invasion assay in PC3 cells after 48 hr of knockdown of YB1, MTA1, CD44, or vimentin followed by 24 hr of cell invasion (n=7, t-test). (e) Matrigel invasion assay in BPH-1 cells after 48 hr of overexpression of YB1 and/or MTA1, followed by cell invasion for 24 hr (n=7, t-test). Rapa: rapamycin. All data represent mean±s.e.m. NS: not statistically significant.

FIG. 3. The 4EBP1-eIF4E axis controls the post-transcriptional expression of mTOR-sensitive invasion genes. (a) Schematic of the pharmacogenetic strategy to inhibit p70S6K1/2 or eIF4E hyperactivation. (b) Representative Western blot from three independent experiments of PC3 4EBP1M cells after 48-hr doxycycline induction of 4EBP1^{M}. (c) Representative Western blot from three independent experiments of PC3 cells after 48-hr DG-2 treatment. (d) Representative Western blot from three independent experiments of PC3 cells after 48 h of 4EBP1/4EBP2 knockdown followed by 24-hr treatment with an ATP site inhibitor of mTOR (see quantification of independent experiments in FIG. 21a). (e) Representative Western blot from three independent experiments of wild-type (WT) and 4EBP1/4EBP2 double knockout (DKO) MEFs treated with an ATP site inhibitor of mTOR for 24 hr. (f) Representative Western blot from two independent experiments of wild-type and mSin1^{-/-} (also called Mapkap1^{tm/Bisu}) MEFs after 24-hr treatment with an ATP site inhibitor of mTOR. (g) Matrigel invasion assay upon 48-hr doxycycline induction of 4EBP1^{M}, or treatment with DG-2 compared to control (n=6 per condition, t-test). All data represent mean±s.e.m.

FIG. 4. mTOR hyperactivation augments translation of YB1, MTA1, CD44, and vimentin mRNAs in a subset of pre-invasive prostate cancer cells in vivo. Left: immunofluorescent images of CK8/DAPI or CK5/DAPI with YB1 (a, b), MTA1 (c, d), or CD44 (e, f) co-staining in 14-month-old wild-type and Pten^{L/L} mouse prostate epithelial cells. White boxes outline the area magnified in the right panel. Right: magnified immunofluorescent images of YB1 (a, b), MTA1 (c, d) and CD44 (e, f) co-stained with DAPI in wild-type and Pten^{L/L} mouse prostate epithelial cells. Dotted lines encircle the cytoplasm (C) and/or the nucleus (N). (g) Representative immunofluorescent images of CK5 or CK8 co-staining with vimentin in 14-month-old wild-type and Pten^{L/L} mouse prostate epithelial cells. S: stroma; yellow arrows indicate perinuclear vimentin. (h) Box plot of YB1 (N=nuclear, C=cytoplasmic), MTA1, and CD44 mean fluorescence intensity (m.f.i.) per CK5⁺ or CK8⁺ prostate epithelial cell in wild-type and Pten^{L/L} mice (three mice per arm, n=43-303 cells quantified per target gene, error bars indicate range (see FIG. 23b); *P<0.0001, **P=0.0004, t-test).

FIG. 5. Complete mTOR inhibition by treatment with an ATP site inhibitor of mTOR prevents prostate cancer invasion and metastasis in vivo. (a) Diagram and images of normal prostate gland, pre-invasive PIN, and invasive prostate cancer. CK8/CK5, luminal/basal epithelial cells, respectively. Yellow arrowheads indicate invasive front. (b) Immunofluorescent images of 14-month-old Pten^{L/L} lymph node (LN) metastasis co-stained with CK8/androgen receptor (AR), CK8/YB1, and CK8/MTA1. (c) Left: human tissue microarray of YB1 protein levels in normal (n=59), PIN (n=5), cancer (n=99), and CRPC (n=3) (ANOVA). Right: immunohistochemistry of YB1 in human CRPC demarcated by the red line (inset shows nuclear and cytoplasmic YB1). (d) Quantification of invasive prostate glands in wild-type and Pten^{L/L} mice before (12-months old) and after (14-months old) 60 days of treatment with an ATP site inhibitor of mTOR (n=6 mice per arm, ANOVA). (e, f) Area and number of CK8/AR+ metastases in draining lymph nodes in 14-month-old Pten^{L/L} mice after 60 days of treatment with an ATP site inhibitor of mTOR (n=6 mice per arm, t-test). (g) Percentage decrease of YB1 (N=nuclear, C=cytoplasmic), MTA1, CD44, or vimentin protein levels (determined by quantitative immunofluorescence, see FIG. 23b) in CK8⁺ or CK5⁺ prostate cells (CK8⁺ only for vimentin) in ATP site inhibitor of mTOR-treated 14-month-old Pten^{L/L} mice normalized to vehicle-treated mice (n=3 mice per arm, t-test). All data represent mean±s.e.m.

FIG. 6. Validation of mTOR inhibitors in PC3 prostate cancer cell line. (a) Schematic of ribosome profiling of human prostate cancer cells. (b) Representative Western blot analysis from 3 independent experiments of PC3 prostate cancer cells treated with rapamycin (50 nM), PP242 (2.5 μM), or ATP site inhibitor of mTOR (200 nM) for 3 hours. (c) Representative [³⁵S]-methionine incorporation in PC3 cells after 6-hour treatment with rapamycin (50 nM) or an ATP site inhibitor of mTOR (200 nM) (left panel). Quantification of [³⁵S]-methionine incorporation (right panel, n=4, mean+SEM). (d) Representative [³⁵S]-methionine incorporation in PC3 cells after 14-hour treatment with rapamycin (50 nM) or an ATP site inhibitor of mTOR (200 nM) (left panel). Quantification of [³⁵S]-methionine incorporation (right panel, n=4, mean+SEM, * P<0.05 ANOVA). (e) Cell cycle analysis of PC3 cells after treatment with rapamycin (50 nM), PP242 (2.5 μM), or an ATP site inhibitor of mTOR (200 nM) for 48 hours (mean+SEM, n=3, * P<0.001 ANOVA). (f) Cell cycle analysis of PC3 cells after 0-, 6-, or 24-hour treatment with an ATP site inhibitor of mTOR (200 nM) (mean+SEM, n=3, * P<0.001 ANOVA). n.s.: not statistically significant. V: vehicle; R: rapamycin; I: ATP site inhibitor of mTOR.

FIG. 7. Inter-experimental correlation of ribosome profiling per treatment condition and tally of mTOR responsive genes. (a) Correlation plots from 2 independent ribosome profiling experiments after a 3-hour treatment with rapamycin (50 nM) or PP242 (2.5 μM). (b) Number of translationally and transcriptionally regulated mRNA targets of mTOR after 3-hour drug treatments. (c) The Pyrimidine Rich Translational Element (PRTE) (SEQ ID NO:145) is present within the 5′ UTRs of 63% of mTOR-responsive translationally regulated mRNAs. (d) Venn diagram of the number of mTOR sensitive genes that possess a PRTE (red), 5′ TOP (green), or both (yellow).

FIG. 8. Read count profiles for eEF2, vimentin, SLC38A2, and PAICS. (a) Ribosome footprint and RNA-Seq profiles for eEF2. Read count profiles are shown for each nucleotide position in the uc002lze.2 transcript, with the eEF2 coding sequence marked. Ribosome footprints were assigned to specific A site nucleotide positions based on their length. (b) Ribosome footprint and RNA-Seq profiles for vimentin. (c) Ribosome footprint and RNA-Seq profiles for SLC38A2. (d) Ribosome footprint and RNA-Seq profiles for PAICS.

FIG. 9. False Discovery Rate computation. (a) The cumulative distribution of log₂ fold-change values is shown for three comparisons, considering only genes passing the minimum read count criterion in that comparison. The DMSO replicate represents a comparison of full biological replicates of the control DMSO-only treatment condition. The rapamycin and PP242 conditions show the ratio of drug-treated to DMSO-treated samples within a single experiment. The fold-change threshold chosen based on PP242 translational repression, described below, is shown. (b) The extremes of the log₂ fold-change cumulative distributions, showing the complementary cumulative distribution function for positive extreme values on the right. The cumulative distribution of fold-change values between the DMSO replicates was used as an estimate of the error distribution for measurements in drug treatment comparisons. That is, the fraction of genes above a given absolute value fold-change level in the comparison of biological replicates should reflect the fraction of genes above that level by chance in any measurement. At a cutoff of log₂ fold-change of +/−1.5, we detect 2.5% (95% CI, 2.1%-2.9% by Agresti-Coull) of genes in the PP242/DMSO comparison and only 0.044% (95% CI, 0.001%-0.172%) of genes in the DMSO replicate comparison. The estimated false discovery rate is therefore q=0.018 in the PP242/DMSO comparison at this fold-change threshold.

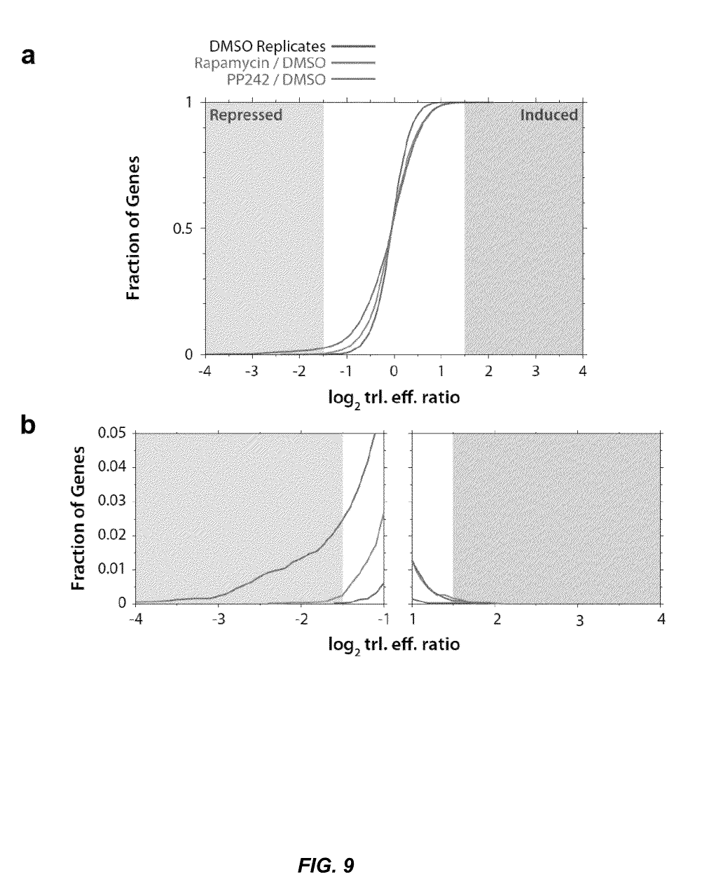

FIG. 10. Transcriptionally regulated mTOR targets. (a and b) qPCR validation of up-regulated or down-regulated transcripts identified by RNA-Seq upon 3-hour PP242 treatment (2.5 μM) in PC3 cells (mean+SEM, n=3). (c) qPCR validation of up-regulated transcript identified by RNA-Seq upon 3-hour rapamycin treatment (50 nM) in PC3 cells (mean+SEM, n=3).

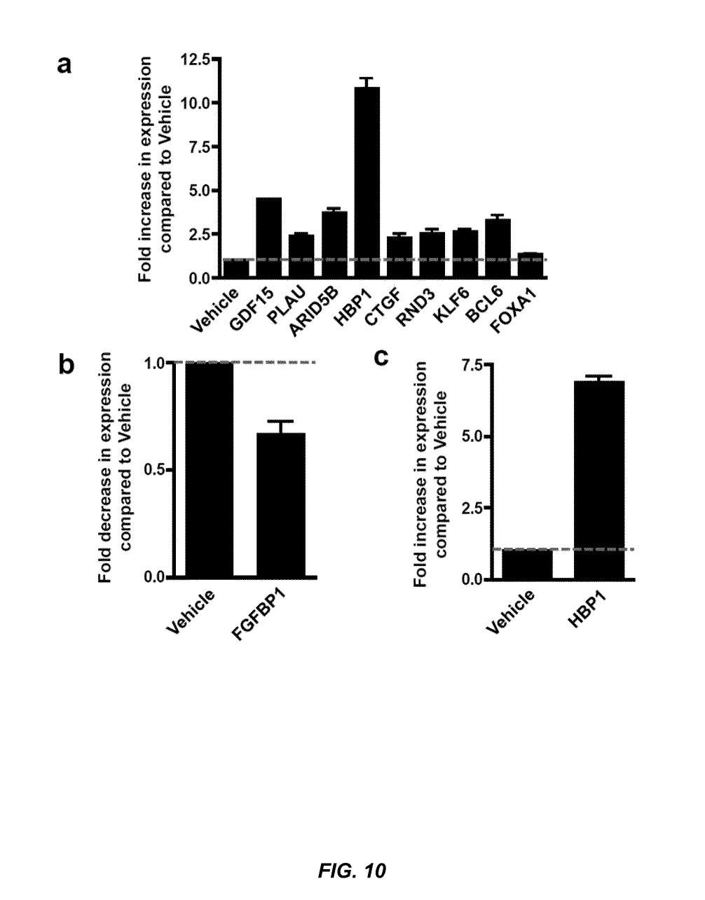

FIG. 11. mTOR-sensitive translationally regulated gene invasion signature. Mutation of the PRTE abrogates sensitivity to eIF4E. (a) 4 known pro-invasion genes and 7 putative pro-invasion genes discovered through ribosome profiling. (b) Schematic of YB1 5′ UTR cloning (WT, transversion mutant, and deletion mutant of the PRTE (position +20-34, uc001chs.2)) into pGL3-Promoter (left panel). Firefly luciferase activity in PC3-4EBP1^{M} cells after a 24-hour pre-treatment with 1 μg/ml doxycycline followed by transfection of respective 5′ UTR constructs (mean+SEM, n=7, * P<0.0001, t-test) (right panel). n.s.: not statistically significant.

FIG. 12. ATP site inhibition of mTOR does not reduce transcript levels of the 4 invasion genes. ATP site inhibitor of mTOR time course. (a) mRNA expression of YB1, MTA1, vimentin, and CD44, relative to β-actin upon treatment with rapamycin (50 nM), PP242 (2.5 μM), or an ATP site inhibitor of mTOR (200 nM) for 48 hours in PC3 cells (mean+SEM, n=3). (b) Representative Western blot of 3 independent experiments showing a time course of invasion gene expression before and after treatment with ATP site inhibitor of mTOR (200 nM) in PC3 cells.

FIG. 13. Polysome analysis after 3-hour ATP site inhibitor of mTOR treatment. (a) Ethidium bromide staining of rRNA species in individual fractions. Fractions 7-13 were determined to be polysome-associated fractions. (b) Overlay of polysome profiles from PC3 cells treated with vehicle (solid line) or ATP site inhibitor of mTOR (100 nM) (dotted line). (c) qPCR analysis of YB1 and rpS19 mRNAs that show differential association in polysome fractions after ATP site inhibitor of mTOR (100 nM) treatment (mean+SEM, n=6). The bottom graph shows that there is no change in β-actin mRNA association in polysome fractions between treatments. P-values (t-test) for each polysome fraction are shown. (d) Representative Western blot of 3 independent experiments showing a time course of eEF2 and rpL28 expression before and after treatment with ATP site inhibitor of mTOR (200 nM) in PC3 cells.

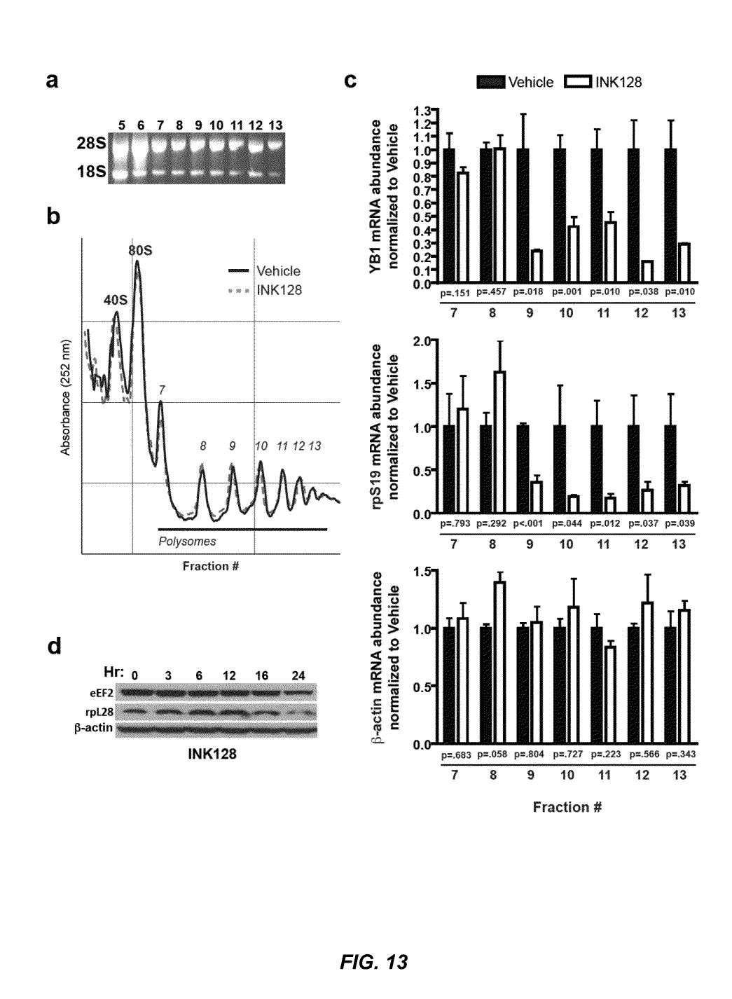

FIG. 14. 4-gene invasion signature is responsive to ATP site inhibitor of mTOR but not rapamycin in metastatic cell lines. (a-b) Representative Western blot (a) and qPCR analysis (b) of MDA-MB-361 cells after 48-hour treatment with ATP site inhibitor of mTOR (200 nM). (c-d) Representative Western blot (c) and qPCR analysis (d) of SKOV3 cells after 48-hour treatment with ATP site inhibitor of mTOR (200 nM). (e-f) Representative Western blot (e) and qPCR analysis (f) of ACHN cells after 48-hour treatment with ATP site inhibitor of mTOR (200 nM). Westerns=representative Western blot of 2 independent experiments. qPCR−n=3. All data represent mean+SEM.

FIG. 15. PTEN gene silencing in the A498 PTEN positive renal carcinoma cell line induces the post-transcriptional expression of the 4-gene invasion signature. (a-b) Representative Western blot (a) and qPCR analysis (b) of A498 cells after stable silencing of PTEN and 24 hour treatment with an ATP site inhibitor of mTOR (200 nM). Western=representative Western blot of 2 independent experiments. qPCR−n=3. All data represent mean+SEM.

FIG. 16. ATP site inhibitor of mTOR inhibits cell migration in PC3 prostate cancer cells as early as 6 hours after drug treatment. (a) Representative wound healing assay of 3 independent experiments in PC3 cells treated with rapamycin (50 nM) or ATP site inhibitor of mTOR (200 nM) for 40 hours. Inset (red box) represents wound at 0 hours. (b) Migration patterns of individual GFP-labeled PC3 cells during hours 3-4 after treatment with rapamycin or ATP site inhibitor of mTOR (34 cells per condition). (c) Average velocity of GFP-labeled PC3 cells during hours 3-4 or 6-7 after treatment with rapamycin (50 nM) or ATP site inhibitor of mTOR (200 nM) (mean+SEM, n=34 cells per condition, * P<0.001, ANOVA).

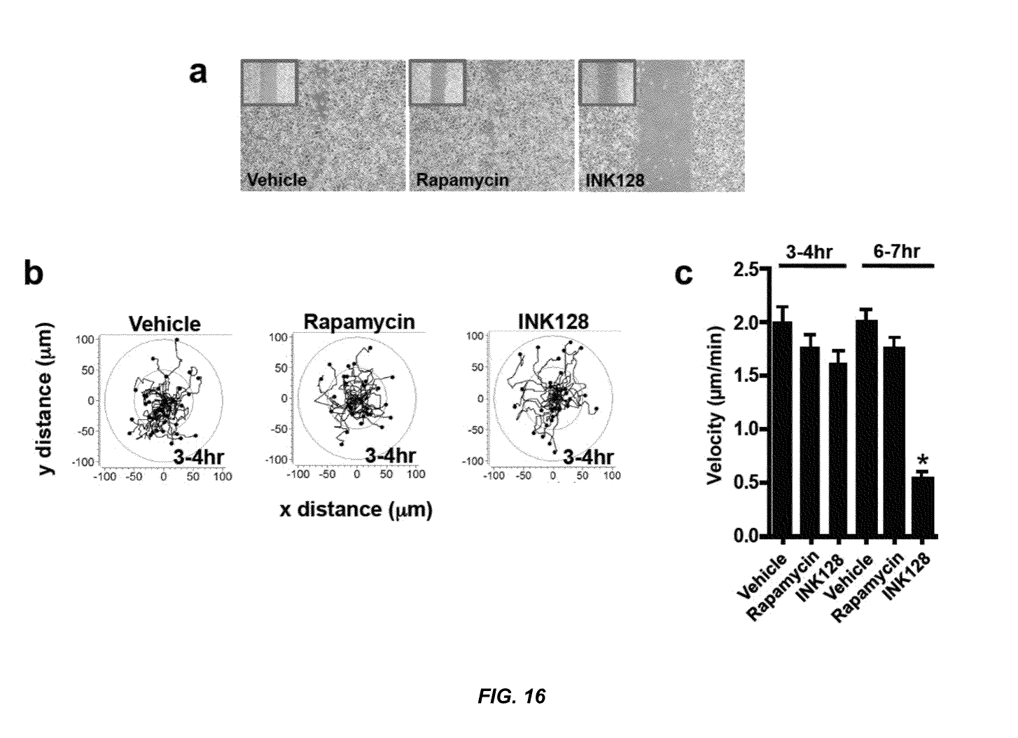

FIG. 17. Knockdown of the 4 invasion genes in PC3 prostate cancer cells. YB1, CD44, MTA1, and Vimentin protein levels after 48 hours of gene silencing in PC3 cells.

FIG. 18. YB1 knockdown and ATP site inhibition of mTOR decreases the protein levels but not mRNA levels of YB1 target genes. (a) Snail1 immunofluorescence in PC3 cells after 48 hours of YB1 gene silencing. Representative Snail1 immunofluorescence (top panels), box plot of Snail1 mean fluorescence intensity per cell (MFI) (n=26 siCtrl cells, n=15 siYB1 cells, * P=0.001, t-test) (bottom panel). (b) Snail1 immunofluorescence in PC3 cells after treatment with rapamycin (50 nM), PP242 (2.5 μM), or ATP site inhibitor of mTOR (200 nM). Representative Snail1 immunofluorescence (left panel), box plot of Snail1 mean fluorescence intensity per cell (MFI) (n=16 vehicle treated cells, n=26 rapamycin treated cells, n=28 PP242 treated cells, n=27 ATP site inhibitor of mTOR treated cells, * P<0.05, ANOVA) (right panel). (c) Representative Western blot (left panel) and quantification of protein levels (right panel) for LEF1 and Twist1 after YB1 gene silencing (mean+SEM, n=6, * P<0.05, t-test). (d) Representative Western blot (left panel) and quantification of protein levels (right panel) for LEF1 and Twist1 after ATP site inhibitor of mTOR treatment (mean+SEM, n=6, * P<0.005, t-test). (e-g) Snail1 (e), LEF1 (f), or Twist1 (g) mRNA expression normalized to β-actin after YB1 gene knockdown or treatment with rapamycin (50 nM), PP242 (2.5 μM) or ATP site inhibitor of mTOR (200 nM) in PC3 cells (mean+SEM, n=3).

FIG. 19. Effects of invasion gene knockdown or overexpression in PC3 and BPH-1 cells, respectively, on the cell cycle. (a) HA-YB1 and Flag-MTA1 protein levels after 48 hours of overexpression in non-transformed BPH-1 prostate epithelial cells (Y=YB1, M=MTA1). (b) Cell cycle analysis in PC3 cells after knockdown of respective genes (mean+SEM, n=3). (c) Cell cycle analysis upon overexpression of YB1 and/or MTA1 in BPH-1 cells (mean+SEM, n=3).

FIG. 20. The 4EBP1^{M} does not augment mTORC1 function or global protein synthesis in PC3 cells. (a) Representative Western blot from 3 independent experiments of phospho-p70S6K^{T389} and phospho-rpS6^{S240/244} after a 48-hour treatment with and without 1 μg/ml doxycycline in PC3-4EBP1^{M} cells. (b) Representative [³⁵S]-methionine incorporation from 2 independent experiments in PC3-4EBP1^{M} cells (48 hours, doxycycline 1 μg/mL) (mean+SEM). (c) Representative cap-binding assay from 2 independent experiments after 48-hour treatment with 1 μg/ml doxycycline in PC3-4EBP1^{M} cells. (d) mRNA expression of YB1, MTA1, Vimentin, and CD44 relative to β-actin after 48-hour treatment with 1 μg/ml doxycycline in PC3-4EBP1^{M} cells (mean+SEM, n=3).

FIG. 21. The 4EBP/eIF4E axis imparts sensitivity to mTOR ATP site inhibition. (a) Quantification of Western blots from 3 independent experiments of PC3 cells after 48 hours of 4EBP1/4EBP2 knockdown followed by 24-hour treatment with an ATP site inhibitor of mTOR (n=3, * p<0.05, ** p<0.01, ANOVA). (b) mRNA expression of YB1, MTA1, vimentin, and CD44 relative to β-actin after 48 hours of gene silencing of 4EBP1 and 4EBP2 followed by a 24-hour treatment with an ATP site inhibitor of mTOR (200 nM) (mean+SEM, n=3). (c) mRNA expression of YB1, MTA1, and CD44 in WT and 4EBP1/4EBP2 DKO MEFs treated with 200 nM ATP site inhibitor of mTOR for 24 hours (mean+SEM, n=3).

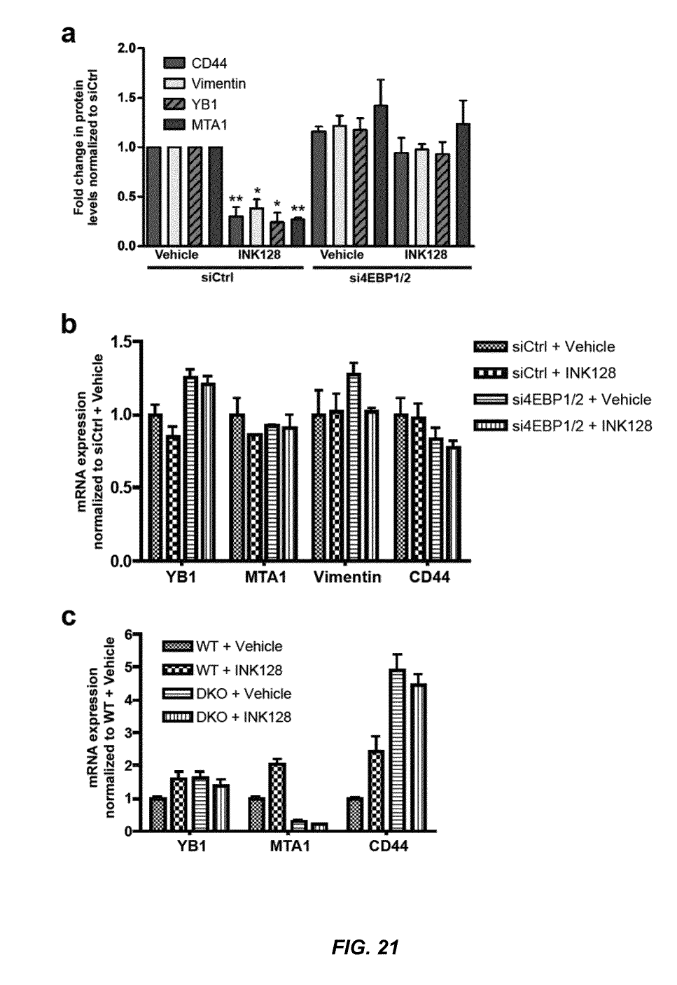

FIG. 22. mTORC2 does not control the expression of the 4-gene invasion signature. (a) mRNA expression of YB1, MTA1, and CD44 relative to β-actin after a 24-hour treatment with ATP site inhibitor of mTOR (200 nM) in mSin1^{-/-} MEFs (mean+SEM, n=3). (b) Representative Western blot analysis from 2 independent experiments of PC3 prostate cancer cells after 48 hours of rictor gene silencing followed by a 24-hour treatment with ATP site inhibitor of mTOR (200 nM). (c) mRNA expression of YB1, MTA1, vimentin, and CD44 relative to β-actin in PC3 prostate cancer cells after 48 hours of rictor gene silencing followed by a 24-hour treatment with ATP site inhibitor of mTOR (200 nM) in PC3 (mean+SEM, n=3). (d) Cell cycle analysis of PC3-4EBP1^{M} cells after treatment with 1 μg/ml doxycycline for 48 hours (mean+SEM, n=3).

FIG. 23. Complete mTOR inhibition decreases the expression of the 4-gene invasion signature at the level of translational control in vivo in PTEN^{L/L} mice. (a) Validation of antibodies used for immunofluorescence after 48-hour gene silencing of respective genes in PC3 cells. (b) Number of individual CK5⁺ and/or CK8⁺ cells measured in 3 separate mice for mean fluorescence intensity of respective protein targets in WT and PTEN^{L/L} mouse prostates. (c) mRNA expression of YB1, MTA1, vimentin, and CD44 relative to β-actin in WT and PTEN^{L/L} mice after 28 days of treatment with ATP site inhibitor of mTOR (1 mg/kg daily) (mean+SEM, n=3 mice per arm). (d) Representative Western blot of MTA1 from whole prostate tissue in WT and PTEN^{L/L} mice after 28 days of treatment with ATP site inhibitor of mTOR (1 mg/kg daily) (left panel) and quantitation relative to β-actin protein levels (right panel) (mean+SEM, n=3 mice per arm, * P=0.02, ** P=0.04, t-test) (e) Representative Western blot of YB1 from whole prostate tissue in WT and PTEN^{L/L} mice after 28 days of treatment with ATP site inhibitor of mTOR (1 mg/kg daily) (left panel) and quantitation relative to β-actin protein levels (right panel) (mean+SEM, n=4 mice per arm, * P=0.002, ** P=0.04, t-test) (f) Semi-quantitative RT-PCR of vimentin and β-actin for WT and PTEN^{L/L} FACS sorted murine prostate luminal epithelial cells (top panel). RT-PCR of a serial dilution of WT prostate luminal epithelial cell (bottom panel) (g) Z-series of perinuclear vimentin in a PTEN^{L/L} CK8⁺ prostate epithelial cell (red: vimentin; blue: DAPI; 0.4 μm per section; yellow arrows point to perinuclear vimentin).

FIG. 24. Preclinical efficacy of complete mTOR blockade in vivo. (a) Mouse weights measured every 3 days over the course of the preclinical trial (mean+SEM, n=3 mice per arm). (b) Representative phospho-specific immunohistochemistry of downstream mTOR targets in the ventral prostate (VP) of 9-month-old WT or PTEN^{L/L} mice after 28 days of treatment with ATP site inhibitor of mTOR (1 mg/kg daily) or RAD001 (10 mg/kg daily) (n=6 mice per treatment arm). Scale bar=100 μm. (c) Representative histology of 9-month-old WT or PTEN^{L/L} mice VP after 28 days of treatment with vehicle, RAD001 (10 mg/kg daily), or ATP site inhibitor of mTOR (1 mg/kg daily). Yellow dotted lines encircle prostate glands. Black triangles refer to prostatic secretions. Scale bar=50 μm. (d) Quantification of PIN+ glands in treated mice (mean+SEM, n=6 mice/arm, * P<0.001, ANOVA). (e) Proliferation measured by phospho-histone H3 positive glands in the prostates of 9-month-old WT or PTEN^{L/L} mice treated with RAD001 (10 mg/kg daily) or ATP site inhibitor of mTOR (1 mg/kg daily) (mean+SEM, n=3 mice per arm, * P<0.01, ANOVA). (f) Apoptosis measured by cleaved caspase 3 (CC3) positive cells in the prostates of 9-month-old WT or PTEN^{L/L} mice treated with RAD001 (10 mg/kg daily) or ATP site inhibitor of mTOR (1 mg/kg daily) (mean+SEM, n=3 mice per arm, * P<0.01, ANOVA) (left panel). Representative CC3 images (right panel). Scale bar=25 μm.

FIG. 25. An ATP site inhibitor of mTOR induces apoptosis in specific cancer cell lines and decreases primary prostate cancer volume in vivo. (a) Apoptosis in LNCaP (n=3) and A498 (n=2) cancer cells after treatment with rapamycin (50 nM), or an ATP site inhibitor of mTOR (200 nM) for 48 hours (mean+SEM, * P<0.001, ** P<0.05, ANOVA, n.s.=not statistically significant). (b) Percentage decrease in ventral and lateral prostate volume in 9-month-old PTEN^{L/L} after a 28-day treatment with vehicle or the ATP site inhibitor of mTOR (1 mg/kg daily) measured by MRI (left panel) (mean+SEM, n=4 mice per arm, * P=0.0008, t-test). Representative MRI images of the PTEN^{L/L} ventral and lateral prostate on day 0 and day 28 of treatment with the ATP site inhibitor of mTOR (right panel) (red dotted lines encircle the ventral and lateral prostate). (c) Additional images of prostate cancer invasion in the PTEN^{L/L} prostate (14-month-old mouse).

### DETAILED DESCRIPTION OF THE INVENTION

### I. Introduction

The present invention relates to methods of generating translational profiles from a biological sample. In some embodiments, the methods of the present invention provide a genome-wide characterization of translationally controlled mRNAs downstream of biological pathways (e.g., oncogenic signaling pathways such as the mTOR pathway). The translational profiles that are generated can be used in identifying agents that modulate the biological pathway or in identifying or validating targets for therapeutic intervention.

### II. Definitions

As used herein, the term “translational profile” refers to the amount of protein that is translated (i.e., translational level) for each gene in a given set of genes in a biological sample. In some embodiments, a translational profile comprises translational levels for a plurality of genes in a biological sample (e.g., in a cell), e.g., for at least about 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 genes or more, or for at least about 1%, 2%, 3%, 4%, 5%, 6%, 7%, 8%, 9%, 10%, 15%, 20%, 25% of all genes in the sample or more. In some embodiments, a translational profile comprises a genome-wide measurement of translational levels in a biological sample.

As used herein, the term “agent” refers to any molecule, either naturally occurring or synthetic, e.g., peptide, protein, oligopeptide (e.g., from about 5 to about 25 amino acids in length, preferably from about 10 to 20 or 12 to 18 amino acids in length, preferably 12, 15, or 18 amino acids in length), small organic molecule (e.g., an organic molecule having a molecular weight of less than about 2500 daltons, e.g., less than 2000, less than 1000, or less than 500 daltons), circular peptide, peptidomimetic, antibody, polysaccharide, lipid, fatty acid, inhibitory RNA (e.g., siRNA or shRNA), polynucleotide, oligonucleotide, aptamer, drug compound, or other compound.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions), complementary sequences, splice variants, and nucleic acid sequences encoding truncated forms of proteins, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, shRNA, siRNA, oligonucleotide, and polynucleotide.

The term “modulate” or “modulator,” as used with reference to modulating an activity of a target gene or signaling pathway, refers to increasing (e.g., activating, facilitating, enhancing, agonizing, sensitizing, potentiating, or upregulating) or decreasing (e.g., preventing, blocking, inactivating, delaying activation, desensitizing, antagonizing, attenuating, or downregulating) the activity of the target gene or signaling pathway. In some embodiments, a modulator increases the activity of the target gene or signaling pathway, e.g., by at least about 1-fold, 2-fold, 3-fold, 4-fold, 5-fold, 6-fold, 7-fold, 8-fold, 9-fold, 10-fold, 15-fold, 20-fold or more. In some embodiments, a modulator decreases the activity of the target gene or signaling pathway, e.g., by at least about 1-fold, 2-fold, 3-fold, 4-fold, 5-fold, 6-fold, 7-fold, 8-fold, 9-fold, 10-fold, 15-fold, 20-fold or more.

A “biological sample” includes blood and blood fractions or products (e.g., serum, plasma, platelets, red blood cells, and the like); sputum or saliva; kidney, lung, liver, heart, brain, nervous tissue, thyroid, eye, skeletal muscle, cartilage, or bone tissue; cultured cells, e.g., primary cultures, explants, and transformed cells, stem cells, stool, urine, etc. Such biological samples also include sections of tissues such as biopsy and autopsy samples, and frozen sections taken for histologic purposes. A biological sample is typically obtained from a “subject,” i.e., a eukaryotic organism, most preferably a mammal such as a primate, e.g., chimpanzee or human; cow; dog; cat; a rodent, e.g., guinea pig, rat, or mouse; rabbit; or a bird; reptile; or fish.

As used herein, the terms “administer,” “administered,” or “administering” refer to methods of delivering agents or compositions to the desired site of biological action. These methods include, but are not limited to, topical delivery, parenteral delivery, intravenous delivery, transdermal delivery, intradermal delivery, transmucosal delivery, intramuscular delivery, oral delivery, nasal delivery, colonical delivery, rectal delivery, intrathecal delivery, ocular delivery, otic delivery, intestinal delivery, or intraperitoneal delivery. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

As used herein, the term “normalize” or “normalizing” refers to adjusting the translational level of one or more genes in a biological sample from a subject (e.g., a sample from a subject having a disease or condition) to a level that is more similar to the translational level of a control sample (e.g., a biological sample from a non-diseased subject). In some embodiments, normalization is evaluated by determining translational levels of one or more genes in a biological sample from a subject (e.g., a sample from a subject having a disease or condition) before and after an agent (e.g., a therapeutic agent) is administered to the subject and comparing the translational levels before and after administration to the translational levels from the control sample.

As used herein, the term “undruggable target” refers to a gene, or a protein encoded by a gene, for which targeted therapy using a drug compound (e.g., a small molecule or antibody) does not successfully interfere with the biological function of the gene or protein encoded by the gene. Typically, an undruggable target is a protein that lacks a binding site for small molecules or for which binding of small molecules does not alter biological function (e.g., ribosomal proteins); a protein for which, despite having a small molecule binding site, successful targeting of said site has proven intractable in practice (e.g., GTP/GDP proteins); or a protein for which selectivity of small molecule binding has not been obtained due to close homology of the binding site with other proteins, and for which binding of the small molecule to these other proteins obviates the therapeutic benefit that is theoretically achievable with binding to the target protein (e.g., protein phosphatases).

### III. Translational Profiling

In one aspect, the present invention relates to the generation and analysis of translational profiles. A translational profile provides information about the identity of genes being translated in a biological sample (e.g., a cell) and/or the amount of protein that is translated (i.e., translational level) for each gene in a given set of genes in the biological sample, thereby providing information about the translational landscape in that biological sample.

In some embodiments, a translational profile comprises translational levels for a plurality of genes in a biological sample, e.g., at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more. In some embodiments, a translational profile comprises translational levels for one or more genes of one or more biological pathways in a biological sample (e.g., pathways such as protein synthesis, cell invasion/metastasis, cell division, apoptosis pathway, signal transduction, cellular transport, post-translational protein modification, DNA repair, and DNA methylation pathways). In some embodiments, a translational profile comprises translational levels for a subset of the genome, e.g., for about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% of the genome or more. In some embodiments, a translational profile comprises a genome-wide measurement of translational levels.

A. Biological Samples

In some embodiments, a biological sample comprises a cell. In some embodiments, the cell is derived from a tissue or organ (e.g., prostate, breast, kidney, lung, liver, heart, brain, nervous tissue, thyroid, eye, skeletal muscle, cartilage, skin, or bone tissue). In some embodiments, the cell is derived from a biological fluid, e.g., blood (e.g., an erythrocyte), lymph (e.g., a monocyte, macrophage, neutrophil, eosinophil, basophil, mast cell, T cell, B cell, and/or NK cell), serum, urine, sweat, tears, or saliva. In some embodiments, the cell is derived from a biopsy (e.g., a skin biopsy, a muscle biopsy, a bone marrow biopsy, a liver biopsy, a gastrointestinal biopsy, a lung biopsy, a nervous system biopsy, or a lymph node biopsy). In some embodiments, the cell is derived from a cultured cell (e.g., a primary cell culture) or a cell line (e.g., PC3, HEK293T, NIH3T3, Jurkat, or Ramos). In some embodiments, the cell is a stem cell or is derived (e.g., differentiated) from a stem cell. In some embodiments, the cell is a cancer stem cell.

In some embodiments, the biological sample comprises a cancer cell (e.g., a cell obtained or derived from a tumor). In some embodiments, the cancer is prostate cancer, breast cancer, bladder cancer, urogenital cancer, lung cancer, renal cell carcinoma, endometrial cancer, melanoma, ovarian cancer, thyroid cancer, or brain cancer. In some embodiments, the cancer is a metastatic cancer.

In some embodiments, the biological sample is from a human subject. In some embodiments, the biological sample is from a non-human mammal (e.g., chimpanzee, dog, cat, pig, mouse, rat, sheep, goat, or horse), avian (e.g., pigeon, penguin, eagle, chicken, duck, or goose), reptile (e.g., snake, lizard, alligator, or turtle), amphibian (e.g., frog, toad, salamander, caecilian, or newt), or fish (e.g., shark, salmon, trout, or sturgeon).

B. Generating Translational Profiles

Various techniques for quantitating translational levels for a given set of genes and generating a translational profile are known in the art and can be used according to the methods of the present invention. These techniques include, but are not limited to, ribosomal profiling, polysome microarray, immunoassay, and mass spectrometry analysis, each of which is detailed below.

### Ribosomal Profiling

In some embodiments, one or more translational profiles are generated by ribosomal profiling. Ribosomal profiling provides a quantitative assessment of translational levels in a sample and can be used to measure translational levels on a genome-wide scale. Generally, ribosomal profiling identifies and/or measures the mRNA associated with ribosomes. Ribosome footprinting is used to isolate and identify the position of active ribosomes on mRNA. Using nuclease digestion, the ribosome position and translated message can be determined by analyzing the approximately 30-nucleotide region that is protected by the ribosome. In some embodiments, ribosome-protected mRNA fragments are analyzed and quantitated by a high-throughput sequencing method. For example, in some embodiments the protected fragments are analyzed by microarray. In some embodiments, the protected fragments are analyzed by deep sequencing; see, e.g., Bentley et al., Nature 456:53-59 (2008). Ribosomal profiling is described, for example, in US 2010/0120625; Ingolia et al., Science 324:218-223 (2009); and Ingolia et al., Nat Protoc 7:1534-1550 (2012); each of which is incorporated herein by reference in its entirety.

Ribosome profiling can comprise methods for detecting a plurality of RNA molecules that are bound to at least one ribosome, wherein the plurality of RNA molecules are associated with the ribosome. In some embodiments, the ribosome profile is of a group of ribosomes, for instance from a polysome. In some embodiments, the ribosome profile is from a group of ribosomes from the same cell or population of cells. For example, in some embodiments, a ribosome profile of a tumor sample can be determined.

In some embodiments, the ribosomal profiling comprises detecting a plurality of RNA molecules bound to at least one ribosome, by (a) contacting the plurality of RNA molecules with an enzymatic degradant or a chemical degradant, thereby forming a plurality of RNA fragments, wherein each RNA fragment comprises an RNA portion protected from the enzymatic degradant or the chemical degradant by a ribosome to which the RNA portion is bound; (b) amplifying the RNA fragments to form a detectable number of amplified nucleic acid fragments; and (c) detecting the detectable number of amplified nucleic acid fragments, thereby detecting the plurality of RNA molecules bound to at least one ribosome.

In some embodiments, nucleic acid fragments (e.g., mRNA fragments) are detected and/or analyzed by deep sequencing. Deep sequencing enables the simultaneous sequencing of multiple fragments, e.g., simultaneous sequencing of at least 500, 1000, 1500, 2000 fragments or more. In a typical deep sequencing protocol, nucleic acids (e.g., mRNA fragments) are attached to the surface of a reaction platform (e.g., flow cell, microarray, and the like). The attached DNA molecules may be amplified in situ and used as templates for synthetic sequencing (i.e., sequencing by synthesis) using a detectable label (e.g., a fluorescent reversible terminator deoxyribonucleotide). Representative reversible terminator deoxyribonucleotides may include 3′-O-azidomethyl-2′-deoxynucleoside triphosphates of adenine, cytosine, guanine and thymine, each labeled with a different recognizable and removable fluorophore, optionally attached via a linker. Where fluorescent tags are employed, after each cycle of incorporation, the identity of the inserted bases may be determined by excitation (e.g., laser-induced excitation) of the fluorophores and imaging of the resulting immobilized growing duplex nucleic acid. The fluorophore, and optionally linker, may be removed by methods known in the art, thereby regenerating a 3′ hydroxyl group ready for the next cycle of nucleotide addition. In some embodiments, the ribosome-protected mRNA fragments are detected and/or analyzed by a sequencing method described in US 2010/0120625, incorporated herein by reference in its entirety.

### Polysome Microarray

In some embodiments, one or more translational profiles are generated by polysome microarray. In a polysome microarray, mRNA is isolated and separated based on the number of associated polysomes. Fractions of mRNA associated with several ribosomes are pooled to form a translationally active pool and are compared to cytosolic mRNA levels. Polysome microarray methods are described, for example, in Melamed and Arava, Methods in Enzymology, 431:177-201 (2007); and Larsson and Nadon, Biotech and Genet Eng Rev, 25:77-92 (2008); each of which is incorporated herein by reference in its entirety.

In some embodiments, polysome fractions having mRNA associated with multiple polysomes (e.g., 3, 4, 5, 10 or more polysomes) are pooled from a biological sample and RNA is isolated and labeled. The RNA samples from the translationally active pool are hybridized to a microarray with a control RNA sample (e.g., an unfractionated RNA sample). Ratios of polysome-to-free RNA are generated for each gene in the microarray to determine the relative levels of ribosomal association for each of the genes.

### Immunoassay

In some embodiments, one or more translational profiles are generated by immunoassay. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997).

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Useful physical formats comprise surfaces having a plurality of discrete, addressable locations for the detection of a plurality of different sequences. Such formats include microarrays and certain capillary devices. See, e.g., Ng et al., J. Cell Mol. Med., 6:329-340 (2002); U.S. Pat. No. 6,019,944. In these embodiments, each discrete surface location may comprise antibodies to immobilize one or more sequences for detection at each location. Surfaces may alternatively comprise one or more discrete particles (e.g., microparticles or nanoparticles) immobilized at discrete locations of a surface, where the microparticles comprise antibodies to immobilize one or more sequences for detection. Other useful physical formats include sticks, wells, sponges, and the like.

Analysis can be carried out in a variety of physical formats. For example, the use of microtiter plates or automation could be used to facilitate the processing of large numbers of samples (e.g., for determining the translational levels of 100, 500, 1000, 5000, 10,000 genes or more).

### Mass Spectrometry Analysis

In some embodiments, one or more translational profiles are generated by mass spectrometry analysis. Mass spectrometry (“MS”) generally involves the ionization of the analyte (e.g., a translated protein) to generate a charged analyte and measuring the mass-to-charge ratios of said analyte. During the procedure the sample containing the analyte is loaded onto a MS instrument and undergoes vaporization. The components of the sample are then ionized by one of a variety of methods.

As a non-limiting example, during Electrospray-MS (ESI) the analyte is initially dissolved in liquid aerosol droplets. Under the influence of high electromagnetic fields and elevated temperature and/or application of a drying gas the droplets get charged and the liquid matrix evaporates. After all liquid matrix is evaporated the charges remain localized at the analyte molecules that are transferred into the Mass Spectrometer. In matrix assisted laser desorption ionization (MALDI) a mixture of analyte and matrix is irradiated by a laser beam. This results in localized ionization of the matrix material and desorption of analyte and matrix. The ionization of the analyte is believed to happen by charge transfer from the matrix material in the gas phase. For a detailed description of ESI and MALDI, see, e.g., Mano N et al. Anal. Sciences 19 (1) (2003) 3-14. For a description of desorption electrospray ionization (DESI), see Takats Z et al. Science 306 (5695) (2004) 471-473. See also Karas, M.; Hillencamp, F. Anal. Chem. 60:2301 1988); Beavis, R. C. Org. Mass Spec. 27:653 (1992); and Creel, H. S. Trends Poly. Sci. 1(11):336 (1993).

Ionized sample components are then separated according to their mass-to-charge ratio in a mass analyzer. Examples of different mass analyzers used in LC/MS include, but are not limited to, single quadrupole, triple quadrupole, ion trap, TOF (time of Flight) and quadrupole-time of flight (Q-TOF).

The use of MS for analyzing proteins is also described, for example, in Mann et al., Annu. Rev. Biochem. 70:437-73 (2001).

C. Differential Translational Profiling

In some embodiments, two or more translational profiles are generated and compared to each other to determine the differences (i.e., increases and/or decreases in translational levels) for each gene in a given set of genes between the two or more translational profiles. The comparison between the two or more translational profiles is referred to as the “differential translational profile.”

In some embodiments, a differential translational profile compares a first translational profile comprising gene translational levels for an experimental biological sample or subject, wherein the experimental biological sample or subject has been contacted with an agent as described herein (e.g., a peptide, protein, RNA, drug molecule, or small organic molecule) with a second translational profile comprising gene translational levels for a control biological sample or subject, e.g., a corresponding biological sample or subject of the same type that has not been contacted with the agent.

In some embodiments, a differential translational profile compares a first translational profile comprising gene translational levels for an experimental biological sample, wherein the experimental biological sample is from a subject having an unknown disease state (e.g., a cancer) or an unknown responsiveness to a therapeutic agent, with a second translational profile comprising gene translational levels for a control biological sample, e.g., a biological sample from a subject known to be positive for a disease state (e.g., a cancer) or from a subject that is a known responder to the therapeutic agent.

In some embodiments, differential profiles are generated for each of the first and second translational profiles, e.g., to compare the differences in translational levels for one or more genes in the presence or absence of a condition, or before and after administration of an agent, for the first translational profile (e.g., a translational profile from an experimental subject or sample) as compared to the second translational profile (e.g., a translational profile from a control subject or sample). For example, in some embodiments, differential profiles are generated for an experimental subject or sample (e.g., a subject having a cancer) before and after administration of a therapeutic agent and for a control subject or sample (e.g., a subject that is a known responder to the therapeutic agent) before and after administration of the therapeutic agent. The first differential profile for the first translational profile (from the experimental subject or sample) is compared to the second differential profile for the second translational profile (from the control subject or sample) to determine the similarities in translational levels of one or more genes for the first differential profile as compared to the second differential profile. Based on the similarities between the differential profiles (e.g., whether the differential profiles are highly similar, or whether the translational level for one or more genes in the first differential profile is about the same as the translational level for the one or more genes in the second differential profile), it can be determined whether or not the experimental subject or control is likely to respond to the therapeutic agent.

### IV. Methods of Identifying Agents that Modulate an Oncogenic Signaling Pathway

In one aspect, the present invention relates to methods of identifying an agent that modulates an oncogenic signaling pathway in a biological sample. In some embodiments, the present invention relates to methods of identifying an agent that inhibits, antagonizes, or downregulates an oncogenic signaling pathway. In some embodiments, the present invention relates to methods of identifying an agent that modulates, i.e., potentiates, agonizes, inhibits, upregulates, an oncogenic signaling pathway.

A. Translational Profiles for Identifying Agents that Modulate an Oncogenic Signaling Pathway

In some embodiments, the method of identifying an agent that modulates an oncogenic signaling pathway comprises:
(a) contacting the biological sample with an agent;
(b) determining a first translational profile for the contacted biological sample,
   wherein the translational profile comprises translational levels for one or more genes having a 5′ terminal oligopyrimidine tract (5′ TOP) and/or a pyrimidine-rich translational element (PRTE); and
(c) comparing the first translational profile to a second translational profile comprising translational levels for the one or more genes in a control sample that has not been contacted with the agent;
   wherein a difference in the translational levels of the one or more genes in the first translation profile as compared to the second translation profile identifies the agent as a modulator of the oncogenic signaling pathway.

In some embodiments, a gene that has a different translational level in the first translational profile as compared to the second translational profile is a gene having a 5′ terminal oligopyrimidine tract (5′ TOP) sequence. A 5′ TOP sequence is a sequence that occurs in the 5′ untranslated region (5′ UTR) of mRNA. This element is comprised of a cytidine residue at the cap site followed by an uninterrupted stretch of up to 13 pyrimidines. Non-limiting examples of genes having a 5′ TOP sequence are shown in Table 1 below. In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes selected from the genes listed in Table 1.

TABLE 1
Translationally regulated mTOR-responsive genes having a 5′ TOP sequence

| Gene | Description | SEQ ID NO |
|---|---|---|
| AP2A1 | adaptor-related protein complex 2, alpha 1 subunit | 92 |
| CCNI | cyclin I | 96 |
| CD44 | CD44 antigen | 123 |
| CHP | calcineurin-like EF hand protein 1 | 116 |
| CRTAP | cartilage associated protein | 31 |
| EEF1A2 | eukaryotic translation elongation factor 1, alpha 2 | 45 |
| EEF1B2 | eukaryotic translation elongation factor 1, beta 2 | 129 |
| EEF1G | eukaryotic translation elongation factor 1, gamma | 34 |
| EEF2 | eukaryotic translation elongation factor 2 | 1 |
| EIF4B | eukaryotic translation initiation factor 4B | 37 |
| GAPDH | glyceraldehyde-3-phosphate dehydrogenase | 58 |
| GNB2L1 | guanine nucleotide binding protein (G protein), beta | 22 |
| | polypeptide 2-like 1 | |
| HNRNPA1 | heterogeneous nuclear ribonucleoprotein A1 | 56 |
| HSPA8 | heat shock 70 kDa protein 8 | 42 |
| IPO7 | importin 7 | 109 |
| LCMT1 | leucine carboxyl methyltransferase 1 | 107 |
| NAP1L1 | nucleosome assembly protein 1-like 1 | 93 |
| PABPC1 | poly(A) binding protein, cytoplasmic 1 | 17 |
| PACS1 | phosphofurin acidic cluster sorting protein 1 | 117 |
| PGM1 | phosphoglucomutase 1 | 121 |
| RABGGTB | Rab geranylgeranyltransferase, beta subunit | 139 |
| RPL10 | ribosomal protein L10 | 13 |
| RPL12 | ribosomal protein L12 | 3 |
| RPL13 | ribosomal protein L13 | 70 |
| RPL14 | ribosomal protein L14 | 53 |
| RPL15 | ribosomal protein L15 | 126 |
| RPL17 | ribosomal protein L17 | 79 |
| RPL22 | ribosomal protein L22 | 91 |
| RPL22L1 | ribosomal protein L22 L1 | 35 |
| RPL23 | ribosomal protein L23 | 74 |
| RPL29 | ribosomal protein L29 | 60 |
| RPL31 | ribosomal protein L31 isoform 2 | 49 |
| RPL32 | ribosomal protein L32 | 33 |
| RPL34 | ribosomal protein L34 | 11 |
| RPL36 | ribosomal protein L36 | 63 |
| RPL36A | ribosomal protein L36A | 66 |
| RPL37 | ribosomal protein L37 | 54 |
| RPL37A | ribosomal protein L37A | 18 |
| RPL39 | ribosomal protein L39 | 43 |
| RPL4 | ribosomal protein L4 | 104 |
| RPL41 | ribosomal protein L41 | 113 |
| RPL5 | ribosomal protein L5 | 86 |
| RPL6 | ribosomal protein L6 | 89 |
| RPL8 | ribosomal protein L8 | 59 |
| RPLP0 | ribosomal protein, large, P0 | 28 |
| RPLP2 | ribosomal protein, large, P2 | 38 |
| RPS10 | ribosomal protein S10 | 77 |
| RPS11 | ribosomal protein S11 | 51 |
| RPS14 | ribosomal protein S14 | 94 |
| RPS15A | ribosomal protein S15A | 21 |
| RPS2 | ribosomal protein S2 | 4 |
| RPS20 | ribosomal protein S20 | 24 |
| RPS3A | ribosomal protein S3A | 61 |
| RPS5 | ribosomal protein S5 | 19 |
| RPS6 | ribosomal protein S6 | 101 |
| RPS9 | ribosomal protein S9 | 29 |
| SECTM1 | secreted and transmembrane 1 | 112 |
| TPT1 | tumor protein, translationally-controlled 1 | 65 |
| UBA52 | ubiquitin A-52 residue ribosomal protein fusion product 1 | 84 |
| VIM | vimentin | 40 |
| ABCB7 | ATP-binding cassette, sub-family B (MDR/TAP), member 7 | 134 |
| ALKBH7 | alkB, alkylation repair homolog 7 | 85 |
| ATP5G2 | ATP synthase, H+ transporting, mitochondrial Fo | 144 |
| | complex, subunit C2 (subunit 9) | |
| EEF1A1 | eukaryotic translation elongation factor 1 alpha 1 | 7 |
| EIF2S3 | eukaryotic translation initiation factor 2, subunit 3 gamma, | 80 |
| | 52 kDa | |
| EIF3H | eukaryotic translation initiation factor 3, subunit H | 98 |
| EIF3L | eukaryotic translation initiation factor 3, subunit L | 108 |
| GLTSCR2 | glioma tumor suppressor candidate region gene 2 | 15 |
| IMPDH2 | IMP (inosine 5′-monophosphate) dehydrogenase 2 | 142 |
| PFDN5 | prefoldin subunit 5 | 130 |
| RPL10A | ribosomal protein L10a | 46 |
| RPL11 | ribosomal protein L11 | 23 |
| RPL13A | ribosomal protein L13a | 5 |
| RPL18 | ribosomal protein L18 | 62 |
| RPL19 | ribosomal protein L19 | 103 |
| RPL21 | ribosomal protein L21 | 20 |
| RPL24 | ribosomal protein L24 | 124 |
| RPL26 | ribosomal protein L26 | 52 |
| RPL27A | ribosomal protein L27A | 12 |
| RPL28 | ribosomal protein L28 | 8 |
| RPL3 | ribosomal protein L3 | 16 |
| RPL30 | ribosomal protein L30 | 81 |
| RPL7A | ribosomal protein L7a | 25 |
| RPLP1 | ribosomal protein, large, P1 | 50 |
| RPS12 | ribosomal protein S12 | 2 |
| RPS13 | ribosomal protein S13 | 105 |
| RPS16 | ribosomal protein S16 | 39 |
| RPS19 | ribosomal protein S19 | 26 |
| RPS21 | ribosomal protein S21 | 27 |
| RPS23 | ribosomal protein S23 | 100 |
| RPS24 | ribosomal protein S24 | 90 |
| RPS25 | ribosomal protein S25 | 75 |
| RPS27 | ribosomal protein S27 | 10 |
| RPS28 | ribosomal protein S28 | 9 |
| RPS29 | ribosomal protein S29 | 73 |
| RPS3 | ribosomal protein S3A | 61 |
| RPS7 | ribosomal protein S7 | 102 |

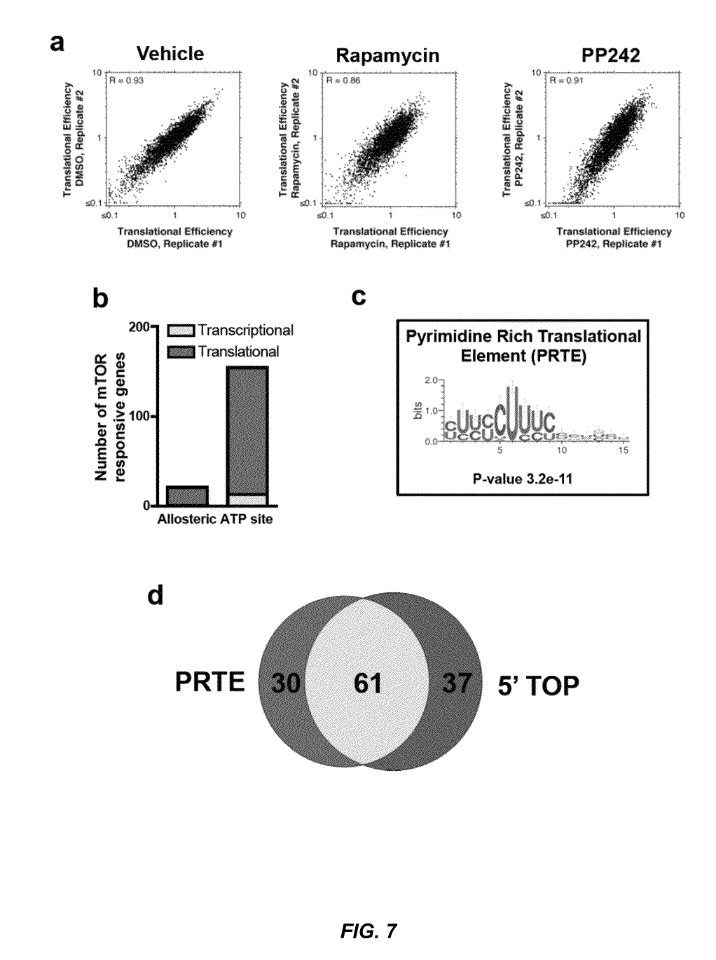

In some embodiments, a gene that has a different translational level in the first translational profile as compared to the second translational profile is a gene having a pyrimidine-rich translational element (PRTE). This element consists of an invariant uridine at its position 6 and does not reside at position +1 of the 5′ UTR. See, e.g., FIG. 7(c). Non-limiting examples of genes having a PRTE sequence are shown in Table 2 below. In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes selected from the genes listed in Table 2.

TABLE 2
Translationally regulated mTOR-responsive genes
having a PRTE sequence

| | | SEQ ID |
|---|---|---|
| Gene | Description | NO |
| EEF2 | eukaryotic translation elongation factor 2 | 1 |
| RPL12 | ribosomal protein L12 | 3 |
| RPS2 | ribosomal protein S2 | 4 |
| RPL18A | ribosomal protein L18a | 6 |
| RPL34 | ribosomal protein L34 | 11 |
| RPL10 | ribosomal protein L10 | 13 |
| EEF1D | eukaryotic translation elongation factor 1 delta | 14 |
| PABPC1 | poly(A) binding protein, cytoplasmic 1 | 17 |
| RPL37A | ribosomal protein L37a | 18 |
| RPS5 | ribosomal protein S5 | 19 |
| RPS15A | ribosomal protein S15a | 21 |
| GNB2L1 | guanine nucleotide binding protein (G protein) | 22 |
| RPS20 | ribosomal protein S20 isoform 1 | 24 |
| RPLP0 | ribosomal protein P0 | 28 |
| RPS9 | ribosomal protein S9 | 29 |
| CRTAP | cartilage associated protein | 31 |
| RPL32 | ribosomal protein L32 | 33 |
| EEF1G | eukaryotic translation elongation factor 1, gamma | 34 |
| RPL22L1 | ribosomal protein L22-like 1 | 35 |
| YB1 | Y-box binding protein 1 | 36 |
| EIF4B | eukaryotic translation initiation factor 4B | 37 |
| RPLP2 | ribosomal protein P2 | 38 |
| VIM | vimentin | 40 |
| HSPA8 | heat shock 70 kDa protein 8 isoform 1 | 42 |
| RPL39 | ribosomal protein L39 | 43 |
| AHCY | adenosylhomocysteinase isoform 1 | 44 |
| EEF1A2 | eukaryotic translation elongation factor 1 alpha 2 | 45 |
| PABPC4 | poly A binding protein, cytoplasmic 4 isoform 1 | 47 |
| RPS4X | ribosomal protein S4, X-linked X isoform | 48 |
| RPL31 | ribosomal protein L31 isoform 2 | 49 |
| RPS11 | ribosomal protein S11 | 51 |
| RPL14 | ribosomal protein L14 | 53 |
| RPL37 | ribosomal protein L37 | 54 |
| RPL7 | ribosomal protein L7 | 55 |
| HNRNPA1 | heterogeneous nuclear ribonucleoprotein A1 | 56 |
| RPS8 | ribosomal protein S8 | 57 |
| GAPDH | glyceraldehyde-3-phosphate dehydrogenase | 58 |
| RPL8 | ribosomal protein L8 | 59 |
| RPL29 | ribosomal protein L29 | 60 |
| RPS3A | ribosomal protein S3a | 61 |
| RPL36 | ribosomal protein L36 | 63 |
| TPT1 | tumor protein, translationally-controlled 1 | 65 |
| RPL36A | ribosomal protein L36a | 66 |
| TKT | transketolase isoform 1 | 68 |
| LMF2 | lipase maturation factor 2 | 69 |
| RPL13 | ribosomal protein L13 | 70 |
| RPL23 | ribosomal protein L23 | 74 |
| TUBB3 | tubulin, beta, 4 | 76 |
| RPS10 | ribosomal protein S10 | 77 |
| FASN | fatty acid synthase | 78 |
| RPL17 | ribosomal protein L17 | 79 |
| ACTG1 | actin, gamma 1 propeptide | 82 |
| COL6A2 | alpha 2 type VI collagen isoform 2C2 | 83 |
| UBA52 | ubiquitin and ribosomal protein L40 precursor | 84 |
| RPL5 | ribosomal protein L5 | 86 |
| PGLS | 6-phosphogluconolactonase | 87 |
| RPL6 | ribosomal protein L6 | 89 |
| RPL22 | ribosomal protein L22 | 91 |
| AP2A1 | adaptor-related protein complex 2, alpha 1 | 92 |
| NAP1L1 | nucleosome assembly protein 1-like 1 | 93 |
| RPS14 | ribosomal protein S14 | 94 |
| CCNI | cyclin I | 96 |
| MTA1 | metastasis associated 1 | 97 |
| RPL9 | ribosomal protein L9 | 99 |
| RPL4 | ribosomal protein L4 | 104 |
| LCMT1 | leucine carboxyl methyltransferase 1 isoform a | 107 |
| IPO7 | importin 7 | 109 |
| PC | pyruvate carboxylase | 110 |
| RPS27A | ubiquitin and ribosomal protein S27a | 111 |
| SECTM1 | secreted and transmembrane 1 precursor | 112 |
| RPL41 | ribosomal protein L41 | 113 |
| TSC2 | tuberous sclerosis 2 isoform 1 | 114 |
| COL18A1 | alpha 1 type XVIII collagen isoform 3 | 115 |
| CHP | calcium binding protein P22 | 116 |
| PACS1 | phosphofurin acidic cluster sorting protein 1 | 117 |
| BRF1 | transcription initiation factor IIIB | 118 |
| PTGES2 | prostaglandin E synthase 2 | 119 |
| PGM1 | phosphoglucomutase 1 | 121 |
| SLC19A1 | solute carrier family 19 member 1 | 122 |
| CD44 | CD44 antigen isoform 1 | 123 |
| RPL15 | ribosomal protein L15 | 126 |
| EEF1B2 | eukaryotic translation elongation factor 1 beta 2 | 129 |
| PNKP | polynucleotide kinase 3′ phosphatase | 131 |
| SEPT8 | septin 8 isoform a | 132 |
| EVPL | envoplakin | 136 |
| MYH14 | myosin, heavy chain 14 isoform 3 | 138 |
| RABGGTB | RAB geranylgeranyltransferase, beta subunit | 139 |
| RPL27 | ribosomal protein L27 | 140 |
| SIGMAR1 | sigma non-opioid intracellular receptor 1 | 143 |

In some embodiments, a gene that has a different translational level in the first translational profile as compared to the second translational profile is a gene having both a 5′ TOP sequence and a PRTE sequence. Non-limiting examples of genes having both a 5′ TOP sequence and a PRTE sequence are shown in Table 3 below. In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes selected from the genes listed in Table 3.

TABLE 3
5′ TOP and PRTE genomic positions in translationally regulated mTOR-
responsive genes having both 5′ TOP and PRTE

| | | | Strand | | PRTE |
|---|---|---|---|---|---|
| Gene | RefSeq ID | Chromosome | (+/−) | 5′ TOP Position | Position |
| AP2A1 | NM_014203 | 19 | + | 50270268 | 50270306 |
| CCNI | NM_006835 | 4 | − | 77997142 | 77997076 |
| CD44 | NM_000610 | 11 | + | 35160717 | 35160813 |
| CHP | NM_007236 | 15 | + | 41523519 | 41523536 |
| CRTAP | NM_006371 | 3 | + | 33155506/ | 33155540 |
| | | | | 33155554 | |
| eEF1A2 | NM_001958 | 20 | − | 62130436 | 62129175 |
| eEF1B2 | NM_021121 | 2 | + | 207024619 | 207024665 |
| eEF1G | NM_001404 | 11 | − | 62341490/ | 62341383 |
| | | | | 62341335 | |
| eEF2 | NM_001961 | 19 | − | 3985461 | 3985423 |
| eIF4B | NM_001417 | 12 | + | 53400240 | 53400250 |
| GAPDH | NM_002046 | 12 | + | 6643684 | 6643717 |
| GNB2L1 | NM_006098 | 5 | − | 180670906 | 180670818 |
| HNRNPA1 | NM_031157 | 12 | + | 54674529 | 54674571 |
| HSPA8 | NM_006597 | 11 | − | 122932844 | 122932806 |
| IPO7 | NM_006391 | 11 | + | 9406199 | 9406255 |
| LCMT1 | NM_016309 | 16 | + | 25123101 | 25123114 |
| NAP1L1 | NM_004537 | 12 | − | 76478465 | 76478429 |
| PABPC1 | NM_002568 | 8 | − | 101734315 | 101734151 |
| PACS1 | NM_018026 | 11 | + | 65837839 | 65837922 |
| PGM1 | NM_002633 | 1 | + | 64059078 | 64059107 |
| RABGGTB | NM_004582 | 1 | + | 76251941 | 76251928 |
| RPL10 | NM_006013 | X | + | 153626718 | 153626846 |
| RPL12 | NM_000976 | 9 | − | 130213677 | 130213648 |
| RPL13 | NM_000977/ | 16 | + | 89627090 | 89627102/ |
| | NM_033251 | | | | 89627202 |
| RPL14 | NM_001034996 | 3 | + | 40498830 | 40498906 |
| RPL15 | NM_002948 | 3 | + | 23958639 | 23958711 |
| RPL17 | NM_000985 | 18 | − | 47018849 | 47017964 |
| RPL22 | NM_000983 | 1 | − | 6259654 | 6259645 |
| RPL22L1 | NM_001099645 | 3 | − | 170587984 | 170587976 |
| RPL23 | NM_000978 | 17 | − | 37009989 | 37010013 |
| RPL29 | NM_000992 | 3 | − | 52029911 | 52029904 |
| RPL31 | NM_001098577 | 2 | + | 101618755 | 101618739 |
| RPL32 | NM_001007074 | 3 | − | 12883040 | 12883002 |
| RPL34 | NM_000995/ | 4 | + | 109541733 | 109541743/ |
| | NM_033625 | | | | 109541769 |
| RPL36 | NM_033643/ | 19 | + | 5690307 | 5690319/ |
| | NM_015414 | | | | 5690493 |
| RPL36A | NM_021029 | X | + | 100645999 | 100645981 |
| RPL37 | NM_000997 | 5 | − | 40835324 | 40835314 |
| RPL37A | NM_000998 | 2 | + | 217363567 | 217363526 |
| RPL39 | NM_001000 | X | − | 118925591 | 118925564 |
| RPL4 | NM_000968 | 15 | − | 66797185 | 66797143 |
| RPL41 | NM_001035267 | 12 | + | 56510417 | 56510539 |
| RPL5 | NM_000969 | 1 | + | 93297597 | 93297656 |
| RPL6 | NM_000970 | 12 | − | 112847409 | 112847256 |
| RPL8 | NM_000973/ | 8 | − | 146017775 | 146017709 |
| | NM_033301 | | | | |
| RPLP0 | NM_053275 | 12 | − | 120638910 | 120638652 |
| RPLP2 | NM_001004 | 11 | + | 809968 | 810006 |
| RPS10 | NM_001014 | 6 | − | 34393846 | 34393715 |
| RPS11 | NM_001015 | 19 | + | 49999690 | 49999677 |
| RPS14 | NM_001025070 | 5 | − | 149829300/ | 149829107 |
| | | | | 149829186 | |
| RPS15A | NM_001030009 | 16 | − | 18801656 | 18801604 |
| RPS2 | NM_002952 | 16 | − | 2014827 | 2014653 |
| RPS20 | NM_001146227 | 8 | − | 56987065 | 56986992 |
| RPS27A | NM_001177413 | 2 | + | 55459824 | 55459920 |
| RPS3A | NM_001006 | 4 | + | 152020780 | 152020789 |
| RPS5 | NM_001009 | 19 | + | 58898636 | 58898691 |
| RPS6 | NM_001010 | 9 | − | 19380234 | 19380207 |
| RPS9 | NM_001013 | 19 | + | 54704726 | 54704775 |
| SECTM1 | NM_003004 | 17 | − | 80291646 | 80291674/ |
| | | | | | 80291639 |
| TPT1 | NM_003295 | 13 | − | 45915318 | 45915222 |
| UBA52 | NM_003333 | 19 | + | 18682670 | 18683218 |
| VIM | NM_003380 | 10 | + | 17271277 | 17271358 |

In some embodiments, the method comprises:
(a) contacting the biological sample with an agent;
(b) determining a first translational profile for the contacted biological sample, wherein the translational profile comprises translational levels for one or more genes selected from the group consisting of SEQ ID NOs:1-144; and
(c) comparing the first translational profile to a second translational profile comprising translational levels for the one or more genes in a control sample that has not been contacted with the agent;
wherein a difference in the translational levels of the one or more genes in the first translation profile as compared to the second translation profile identifies the agent as a modulator of the oncogenic signaling pathway.

In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes selected from the group consisting of SEQ ID NOs:1-144. SEQ ID NOs:1-144 are listed in Table 4 below:

TABLE 4
Translationally regulated mTOR-responsive genes

| | | SEQ ID |
|---|---|---|
| Gene | Description | NO |
| EEF2 | eukaryotic translation elongation factor 2 | 1 |
| RPS12 | ribosomal protein S12 | 2 |
| RPL12 | ribosomal protein L12 | 3 |
| RPS2 | ribosomal protein S2 | 4 |
| RPL13A | ribosomal protein L13a | 5 |
| RPL18A | ribosomal protein L18a | 6 |
| EEF1A1 | eukaryotic translation elongation factor 1 alpha 1 | 7 |
| RPL28 | ribosomal protein L28 isoform 1 | 8 |
| RPS28 | ribosomal protein S28 | 9 |
| RPS27 | ribosomal protein S27 | 10 |
| RPL34 | ribosomal protein L34 | 11 |
| RPL27A | ribosomal protein L27a | 12 |
| RPL10 | ribosomal protein L10 | 13 |
| EEF1D | eukaryotic translation elongation factor 1 delta | 14 |
| GLTSCR2 | glioma tumor suppressor candidate region gene 2 | 15 |
| RPL3 | ribosomal protein L3 isoform a | 16 |
| PABPC1 | poly(A) binding protein, cytoplasmic 1 | 17 |
| RPL37A | ribosomal protein L37a | 18 |
| RPS5 | ribosomal protein S5 | 19 |
| RPL21 | ribosomal protein L21 | 20 |
| RPS15A | ribosomal protein S15a | 21 |
| GNB2L1 | guanine nucleotide binding protein (G protein) | 22 |
| RPL11 | ribosomal protein L11 | 23 |
| RPS20 | ribosomal protein S20 isoform 1 | 24 |
| RPL7A | ribosomal protein L7a | 25 |
| RPS19 | ribosomal protein S19 | 26 |
| RPS21 | ribosomal protein S21 | 27 |
| RPLP0 | ribosomal protein P0 | 28 |
| RPS9 | ribosomal protein S9 | 29 |
| RPS3 | ribosomal protein S3 | 30 |
| CRTAP | cartilage associated protein | 31 |
| FAM128B | hypothetical protein LOC80097 | 32 |
| RPL32 | ribosomal protein L32 | 33 |
| EEF1G | eukaryotic translation elongation factor 1, gamma | 34 |
| RPL22L1 | ribosomal protein L22-like 1 | 35 |
| YB1 | Y-box binding protein 1 | 36 |
| EIF4B | eukaryotic translation initiation factor 4B | 37 |
| RPLP2 | ribosomal protein P2 | 38 |
| RPS16 | ribosomal protein S16 | 39 |
| VIM | vimentin | 40 |
| GAMT | guanidinoacetate N-methyltransferase isoform b | 41 |
| HSPA8 | heat shock 70 kDa protein 8 isoform 1 | 42 |
| RPL39 | ribosomal protein L39 | 43 |
| AHCY | adenosylhomocysteinase isoform 1 | 44 |
| EEF1A2 | eukaryotic translation elongation factor 1 alpha 2 | 45 |
| RPL10A | ribosomal protein L10a | 46 |
| PABPC4 | poly A binding protein, cytoplasmic 4 isoform 1 | 47 |
| RPS4X | ribosomal protein S4, X-linked X isoform | 48 |
| RPL31 | ribosomal protein L31 isoform 2 | 49 |
| RPLP1 | ribosomal protein P1 isoform 1 | 50 |
| RPS11 | ribosomal protein S11 | 51 |
| RPL26 | ribosomal protein L26 | 52 |
| RPL14 | ribosomal protein L14 | 53 |
| RPL37 | ribosomal protein L37 | 54 |
| RPL7 | ribosomal protein L7 | 55 |
| HNRNPA1 | heterogeneous nuclear ribonucleoprotein A1 | 56 |
| RPS8 | ribosomal protein S8 | 57 |
| GAPDH | glyceraldehyde-3-phosphate dehydrogenase | 58 |
| RPL8 | ribosomal protein L8 | 59 |
| RPL29 | ribosomal protein L29 | 60 |
| RPS3A | ribosomal protein S3a | 61 |
| RPL18 | ribosomal protein L18 | 62 |
| RPL36 | ribosomal protein L36 | 63 |
| AGRN | agrin precursor | 64 |
| TPT1 | tumor protein, translationally-controlled 1 | 65 |
| RPL36A | ribosomal protein L36a | 66 |
| SLC25A5 | adenine nucleotide translocator 2 | 67 |
| TKT | transketolase isoform 1 | 68 |
| LMF2 | lipase maturation factor 2 | 69 |
| RPL13 | ribosomal protein L13 | 70 |
| CTSH | cathepsin H isoform b | 71 |
| FAM83H | FAM83H | 72 |
| RPS29 | ribosomal protein S29 isoform 2 | 73 |
| RPL23 | ribosomal protein L23 | 74 |
| RPS25 | ribosomal protein S25 | 75 |
| TUBB3 | tubulin, beta, 4 | 76 |
| RPS10 | ribosomal protein S10 | 77 |
| FASN | fatty acid synthase | 78 |
| RPL17 | ribosomal protein L17 | 79 |
| EIF2S3 | eukaryotic translation initiation factor 2, S3 | 80 |
| RPL30 | ribosomal protein L30 | 81 |
| ACTG1 | actin, gamma 1 propeptide | 82 |
| COL6A2 | alpha 2 type VI collagen isoform 2C2 | 83 |
| UBA52 | ubiquitin and ribosomal protein L40 precursor | 84 |
| ALKBH7 | spermatogenesis associated 11 precursor | 85 |
| RPL5 | ribosomal protein L5 | 86 |
| PGLS | 6-phosphogluconolactonase | 87 |
| CSDA | cold shock domain protein A | 88 |
| RPL6 | ribosomal protein L6 | 89 |
| RPS24 | ribosomal protein S24 isoform d | 90 |
| RPL22 | ribosomal protein L22 | 91 |
| AP2A1 | adaptor-related protein complex 2, alpha 1 | 92 |
| NAP1L1 | nucleosome assembly protein 1-like 1 | 93 |
| RPS14 | ribosomal protein S14 | 94 |
| ETHE1 | ETHE1 protein | 95 |
| CCNI | cyclin I | 96 |
| MTA1 | metastasis associated 1 | 97 |
| EIF3H | eukaryotic translation initiation factor 3, H | 98 |
| RPL9 | ribosomal protein L9 | 99 |
| RPS23 | ribosomal protein S23 | 100 |
| RPS6 | ribosomal protein S6 | 101 |
| RPS7 | ribosomal protein S7 | 102 |
| RPL19 | ribosomal protein L19 | 103 |
| RPL4 | ribosomal protein L4 | 104 |
| RPS13 | ribosomal protein S13 | 105 |
| C21orf66 | GC-rich sequence DNA-binding factor candidate | 106 |
| LCMT1 | leucine carboxyl methyltransferase 1 isoform a | 107 |
| EIF3L | eukaryotic translation initiation factor 3, L | 108 |
| IPO7 | importin 7 | 109 |
| PC | pyruvate carboxylase | 110 |
| RPS27A | ubiquitin and ribosomal protein S27a | 111 |
| SECTM1 | secreted and transmembrane 1 precursor | 112 |
| RPL41 | ribosomal protein L41 | 113 |
| TSC2 | tuberous sclerosis 2 isoform 1 | 114 |
| COL18A1 | alpha 1 type XVIII collagen isoform 3 | 115 |
| CHP | calcium binding protein P22 | 116 |
| PACS1 | phosphofurin acidic cluster sorting protein 1 | 117 |
| BRF1 | transcription initiation factor IIIB | 118 |
| PTGES2 | prostaglandin E synthase 2 | 119 |
| C2orf79 | hypothetical protein LOC391356 | 120 |
| PGM1 | phosphoglucomutase 1 | 121 |
| SLC19A1 | solute carrier family 19 member 1 | 122 |
| CD44 | CD44 antigen isoform 1 | 123 |
| RPL24 | ribosomal protein L24 | 124 |
| NCLN | nicalin | 125 |
| RPL15 | ribosomal protein L15 | 126 |
| CLPTM1 | cleft lip and palate associated transmembrane | 127 |
| ECSIT | evolutionarily conserved signaling intermediate | 128 |
| EEF1B2 | eukaryotic translation elongation factor 1 beta 2 | 129 |
| PFDN5 | prefoldin subunit 5 isoform alpha | 130 |
| PNKP | polynucleotide kinase 3′ phosphatase | 131 |
| SEPT8 | septin 8 isoform a | 132 |
| CIRBP | cold inducible RNA binding protein | 133 |
| ABCB7 | ATP-binding cassette, sub-family B, member 7 | 134 |
| ARD1A | alpha-N-acetyltransferase 1A | 135 |
| EVPL | envoplakin | 136 |
| LAMA5 | laminin alpha 5 | 137 |
| MYH14 | myosin, heavy chain 14 isoform 3 | 138 |
| RABGGTB | RAB geranylgeranyltransferase, beta subunit | 139 |
| RPL27 | ribosomal protein L27 | 140 |
| RPS15 | ribosomal protein S15 | 141 |
| IMPDH2 | inosine monophosphate dehydrogenase 2 | 142 |
| SIGMAR1 | sigma non-opioid intracellular receptor 1 | 143 |
| ATP5G2 | ATP synthase, H+ transporting, mitochondrial F0 | 144 |

In some embodiments, the first and/or second translational profile comprises 1, 2, 3, 4, 5, 6, 7, 8, 9, 10 or more genes that are functionally classified as a protein synthesis gene, a cell invasion/metastasis gene, a metabolism gene, a signal transduction gene, a cellular transport gene, a post-translational modification gene, an RNA synthesis and processing gene, a regulation of cell proliferation gene, a development gene, an apoptosis gene, a DNA repair gene, a DNA methylation gene, or an amino acid biosynthesis gene. In some embodiments, the first and/or second translational profile comprises 1, 2, 3, 4, 5, 6, 7, 8, 9, 10 or more genes from each of two, three, four, five, or more of these functional categories of genes. In some embodiments, first and/or second translational profile comprises 1, 2, 3, 4, 5, 6, 7, 8, 9, 10 more genes that are functionally classified as a cell invasion or metastasis gene. In some embodiments, the first and/or second translational profile comprises one or more of the cell invasion/metastasis genes YB1, vimentin, MTA1, and CD44. In some embodiments, the first and/or second translational profile comprises YB1, vimentin, MTA1, and CD44.

In some embodiments, the method comprises:
(a) contacting the biological sample with an agent;
(b) determining a first translational profile for the contacted biological sample, wherein the translational profile comprises a measurement of gene translational levels for a substantial portion of the genome;
(c) comparing the first translational profile to a second translational profile comprising a measurement of gene translational levels for the substantial portion of the genome translational levels for the one or more genes in a control sample that has not been contacted with the agent;
(d) identifying in the first translational profile a plurality of genes having decreased translational levels as compared to the translational levels of the plurality of genes in the second translational profile; and
(e) determining whether, for the plurality of genes identified in step (d), there is a common consensus sequence and/or regulatory element in the untranslated regions (UTRs) of the genes that is shared by at least 10% of the plurality of genes identified in step (d);
wherein a decrease in the translational levels of at least 10% of the genes sharing the common consensus sequence and/or UTR regulatory element in the first translational profile as compared to the second translational profile identifies the agent as an inhibitor of an oncogenic signaling pathway.

As used herein, the term “substantial portion of the genome,” with reference to a biological sample, can refer to an empirical number of genes being measured in the biological sample or to a percentage of the genes in the genome being measured in the biological sample. In some embodiments, a substantial portion of the genome comprises at least 500 genes, e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more. In some embodiments, a substantial portion of the genome comprises at least about 0.01%, at least about 0.05%, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, or at least about 50% of all genes in the genome for the biological sample.

In some embodiments, the oncogenic signaling pathway that is modulated is the mammalian target of rapamycin (mTOR) pathway, the PI3K pathway, the AKT pathway, the Ras pathway, the Myc pathway, the Wnt pathway, or the BRAF pathway. In some embodiments, the oncogenic signaling pathway that is modulated is the mTOR pathway.

In some embodiments, there is at least a two-fold difference (e.g., at least two-fold, at least three-fold, at least four-fold, at least five-fold, at least six-fold, at least seven-fold, at least eight-fold, at least nine-fold, at least ten-fold difference or more) in translational level for the one or more genes in the first translational profile as compared to the second translational profile. In some embodiments, there is at least a two-fold difference in translational level for 1, 2, 3, 4, 5, 6, 7, 8, 9, 10, 11, 12, 13, 14, 15, 16, 17, 18, 19, 20, 30, 40, 50, 60, 70, 80, 90, 100 or more genes in the first translational profile as compared to the second translational profile. In some embodiments, the translational level of one or more genes is decreased in the first translational profile as compared to the second translational profile. In some embodiments, the translational level of one or more genes in the first translational profile is decreased by at least two-fold, at least three-fold, at least four-fold, at least five-fold, at least six-fold, at least seven-fold, at least eight-fold, at least nine-fold, at least ten-fold or more as compared to the second translational profile. In some embodiments, the translational level of one or more genes is increased in the first translational profile as compared to the second translational profile. In some embodiments, the translational level of one or more genes in the first translational profile is increased by at least two-fold, at least three-fold, at least four-fold, at least five-fold, at least six-fold, at least seven-fold, at least eight-fold, at least nine-fold, at least ten-fold or more as compared to the second translational profile. In some embodiments, the translational level of one or more genes is decreased (e.g., by at least two-fold, at least three-fold, at least four-fold, at least five-fold, at least six-fold, at least seven-fold, at least eight-fold, at least nine-fold, at least ten-fold or more) in the first translational profile, while the translational level of another one or more genes is increased (e.g., by at least two-fold, at least three-fold, at least four-fold, at least five-fold, at least six-fold, at least seven-fold, at least eight-fold, at least nine-fold, at least ten-fold or more) in the first translational profile, as compared to the second translational profile.

B. Agents

In some embodiments, an agent that can be used according to the methods of the present invention is a peptide, protein, oligopeptide, circular peptide, peptidomimetic, antibody, polysaccharide, lipid, fatty acid, inhibitory RNA (e.g., siRNA, miRNA, or shRNA), polynucleotide, oligonucleotide, aptamer, small organic molecule, or drug compound. The agent can be either synthetic or naturally-occurring.

In some embodiments, the agent acts as a specific regulator of translational machinery or a component of translational machinery that alters the program of protein translation in cells (e.g., a small molecule inhibitor or inhibitory RNA). In some embodiments, the agent binds at the active site of a protein (e.g., an ATP site inhibitor of mTOR).

In some embodiments, multiple agents (e.g., 2, 3, 4, 5, or more agents) are used. In some embodiments, multiple agents are administered to a subject or contacted to a biological sample sequentially. In some embodiments, multiple agents are administered to a subject or contacted to a biological sample concurrently.

The agents described herein can be used at varying concentrations. In some embodiments, an agent is administered to a subject or contacted to a biological sample at a concentration that is known or expected to be a therapeutic dose. In some embodiments, an agent is administered to a subject or contacted to a biological sample at a concentration that is known or expected to be a sub-therapeutic dose. In some embodiments, an agent is administered to a subject or contacted to a biological sample at a concentration that is lower than a concentration that would typically be administered to an organism or applied to a sample, e.g., at a concentration that is 2, 3, 4, 5, 6, 7, 8, 9, 10, 15, 20, 25, 30, 35, 40, 45, 50, 60, 70, 80, 90, or 100 times less than the concentration that would typically be administered to an organism or applied to a sample.

In some embodiments, an agent can be identified from a library of agents. In some embodiments, the library of agents comprises at least about 5, 10, 15, 20, 25, 30, 35, 40, 45, 50, 60, 70, 80, 90, 100, 200, 300, 400, 500, 600, 700, 800, 900, 1000, 5000, 10,000, 20,000, 30,000, 40,000, 50,000 agents or more. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.). In some embodiments, the library is a combinatorial chemical or peptide library. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks. The preparation and screening of chemical libraries is well known to those of skill in the art (see, e.g., Beeler et al., Curr Opin Chem. Biol., 9:277 (2005); and Shang et al., Curr Opin Chem. Biol., 9:248 (2005)).

In some embodiments, an agent for use in the methods of the present invention (e.g., an agent that modulates an oncogenic signaling pathway) can be identified by screening a library containing a large number of potential therapeutic compounds. The library can be screened in one or more assays, as described herein, to identify those library members that display a desired characteristic activity. The compounds thus identified can serve as conventional “lead compounds” (e.g., for identifying other potential therapeutic compounds) or can themselves be used as potential or actual therapeutics. Libraries of use in the present invention can be composed of amino acid compounds, nucleic acid compounds, carbohydrates, or small organic compounds. Carbohydrate libraries have been described in, for example, Liang et al., Science, 274:1520-1522 (1996); and U.S. Pat. No. 5,593,853.

Representative amino acid compound libraries include, but are not limited to, peptide libraries (see, e.g., U.S. Pat. Nos. 5,010,175; 6,828,422; and 6,844,161; Furka, Int. J. Pept. Prot. Res., 37:487-493 (1991); Houghton et al., Nature, 354:84-88 (1991); and Eichler, Comb Chem High Throughput Screen., 8:135 (2005)), peptoids (PCT Publication No. WO 91/19735), encoded peptides (PCT Publication No. WO 93/20242), random bio-oligomers (PCT Publication No. WO 92/00091), vinylogous polypeptides (Hagihara et al., J. Amer. Chem. Soc., 114:6568 (1992)), nonpeptidal peptidomimetics with 13-D-glucose scaffolding (Hirschmann et al., J. Amer. Chem. Soc., 114:9217-9218 (1992)), peptide nucleic acid libraries (see, e.g., U.S. Pat. No. 5,539,083), antibody libraries (see, e.g., U.S. Pat. Nos. 6,635,424 and 6,555,310; PCT Application No. PCT/US96/10287; and Vaughn et al., Nature Biotechnology, 14:309-314 (1996)), and peptidyl phosphonates (Campbell et al., J. Org. Chem., 59:658 (1994)).

Representative nucleic acid compound libraries include, but are not limited to, genomic DNA, cDNA, mRNA, inhibitory RNA (e.g., RNAi, siRNA), and antisense RNA libraries. See, e.g., Ausubel, Current Protocols in Molecular Biology, eds. 1987-2005, Wiley Interscience; and Sambrook and Russell, Molecular Cloning: A Laboratory Manual, 2000, Cold Spring Harbor Laboratory Press. Nucleic acid libraries are described in, for example, U.S. Pat. Nos. 6,706,477; 6,582,914; and 6,573,098. cDNA libraries are described in, for example, U.S. Pat. Nos. 6,846,655; 6,841,347; 6,828,098; 6,808,906; 6,623,965; and 6,509,175. RNA libraries, for example, ribozyme, RNA interference, or siRNA libraries, are described in, for example, Downward, Cell, 121:813 (2005) and Akashi et al., Nat. Rev. Mol. Cell. Biol., 6:413 (2005). Antisense RNA libraries are described in, for example, U.S. Pat. Nos. 6,586,180 and 6,518,017.

Representative small organic molecule libraries include, but are not limited to, diversomers such as hydantoins, benzodiazepines, and dipeptides (Hobbs et al., Proc. Nat. Acad. Sci. USA, 90:6909-6913 (1993)); analogous organic syntheses of small compound libraries (Chen et al., J. Amer. Chem. Soc., 116:2661 (1994)); oligocarbamates (Cho et al., Science, 261:1303 (1993)); benzodiazepines (e.g., U.S. Pat. No. 5,288,514; and Baum, C&EN, January 18, page 33 (1993)); isoprenoids (e.g., U.S. Pat. No. 5,569,588); thiazolidinones and metathiazanones (e.g., U.S. Pat. No. 5,549,974); pyrrolidines (e.g., U.S. Pat. Nos. 5,525,735 and 5,519,134); morpholino compounds (e.g., U.S. Pat. No. 5,506,337); tetracyclic benzimidazoles (e.g., U.S. Pat. No. 6,515,122); dihydrobenzpyrans (e.g., U.S. Pat. No. 6,790,965); amines (e.g., U.S. Pat. No. 6,750,344); phenyl compounds (e.g., U.S. Pat. No. 6,740,712); azoles (e.g., U.S. Pat. No. 6,683,191); pyridine carboxamides or sulfonamides (e.g., U.S. Pat. No. 6,677,452); 2-aminobenzoxazoles (e.g., U.S. Pat. No. 6,660,858); isoindoles, isooxyindoles, or isooxyquinolines (e.g., U.S. Pat. No. 6,667,406); oxazolidinones (e.g., U.S. Pat. No. 6,562,844); and hydroxylamines (e.g., U.S. Pat. No. 6,541,276).

Devices for the preparation of libraries are commercially available. See, e.g., 357 MPS and 390 MPS from Advanced Chem. Tech (Louisville, Ky.), Symphony from Rainin Instruments (Woburn, Mass.), 433A from Applied Biosystems (Foster City, Calif.), and 9050 Plus from Millipore (Bedford, Mass.).

C. Undruggable Targets

In some embodiments, the methods of the present invention relate to identifying an agent that modulates an undruggable target. It is estimated that only about 10-15% of human proteins are disease modifying, and of these proteins, as many as 85-90% are “undruggable,” meaning that even though theoretical therapeutic benefits may be experimentally observed for these target proteins (e.g., in vitro or in a model system in vivo using techniques such as shRNA), targeted therapy using a drug compound (e.g., a small molecule or antibody) does not successfully interfere with the biological function of the protein (or of the gene encoding the protein). Typically, an undruggable target is a protein that lacks a binding site for small molecules or for which binding of small molecules does not alter biological function (e.g., ribosomal proteins); a protein for which, despite having a small molecule binding site, successful targeting of said site has proven intractable in practice (e.g., GTP/GDP proteins); or a protein for which selectivity of small molecule binding has not been obtained due to close homology of the binding site with other proteins, and for which binding of the small molecule to these other proteins obviates the therapeutic benefit that is theoretically achievable with binding to the target protein (e.g., protein phosphatases). By preferentially inhibiting the synthesis of such a target protein by selectively inhibiting programmed translation of a small set of proteins (e.g., about 1, 2, 3, 4, 5, 6, 7, 8, 9, 10, 15, or 20 proteins), it is possible to modulate (e.g., inhibit) the activity of the “undruggable” target protein.

In some embodiments, a method of identifying an agent that modulates an undruggable target comprises:
(a) contacting a biological sample with an agent;
(b) determining a first translational profile for the contacted biological sample, wherein the translational profile comprises translational levels for a plurality of genes; and
(c) comparing the first translational profile to a second translational profile comprising translational levels for the plurality of genes in a control sample that has not been contacted with the agent;
wherein identifying one or more genes of a biological pathway as differentially translated in the first translational profile as compared to the second translational profile identifies the agent as modulating the activity of the undruggable target, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and DNA methylation pathway.

In some embodiments, one or more genes from each of at least two, at least three, at least four, at least five, or more of the biological pathways is differentially translated in the first translational profile as compared to the second translational profile. In some embodiments, two, three, four, five or more genes (e.g., 1, 2, 3, 4, 5, 6, 7, 8, 9, 10, 11, 12, 13, 14, 15, 16, 17, 18, 19, 20, 30, 40, 50, 60, 70, 80, 90, 100 or more genes) from one or more of the biological pathways are differentially translated in the first translational profile as compared to the second translational profile. Non-limiting examples of protein synthesis, cell invasion/metastasis, cell division, apoptosis pathway, signal transduction, cellular transport, post-translational protein modification, DNA repair, and DNA methylation pathways are described herein.

In some embodiments, the first and/or second translational profile comprises translational levels for a plurality of genes in the biological sample. In some embodiments, the first and/or second translational profile comprises translational levels for at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more in the biological sample. In some embodiments, the first and/or second translational profile comprises translational levels for at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% of all genes in the biological sample or more. In some embodiments, the first and/or second translational profile comprises a genome-wide measurement of gene translational levels in the biological sample.

In some embodiments, there is at least a two-fold difference in translational level for the one or more genes (e.g., for 1, 2, 3, 4, 5, 6, 7, 8, 9, 10, 11, 12, 13, 14, 15, 16, 17, 18, 19, 20, 30, 40, 50, 60, 70, 80, 90, 100 or more genes) in the first translational profile as compared to the second translational profile. In some embodiments, there is at least a three-fold difference, at least a four-fold difference, at least a five-fold difference, at least a six-fold difference, at least a seven-fold difference, at least an eight-fold difference, at least a nine-fold difference, at least a ten-fold difference or more in the translational level for the one or more genes in the first translational profile as compared to the second translational profile. In some embodiments, the translational level of the one or more genes is decreased in the first translational profile as compared to the second translational profile. In some embodiments, the translational level of the one or more genes is increased in the first translational profile as compared to the second translational profile. In some embodiments, the translational level of one or more genes is decreased in the first translational profile, while the translational level of another one or more genes is increased in the first translational profile, as compared to the second translational profile.

In some embodiments, the agent is an RNA molecule. In some embodiments, the agent is an shRNA, siRNA, or miRNA molecule.

D. Synthesizing and Validating Agents Based on Identified Agents

In some embodiments, an agent that is identified as modulating an oncogenic signaling pathway is optimized in order to improve the agent's biological and/or pharmacological properties. To optimize the agent, structurally related analogs of the agent can be chemically synthesized to systematically modify the structure of the initially-identified agent.

For chemical synthesis, solid phase synthesis can be used for compounds such as peptides, nucleic acids, organic molecules, etc., since in general solid phase synthesis is a straightforward approach with excellent scalability to commercial scale. Techniques for solid phase synthesis are described in the art. See, e.g., Seneci, Solid Phase Synthesis and Combinatorial Technologies (John Wiley & Sons 2002); Barany & Merrifield, Solid-Phase Peptide Synthesis, pp. 3-284 in The Peptides: Analysis, Synthesis, Biology, Vol. 2 (E. Gross and J. Meienhofer, eds., Academic Press 1979).

The synthesized structurally related analogs can be screened to determine whether the analogs induce a similar translational profile when contacted to a biological sample as compared to the initial agent from which the analog was derived. In some embodiments, a selected-for structurally related analog is one that induces an identical or substantially identical translational profile in a biological sample as the initial agent from which the structurally related analog was derived.

A structurally related analog that is determined to induce a sufficiently similar translational profile in a biological sample as the initial agent from which the structurally related analog was derived can be further screened for biological and pharmacological properties, including but not limited to oral bioavailability, half-life, metabolism, toxicity, and pharmacodynamic activity (e.g., duration of the therapeutic effect) according to methods known in the art. Typically, the screening of the structurally related analogs is performed in vivo in an appropriate animal model (e.g., a mammal such as a mouse or rat). Animal models for analyzing pharmacological and pharmacokinetic properties, including animal models for various disease states, are well known in the art and are commercially available, e.g., from Charles River Laboratories Intl, Inc. (Wilmington, Mass.).

In some embodiments, an agent that is identified as having a suitable biological profile, or a structurally related analog thereof, is used for the preparation of a medicament for the treatment of a disease or condition associated with the modulation of the biological pathway (e.g., a cancer associated with the modulation of the mTOR pathway).

### V. Methods of Validating a Target for Therapeutic Intervention

In another aspect, the present invention provides methods of validating a target for therapeutic intervention. In some embodiments, the method comprises:
(a) contacting a biological sample with an agent that modulates the target;
(b) determining a first translational profile for the contacted biological sample, wherein the first translational profile comprises translational levels for a plurality of genes; and
(c) comparing the first translational profile to a second translational profile comprising translational levels for the plurality of genes in a control sample that has not been contacted with the agent;
wherein identifying one or more genes of a biological pathway as differentially translated in the first translational profile as compared to the second translational profile validates the target for therapeutic intervention, wherein said biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway.

In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes in one or more biological pathways selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway. In some embodiments, one or more genes from each of at least two of the biological pathways is differentially translated in the first translational profile as compared to the second translational profile. In some embodiments, one or more genes from each of at least three of the biological pathways is differentially translated in the first translational profile as compared to the second translational profile. In some embodiments, the biological pathway, or one of the biological pathways, is the mTOR pathway.

In some embodiments, translational levels are compared for the first and second translational profiles for one or more genes in a protein synthesis pathway. Examples of protein synthesis pathway genes include, but are not limited to, EEF2, RPS12, RPL12, RPS2, RPL13A, RPL18A, EEF1A1, RPL28, RPS28, and RPS27. In some embodiments, translational levels are compared for the first and second translational profiles for one or more genes in a cell invasion/metastasis pathway. Examples of cell invasion/metastasis pathway genes include, but are not limited to, YB1, MTA1, Vimentin, and CD44. In some embodiments, translational levels are compared for the first and second translational profiles for one or more genes in a cell division pathway. Examples of cell division pathway genes include, but are not limited to, CCNI. In some embodiments, translational levels are compared for the first and second translational profiles for one or more genes in an apoptosis pathway. Examples of apoptosis pathway genes include, but are not limited to, ARF, FADD, TNFRSF21, BAX, DAPK, TMS-1, BCL2, RASSF1A, and TERT. In some embodiments, translational levels are compared for the first and second translational profiles for one or more genes in a signal transduction pathway. Examples of signal transduction pathway genes include, but are not limited to, MAPK, MYC, RAS, and RAF. In some embodiments, translational levels are compared for the first and second translational profiles for one or more genes in a cellular transport pathway. Examples of cellular transport pathway genes include, but are not limited to, SLC25A5. In some embodiments, translational levels are compared for the first and second translational profiles for one or more genes in a post-translational protein modification pathway. Examples of post-translational protein modification pathway genes include, but are not limited to, LCMT1 and RABGGTB. In some embodiments, translational levels are compared for the first and second translational profiles for one or more genes in a DNA repair pathway. Examples of DNA repair pathway genes include, but are not limited to, PNKP. In some embodiments, translational levels are compared for the first and second translational profiles for one or more genes in a DNA methylation pathway. Examples of DNA methylation pathway genes include, but are not limited to, AHCY.

In some embodiments, the one or more genes has a 5′ TOP sequence, a PRTE sequence, or both a 5′ TOP sequence and a PRTE sequence. In some embodiments, the one or more genes is selected from the genes listed in Table 1, Table 2, and/or Table 3. In some embodiments, the one or more genes is selected from the group consisting of SEQ ID NOs:1-144.

In some embodiments, the target for therapeutic intervention is a part of an oncogenic signaling pathway. In some embodiments, the oncogenic signaling pathway is the mammalian target of rapamycin (mTOR) pathway, the PI3K pathway, the AKT pathway, the Ras pathway, the Myc pathway, the Wnt pathway, or the BRAF pathway. In some embodiments, the oncogenic signaling pathway that is modulated is the mTOR pathway.

Agents that can be used to validate a target for therapeutic intervention include any agent described herein (e.g., in Section IV(B) above), and include but are not limited to, peptides, proteins, oligopeptides, circular peptides, peptidomimetics, antibodies, polysaccharides, lipids, fatty acids, inhibitory RNAs (e.g., siRNA, miRNA, or shRNA), polynucleotides, oligonucleotides, aptamers, small organic molecules, or drug compounds. In some embodiments, the agent is a small organic molecule. In some embodiments, the agent is a peptide or protein. In some embodiments, the agent is an RNA or inhibitory RNA.

The translational profiles that are generated for validating a target for therapeutic intervention can be generated according to any of the methods described herein. In some embodiments, the translational profiles are generated by ribosomal profiling. In some embodiments, the translational profiles are generated by polysome microarray. In some embodiments, the translational profiles are generated by immunoassay. In some embodiments, the translational profiles comprise translational levels for 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more in the biological sample. In some embodiments, the first and/or second translational profile comprises translational levels for at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more of all genes in the biological sample. In some embodiments, the translational profiles comprise genome-wide measurements of gene translational levels.

In some embodiments, a target is validated when one or more genes of one or more biological pathways is differentially translated by at least two-fold (e.g., at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, a target is validated when the translational level for one or more genes of one or more biological pathways is decreased by at least two-fold (e.g., at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, a target is validated when the translational level for one or more genes of one or more biological pathways is increased by at least two-fold (e.g., at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, less than 20% of the genes in the genome are differentially translated by at least two-fold in the first translational profile as compared to the second translational profile. In some embodiments, less than 5% of the genes in the genome are differentially translated by at least two-fold in the first translational profile as compared to the second translational profile. In some embodiments, less than 1% of the genes in the genome are differentially translated by at least two-fold in the first translational profile as compared to the second translational profile.

### VI. Methods of Identifying Drug Candidate Molecules

In another aspect, the present invention comprises a method of identifying a drug candidate molecule. In some embodiments, the method comprises:
(a) contacting a biological sample with the drug candidate molecule;
(b) determining a translational profile for the contacted biological sample, wherein the translational profile comprises translational levels for a plurality of genes; and
(c) comparing the first translational profile to a second translational profile comprising translational levels for the plurality of genes in a control sample that has not been contacted with the drug candidate molecule,
wherein the drug candidate molecule is identified as suitable for use in a therapeutic intervention when one or more genes of a biological pathway is differentially translated in the first translational profile as compared to the second translational profile, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and DNA methylation pathway.

In some embodiments, the one or more genes (e.g., 1, 2, 3, 4, 5, 6, 7, 8, 9, 10 or more genes) have a 5′ TOP sequence, a PRTE sequence, or both a 5′ TOP sequence and a PRTE sequence. In some embodiments, the one or more genes is selected from the genes listed in Table 1, Table 2, and/or Table 3. In some embodiments, the one or more genes (e.g., 1, 2, 3, 4, 5, 6, 7, 8, 9, 10 or more genes) are selected from the group consisting of SEQ ID NOs:1-144. In some embodiments, one or more genes from each of at least two of the biological pathways is differentially translated in the first translational profile as compared to the second translational profile. In some embodiments, one or more genes from each of at least three of the biological pathways is differentially translated in the first translational profile as compared to the second translational profile.

In some embodiments, translational levels are compared for the first and second translational profiles for one or more genes in a protein synthesis pathway. Examples of protein synthesis pathway genes include, but are not limited to, EEF2, RPS12, RPL12, RPS2, RPL13A, RPL18A, EEF1A1, RPL28, RPS28, and RPS27. In some embodiments, translational levels are compared for the first and second translational profiles for one or more genes in a cell invasion/metastasis pathway. Examples of cell invasion/metastasis pathway genes include, but are not limited to, YB1, MTA1, Vimentin, and CD44. In some embodiments, translational levels are compared for the first and second translational profiles for one or more genes in a cell division pathway. Examples of cell division pathway genes include, but are not limited to, CCNI. In some embodiments, translational levels are compared for the first and second translational profiles for one or more genes in an apoptosis pathway. Examples of apoptosis pathway genes include, but are not limited to, ARF, FADD, TNFRSF21, BAX, DAPK, TMS-1, BCL2, RASSF1A, and TERT. In some embodiments, translational levels are compared for the first and second translational profiles for one or more genes in a signal transduction pathway. Examples of signal transduction pathway genes include, but are not limited to, MAPK, MYC, RAS, and RAF. In some embodiments, translational levels are compared for the first and second translational profiles for one or more genes in a cellular transport pathway. Examples of cellular transport pathway genes include, but are not limited to, SLC25A5. In some embodiments, translational levels are compared for the first and second translational profiles for one or more genes in a post-translational protein modification pathway. Examples of post-translational protein modification pathway genes include, but are not limited to, LCMT1 and RABGGTB. In some embodiments, translational levels are compared for the first and second translational profiles for one or more genes in a DNA repair pathway. Examples of DNA repair pathway genes include, but are not limited to, PNKP. In some embodiments, translational levels are compared for the first and second translational profiles for one or more genes in a DNA methylation pathway. Examples of DNA methylation pathway genes include, but are not limited to, AHCY.

The translational profiles that are generated for identifying a drug candidate molecule can be generated according to any of the methods described herein. In some embodiments, the translational profiles are generated by ribosomal profiling. In some embodiments, the translational profiles are generated by polysome microarray. In some embodiments, the translational profiles are generated by immunoassay. In some embodiments, the translational profiles comprise translational levels for at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more in the biological sample. In some embodiments, the first and/or second translational profile comprises translational levels for at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more of all genes in the biological sample. In some embodiments, the translational profiles comprise genome-wide measurements of gene translational levels.

In some embodiments, a drug candidate molecule is identified as suitable for use in a therapeutic intervention when one or more genes of one or more biological pathways is differentially translated by at least two-fold (e.g., at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, a drug candidate molecule is identified as suitable for use in a therapeutic intervention when the translational level for one or more genes of one or more biological pathways is decreased by at least two-fold (e.g., at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, a drug candidate molecule is identified as suitable for use in a therapeutic intervention when the translational level for one or more genes of one or more biological pathways is increased by at least two-fold (e.g., at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, less than 20% of the genes in the genome are differentially translated by at least two-fold in the first translational profile as compared to the second translational profile. In some embodiments, less than 5% of the genes in the genome are differentially translated by at least two-fold in the first translational profile as compared to the second translational profile. In some embodiments, less than 1% of the genes in the genome are differentially translated by at least two-fold in the first translational profile as compared to the second translational profile.

Drug candidate molecules are not limited by therapeutic category, and can include, for example, analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agent, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolytics, lipid regulating agents, anti-anginal agents, Cox-2 inhibitors, leukotriene inhibitors, macrolides, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and the like, as well as mixtures thereof.

In some embodiments, the method further comprises comparing the translational profile for the contacted biological sample with a control translational profile for a second biological sample that has been contacted with a known therapeutic agent. In some embodiments, the known therapeutic agent is a known inhibitor of an oncogenic pathway. In some embodiments, the known therapeutic agent is a known inhibitor of the mammalian target of rapamycin (mTOR) pathway, the PI3K pathway, the AKT pathway, the Ras pathway, the Myc pathway, the Wnt pathway, or the BRAF pathway. In some embodiments, the known therapeutic agent is a known inhibitor of the mTOR pathway.

In some embodiments, the methods of identifying a drug candidate molecule as described herein are used to compare a group of drug candidate molecules and select one drug candidate molecule or a smaller subgroup of drug candidate molecules from this group. In some embodiments, the methods described herein are used to compare drug candidate molecules and select one candidate molecule or a subgroup of drug candidate molecules which alter the translation of a relatively smaller number of proteins, as compared to the number of proteins for which translational is altered for the larger group of drug candidate molecules. In some embodiments, the methods described herein are used to compare drug candidate molecules and select one candidate molecule or a subgroup of drug candidate molecules for which altered translation resides in a relatively smaller number of pathways, as compared to the number of pathways for which translation is altered for the larger group of drug candidate molecules. In some embodiments, the methods described herein are used to compare drug candidate molecules and select one candidate molecule or a subgroup of drug candidate molecules which alter the translation of several proteins within one specific pathway, as compared to the larger group of drug candidate molecules for which a smaller number of proteins within that one specific pathway have altered translation.

### VII. Therapeutic Methods

In yet another aspect, the present invention provides therapeutic methods for identifying subjects for treatment and treating subjects in need thereof. In some embodiments, the present invention relates to methods of identifying a subject as a candidate for treatment, e.g., for treatment with an mTOR inhibitor. In some embodiments, the present invention relates to methods of treating a subject, e.g., a subject having a cancer.

A. Identifying Subjects for Treatment

In some embodiments, the present invention relates to a method of identifying a subject as a candidate for treatment with an mTOR inhibitor. In some embodiments, the method comprises:
(a) determining a first translational profile in a sample from the subject, wherein the first translational profile comprises translational levels for one or more genes having a 5′ terminal oligopyrimidine tract (5′ TOP) and/or a pyrimidine-rich translational element (PRTE); and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
wherein a translational level of the one or more genes in the first translational profile that is at least as high as the translational level of the one or more genes in the second translational profile identifies the subject as a candidate for treatment with the mTOR inhibitor.

In some embodiments, the one or more genes (e.g., the 1, 2, 3, 4, 5, 6, 7, 8, 9, 10, 11, 12, 13, 14, 15, 16, 17, 18, 19, 20 or more genes) are selected from the genes listed in any of Table 1, Table 2, or Table 3.

In some embodiments, a method of identifying a subject as a candidate for treatment with an mTOR inhibitor comprises:
(a) determining a first translational profile in a sample from the subject, wherein the first translational profile comprises translational levels for one or more genes selected from the group consisting of SEQ ID NOs:1-144; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
wherein a translational level of the one or more genes in the first translational profile that is at least as high as the translational level of the one or more genes in the second translational profile identifies the subject as a candidate for treatment with the mTOR inhibitor.

In some embodiments, the one or more genes (e.g., the 1, 2, 3, 4, 5, 6, 7, 8, 9, 10, 11, 12, 13, 14, 15, 16, 17, 18, 19, 20 or more genes) are cell invasion/metastasis genes. In some embodiments, the one or more genes are selected from YB1, vimentin, MTA1, and CD44.

In some embodiments, a method of identifying a subject as a candidate for treatment with an mTOR inhibitor comprises:
(a) determining a first translational profile in a sample from the subject, wherein the first translational profile comprises translational levels for one or more genes of a biological pathway, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
wherein a translational level of the one or more genes in the first translational profile that is at least as high as the translational level of the one or more genes in the second translational profile identifies the subject as a candidate for treatment with the mTOR inhibitor.

In some embodiments, the methods of the present invention relate to a method of identifying a subject as a candidate for treatment with a therapeutic agent. In some embodiments, the method comprises:
(a) determining a first translational profile in a sample from the subject,
wherein the translational profile comprises translational levels for one or more genes of a biological pathway, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the therapeutic agent prior to administration of the therapeutic agent to the known responder;
wherein a translational level of the one or more genes that is at least as high as the translational level of the one or more genes in the second translational profile identifies the subject as a candidate for treatment with the therapeutic agent.

In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes in one or more biological pathways. In some embodiments, the translational level of one or more genes from each of at least two of the biological pathways is at least as high in the first translational profile as compared to the second translational profile. In some embodiments, the translational level of one or more genes from each of at least three of the biological pathways is at least as high in the first translational profile as compared to the second translational profile.

In some embodiments, the first and/or second translational profiles comprise translational levels for at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more in the biological sample.

In some embodiments, the first and/or second translational profile comprises translational levels for at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more of all genes in the biological sample. In some embodiments, the translational profiles comprise genome-wide measurements of gene translational levels. In some embodiments, the translational level of the one or more genes is increased by at least two-fold (e.g., at least two-fold, at least three-fold, at least four-fold, at least five-fold, at least six-fold, at least seven-fold, at least eight-fold, at least nine-fold, at least ten-fold or more) in the first translational profile as to the second translational profile.

In some embodiments, the disease is a cancer. Non-limiting examples of cancers that can be treated according to the methods of the present invention include, but are not limited to, anal carcinoma, bladder carcinoma, breast carcinoma, cervix carcinoma, chronic lymphocytic leukemia, chronic myelogenous leukemia, endometrial carcinoma, hairy cell leukemia, head and neck carcinoma, lung (small cell) carcinoma, multiple myeloma, non-Hodgkin's lymphoma, follicular lymphoma, ovarian carcinoma, brain tumors, colorectal carcinoma, hepatocellular carcinoma, Kaposi's sarcoma, lung (non-small cell carcinoma), melanoma, pancreatic carcinoma, prostate carcinoma, renal cell carcinoma, and soft tissue sarcoma.

In some embodiments, the disease is an inflammatory disease (e.g., an autoimmune disease, arthritis, or MS). In some embodiments, the disease is a neurodegenerative disease (e.g., Parkinson's disease or Alzheimer's disease). In some embodiments, the disease is a metabolic disease (e.g., diabetes, metabolic syndrome, or a cardiovascular disease). In some embodiments, the disease is a viral infection. In some embodiments, the disease is a cardiomyopathy.

In some embodiments, a disease is associated with one or more altered biological pathways. In some embodiments, wherein a cell communication pathway is altered, the disease is an immune or inflammatory disease, a neurodegenerative disease, a cancer, a metabolic disorder, or a viral disease. In some embodiments, wherein a cell communication pathway is altered, the disease is an immune or inflammatory disease (e.g., an autoimmune disease, arthritis, or MS).

In some embodiments, wherein a cellular process pathway is altered, the disease is an immune or inflammatory disease (e.g., an autoimmune disease, arthritis, or MS), a neurodegenerative disease (e.g., Parkinson's disease or Alzheimer's disease), a cancer, a metabolic disorder, or a viral disease.

In some embodiments, wherein an immune system process pathway is altered, the disease is an immune or inflammatory disease, a neurodegenerative disease, a cancer, a metabolic disorder, or a viral disease. In some embodiments, wherein an immune system process pathway is altered, the disease is an immune or inflammatory disease (e.g., an autoimmune disease, arthritis, or MS).

In some embodiments, wherein a response to stimulus pathway is altered, the disease is an immune or inflammatory disease, a neurodegenerative disease, a metabolic disorder, or a viral disease. In some embodiments, wherein a response to stimulus pathway is altered, the disease is an immune or inflammatory disease (e.g., an autoimmune disease, arthritis, or MS) or a viral disease.

In some embodiments, wherein a transport pathway is altered, the disease is an immune or inflammatory disease, a neurodegenerative disease, or a metabolic disorder. In some embodiments, wherein a transport pathway is altered, the disease is an immune or inflammatory disease (e.g., an autoimmune disease, arthritis, or MS) or a metabolic disorder (e.g., diabetes, metabolic syndrome, or a cardiovascular disease).

In some embodiments, wherein a metabolic process pathway is altered, the disease is a neurodegenerative disease, a cancer, or a metabolic disorder. In some embodiments, wherein a metabolic process pathway is altered, the disease is a metabolic disorder (e.g., diabetes, metabolic syndrome, or a cardiovascular disease).

In some embodiments, a metabolic process pathway is a carbohydrate metabolic process pathway, a lipid metabolic process pathway, a nucleobase, nucleoside, or nucleotide pathway, or a protein metabolic process pathway (e.g., a proteolysis pathway, a protein complex assembly pathway, a protein folding pathway, a protein modification process pathway, or a translation pathway). In some embodiments, wherein a carbohydrate metabolic process pathway is altered, the disease is a neurodegenerative disease or a metabolic disorder. In some embodiments, wherein a lipid metabolic process pathway is altered, the disease is an immune or inflammatory disease, a neurodegenerative disease, or a metabolic disorder. In some embodiments, wherein a nucleobase, nucleoside, or nucleotide pathway is altered, the disease is a cancer or a viral disease. In some embodiments, wherein a protein metabolic process pathway is altered, the disease is an immune or inflammatory disease, a neurodegenerative disease, a cancer, a metabolic disorder, or a viral disease. In some embodiments, wherein a proteolysis process pathway is altered, the disease is an immune or inflammatory disease, a neurodegenerative disease, a cancer, or a metabolic disorder. In some embodiments, wherein a protein complex assembly pathway is altered, the disease is a metabolic disorder. In some embodiments, wherein a protein folding pathway is altered, the disease is a neurodegenerative disease. In some embodiments, wherein a protein modification process pathway is altered, the disease is an immune or inflammatory disease, a neurodegenerative disease, a cancer, a metabolic disorder, or a viral disease. In some embodiments, wherein a protein translation pathway is altered, the disease is an immune or inflammatory disease, a neurodegenerative disease, or a cancer.

In some embodiments, the method further comprises administering a therapeutic agent to the identified subject. In some embodiments, the method further comprises administering an mTOR inhibitor to the identified subject.

B. Administration of Therapeutic Agents

In some embodiments, the present invention relates to a method of treating a subject having a cancer. In some embodiments, the method comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes having a 5′ terminal oligopyrimidine tract (5′ TOP) and/or a pyrimidine-rich translational element (PRTE); and wherein the control sample is from a known responder to the mTOR inhibitor prior to administration of the mTOR inhibitor to the known responder;
thereby treating the cancer in the subject.

In some embodiments, the method of treating a subject having a cancer comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes selected from the group consisting of SEQ ID NOs:1-144; and wherein the control sample is from a known responder to the mTOR inhibitor prior to administration of the mTOR inhibitor to the known responder;
thereby treating the cancer in the subject.

In some embodiments, the method of treating a subject having a cancer comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes of a biological pathway selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the mTOR inhibitor prior to administration of the mTOR inhibitor to the known responder;
thereby treating the cancer in the subject.

In some embodiments, the present invention relates to a method of treating a subject in need thereof. In some embodiments, the method comprises:
administering a therapeutic agent to a subject that has been selected as having a first translational profile comprising a translational level of one or more genes that is at least as high as the translational level of the one or more genes in a second translational profile;
wherein the first and second translational profiles comprise translational levels for one or more genes of a biological pathway selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the therapeutic agent prior to administration of the therapeutic agent to the known responder;
thereby treating the subject.

A subject is selected for therapeutic treatment based on any of the translational profiling methods as described herein. In some embodiments, the subject has a disease. In some embodiments, the disease is an inflammatory disease. In some embodiments, the disease is a neurodegenerative disease. In some embodiments, the disease is a metabolic disease. In some embodiments, the disease is viral infection. In some embodiments, the disease is a cardiomyopathy. In some embodiments, the disease is cancer. Non-limiting examples of cancers that can be treated according to the methods of the present invention include, but are not limited to, anal carcinoma, bladder carcinoma, breast carcinoma, cervix carcinoma, chronic lymphocytic leukemia, chronic myelogenous leukemia, endometrial carcinoma, hairy cell leukemia, head and neck carcinoma, lung (small cell) carcinoma, multiple myeloma, non-Hodgkin's lymphoma, follicular lymphoma, ovarian carcinoma, brain tumors, colorectal carcinoma, hepatocellular carcinoma, Kaposi's sarcoma, lung (non-small cell carcinoma), melanoma, pancreatic carcinoma, prostate carcinoma, renal cell carcinoma, and soft tissue sarcoma. In some embodiments, the cancer is prostate cancer, breast cancer, bladder cancer, lung cancer, renal cell carcinoma, endometrial cancer, melanoma, ovarian cancer, thyroid cancer, or brain cancer. In some embodiments, the cancer is an invasive cancer.

A therapeutic agent for use according to any of the methods of the present invention can be any composition that has or may have a pharmacological activity. Agents include compounds that are known drugs, compounds for which pharmacological activity has been identified but which are undergoing further therapeutic evaluation, and compounds that are members of collections and libraries that are screened for a pharmacological activity. In some embodiments, the therapeutic agent is an anti-cancer, e.g., an anti-signaling agent (e.g., a cytostatic drug) such as a monoclonal antibody or a tyrosine kinase inhibitor; an anti-proliferative agent; a chemotherapeutic agent (i.e., a cytotoxic drug); a hormonal therapeutic agent; and/or a radiotherapeutic agent.

Generally, the therapeutic agent is administered at a therapeutically effective amount or dose. A therapeutically effective amount or dose will vary according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of a therapeutic agent can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art.

In some embodiments, a therapeutic agent is formulated as a pharmaceutical composition. In some embodiments, a pharmaceutical composition incorporates particulate forms, protective coatings, protease inhibitors, or permeation enhancers for various routes of administration, including parenteral, pulmonary, nasal and oral. The pharmaceutical compositions can be administered in a variety of unit dosage forms depending upon the method/mode of administration. Suitable unit dosage forms include, but are not limited to, powders, tablets, pills, capsules, lozenges, suppositories, patches, nasal sprays, injectables, implantable sustained-release formulations, etc.

In some embodiments, a pharmaceutical composition comprises an acceptable carrier and/or excipients. A pharmaceutically acceptable carrier includes any solvents, dispersion media, or coatings that are physiologically compatible and that preferably does not interfere with or otherwise inhibit the activity of the therapeutic agent. Preferably, the carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, 21st Edition, Philadelphia, Pa. Lippincott Williams & Wilkins, 2005. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

C. Normalizing Translational Profiles in a Subject

In another aspect, the methods of the present invention relate to normalizing a translational profile in a subject. In some embodiments, the present invention provides a method of identifying an agent for normalizing a translational profile in a subject. In some embodiments, the method comprises:
(a) determining a first translational profile for a first biological sample from the subject, wherein the first translational profile comprises translational levels for a plurality of genes;
(b) comparing the first translational profile to a second translational profile comprising translational levels for the plurality of genes, wherein the second translational profile is from a control sample, wherein the control sample is from a non-diseased subject;
(c) identifying one or more genes of a biological pathway as differentially translated in the first translational profile as compared to the second translational profile, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway;
(d) contacting a second biological sample from the subject with the agent;
(e) determining a third translational profile for the second biological sample, wherein the third translational profile comprises translational levels for the one or more genes identified as differentially translated in the first translational profile as compared to the second translational profile; and
(f) comparing the translational levels for the one or more genes in the third translational profile to the translational levels for the one or more genes in the first and second translational profiles;
wherein a translational level for the one or more genes in the third translational profile that is closer to the translational level for the one or more genes in the second translational profile than to the translational level for the one or more genes in the first translational profile identifies the agent as an agent for normalizing the translational profile in the subject.

In some embodiments, the present invention provides a method of normalizing a translational profile in a subject. In some embodiments, the method comprises:
administering to the subject an agent that has been selected as an agent that normalizes the translational profile in the subject, wherein the agent is selected by:
   (a) determining a first translational profile for a first biological sample from the subject, wherein the first translational profile comprises translational levels for a plurality of genes;
   (b) comparing the first translational profile to a second translational profile comprising translational levels for the plurality of genes, wherein the second translational profile is from a control sample, wherein the control sample is from a non-diseased subject;
   (c) identifying one or more genes of a biological pathway as differentially translated in the first translational profile as compared to the second translational profile, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway;
   (d) contacting a second biological sample form the subject with the agent;
   (e) determining a third translational profile for the second biological sample, wherein the third translational profile comprises translational levels for the one or more genes identified as differentially translated in the first translational profile as compared to the second translational profile; and
   (f) comparing the translational levels for the one or more genes in the third translational profile to the translational levels for the one or more genes in the first and second translational profiles; wherein a translational level for the one or more genes in the third translational profile that is closer to the translational level for the one or more genes in the second translational profile than to the translational level for the one or more genes in the first translational profile identifies the agent as an agent for normalizing the translational profile in the subject;
thereby normalizing the translational profile in the subject.

In some embodiments, one or more genes from each of at least two of the biological pathways is differentially translated in the first translational profile as compared to the second translational profile. In some embodiments, one or more genes from each of at least three of the biological pathways is differentially translated in the first translational profile as compared to the second translational profile. In some embodiments, there is at least a two-fold difference (e.g., at least two-fold, at least three-fold, at least four-fold, at least five-fold, at least six-fold, at least seven-fold, at least eight-fold, at least nine-fold, at least ten-fold difference or more) in translational level for the one or more genes in the first translational profile as compared to the second translational profile. In some embodiments, the first, second, and/or third translational profiles comprise translational levels for a subset of the genome, e.g., for about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% of the genome or more. In some embodiments, the first, second, and/or third translational profiles comprise a genome-wide measurement of gene translational levels.

The agent can be any agent as described herein. In some embodiments, the agent is a peptide, protein, inhibitory RNA, or small organic molecule.

For comparing translational levels or translational profiles multiple profiles, for example for determining to which translational profile a given experimentation translational profile is “closer” to, in some embodiments, the experimental translational profile has at least a 1.5 log₂ change (e.g., at least 1.5, at least 2.5, at least 3, at least 4, at least 5, at least 6, at least 7, at least 8, at least 9, at least 10 or more log₂ change, e.g., increase or decrease) in translational levels for one or more genes or for a set of selected marker genes. In some embodiments, the experimental translational profile has at least a 2.5 log₂ change in translational levels for one or more genes or for a set of selected marker genes. In some embodiments, the experimental translational profile has at least a 3 log₂ change in translational levels for one or more genes or for a set of selected marker genes. In some embodiments, the experimental profile has at least a 1.1 log₂ change in translational levels for at least 20%, at least 30%, at least 40%, at least 50%, at least 60%, at least 70%, at least 80%, or at least 90% or more of a set of selected marker genes or for the entire set of selected marker genes. In some embodiments, the experimental profile has at least a 2 log₂ change in translational levels for at least 20%, at least 30%, at least 40%, at least 50%, at least 60%, at least 70%, at least 80%, or at least 90% or more of a set of selected marker genes or for the entire set of selected marker genes. In some embodiments, the experimental profile has at least a 2.5 log₂ change in translational levels for at least 20%, at least 30%, at least 40%, at least 50%, at least 60%, at least 70%, at least 80%, or at least 90% or more of a set of selected marker genes or for the entire set of selected marker genes. In some embodiments, the experimental profile has at least a 4 log₂ change in translational levels for at least 20%, at least 30%, at least 40%, at least 50%, at least 60%, at least 70%, at least 80%, or at least 90% or more of a set of selected marker genes or for the entire set of selected marker genes.

In some embodiments, the subject in need thereof is a subject having a pathogenic condition in which protein translation is known or suspected to be aberrant. In some embodiments, the subject has a condition in which aberrant translation is known to be causative for the pathogenic condition.

### VIII. Examples

The following examples are offered to illustrate, but not to limit the claimed invention.

### Example 1

### Generation of a Comprehensive Map of Translationally Controlled mTOR Targets in Cancer Using Ribosome Profiling

Downstream of the phosphatidylinositol-3-OH kinase (PI(3)K)-AKT signalling pathway, mTOR assembles with either raptor or rictor to form two distinct complexes: mTORC1 and mTORC2. The major regulators of protein synthesis downstream of mTORC1 are 4EBP1 (also called EIF4EBP1) and p70S6K1/2. 4EBP1 negatively regulates eIF4E, a key rate-limiting initiation factor for cap-dependent translation. Phosphorylation of 4EBP1 by mTORC1 leads to its dissociation from eIF4E, allowing translation initiation complex formation at the 5′ end of mRNAs. The mTOR-dependent phosphorylation of p70S6K1/2 also promotes translation initiation as well as elongation. In this example, ribosome profiling delineates the translational landscape of the cancer genome at a codon-by-codon resolution upon pharmacological inhibition of mTOR. This method provides a genome-wide characterization of translationally controlled mRNAs downstream of oncogenic mTOR signalling and delineates their functional roles in cancer development.

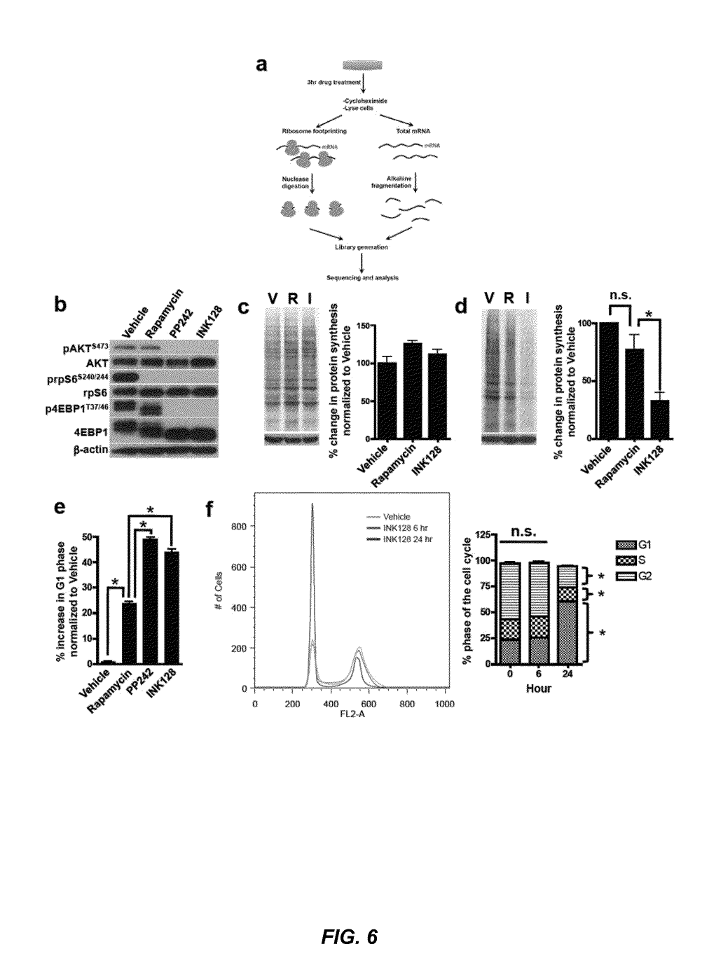

mTOR is deregulated in nearly 100% of advanced human prostate cancers, and genetic findings in mouse models implicate mTOR hyperactivation in prostate cancer initiation. Given the critical role for mTOR in prostate cancer, PC3 human prostate cancer cells, in which mTOR is constitutively hyperactivated, were used to delineate translationally controlled gene expression networks upon complete or partial mTOR inhibition. Ribosome profiling was optimized to assess quantitatively ribosome occupancy genome-wide in cancer cells. In brief, ribosome-protected mRNA fragments were deep-sequenced to determine the number of ribosomes engaged in translating specific mRNAs (see FIG. 6a and Example 6 (“Methods”) below).

Treatment of PC3 cells with an mTOR ATP site inhibitor, PP242 (Feldman et al., PLoS Biol. 7:e38 (2009); Hsieh et al., Cancer Cell 17:249-261 (2010)), significantly inhibited the activity of the three primary downstream mTOR effectors 4EBP1, p70S6K1/2 and AKT. On the contrary, rapamycin, an allosteric mTOR inhibitor, only blocked p70S6K1/2 activity in these cells (FIG. 6b). Short 3-hr drug treatments, which precede alterations in de novo protein synthesis, were used to capture direct changes in mTOR-dependent gene expression by ribosome profiling and to minimize compensatory feedback mechanisms (FIG. 6c-f).

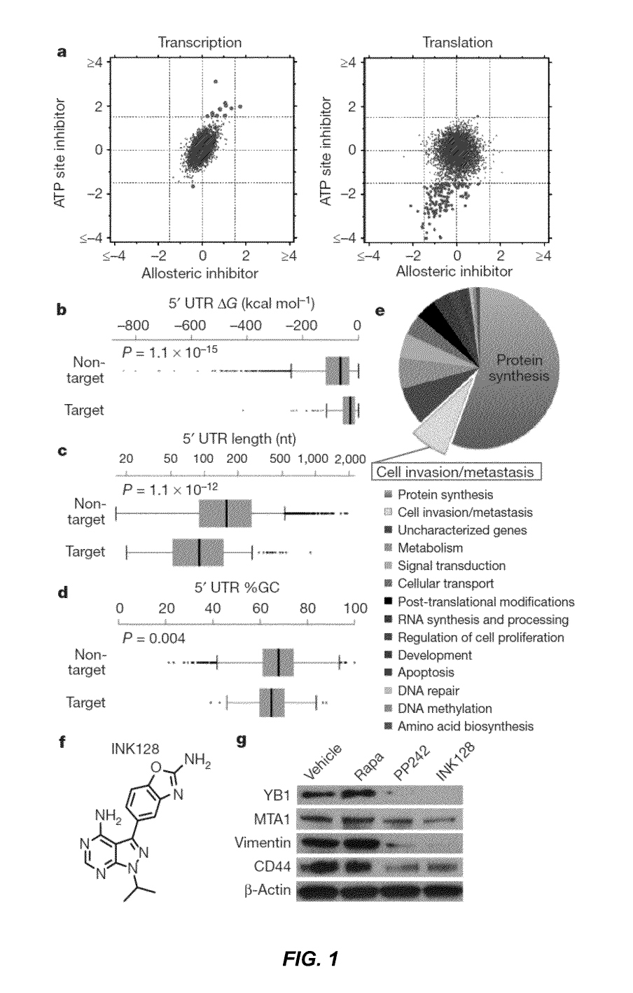

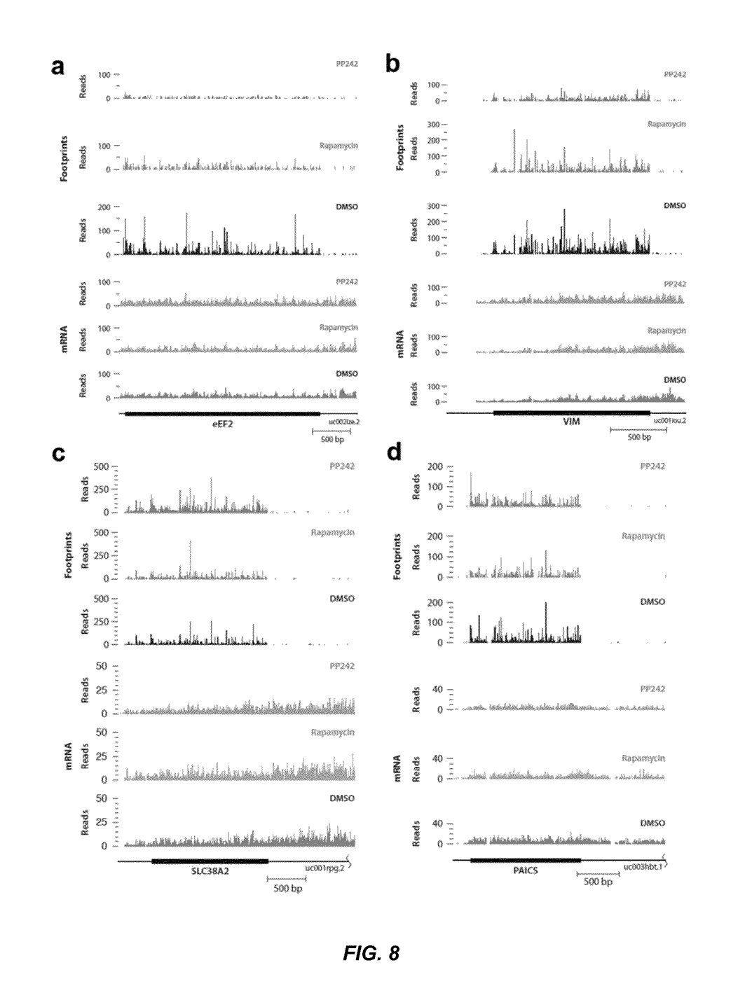

Ribosome profiling revealed 144 target mRNAs were selectively decreased at the translational level upon PP242 treatment (log₂≦1.5 (false discovery rate <0.05)) as compared to rapamycin treatment, with limited changes in transcription (FIGS. 1a, 7a-b, and 8-10, Table 3, Table 5, Table 6, and Table 7). The fact that at this time point rapamycin treatment did not markedly affect gene expression is consistent with incomplete, allosteric, inhibition of mTOR activity (FIG. 6b). By monitoring footprints of translating 80S ribosomes, these findings showed that the effects of PP242 were largely at the level of translation initiation and not elongation (FIG. 8). It has been proposed that mRNAs translationally regulated by mTOR may contain long 5′ untranslated regions (5′ UTRs) with complex RNA secondary structures. On the contrary, ribosome profiling revealed that mTOR-responsive 5′ UTRs possess less complex features (FIG. 1b-d), providing a unique data set to investigate the nature of regulatory elements that render these mRNAs mTOR-sensitive. It has been previously shown that some mTOR translationally regulated mRNAs, most notably those involved in protein synthesis, possess a 5′ terminal oligopyrimidine tract (5′ TOP) that is regulated by distinct trans-acting factors. Of the 144 mTOR-sensitive target genes, 68% possessed a 5′ TOP (see Table 1). Additionally, another 5′ UTR consensus sequence, termed a pyrimidine-rich translational element (PRTE), was identified within the 5′ UTRs of 63% of mTOR target mRNAs (P=3.2×10⁻¹¹). This PRTE element, unlike the 5′ TOP sequence, consists of an invariant uridine at position 6 flanked by pyrimidines and does not reside at position +1 of the 5′ UTR (FIG. 7c and Table 2). 89% of the mTOR-responsive genes were found to possess a PRTE and/or 5′ TOP, making the presence of one or both sequences a strong predictor for mTOR sensitivity (FIG. 7d and Table 3). Notably, mRNA isoforms arising from distinct transcription start sites may possess both a 5′ TOP and a PRTE. Given the significant number of mRNAs that contain both the PRTE and 5′ TOP, a functional interplay may exist between these regulatory elements. Additionally, these findings show that the PRTE imparts translational control specificity to 4EBP1 activity.

Surprisingly, mTOR-sensitive genes stratified into unique functional categories that may promote cancer development and progression, including cellular invasion (P=0.009), cell proliferation (P=0.04), metabolism (P=0.0002) and regulators of protein modification (P=0.01) (FIG. 1e). The largest fraction of mTOR-responsive mRNAs clustered into a node consisting of key components of the translational apparatus: 70 ribosomal proteins, 6 elongation factors, and 4 translation initiation factors (P=7.5×10⁻⁸²) (FIG. 1e). Therefore, this class of mTOR-responsive mRNAs may represent an important regulon that sustains the elevated protein synthetic capacity of cancer cells.

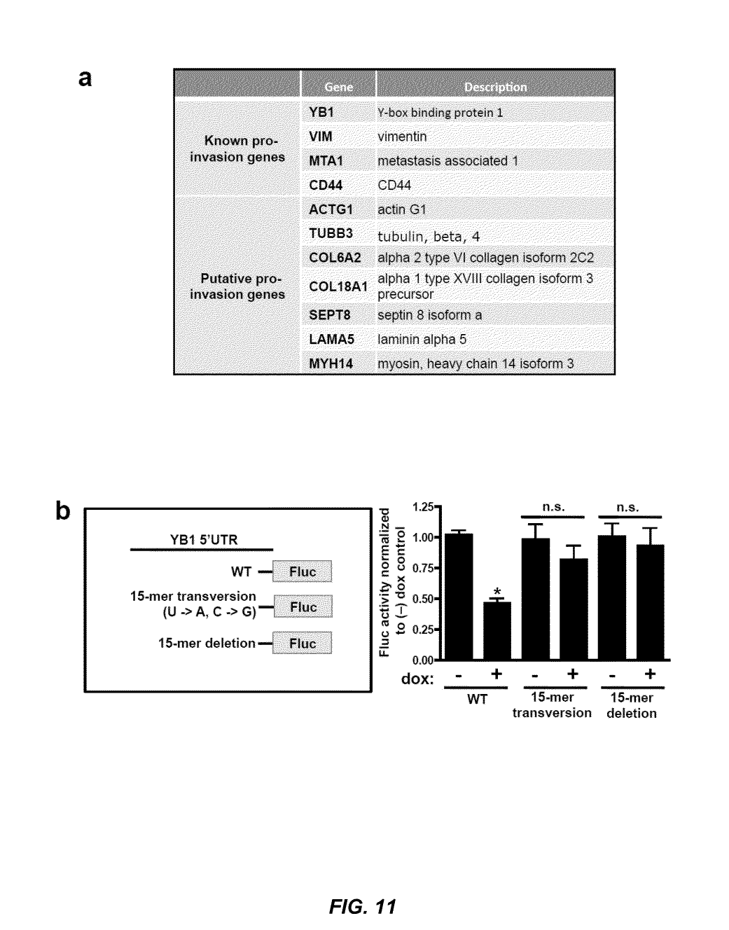

The second largest node of mTOR translationally regulated genes comprised bona fide cell invasion and metastasis mRNAs and putative regulators of this process (FIG. 1e). This group included YB1 (Y-box binding protein 1; also called YBX1), vimentin, MTA1 (metastasis associated 1) and CD44 (FIG. 11a). YB1 regulates the post-transcriptional expression of a network of invasion genes. Vimentin, an intermediate filament protein, is highly upregulated during the epithelial-to-mesenchymal transition associated with cellular invasion. MTA1, a putative chromatin-remodeling protein, is overexpressed in invasive human prostate cancer and has been shown to drive cancer metastasis by promoting neoangiogenesis. CD44 is commonly overexpressed in tumor-initiating cells and is implicated in prostate cancer metastasis. Consistent with their status as mTOR-sensitive genes, YB1, vimentin, MTA1 and CD44 all possess a PRTE (Table 2). Vimentin and CD44 also possess a 5′ TOP (Table 3). To test the functional role of the PRTE in mediating translational control, the PRTE was mutated within the 5′ UTR of YB1, which rendered the YB1 5′ UTR insensitive to inhibition by 4EBP1 (FIG. 11b). These findings highlight a novel cis-regulatory element that may modulate translational control of subsets of mRNAs upon mTOR activation. Moreover, ribosome profiling reveals unexpected transcript-specific translational control, mediated by oncogenic mTOR signaling, including a distinct set of pro-invasion and metastasis genes.

TABLE 5
Mean list of translationally regulated PP242-responsive genes

| | | Rapamycin | | PP242 | |
|---|---|---|---|---|---|
| Gene | Description | mRNA | TrlEff | mRNA | TrlEff |
| EEF2 | eukaryotic translation elongation | 0.39 | −1.12 | 0.76 | −3.60 |
| | factor 2 | | | | |
| EEF1A1 | eukaryotic translation elongation | 0.43 | −1.58 | 0.36 | −3.21 |
| | factor 1 alpha 1 | | | | |
| RPL13A | ribosomal protein L13a | 0.15 | −1.25 | 0.30 | −3.10 |
| RPS12 | ribosomal protein S12 | 0.11 | −1.22 | 0.04 | −3.00 |
| RPL12 | ribosomal protein L12 | 0.07 | −0.94 | 0.12 | −2.95 |
| RPS27 | ribosomal protein S27 | 0.10 | −1.54 | 0.07 | −2.71 |
| RPS28 | ribosomal protein S28 | 0.01 | −0.80 | 0.28 | −2.67 |
| RPL18A | ribosomal protein L18a | 0.17 | −0.82 | 0.23 | −2.63 |
| RPL34 | ribosomal protein L34 | 0.11 | −1.12 | 0.04 | −2.63 |
| RPL28 | ribosomal protein L28 isoform 1 | 0.24 | −1.09 | 0.22 | −2.54 |
| RPL27A | ribosomal protein L27a | 0.06 | −0.96 | 0.07 | −2.53 |
| CRTAP | cartilage associated protein | 0.29 | −1.17 | 0.33 | −2.50 |
| RPL10 | ribosomal protein L10 | 0.09 | −0.79 | 0.25 | −2.46 |
| RPS20 | ribosomal protein S20 isoform 1 | 0.18 | −1.35 | −0.01 | −2.46 |
| RPL21 | ribosomal protein L21 | 0.14 | −1.25 | −0.04 | −2.45 |
| RPL3 | ribosomal protein L3 isoform a | 0.18 | −1.08 | 0.22 | −2.44 |
| RPL39 | ribosomal protein L39 | 0.17 | −1.65 | −0.15 | −2.41 |
| RPL37A | ribosomal protein L37a | 0.08 | −1.02 | 0.01 | −2.38 |
| VIM | vimentin | 0.36 | −0.40 | 0.67 | −2.38 |
| EEF1D | eukaryotic translation elongation | 0.18 | −0.84 | 0.35 | −2.37 |
| | factor 1 delta | | | | |
| GNB2L1 | Guanine nucleotide binding protein | 0.19 | −0.77 | 0.27 | −2.35 |
| | (G protein) | | | | |
| RPS19 | ribosomal protein S19 | 0.15 | −0.74 | 0.23 | −2.34 |
| RPL32 | ribosomal protein L32 | 0.22 | −0.97 | 0.11 | −2.33 |
| RPS15A | ribosomal protein S15a | 0.07 | −0.96 | 0.07 | −2.31 |
| RPL11 | ribosomal protein L11 | 0.09 | −1.08 | 0.14 | −2.31 |
| RPL7A | ribosomal protein L7a | 0.17 | −0.74 | 0.15 | −2.30 |
| YB1 | Y-box binding protein 1 | 0.11 | −0.59 | 0.24 | −2.30 |
| RPS9 | ribosomal protein S9 | 0.10 | −0.60 | 0.34 | −2.27 |
| EIF4B | eukaryotic translation initiation | 0.55 | −1.21 | 0.61 | −2.27 |
| | factor 4B | | | | |
| EEF1G | eukaryotic translation elongation | 0.21 | −1.15 | 0.15 | −2.26 |
| | factor 1, gamma | | | | |
| RPS2 | ribosomal protein S2 | 0.07 | −0.56 | 0.20 | −2.25 |
| RPS5 | ribosomal protein S5 | 0.14 | −0.77 | 0.23 | −2.25 |
| HSPA8 | heat shock 70 kDa protein 8 isoform 1 | −0.21 | −0.46 | −0.40 | −2.25 |
| RPS3A | ribosomal protein S3a | 0.22 | −1.15 | −0.06 | −2.17 |
| RPS3 | ribosomal protein S3 | 0.22 | −0.92 | 0.24 | −2.16 |
| RPL10A | ribosomal protein L10a | 0.16 | −0.94 | 0.14 | −2.16 |
| RPS25 | ribosomal protein S25 | 0.04 | −0.89 | −0.04 | −2.13 |
| GLTSCR2 | glioma tumor suppressor candidate | 0.31 | −0.68 | 0.70 | −2.12 |
| | region gene 2 | | | | |
| HNRNPA1 | heterogeneous nuclear | 0.18 | −0.86 | 0.27 | −2.12 |
| | ribonucleoprotein A1 | | | | |
| RPLP2 | ribosomal protein P2 | 0.26 | −1.18 | 0.14 | −2.10 |
| RPL31 | ribosomal protein L31 isoform 2 | −0.02 | −0.62 | 0.05 | −2.10 |
| PABPC1 | poly(A) binding protein, | 0.35 | −1.44 | 0.16 | −2.09 |
| | cytoplasmic 1 | | | | |
| RPS21 | ribosomal protein S21 | −0.01 | −0.60 | 0.09 | −2.09 |
| RPS4X | ribosomal protein S4, X-linked X | 0.18 | −1.15 | 0.12 | −2.06 |
| | isoform | | | | |
| RPLP1 | ribosomal protein P1 isoform 1 | 0.28 | −1.09 | 0.12 | −2.06 |
| RPL7 | ribosomal protein L7 | 0.15 | −1.06 | 0.01 | −2.02 |
| RPL26 | ribosomal protein L26 | 0.15 | −1.11 | 0.02 | −2.00 |
| PABPC4 | poly A binding protein, cytoplasmic | 0.24 | −0.80 | 0.40 | −1.98 |
| | 4 isoform 1 | | | | |
| RPL36A | ribosomal protein L36a | 0.13 | −1.11 | −0.01 | −1.98 |
| EEF1A2 | eukaryotic translation elongation | 0.03 | −0.03 | 0.40 | −1.94 |
| | factor 1 alpha 2 | | | | |
| TPT1 | tumor protein, translationally- | 0.24 | −1.22 | 0.01 | −1.94 |
| | controlled 1 | | | | |
| AHCY | adenosylhomocysteinase isoform 1 | 0.20 | −0.23 | 0.38 | −1.93 |
| RPL22L1 | ribosomal protein L22-like 1 | 0.15 | −0.68 | 0.39 | −1.90 |
| GAPDH | glyceraldehyde-3-phosphate | 0.17 | −0.27 | 0.28 | −1.90 |
| | dehydrogenase | | | | |
| RPL30 | ribosomal protein L30 | 0.11 | −0.99 | 0.01 | −1.89 |
| RPS11 | ribosomal protein S11 | 0.11 | −0.59 | 0.20 | −1.88 |
| RPL29 | ribosomal protein L29 | 0.10 | −0.50 | 0.20 | −1.88 |
| RPL14 | ribosomal protein L14 | 0.07 | −0.68 | −0.02 | −1.85 |
| RPL36 | ribosomal protein L36 | 0.09 | −0.43 | 0.28 | −1.85 |
| EIF2S3 | eukaryotic translation initiation | 0.33 | −1.04 | 0.15 | −1.85 |
| | factor 2, S3 | | | | |
| RPL23 | ribosomal protein L23 | 0.09 | −0.92 | 0.07 | −1.82 |
| RPS16 | ribosomal protein S16 | 0.13 | −0.38 | 0.19 | −1.81 |
| SLC25A5 | adenine nucleotide translocator 2 | 0.21 | −0.30 | 0.15 | −1.80 |
| RPL17 | ribosomal protein L17 | 0.05 | −0.93 | 0.07 | −1.80 |
| RPL37 | ribosomal protein L37 | 0.11 | −0.68 | 0.10 | −1.79 |
| RPL8 | ribosomal protein L8 | 0.12 | −0.40 | 0.29 | −1.79 |
| NAP1L1 | nucleosome assembly protein 1-like 1 | 0.24 | −0.97 | 0.15 | −1.79 |
| RPS10 | ribosomal protein S10 | 0.16 | −0.69 | 0.19 | −1.78 |
| IPO7 | importin 7 | 0.20 | −0.83 | 0.26 | −1.75 |
| RPS8 | ribosomal protein S8 | 0.09 | −0.44 | 0.14 | −1.74 |
| RPL5 | ribosomal protein L5 | 0.17 | −1.11 | 0.06 | −1.73 |
| RPS24 | ribosomal protein S24 isoform d | 0.11 | −1.16 | −0.01 | −1.73 |
| EEF1B2 | eukaryotic translation elongation | 0.12 | −1.10 | −0.06 | −1.70 |
| | factor 1 beta 2 | | | | |
| RPL6 | ribosomal protein L6 | 0.09 | −0.68 | 0.06 | −1.68 |
| RPS23 | ribosomal protein S23 | 0.15 | −1.19 | −0.03 | −1.68 |
| RPL18 | ribosomal protein L18 | 0.08 | −0.42 | 0.18 | −1.65 |
| RPS29 | ribosomal protein S29 isoform 2 | −0.01 | −0.69 | 0.11 | −1.65 |
| RPS6 | ribosomal protein S6 | 0.14 | −1.06 | −0.02 | −1.65 |
| RPL22 | ribosomal protein L22 | 0.08 | −0.89 | 0.00 | −1.64 |
| UBA52 | ubiquitin and ribosomal protein L40 | 0.12 | −0.22 | 0.18 | −1.62 |
| RPLP0 | ribosomal protein PO | 0.15 | −0.42 | 0.12 | −1.61 |
| RPS27A | ubiquitin and ribosomal protein | 0.16 | −0.89 | −0.04 | −1.61 |
| | S27a | | | | |
| RPL9 | ribosomal protein L9 | 0.16 | −1.00 | −0.08 | −1.59 |
| TKT | transketolase isoform 1 | 0.02 | −0.11 | 0.33 | −1.58 |
| RPL13 | ribosomal protein L13 | 0.14 | −0.38 | 0.26 | −1.56 |
| EIF3H | eukatyotic translation initiation | 0.16 | −0.79 | 0.09 | −1.54 |
| | factor 3, | | | | |
| RPS13 | ribosomal protein S13 | 0.07 | −0.82 | −0.08 | −1.54 |
| RPS7 | ribosomal protein S7 | 0.11 | −0.76 | −0.04 | −1151 |
| RPS14 | ribosomal protein S14 | 0.10 | −0.60 | 0.16 | −1.50 |
| RPL4 | ribosomal protein L4 | 0.22 | −0.85 | 0.10 | −1.50 |
| FAM128B | hypothetical protein LOC80097 | 0.06 | 0.27 | 0.43 | −1.47 |
| EIF3L | eukaryotic translation initiation | 0.28 | −0.85 | 0.21 | −1.47 |
| | factor 3L | | | | |
| RABGGTB | RAB geranylgeranyltransferase, | −0.20 | −0.84 | 0.20 | −1.46 |
| | beta subunit | | | | |
| FASN | fatty acid synthase | −0.37 | 0.47 | 0.30 | −1.42 |
| RPL24 | ribosomal protein L24 | 0.11 | −0.63 | 0.00 | −1.41 |
| ACTG1 | actin, gamma 1 propeptide | 0.02 | −0.07 | 0.28 | −1.40 |
| PFDN5 | prefoldin subunit 5 isoform alpha | 0.11 | −0.51 | 0.04 | −1.38 |
| LMF2 | lipase maturation factor 2 | 0.22 | 0.39 | 0.62 | −1.36 |
| RPL19 | ribosomal protein L19 | 0.14 | −0.66 | 0.11 | −1.35 |
| PGM1 | phosphoglucomutase 1 | 0.40 | −0.55 | 0.23 | −1.35 |
| CCNI | cyclin I | 0.29 | −0.45 | 0.24 | −1.33 |
| IMPDH2 | inosine monophosphate | 0.11 | −0.39 | 0.21 | −1.33 |
| | dehydrogenase 2 | | | | |
| AP2A1 | adaptor-related protein complex 2, | 0.09 | −0.04 | 0.42 | −1.32 |
| | alpha 1 | | | | |
| AGRN | agrin precursor | 0.01 | 0.51 | 0.50 | −1.29 |
| COL6A2 | alpha 2 type VI collagen isoform | −0.08 | 0.43 | 0.57 | −1.29 |
| | 2C2 | | | | |
| CD44 | CD44 antigen isoform 1 | 0.34 | −0.46 | 0.43 | −1.29 |
| RPL41 | ribosomal protein L41 | 0.04 | −1.15 | −0.01 | −1.28 |
| ALKBH7 | spermatogenesis associated 11 | 0.06 | 0.28 | 0.51 | −1.27 |
| | precursor | | | | |
| RPL27 | ribosomal protein L27 | 0.05 | −0.33 | −0.13 | −1.23 |
| RPL15 | ribosomal protein L15 | 0.11 | −0.51 | 0.19 | −1.20 |
| RPS15 | ribosomal protein S15 | −0.01 | 0.03 | 0.21 | −1.19 |
| CLPTM1 | cleft lip and palate associated | 0.07 | 0.26 | 0.41 | −1.13 |
| | transmembrane | | | | |
| FAM83H | FAM83H | −0.17 | 0.71 | 0.33 | −1.11 |
| PGLS | 6-phosphogluconolactonase | 0.03 | 0.20 | 0.21 | −1.11 |
| MTA1 | metastasis associated 1 | 0.00 | −0.05 | 0.21 | −1.09 |
| TSC2 | tuberous sclerosis 2 isoform 1 | −0.15 | 0.34 | 0.21 | −1.09 |
| PACS1 | phosphofurin acidic cluster sorting | 0.07 | 0.04 | 0.45 | −1.09 |
| | protein 1 | | | | |
| CIRBP | cold inducible RNA binding protein | 0.14 | 0.10 | 0.54 | −1.08 |
| SLC19A1 | solute carrier family 19 member 1 | −036 | 0.23 | 0.10 | −1.07 |
| ECSIT | evolutionarily conserved signaling | −0.04 | 0.41 | 0.26 | −1.06 |
| | intermediate | | | | |
| ARD1A | alpha-N-acetyltransferase 1A | −0.04 | 0.01 | 0.03 | −1.05 |
| C21orf66 | GC-rich sequence DNA-binding | −0.30 | −0.09 | −0.31 | −1.03 |
| | factor candidate | | | | |
| ATP5G2 | ATP synthase, H+ transporting, | 0.29 | −0.28 | 0.17 | −1.01 |
| | mitochondrial F0 | | | | |
| LAMA5 | laminin alpha 5 | −0.32 | 0.87 | 0.40 | −0.94 |
| PNKP | polynucleotide kinase 3′ | −0.24 | 0.74 | 0.33 | −0.79 |
| | phosphatase | | | | |
| EVPL | envoplakin | −0.08 | 0.30 | 0.38 | −0.79 |
| NCLN | nicalin | −0.05 | 0.67 | 0.29 | −0.76 |
| PTGES2 | prostaglandin E synthase 2 | −0.19 | 0.52 | 0.17 | −0.65 |
| GAMT | guanidinoacetate N- | n/a | n/a | n/a | n/a |
| | methyltransferase isoform b | | | | |
| CTSH | cathepsin H isoform b | n/a | n/a | n/a | n/a |
| TUBB3 | tubulin, beta, 4 | n/a | n/a | n/a | n/a |
| CSDA | cold shock domain protein A | n/a | n/a | n/a | n/a |
| ETHE1 | ETHE1 protein | n/a | n/a | n/a | n/a |
| LCMT1 | leucine carboxyl methyltransferase | n/a | n/a | n/a | n/a |
| | 1 isoform a | | | | |
| PC | pyruvate carboxylase | n/a | n/a | n/a | n/a |
| SECTM1 | secreted and transmembrane 0 | n/a | n/a | n/a | n/a |
| COL18A1 | alpha 1 type XVIII collagen | n/a | n/a | n/a | n/a |
| | isoform 3 | | | | |
| CHP | calcium binding protein P22 | n/a | n/a | n/a | n/a |
| BRF1 | transcription initiation factor IIIB | n/a | n/a | n/a | n/a |
| C2orf79 | hypothetical protein LOC391356 | n/a | n/a | n/a | n/a |
| SEPT8 | septin 8 isoform a | n/a | n/a | n/a | n/a |
| ABCB7 | ATP-binding cassette, sub-family | n/a | n/a | n/a | n/a |
| | B, member 7 | | | | |
| MYH14 | myosin, heavy chain 14 isoform 3 | n/a | n/a | n/a | n/a |
| SIGMAR1 | sigma non-opioid intracellular | n/a | n/a | n/a | n/a |
| | receptor 1 | | | | |
| C3orf38 | hypothetical protein LOC285237 | n/a | n/a | n/a | n/a |

TABLE 6
List of rapamycin-sensitive translationally regulated genes after 3-hour treatment
with rapamycin (50 nM) or PP242 (2.5 μM) in PC3 cells.

| | | Rapamycin | | PP242 | |
|---|---|---|---|---|---|
| Gene | Description | mRNA | TrlEff | mRNA | TrlEff |
| MAPK6 | mitogen-activated protein kinase 6 | 0.13 | −2.43 | 0.10 | −0.29 |
| RPL39 | ribosomal protein L39 | 0.30 | −2.11 | −0.42 | −2.53 |
| RPS20 | ribosomal protein S20 isoform 1 | 0.14 | −1.79 | −0.10 | −2.78 |
| PRKD3 | protein kinase D3 | −0.22 | −1.72 | −0.46 | 0.68 |
| UBTD2 | dendritic cell-derived ubiquitin- | 0.19 | −1.64 | 0.25 | 0.27 |
| | like protein | | | | |
| RPL28 | ribosomal protein L28 isoform 1 | 0.64 | −1.59 | 0.55 | −3.48 |
| RBPJ | recombining binding protein | 1.09 | −1.58 | 0.17 | −0.03 |
| | suppressor of | | | | |
| EEF1A1 | eukaryotic translation elongation | 0.46 | −1.57 | 0.29 | −3.53 |
| | factor 1 alpha | | | | |
| UCHL5 | ubiquitin carboxyl-terminal | −0.08 | −1.56 | −0.51 | 0.40 |
| | hydrolase L5 | | | | |
| RPS27 | ribosomal protein S27 | 0.07 | −1.55 | 0.06 | −3.35 |
| SDCCAG10 | serologically defined colon cancer | −0.19 | −1.50 | −0.37 | 0.23 |
| | antigen 10 | | | | |
| MAPKAPK2 | mitogen-activated protein kinase- | −0.21 | 1.50 | −0.22 | 0.92 |
| | activated | | | | |
| NFATC21P | nuclear factor of activated T-cells, | −0.16 | 1.54 | 0.08 | 0.35 |
| | 2IP | | | | |
| GTPBP3 | GTP binding protein 3 | −0.73 | 1.56 | 0.15 | −0.83 |
| | (mitochondrial) isoform V | | | | |
| C17orf28 | hypothetical protein LOC283987 | −0.44 | 1.66 | 0.21 | −0.20 |
| VHL | von Hippel-Lindau tumor | −0.23 | 1.67 | 0.43 | 0.52 |
| | suppressor isoform 1 | | | | |
| DDX51 | DEAD (Asp-Glu-Ala-Asp) box | −0.24 | 1.68 | 0.17 | −0.51 |
| | polypeptide 51 | | | | |
| DGCR2 | integral membrane protein | −0.66 | 1.69 | 0.05 | 0.02 |
| | DGCR2 | | | | |
| CCNA1 | cyclin A1 isoform a | −0.51 | 1.81 | −0.33 | 0.66 |
| NR2F1 | nuclear receptor subfamily 2, | 0.05 | 1.94 | 0.87 | −0.09 |
| | group F, member 1 | | | | |
| ACD | adrenocortical dysplasia homolog | −0.96 | 2.06 | 0.20 | −1.02 |
| | isoform 1 | | | | |

TABLE 7
PP242 and rapamycin transcriptional targets.

| | | |
|---|---|---|
| Gene | Description | mRNA |

A. PP242 sensitive transcriptionally regulated genes upon 3-hour
treatment with PP242 (2.5 μM) in PC3 cells*

| | | |
|---|---|---|
| FGFBP1 | fibroblast growth factor binding protein 1 | −1.75 |
| BRIX1 | ribosome biogenesis protein BRX1 homolog | −1.51 |
| FOXA1 | forkhead box A1 | 1.45 |
| CYR61 | cysteine-rich, angiogenic inducer, 61 precursor | 1.47 |
| MT2A | metallothionein 2A | 1.47 |
| SOX4 | SRY (sex determining region Y)-box 4 | 1.51 |
| BCL6 | B-cell lymphoma 6 protein isoform 1 | 1.59 |
| KLF6 | Kruppel-like factor 6 isoform A | 1.75 |
| RND3 | ras homolog gene family, member E precursor | 1.78 |
| CTGF | connective tissue growth factor precursor | 1.80 |
| HBP1 | HMG-box transcription factor 1 | 1.88 |
| ARID5B | AT rich interactive domain 5B (MRF1-like) | 1.93 |
| PLAU | plasminogen activator, urokinase isoform 1 | 2.04 |
| GDF15 | growth differentiation factor 15 | 3.02 |

B. Rapamycin sensitive transcriptionally regulated genes upon 3-hour
treatment with rapamycin (50 nM) in PC3 cells*

| | | |
|---|---|---|
| HBP1 | HMG-box transcription factor 1 | −1.75 |

*log₂ fold change

### Example 2

### Translation of Pro-Invasion mRNAs by mTOR

To extend the use of the mTOR pharmacological tools used in ribosome profiling towards functional characterization of the newly identified mTOR-sensitive cell invasion gene signature, a new clinical-grade mTOR ATP site inhibitor was developed that was derived from the PP242 chemical scaffold. In brief, a structure-guided optimization of pyrazolopyrimidine derivatives was performed that improved oral bioavailability while retaining mTOR kinase potency and selectivity. The ATP site inhibitor of mTOR was selected for clinical studies on the basis of its high potency (1.4 nM inhibition constant (Kᵢ)), selectivity for mTOR, low molecular mass, and favorable pharmaceutical properties.

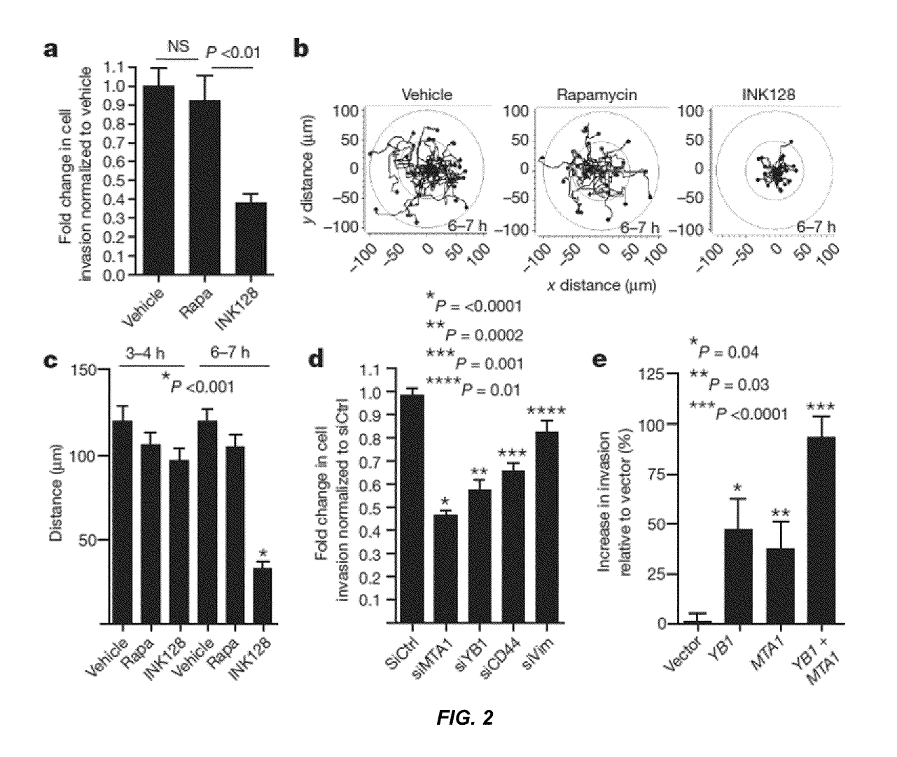

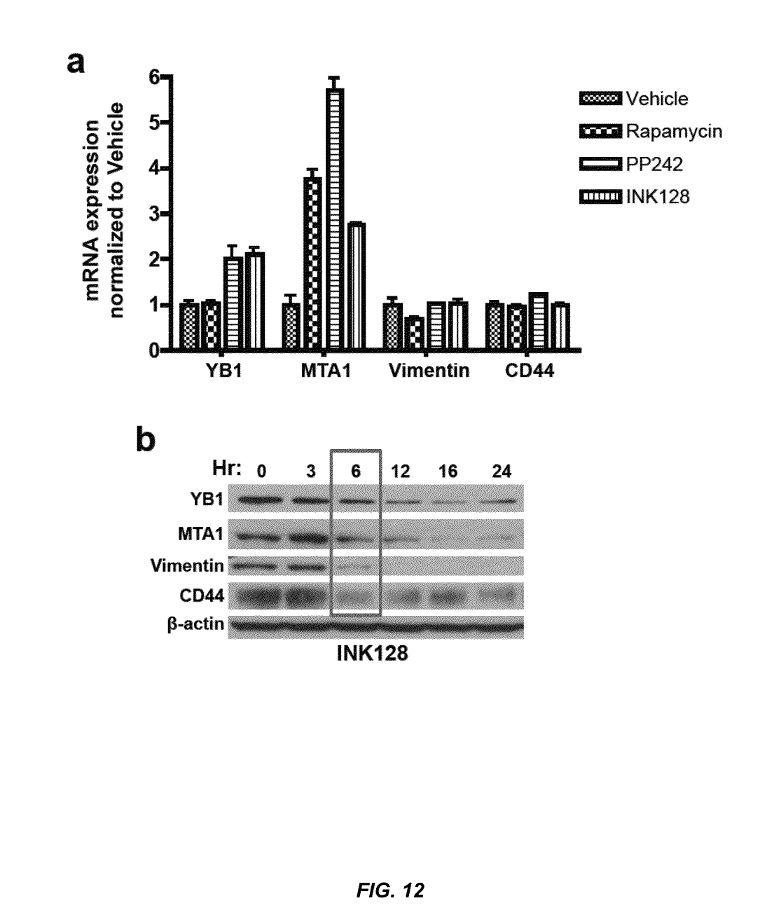

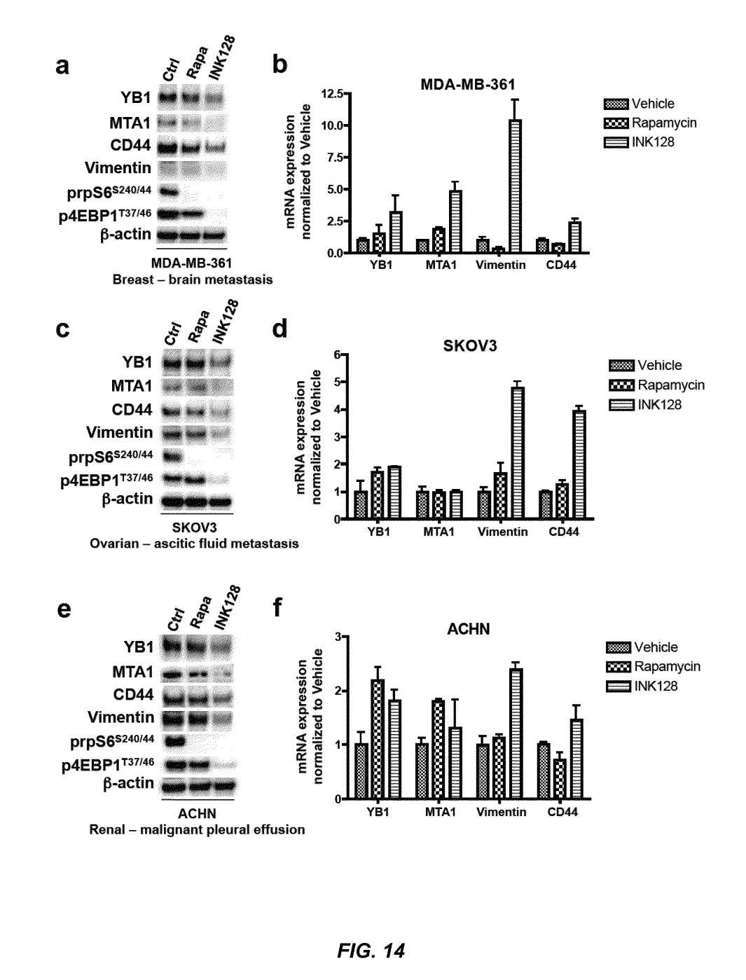

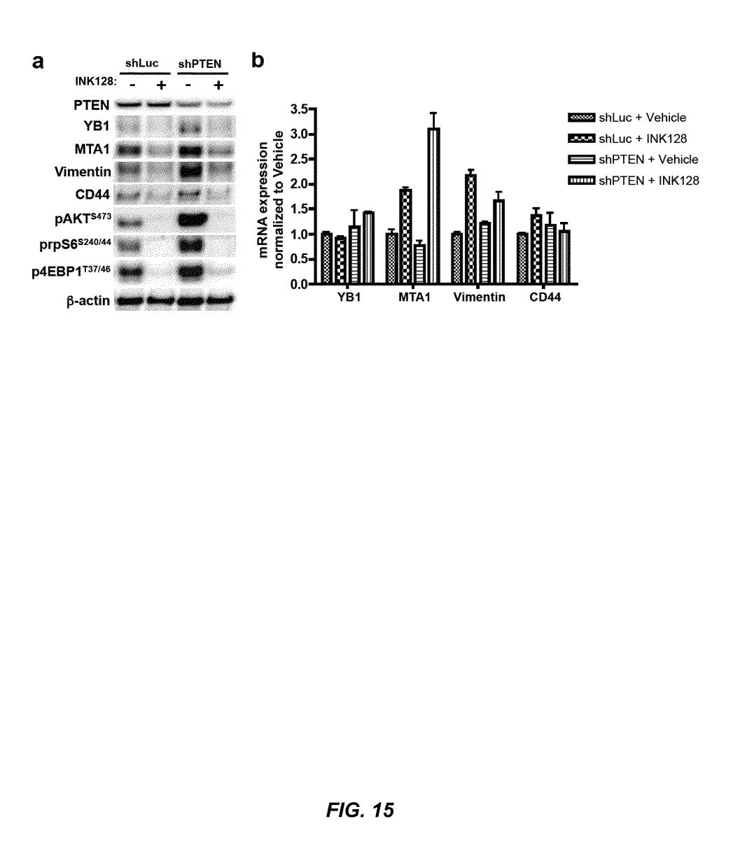

Using either PP242 or the new (or optimized) ATP site inhibitor of mTOR, a selective decrease in the expression of YB1, MTA1, vimentin, and CD44 was observed at the protein but not transcript level in PC3 cells starting at 6 hr of treatment, which preceded any decrease in de novo protein synthesis (FIGS. 1f, 6c-d, 12, and 13). In contrast, rapamycin treatment did not alter their expression (FIGS. 1g and 12a). Similar findings were observed using a broad panel of metastatic cell lines of distinct histological origins (FIG. 14). The four-gene invasion signature (YB1, MTA1, vimentin and CD44) was positively regulated by mTOR hyperactivation, as silencing PTEN expression increased their protein but not mRNA expression levels (FIG. 15). Next, the effects of mTOR ATP site inhibitors on prostate cancer cell migration and invasion were investigated. The ATP site inhibitor of mTOR, but not rapamycin, decreased the invasive potential of PC3 prostate cancer cells (FIG. 2a). Furthermore, the ATP site inhibitor of mTOR inhibited cancer cell migration starting at 6 hr of treatment, precisely correlating with when decreases in the expression of pro-invasion genes were evident, but preceding any changes in the cell cycle or overall global protein synthesis (FIGS. 2b-c, 6c, 6e, 6f, 12b, and 16).

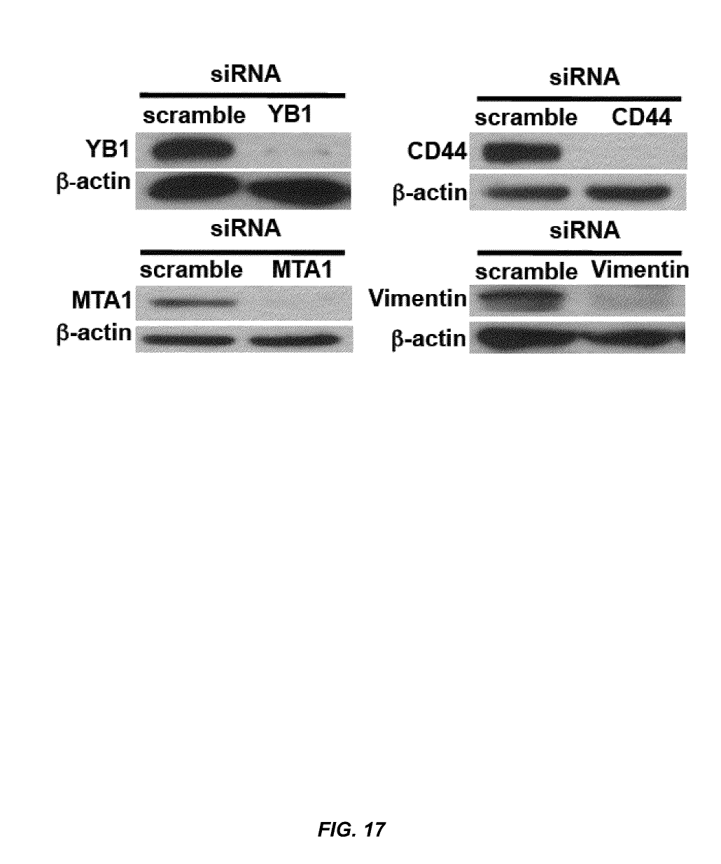

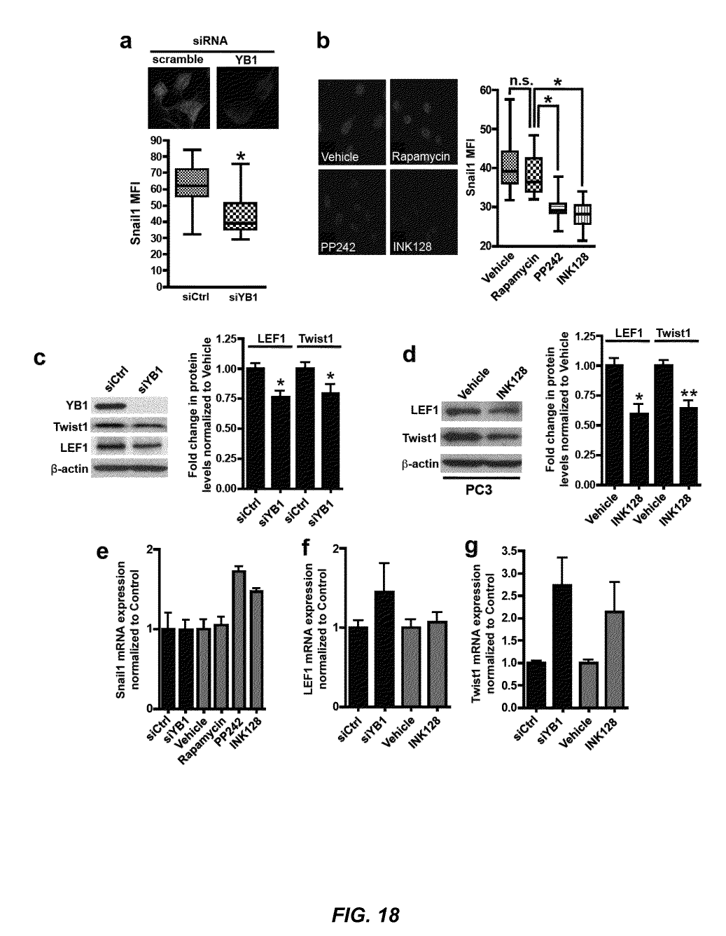

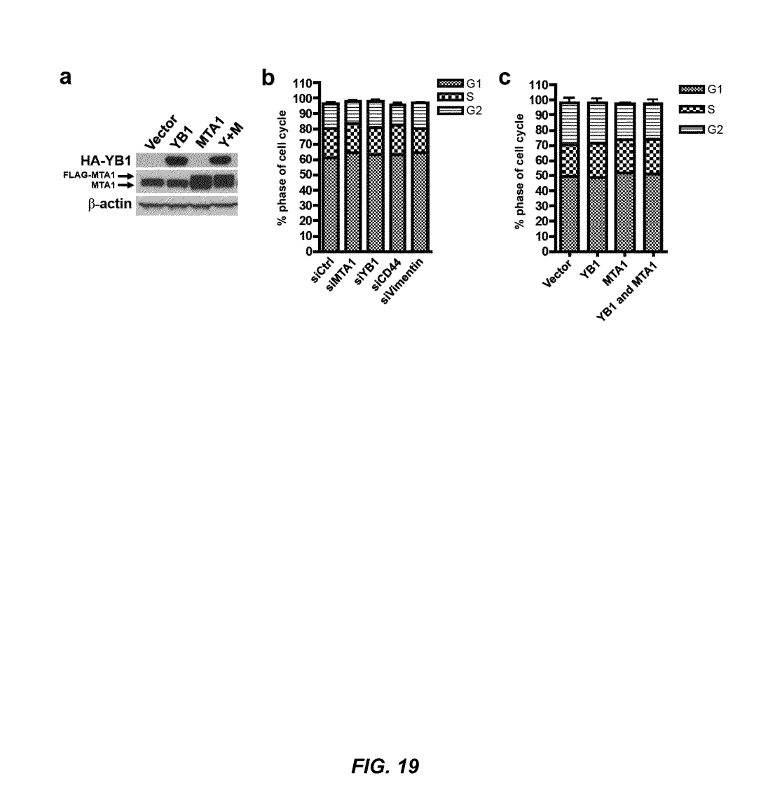

Among the genes comprising the pro-invasion signature, YB1 has been shown to act directly as a translation factor that controls expression of a larger set of genes involved in breast cancer cell invasion. Notably, YB1 translationally-regulated target mRNAs, including SNAIL1 (also called SNAI), LEF1 and TWIST1, decreased at the protein but not transcript level upon YB1 knockdown in PC3 cells (FIGS. 17 and 18). To determine the functional role of YB1 in prostate cancer cell invasion, YB1 gene expression was silenced in PC3 cells and a 50% reduction in cell invasion was observed (FIG. 2d). Similarly, knockdown of MTA1, CD44, or vimentin also inhibited prostate cancer cell invasion (FIGS. 2d and 17). These mTOR target mRNAs may be sufficient to endow primary prostate cells with invasive features, as overexpression of YB1 and/or MTA1 (FIG. 19a) in BPH-1 cells, an untransformed prostate epithelial cell line, increased the invasive capacity of these cells in an additive manner (FIG. 2e). Notably, the effects of YB1 and MTA1 on cell invasion were independent from any effect on cell proliferation in both knockdown or overexpression studies (FIG. 19b-c). Therefore, translational control of pro-invasion mRNAs by oncogenic mTOR signaling alters the ability of epithelial cells to migrate and invade, a key feature of cancer metastasis.

### Example 3

### Dissecting mTOR Translational Effectors

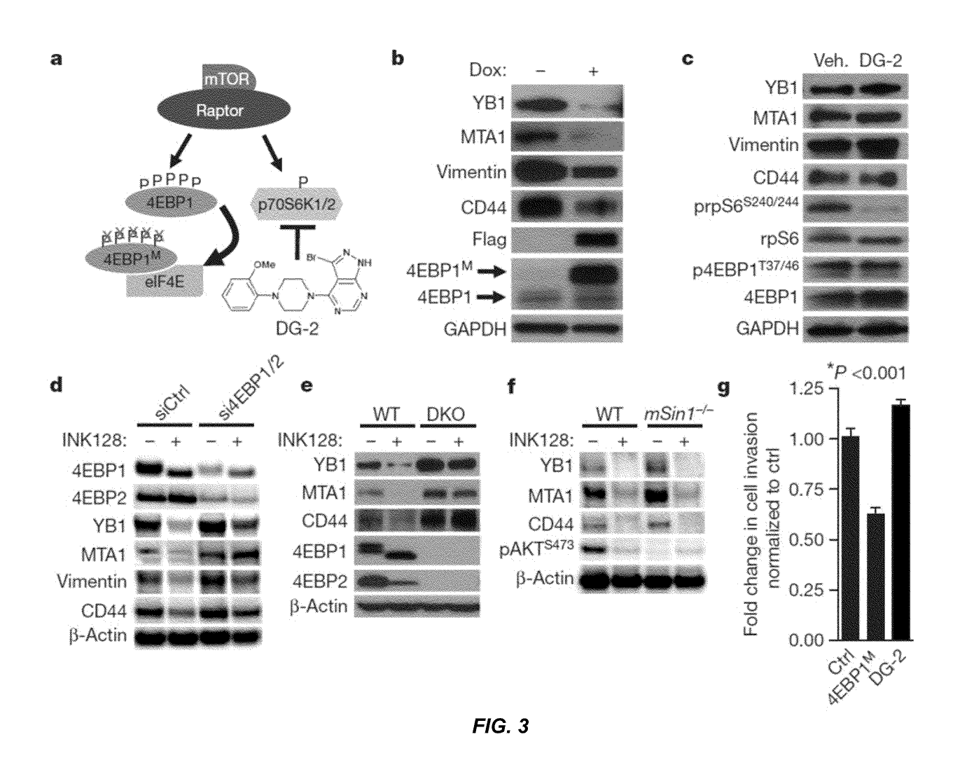

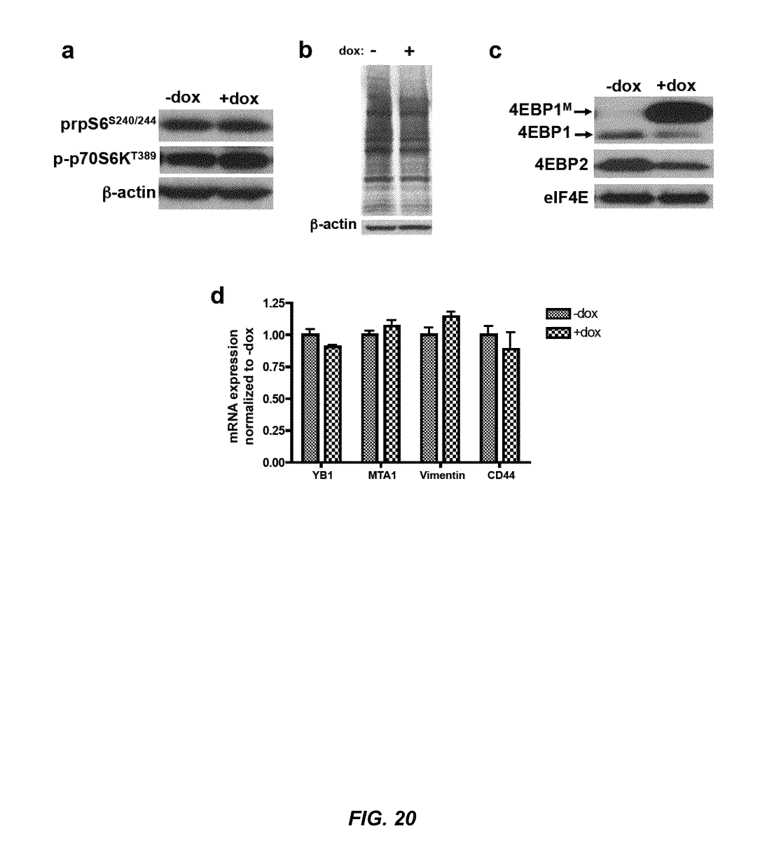

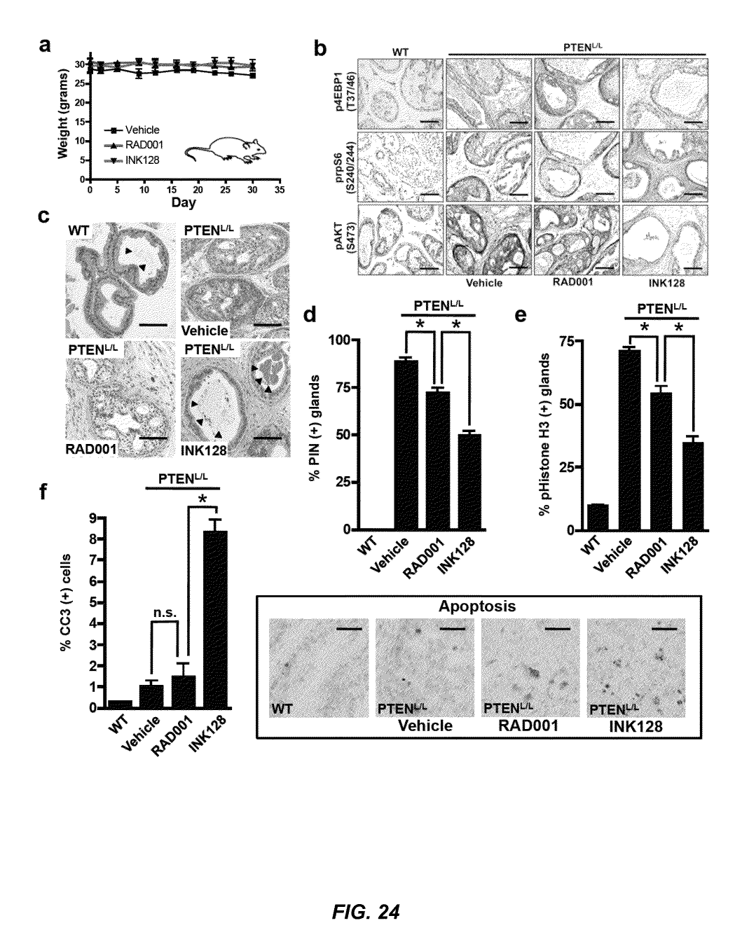

To determine the molecular mechanism by which pro-invasion genes are regulated at the translational level and why these mRNAs are sensitive to an ATP site inhibitor of mTOR but not rapamycin, we investigated whether the downstream translational regulators mTORC1, 4EBP1, and/or p70S6K1/2 controlled the expression of these mTOR-sensitive targets. A human prostate cancer cell line was generated that stably expressed a doxycycline-inducible dominant-negative mutant of 4EBP1 (4EBP1^{M}) (FIG. 3a) (Hsieh et al., Cancer Cell 17:249-261 (2010)). This mutant binds to eIF4E, decreasing its hyperactivation without inhibiting general mTORC1 function (FIG. 20a). Notably, expression of 4EBP1^{M} did not alter global protein synthesis (FIG. 20b), probably because endogenous 4EBP1 and 4EBP2 proteins retain their ability to bind to eIF4E (FIG. 24c). Upon induction of 4EBP1^{M}, YB1, vimentin, CD44 and MTA1 decreased at the protein but not mRNA level (FIGS. 3b-c and 24d).

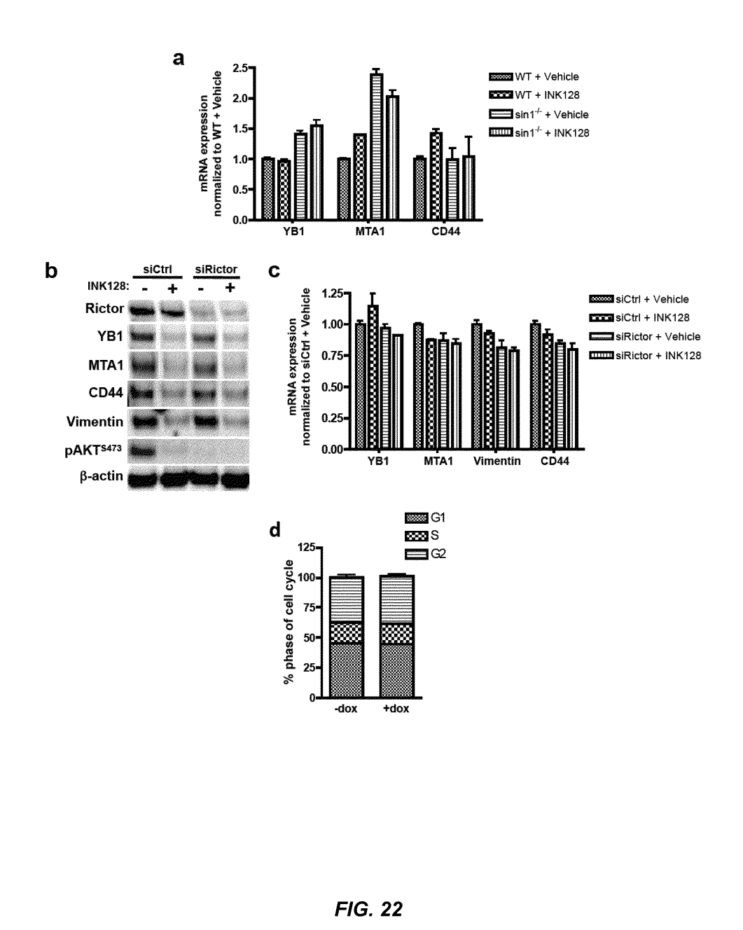

Next, we tested whether an ATP site inhibitor of mTOR decreases expression of the four invasion genes through the 4EBP-eIF4E axis. Notably, knockdown of 4EBP1 and 4EBP2 in PC3 cells or using 4EBP1 and 4EBP2 double knockout mouse embryonic fibroblasts (MEFs) (Dowling et al., Science 328:1172-1176 (2010)) reduced the ability of the ATP site inhibitor of mTOR to decrease expression of these pro-invasion mRNAs (FIGS. 3d-e and 21). Furthermore, ablation of mTORC2 activity had no effect on the expression of these mRNAs or responsiveness to ATP site inhibitor of mTOR (FIGS. 3f and 22a-c). Next, we determined the effect of 4EBP1^{M} on human prostate cancer cell invasion. The expression of 4EBP1^{M} resulted in a significant decrease in prostate cancer cell invasion without affecting the cell cycle, whereas DG-2 had no effect (FIGS. 3g and 22d). These findings demonstrate that eIF4E hyperactivation downstream of oncogenic mTOR regulates translational control of the pro-invasion mRNAs and provides an explanation for the selective targeting of this gene signature by mTOR ATP site inhibitors.

### Example 4

### Examining Cell Invasion Networks In Vivo

Both CK5⁺ and CK8⁺ prostate epithelial cells have been implicated in the initiation of prostate cancer upon loss of PTEN (Wang et al., Nature 461:495-500 (2009); Mulholland et al., Cancer Res. 69:8555-8562 (2009)). Pten^{loxp/loxp}; Pb-cre (Pten^{L/L}) mice are an ideal model of prostate cancer because they display distinct stages of cancer development (prostatic intraepithelial neoplasia, invasive adenocarcinoma, and metastasis) (Wang et al., Cancer Cell 4:209-221 (2003)). However, the expression patterns of YB1, vimentin, CD44 and MTA1 in prostate basal (CK5⁺) and luminal (CK8⁺) epithelial cells have not been characterized.

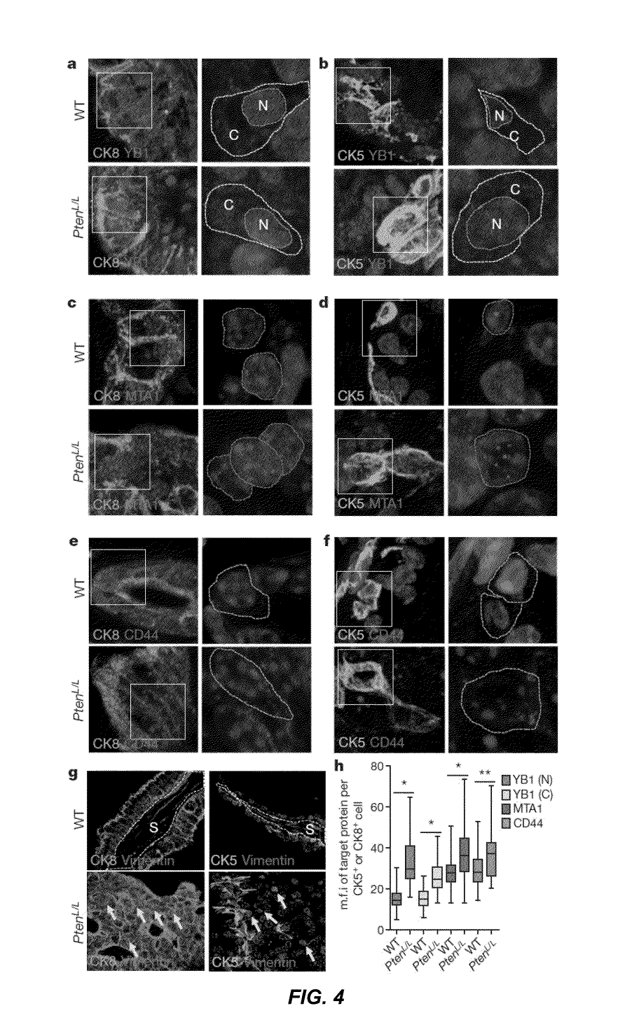

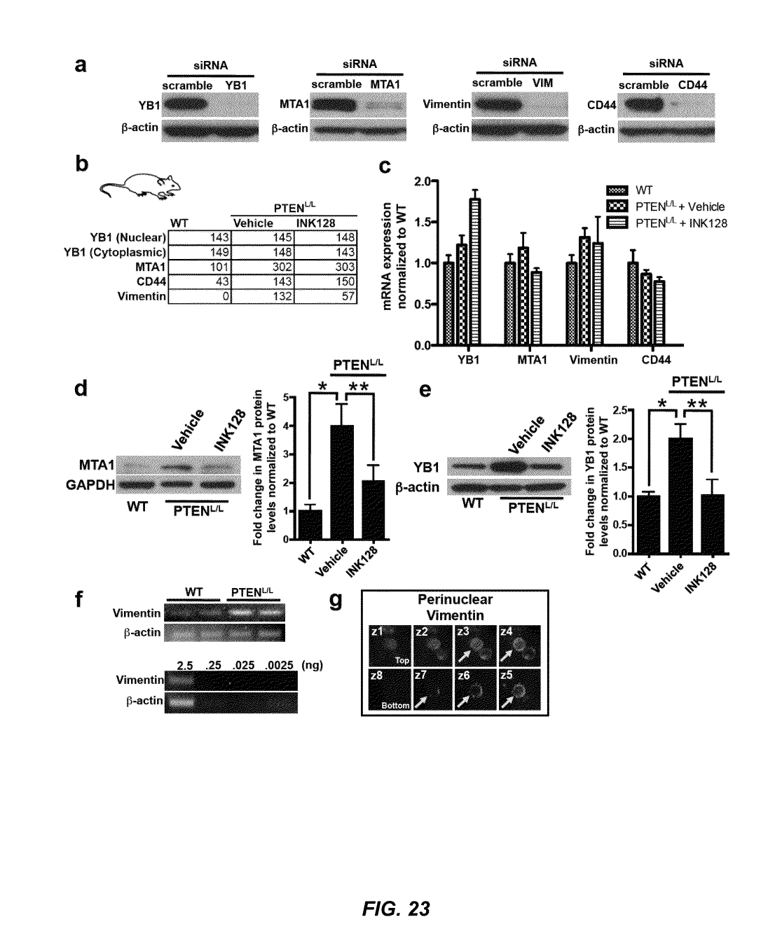

We therefore analyzed their expression patterns in the Pten^{L/L} prostate cancer mouse model, where mTOR is constitutively hyperactivated. YB1 localized to the cytoplasm and nucleus of CK5⁺ and CK8⁺ prostate epithelial cells, consistent with its ability to shuttle between the two cellular compartments (FIGS. 4a-b, 23a-b). MTA1 expression was exclusively nuclear in both cell types (FIG. 4c-d). CD44 expression was observed within a subset of CK5⁺ and CK8⁺ epithelial cells (FIG. 4e-f). CD44, together with other cell-surface markers, has been used to isolate a rare prostate stem-cell population (Leong et al., Nature 456:804-818 (2008)). In contrast, vimentin was not detected in either cell type (FIG. 4g). Next, the impact of mTOR hyperactivation on the expression pattern of the pro-invasion gene signature was determined. YB1, MTA1, and CD44 protein, but not transcript, levels were significantly increased in both Pten^{L/L} luminal and basal epithelial cells compared to wild-type (FIGS. 4h and 23c-e). These studies reveal a unique, translationally controlled signature of gene expression downstream of mTOR hyperactivation in a cancer-initiating subset of pro-state epithelial cells.

### Example 5

### Targeting Prostate Cancer Metastasis

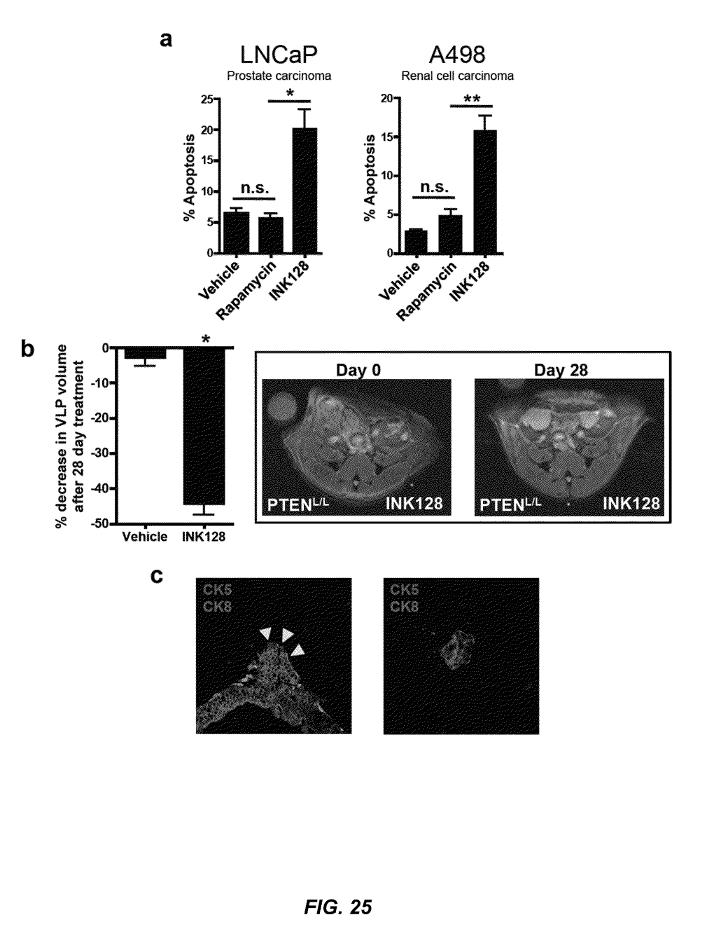

In a preclinical trial of RAD001 (rapalog) versus an ATP site inhibitor of mTOR in Pten^{L/L} mice, 4EBP1 and p70S6K1/2 phosphorylation was completely restored to wild-type levels after treatment with the ATP site inhibitor of mTOR, whereas RAD001 only decreased p70S6K1/2 phosphorylation levels (FIG. 24a-b). Next, the cellular consequences of complete versus partial mTOR inhibition during distinct stages of prostate cancer were determined. Treatment with the ATP site inhibitor of mTOR resulted in a 50% decrease in prostatic intraepithelial neoplasia (PIN) lesions in Pten^{L/L} mice that was associated with decreased proliferation and a tenfold increase in apoptosis (FIG. 24d-f). Notably, the unique cytotoxic properties of ATP site inhibitor of mTOR treatment in Pten^{L/L} mice were evidenced by a marked reduction in prostate cancer volume. In addition, and consistent with these findings, the ATP site inhibitor of mTOR induced programmed cell death in multiple cancer cell lines (FIG. 25a-b). In contrast, RAD001 treatment mainly had cytostatic effects leading to only partial regression of PIN lesions associated with a limited decrease in cell proliferation and no significant effect on apoptosis (FIG. 28c-f).

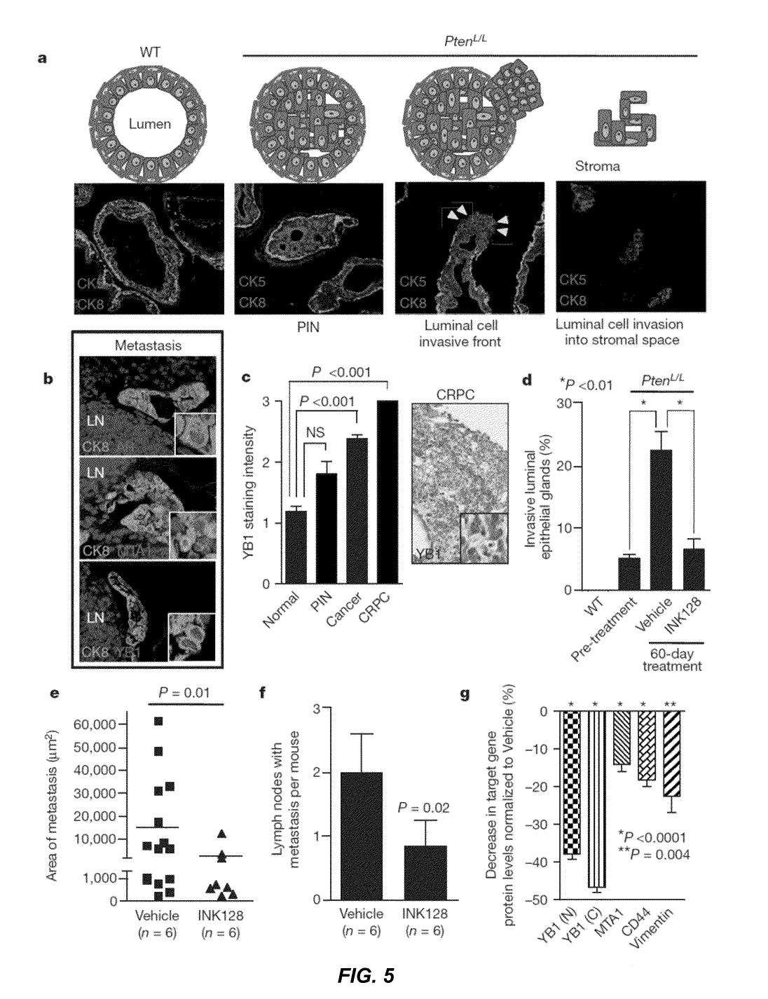

The preclinical trial was extended by examining the effects of the ATP site inhibitor of mTOR treatment on the pro-invasion gene signature and prostate cancer metastasis, which is incurable and the primary cause of patient mortality. Cell invasion is the critical first step in metastasis, required for systemic dissemination. In Pten^{L/L} mice after the onset of PIN, a subset of prostate glands showed characteristics of luminal epithelial cell invasion by 12 months (FIGS. 5a and 25c). After 12 months of age, Pten^{L/L} mice developed lymph-node metastases and these cells maintained strong YB1 and MTA1 expression (FIG. 5b). These findings were extended directly to human prostate cancer patient specimens, in which it was observed that YB1 expression levels increased in a stepwise fashion from normal prostate to castration-resistant prostate cancer (CRPC), an advanced form of the disease associated with increased metastatic potential (FIG. 5c). Similar increases have been observed in MTA1 levels (Hofer et al., Cancer Res. 64:825-829 (2004)).

In human prostate cancer, high-grade primary tumors that display invasive features are more likely to develop systemic metastasis than low-grade non-invasive tumors. Remarkably, treatment with the ATP site inhibitor of mTOR completely blocked the progression of invasive prostate cancer locally in the prostate gland, and profoundly inhibited the total number and size of distant metastases (FIG. 5d-f). This was associated with a marked decrease in the expression of YB1, vimentin, CD44, and MTA1 at the protein, but not transcript, level in specific epithelial cell types within pre-invasive PIN lesions in Pten^{L/L} mice (FIG. 5g and FIG. 23c). Together, these findings reveal an unexpected role for oncogenic mTOR signaling in control of a pro-invasion translational program that, along with the lethal metastatic form of prostate cancer, can be efficiently targeted with clinically relevant mTOR ATP site inhibitors. These findings also demonstrate that translational profiling can be used to identify or validate targets for therapeutic intervention, such as genes that are modulated in cancer.

### Example 6

### Methods

Mice.

Pten^{loxp/loxp} and Pb-cre mice where obtained from Jackson Laboratories and Mouse Models of Human Cancers Consortium (MMHCC), respectively, and maintained in the C57BL/6 background. Mice were maintained under specific pathogen-free conditions, and experiments were performed in compliance with institutional guidelines as approved by the Institutional Animal Care and Use Committee of UCSF.

Cell Culturing and Reagents.

Human cell lines were obtained from the ATCC and maintained in the appropriate medium with supplements as suggested by ATCC. Wild-type, mSin1^{-/-}, and 4EBP1/4EBP2 double knockout MEFs were cultured as previously described (Dowling et al., Science 328:1172-1176 (2010); Jacinto et al., Cell 127:125-137 (2006). SMARTvector 2.0 (Thermo Scientific) lentiviral shRNA constructs were used to knock down PTEN (SH-003023-02-10). For generation of GFP-labeled PC3 cells, SMARTvector 2.0 lentiviral empty vector control particles that contained TurboGFP (S-004000-01) were used. Control (D-001810-01), YB1 (L-010213), MTA1 (L-004127), CD44 (L-009999), vimentin (L-003551), rictor (LL-016984), 4EBP1 (L-003005), and 4EBP2 (L-018671) pooled siRNAs were purchased from Thermo Scientific. Intellikine provided the ATP site inhibitor of mTOR and PP242, which were used at 200 nM and 2.5 μM in cell-based assays unless otherwise specified. RAD001 was obtained from LC Laboratories. DG-2 was provided by K. Shokat and used at 20 μM in cell-based assays. Rapamycin was purchased from Calbiochem and used at 50 nM in cell-based assays. Doxycyline (Sigma) was used at 1 μg ml⁻¹ in 4EBP1^{M} induction assays. Lipofectamine 2000 (Invitrogen) was used to transfect cancer cell lines with siRNA. Amaxa Cell Line Nucleofector Kit R (Lonza) was used to electroporate BPH-1 cells with overexpression vectors. The 4EBP1^{M} has been previously described (Hsieh et al., Cancer Cell 17:249-261 (2010)).

Plasmids.

pcDNA3-HA-YB1 was provided by V. Evdokimova. pCMV6-Myk-DDK-MTA1 was purchased from Origene. pGL3-Promoter was purchased from Promega. To clone the 5′ UTR of YB1 into pGL3-Promoter, the entire 5′ UTR sequence of YB1 was amplified from PC3 cDNA. PCR fragments were digested with HindIII and NcoI and ligated into the corresponding sites of pGL3-Promoter. The PRTE sequence at position +20-34 in the YB1 5′ UTR (UCSC kgID uc001chs.2) was mutated using the QuikChange Site-Directed Mutagenesis Kit following the manufacturer's protocol (Stratagene).

Ribosome Profiling.

PC3 cells were treated with rapamycin (50 nM; Calbiochem) or PP242 (2.5 μM; Intellikine) for 3 hr. Cells were subsequently treated with cycloheximide (100 μg ml⁻¹; Sigma) and detergent lysis was performed in the dish. The lysate was treated with DNase and clarified, and a sample was taken for RNA-seq analysis. Lysates were subjected to ribosome footprinting by nuclease treatment. Ribosome-protected fragments were purified, and deep sequencing libraries were generated from these fragments, as well as from poly(A) mRNA purified from non-nuclease-treated lysates. These libraries were analyzed by sequencing on an Illumina GAII.

Each sequencing run resulted in approximately 20-25 million raw reads per sample, of which 5-12 million unique reads were used for subsequent analysis. Ribosome footprint and RNA-seq sequencing reads were aligned against a library of transcripts from the UCSC Known Genes database GRCh37/hg19. The first 25 nucleotides of each read were aligned using Bowtie and this initial alignment was then extended to encompass the full fragment-derived portion of the sequencing read while excluding the linker sequence. Read density profiles were then constructed for the canonical transcript of each gene, using only reads with 0 or 1 total mismatches between the read sequence and the reference sequence, comprised of the transcript fragment followed by the linker sequence. Footprint reads were assigned to an A site nucleotide at position +15 to +17 of the alignment, based on the total fragment length; mRNA reads were assigned to the first nucleotide of the alignment. The average read density per codon was then computed for the coding sequence of each transcript, excluding the first 15 and last 5 codons, which can display atypical ribosome accumulation.

Average read density was used as a measure of mRNA abundance (RNA-seq reads) and of protein synthesis (ribosome profiling reads). For most analyses, genes were filtered to require at least 256 reads in the relevant RNA-seq samples. Translational efficiency was computed as the ratio of ribosome footprint read density to RNA-seq read density, scaled to normalize the translational efficiency of the median gene to 1.0 after excluding regulated genes (log₂ fold-change ±1.5 after normalizing for the all-gene median). Changes in protein synthesis, mRNA abundance and translational efficiency were similarly computed as the ratio of read densities between different samples, normalized to give the median gene a ratio of 1.0. This normalization corrects for differences in the absolute number of sequencing reads obtained for different libraries. 3,977 (replicate 1), and 5,333 (replicate 2) unique mRNAs passed a preset read threshold of 256 reads for single-gene quantification for all treatment conditions.

Western Blot Analysis.

Western blot analysis was performed as previously described (Hsieh et al., Cancer Cell 17:249-261 (2010)) with antibodies specific to phospho-AKT^{S473} (Cell Signaling), AKT (Cell Signaling), phospho-p70S6K^{T389} (Cell Signaling), phospho-rpS6^{S240/244} (Cell Signaling), rpS6 (Cell Signaling), phospho-4EBP1^{T37/46} (Cell Signaling), 4EBP1 (Cell Signaling), 4EBP2 (Cell Signaling), YB1 (Cell Signaling), CD44 (Cell Signaling), LEF1 (Cell Signaling), PTEN (Cell Signaling), eEF2 (Cell Signaling), GAPDH (Cell Signaling), vimentin (BD Biosciences), eIF4E (BD Biosciences), Flag (Sigma), β-actin (Sigma), MTA1 (Santa Cruz Biotechnology), Twist (Santa Cruz Biotechnology), rpL28 (Santa Cruz Biotechnology), HA (Covance) and rictor (Bethyl Laboratory).

qPCR Analysis.

RNA was isolated using the manufacturer's protocol for RNA extraction with TRIzol Reagent (Invitrogen) using the Pure Link RNA mini kit (Invitrogen). RNA was DNase-treated with Pure Link Dnase (Invitrogen). DNase-treated RNA was transcribed to cDNA with SuperScript III First-Strand Synthesis System for RT-PCR (Invitrogen), and 1 μl of cDNA was used to run a SYBR green detection qPCR assay (SYBR Green Supermix and MyiQ2, Biorad). Primers were used at 200 nM.

5′ UTR Analysis.

5′ UTRs of the 144 downregulated mTOR target genes were obtained using the known gene ID from the UCSC Genome Browser (GRCh37/hg19). Target versus non-target mRNAs were compared for 5′ UTR length, % G+C content and Gibbs free energy by the Wilcoxon two-sided test. Multiple Eₘ (expectation maximization) for Motif Elicitation (MEME) and Find Individual Motif Occurrences (FIMO) was used to derive the PRTE and determine its enrichment in the 144 mTOR-sensitive genes compared a background list of 3,000 genes. The Database of Transcriptional Start Sites (DBTSS Release 8.0) was used to identify putative 5′ TOP genes and putative transcription start sites in the 144 mTOR target genes.

Luciferase Assay.

PC3 4EBP1^{M} cells were treated with 1 μg ml⁻¹ doxycycline (Sigma) for 24 hr. Cells were transfected with various pGL3-Promoter constructs using lipofectamine 2000 (Invitrogen). After 24 hr, cells were collected. 20% of the cells were aliquoted for RNA isolation. The remaining cells were used for the luciferase assay per the manufacturer's protocol (Promega). Samples were measured for luciferase activity on a Glomax 96-well plate luminometer (Promega). Firefly luciferase activity was normalized to luciferase mRNA expression levels.

Kinase Assays.

mTOR activity was assayed using LanthaScreen Kinase kit reagents (Invitrogen) according to the manufacturer's protocol. PI(3)K α, β, γ, and δ activity were assayed using the PI(3)K HTRF assay kit (Millipore) according to the manufacturer's protocol. The concentration of ATP site inhibitor of mTOR necessary to achieve inhibition of enzyme activity by 50% (IC₅₀) was calculated using concentrations ranging from 20 μM to 0.1 nM (12-point curve). IC₅₀ values were determined using a nonlinear regression model (GraphPad Prism 5).

Cell Proliferation Assay.

PC3 cells were treated with the appropriate drug for 48 hr, and proliferation was measured using Cell Titer-Glo Luminescent reagent (Promega) per the manufacturer's protocol. The concentration of ATP site inhibitor of mTOR necessary to achieve inhibition of cell growth by 50% (IC₅₀) was calculated using concentrations ranging from 20.0 μM to 0.1 nM (12-point curve).

Mouse Xenograft Study.

Nude mice were inoculated subcutaneously in the right subscapular region with 5×10⁶ MDA-MB-361 cells. After tumors reached a size of 150-200 mm³, mice were randomly assigned into vehicle control or treatment groups. The ATP site inhibitor of mTOR was formulated in 5% polyvinylpropyline, 15% NMP, 80% water and administered by oral gavage at 0.3 mg kg⁻¹ and 1 mg kg⁻¹ daily.

Pharmacokinetic Analysis.

The area under the plasma drug concentration versus time curves, AUC₍₀₋ₜₗₐₛₜ₎ and AUC_{(0-inf)}, were calculated from concentration data using the linear trapezoidal rule. The terminal t_{1/2} in plasma was calculated from the elimination rate constant (lz), estimated as the slope of the log-linear terminal portion of the plasma concentration versus time curve, by linear regression analysis. The bioavailability (F) was calculated using F=AUC_{(0-tlast),po}D_{i.v.})/AUC_{(0-last),iv}D_{p.o.})×100%, where D_{i.v.} and D_{p.o.} are intravenous and oral doses, respectively. Cₘₐₓ was a highest drug concentration in plasma after oral administration. Tₘₐₓ was the time at which Cₘₐₓ is observed after extravascular administration of drug. Tₗₐₛₜ was the last time point a quantifiable drug concentration can be measured.

Metabolic Stability Assay.

In vitro metabolic stability of the ATP site inhibitor of mTOR was evaluated after incubation with liver microsomes or liver S9 fractions from various species in the presence of NADPH. The half-life of the ATP site inhibitor of mTOR was estimated by log linear regression analysis.

CYP Assay.

The ATP site inhibitor of mTOR inhibition of CYP450 isoforms in human liver microsomes was determined with isoform-specific substrates at concentrations approximately equal to the concentration at which the rate of the reaction is half-maximal (Kₘ) for the individual isoforms: CYP1A2, CYP2C8, CYP2C9, CYP2C19, CYP2D6 and CYP3A4.

Pharmaceutical Property Assays.

The percentage of protein binding of the ATP site inhibitor of mTOR was determined in mouse, rat, dog, monkey, and human plasma. The IC₅₀ for the inhibitory effect of the ATP site inhibitor of mTOR on hERG potassium channel was determined. A Bacterial Reverse Mutation Assay (Ames test) was conducted at BioReliance.

Polysome Analysis.

PC3 cells were treated for 3 hr with either DMSO or the ATP site inhibitor of mTOR (100 nM). Cells were re-suspended in PBS containing 100 μml⁻¹ cycloheximide (Sigma) and incubated on ice for 10 min. Cells were centrifuged at 300 g for 5 min at 4° C. and lysed in 10 mM Tris-HCl pH 8, 140 mM NaCl, 5 mM MgCl₂, 640 U ml⁻¹ Rnasin, 0.05% NP-40, 250 μg ml⁻¹ cycloheximide, 20 mM DTT, and protease inhibitors. Samples were incubated for 20 min on ice, then centrifuged once for 5 min at 3,300 g and once for 5 min at 9,300 g, isolating the supernatant after each centrifugation. Lysates were loaded onto 10-50% sucrose gradients containing 0.1 mg ml⁻¹ heparin and 2 mM DTT and centrifuged at 37,000 r.p.m. for 2.5 hr at 4° C. The sample was subsequently fractionated on a gradient fractionation system (ISCO). RNA was extracted from all fractions and run on a TBE-agarose gel to visualize 18S and 28S rRNA. Fractions 7-13 were found to correspond to the polysome fractions and were used for further qPCR analysis.

[³⁵S] metabolic labeling. PC3 or PC3 4EBP1^{M} cells with or without indicated treatment were incubated with 30 μCi of [³⁵S]-methionine for 1 hr after pre-incubation in methionine-free DMEM (Invitrogen). Cells were prepared using a standard protein lysate protocol, resolved on a 10% SDS polyacrylamide gel and transferred onto a PVDF membrane (Bio-Rad). The membrane was exposed to autoradiography film (Denville) for 24 hr and developed.

Cell Cycle Analysis.

Appropriately treated PC3, BPH-1, or PC3-4EBP1^{M} cells were fixed in 70% ethanol overnight at −20° C. Cells were subsequently washed with PBS and treated with RNase (Roche) for 30 min. After this incubation, the cells were permeabilized and treated with 50 μg ml⁻¹ propidium iodide (Sigma) in a solution of 0.1% Tween, 0.1% sodium citrate. Cell cycle data was acquired using a BD FACS Caliber (BD Biosciences) and analyzed with FlowJo (v.9.1).

Apoptosis analysis. Appropriately treated LNCaP and A498 cells were labeled with Annexin V-FITC (BD Biosciences) and propidium iodide (Sigma) following the manufacturer's instructions. PI/Annexin data was acquired using a BD FACS Caliber (BD Biosciences) and analyzed with FlowJo (v.9.1).

Matrigel Invasion Assay.

BioCoat Matrigel Invasion Chambers (modified Boyden Chamber Assay; BD Biosciences) were used according to the manufacturer's instructions.

Real-Time Imaging of Cell Migration.

Real-time imaging of GFP-labeled PC3 cells was performed in poly-D-lysine-coated chamber cover glass slides (Lab-Tek). PC3 GFP cells were plated and allowed to adhere for 24 hr. Wells were wounded with a P200 pipette tip. The chamber slides were imaged with an IX81 Olympus wide-field fluorescence microscope equipped with a CO₂- and temperature-controlled chamber and time-lapse tracking system. Images from DIC and GFP channels were taken every 2 min and processed using ImageJ and analyzed for cell migration with Manual Tracking, using local maximum centering correction to maintain a centroid xy coordinate for each cell per frame over time. Tracking data was subsequently processed with the Chemotaxis and Migration tool from ibidi to create xy coordinate plots, velocity, and distance measurements.

Snail1 Immunocytochemistry.

Appropriately transfected or treated PC3 cells were plated on a poly-L-lysine-coated chamber slide (Lab-Tek) and cultured for 48 hr. Cells were fixed with 4% paraformaldehyde (EMS), rinsed with PBS, and permeabilized with 0.1% Triton X-100. The samples were blocked in 5% goat serum and then incubated with anti-Snail1 antibody (Cell Signaling) in 5% goat serum for 2 hr at room temperature. Cells were washed with PBS and incubated with Alexa 594 anti-mouse antibody (Invitrogen) and DAPI (Invitrogen) for 2 hr at room temperature. Specimens were again washed with PBS and subsequently mounted with Aqua Poly/Mount (Polysciences). Image capture and quantification were completed as described below (see “Immunofluorescence”).

Cap-binding assay. PC3 4EBP1^{M} cells were induced with doxycycline (1 μg ml⁻¹, Sigma) for 48 hr, then collected and lysed in buffer A (10 mM Tris-HCl pH 7.6, 150 mM KCl, 4 mM MgCl₂, 1 mM DTT, 1 mM EDTA, and protease inhibitors, supplemented with 1% NP-40). Cell lysates were incubated overnight at 4° C. with 50 ml of the mRNA cap analogue m⁷GTP-sepharose (GE Healthcare) in buffer A. The beads were washed with buffer A supplemented with 0.5% NP-40. Protein complexes were dissociated using 1× sample buffer, and resolved by SDS-PAGE and western blotted with the appropriate antibodies.

Pharmacological Treatment of Pten^{L/L} Mice and MRI Imaging.

Nine- and twelve-month-old Pten^{L/L} mice were averaged daily with either vehicle (see “Mouse xenograft study”), RAD001 (10 mg kg⁻¹; LC Laboratories), or an ATP site inhibitor of mTOR (1 mg kg⁻¹; Intellikine) for the indicated times. Weight measurements were taken every 3 days to monitor for toxicity. For the 28-day study, mice were imaged via MRI at day 0 and day 28 in a 14-T GE MR scanner (GE Healthcare).

Prostate Tissue Processing.

Whole mouse prostates were removed from wild-type and Pten^{L/L} mice, microdissected, and frozen in liquid nitrogen. Frozen tissues were subsequently manually disassociated using a biopulverizer (Biospec) and additionally processed for protein and mRNA analysis as described above.

Immunofluorescence.

Prostates and lymph nodes were dissected from mice within 2 hr of the indicated treatment and fixed in 10% formalin overnight at 4° C. Tissues were subsequently dehydrated in ethanol (Sigma) at room temperature, mounted into paraffin blocks, and sectioned at 5 μm. Specimens were de-paraffinized and rehydrated using CitriSolv (Fisher) followed by serial ethanol washes. Antigen unmasking was performed on each section using Citrate pH 6 (Vector Labs) in a pressure cooker at 125° C. for 10-30 min. Sections were washed in distilled water followed by TBS washes. The sections were then incubated in 5% goat serum, 1% BSA in TBS for 1 hr at room temperature. Various primary antibodies were used, including those specific for keratin 5 (Covance), cytokeratin 8 (Abcam and Covance), YB1 (Abcam), vimentin (Abcam), MTA1 (Cell signaling), CD44 (BD Pharmingen), and the androgen receptor (Epitomics), which were diluted 1:50-1:500 in blocking solution and incubated on sections overnight at 4° C. Specimens were then washed in TBS and incubated with the appropriate Alexa 488 and 594 labeled secondary (Invitrogen) at 1:500 for 2 hr at room temperature, with the exception of YB1 which was incubated with biotinylated anti-rabbit secondary (Vector) followed by incubation with Alexa 594 labeled Streptavidin (Invitrogen). A final set of washes in TBS was completed at room temperature followed by mounting with DAPI Hardset Mounting Medium (Vector Lab). A Zeiss Spinning Disc confocal (Zeiss, CSU-X1) was used to image the sections at 40×-100×. Individual prostate cells were quantified for mean fluorescence intensity (m.f.i.) using the Axiovision (Zeiss, Release 4.8) densitometric tool.

Lymph Node Metastasis Measurements.

Mouse lymph nodes were processed as described above and stained for CK8 and androgen receptor. Lymph nodes were imaged using a Zeiss AX10 microscope. Metastases were identified and areas were measured using the Axiovision (Zeiss, Release 4.8) measurement tool.

Semi-Quantitative RT-PCR.

Whole prostates were removed from wild-type and Pten^{L/L} mice, microdissected, dissociated into single-cell suspension, and stained for epithelial cell markers as previously described (Lukacs et al., Nature Protocols 5:702-713 (2010)) using fluorescence-conjugated antibodies for CD49f, Sca-1, CD31, CD45, and Ter119 (BD Biosciences). Luminal epithelial cells were sorted using a FACS Aria (BD Biosciences). Cell pellets were resuspended in 500 μl TRIzol Reagent and RNA was isolated and transcribed into cDNA as described above. Semi-quantitative PCR analysis was performed using oligonucleotides for vimentin and β-actin at 200 nM in a 25 μl reaction with 12.5 μl GoTaq (Promega) for 32 and 33 cycles, respectively, which were within the linear range (FIG. 230.

Immunohistochemistry.

Immunohistochemistry was performed as described above (see “Immunofluorescence”) with the exception that immediately after antigen presentation and TBS washes, specimens were incubated in 3% hydrogen peroxide in TBS followed by TBS washes. The following primary antibodies were used: phospho-AKT^{S473} (Cell Signaling), phospho-rpS6^{S240/244} (Cell Signaling), phospho-4EBP1^{T37/46} (Cell Signaling), phospho-histone H3 (Upstate), and cleaved caspase (Cell Signaling). This was followed by TBS washes and incubation with the appropriate biotinylated secondary antibody (Vector Lab) for 30 min at room temperature. An ABC-HRP Kit (Vector Lab) was used to amplify the signal, followed by a brief incubation in hydrogen peroxide. The protein of interest was detected using DAB (Sigma). Specimens were counterstained with haematoxylin (Thermo Scientific), dehydrated with Citrisolv (Fisher), and mounted with Cytoseal XYL (Vector Lab).

Haematoxylin and Eosin Staining.

Paraffin-embedded prostate specimens were deparaffinized and rehydrated as described above (see “Immunofluorescence”), stained with haematoxylin (Thermo Scientific), and washed with water. This was followed by a brief incubation in differentiation RTU (VWR) and two washes with water followed by two 70% ethanol washes. The samples were then stained with eosin (Thermo Scientific) and dehydrated with ethanol followed by CitriSolv (Fisher). Slides were mounted with Cytoseal XYL (Richard Allan Scientific).

Oligonucleotides.

YB1 5′ UTR cloning and site-directed mutagenesis oligonucleotides are as follows. YB1 5′ UTR cloning: forward 5′-GCTACAAGCTTGGGCTTATCCCGCCT-3′ (SEQ ID NO:146), reverse 5′-TCGATCCATGGGGTTGCGGTGATGGT-3′ (SEQ ID NO:147); deletion (20-34): forward 5′-TGGGCTTATCCCGCCTGTCCTTCGATCGGTAGCGGGAGCG-3′ (SEQ ID NO:148), reverse 5′-CGCTCCCGCTACCGATCGAAGGACAGGCGGGATAAGCCCA-3′ (SEQ ID NO:149); transversion (20-34): forward 5′-TGGGCTTATCCCGCCTGTCCGCGGTAAGAGCGATCTTCGATCGGTAGCGGGAGCG-3′ (SEQ ID NO:150), reverse 5′-CGCTCCCGCTACCGATCGAAGATCGCTCTTACCGCGGACAGGCGGGATAAGCCCA-3′ (SEQ ID NO:151).

Human qPCR oligonucleotides are as follows. β-actin forward 5′-GCAAAGACCTGTACGCCAAC-3′ (SEQ ID NO:152), reverse 5′-AGTACTTGCGCTCAGGAGGA-3′ (SEQ ID NO:153); CD44 forward 5′-CAACAACACAAATGGCTGGT-3′ (SEQ ID NO:154), reverse 5′-CTGAGGTGTCTGTCTCTTTCATCT-3′ (SEQ ID NO:155); vimentin forward 5′-GGCCCAGCTGTAAGTTGGTA-3′ (SEQ ID NO:156), reverse 5′-GGAGCGAGAGTGGCAGAG-3′ (SEQ ID NO:157); Snail1 forward 5′-CACTATGCCGCGCTCTTTC-3′ (SEQ ID NO:158), reverse 5′-GCTGGAAGGTAAACTCTGGATTAGA-3′ (SEQ ID NO:159); YB1 forward 5′-TCGCCAAAGACAGCCTAGAGA-3′ (SEQ ID NO:160), reverse 5′-TCTGCGTCGGTAATTGAAGTTG-3′ (SEQ ID NO:161); MTA1 forward 5′-CAAAGTGGTGTGCTTCTACCG-3′ (SEQ ID NO:162), reverse 5′-CGGCCTTATAGCAGACTGACA-3′ (SEQ ID NO:163); PLAU forward 5′-TTGCTCACCACAACGACATT-3′ (SEQ ID NO:164), reverse 5′-GGCAGGCAGATGGTCTGTAT-3′ (SEQ ID NO:165); FGFBP1 forward 5′-ACTGGATCCGTGTGCTCAG-3′ (SEQ ID NO:166), reverse 5′-GAGCAGGGTGAGGCTACAGA-3′ (SEQ ID NO:167); ARID5B forward 5′-TGGACTCAACTTCAAAGACGTTC-3′ (SEQ ID NO:168), reverse 5′-ACGTTCGTTTCTTCCTCGTC-3′ (SEQ ID NO:169); CTGF forward 5′-CTCCTGCAGGCTAGAGAAGC-3′ (SEQ ID NO:170), reverse 5′-GATGCACTTTTTGCCCTTCTT-3′ (SEQ ID NO:171); RND3 forward 5′-AAAAACTGCGCTGCTCCAT-3′ (SEQ ID NO:172), reverse 5′-TCAAAACTGGCCGTGTAATTC-3′ (SEQ ID NO:173); KLF6 forward 5′-AAAGCTCCCACTTGAAAGCA-3′ (SEQ ID NO:174), reverse 5′-CCTTCCCATGAGCATCTGTAA-3′ (SEQ ID NO:175); BCL6 forward 5′-TTCCGCTACAAGGGCAAC-3′ (SEQ ID NO:176), reverse 5′-TGCAACGATAGGGTTTCTCA-3′ (SEQ ID NO:177); FOXA1 forward 5′-AGGGCTGGATGGTTGTATTG-3′ (SEQ ID NO:178), reverse 5′-ACCGGGACGGAGGAGTAG-3′ (SEQ ID NO:179); GDF15 forward 5′-CCGGATACTCACGCCAGA-3′ (SEQ ID NO:180), reverse 5′-AGAGATACGCAGGTGCAGGT-3′ (SEQ ID NO:181); HBP1 forward 5′-GCTGGTGGTGTTGTCGTG-3′ (SEQ ID NO:182), reverse 5′-CATGTTATGGTGCTCTGACTGC-3′ (SEQ ID NO:183); Twist1 forward 5′-CATCCTCACACCTCTGCATT-3′ (SEQ ID NO:184), reverse 5′-TTCCTTTCAGTGGCTGATTG-3′ (SEQ ID NO:185); LEF1 forward 5′-CCTTGGTGAACGAGTCTGAAATC-3′ (SEQ ID NO:186), reverse 5′-GAGGTTTGTGCTTGTCTGGC-3′ (SEQ ID NO:187); rpS19 forward 5′-GCTGGCCAAACATAAAGAGC-3′ (SEQ ID NO:188), reverse 5′-CTGGGTCTGACACCGTTTCT-3′ (SEQ ID NO:189); 5S rRNA forward 5′-GCCCGATCTCGTCTGATCT-3′ (SEQ ID NO:190), reverse 5′-AGCCTACAGCACCCGGTATT-3′ (SEQ ID NO:191); firefly luciferase forward 5′-AATCAAAGAGGCGAACTGTG-3′ (SEQ ID NO:192), reverse 5′-TTCGTCTTCGTCCCAGTAAG-3′ (SEQ ID NO:193).

Mouse qPCR oligonucleotides are as follows. β-actin forward 5′-CTAAGGCCAACCGTGAAAAG-3′ (SEQ ID NO:194), reverse 5′-ACCAGAGGCATACAGGGACA-3′ (SEQ ID NO:195); Yb1 forward 5′-GGGTTACAGACCACGATTCC-3′ (SEQ ID NO:196), reverse 5′-GGCGATACCGACGTTGAG-3′ (SEQ ID NO:197); vimentin forward 5′-TCCAGCAGCTTCCTGTAGGT-3′ (SEQ ID NO:198), reverse 5′-CCCTCACCTGTGAAGTGGAT-3′ (SEQ ID NO:199); Cd44 forward 5′-ACAGTACCTTACCCACCATG-3′ (SEQ ID NO:200), reverse 5′-GGATGAATCCTCGGAATTAC-3′ (SEQ ID NO:201); Mta1 forward 5′-AGTGCGCCTAATCCGTGGTG-3′ (SEQ ID NO:202), reverse 5′-CTGAGGATGAGAGCAGCTTTCG-3′ (SEQ ID NO:203).

siRNA/shRNA sequences are as follows. Control (D-001810-01) 5′-UGGUUUACAUGUCGACUAA-3′ (SEQ ID NO:204); vimentin (L-003551) 5′-UCACGAUGACCUUGAAUAA-3′ (SEQ ID NO:205), 5′-GGAAAUGGCUCGUCACCUU-3′ (SEQ ID NO:206), 5′-GAGGGAAACUAAUCUGGAU-3′ (SEQ ID NO:207), 5′-UUAAGACGGUUGAAACUAG-3′ (SEQ ID NO:208); YB1 (L-010213) 5′-CUGAGUAAAUGCCGGCUUA-3′ (SEQ ID NO:209), 5′-CGACGCAGACGCCCAGAAA-3′ (SEQ ID NO:210), 5′-GUAAGGAACGGAUAUGGUU-3′ (SEQ ID NO:211), 5′-GCGGAGGCAGCAAAUGUUA-3′ (SEQ ID NO:212); MTA1 (L-004127) 5′-UCACGGACAUUCAGCAAGA-3′ (SEQ ID NO:213), 5′-GGACCAAACCGCAGUAACA-3′ (SEQ ID NO:214), 5′-GCAUCUUGUUGGACAUAUU-3′ (SEQ ID NO:215), 5′-CCAGCAUCAUUGAGUACUA-3′ (SEQ ID NO:216); CD44 (L-009999) 5′-GAAUAUAACCUGCCGCUUU-3′ (SEQ ID NO:217), 5′-CAAGUGGACUCAACGGAGA-3′(SEQ ID NO:218), 5′-CGAAGAAGGUGUGGGCAGA-3′ (SEQ ID NO:219), 5′-GAUCAACAGUGGCAAUGGA-3′ (SEQ ID NO:220); 4EBP1 (L-003005) 5′-CUGAUGGAGUGUCGGAACU-3′ (SEQ ID NO:221), 5′-CAUCUAUGACCGGAAAUUC-3′ (SEQ ID NO:222), 5′-GCAAUAGCCCAGAAGAUAA-3′ (SEQ ID NO:223), 5′-GAGAUGGACAUUUAAAGCA-3′ (SEQ ID NO:224); 4EBP2 (L-018671) 5′-GCAGCUACCUCAUGACUAU-3′ (SEQ ID NO:225), 5′-GGAGGAACUCGAAUCAUUU-3′ (SEQ ID NO:226), 5′-GCAAUUCUCCCAUGGCUCA-3′ (SEQ ID NO:227), 5′-UUGAACAACUUGAACAAUC-3′ (SEQ ID NO:228); rictor (LL-016984) 5′-GACACAAGCACUUCGAUUA-3′ (SEQ ID NO:229), 5′-GAAGAUUUAUUGAGUCCUA-3′ (SEQ ID NO:230), 5′-GCGAGCUGAUGUAGAAUUA-3′ (SEQ ID NO:231), 5′-GGGAAUACAACUCCAAAUA-3′ (SEQ ID NO:232); PTEN SH-003023-01-10 5′-GCTAAGAGAGGTTTCCGAA-3′ (SEQ ID NO:233), SH-003023-02-10 5′-AGACTGATGTGTATACGTA-3′ (SEQ ID NO:234).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for identifying an agent that modulates an oncogenic signaling pathway in a biological sample, the method comprising:
(a) contacting the biological sample with an agent;
(b) determining a first translational profile for the contacted biological sample, wherein the translational profile comprises translational levels for one or more genes having a 5′ terminal oligopyrimidine tract (5′ TOP) and/or a pyrimidine-rich translational element (PRTE); and
(c) comparing the first translational profile to a second translational profile comprising translational levels for the one or more genes in a control sample that has not been contacted with the agent;
wherein a difference in the translational levels of the one or more genes in the first translation profile as compared to the second translation profile identifies the agent as a modulator of the oncogenic signaling pathway.

2. The method of claim 1, wherein the one or more genes are selected from the genes listed in Table 1, Table 2, and/or Table 3.

3. The method of claim 1, wherein the one or more genes are cell invasion and/or metastasis genes.

4. The method of claim 1, wherein the one or more genes are selected from Y-box binding protein 1 (YB1), vimentin, metastasis associated 1 (MTA1), and CD44.

5. A method for identifying an agent that modulates an oncogenic signaling pathway in a biological sample, the method comprising:
(a) contacting the biological sample with an agent;
(b) determining a first translational profile for the contacted biological sample, wherein the translational profile comprises translational levels for one or more genes selected from the group consisting of SEQ ID NOs:1-144; and
(c) comparing the first translational profile to a second translational profile comprising translational levels for the one or more genes in a control sample that has not been contacted with the agent;
wherein a difference in the translational levels of the one or more genes in the first translation profile as compared to the second translation profile identifies the agent as a modulator of the oncogenic signaling pathway.

6. The method of claim 1, wherein the oncogenic signaling pathway is the mammalian target of rapamycin (mTOR) pathway, the PI3K pathway, the AKT pathway, the Ras pathway, the Myc pathway, the Wnt pathway, or the BRAF pathway.

7. The method of claim 1, wherein the oncogenic signaling pathway is the mTOR pathway.

8. The method of claim 1, wherein the translational level for the one or more genes is decreased for the first translational profile as compared to the second translational profile, thereby identifying the agent as an inhibitor of the oncogenic signaling pathway.

9. (canceled)

10. The method of claim 1, wherein the translational level for the one or more genes is increased for the first translational profile as compared to the second translational profile, thereby identifying the agent as a potentiator of the oncogenic signaling pathway.

11. (canceled)

12. The method of claim 1, wherein the first and/or second translational profiles are generated using ribosomal profiling, polysome microarray, immunoassay, or a combination thereof.

13-14. (canceled)

15. The method of claim 1, wherein the first and/or second translation profile comprises measuring the translational levels of at least 500 genes in the sample or comprises a genome-wide measurement of gene translational levels.

16. (canceled)

17. The method of claim 1, wherein the biological sample comprises a human cell.

18-19. (canceled)

20. The method of claim 17, wherein the cell is a cancer cell from prostate cancer, breast cancer, bladder cancer, lung cancer, renal cell carcinoma, endometrial cancer, melanoma, ovarian cancer, thyroid cancer, or brain cancer.

21. The method of claim 1, wherein the identified agent binds to a 5′ TOP or PRTE sequence in the one or more genes having a different translational level in the first translational profile as compared to the second translational profile.

22. The method of claim 1, wherein the identified agent inhibits the activity of a downstream effector of the oncogenic signaling pathway, wherein the effector is 4EBP1, p70S6K1/2, or AKT.

23. The method of claim 1, further comprising chemically synthesizing a structurally related agent derived from the identified agent.

24-26. (canceled)

27. A method of validating a target for therapeutic intervention, the method comprising:
(a) contacting a biological sample with an agent that modulates the target;
(b) determining a first translational profile for the contacted biological sample, wherein the first translational profile comprises translational levels for a plurality of genes; and
(c) comparing the first translational profile to a second translational profile comprising translational levels for the plurality of genes in a control sample that has not been contacted with the agent;
wherein identifying one or more genes of a biological pathway as differentially translated in the first translational profile as compared to the second translational profile validates the target for therapeutic intervention, wherein said biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway.

28. The method of claim 27, wherein the one or more genes have a 5′ terminal oligopyrimidine tract (5′ TOP) and/or a pyrimidine-rich translational element (PRTE).

29. The method of claim 27, wherein the one or more genes are selected from the group consisting of SEQ ID NOs:1-144.

30. The method of claim 27, wherein the target for therapeutic intervention is part of an oncogenic signaling pathway.

31. The method of claim 27, wherein the oncogenic signaling pathway is the mammalian target of rapamycin (mTOR) pathway.

32. The method of claim 27, wherein one or more genes from each of at least two or at least three of the biological pathways is differentially translated in the first translational profile as compared to the second translational profile.

33-34. (canceled)

35. The method of claim 27, wherein the first and/or second translational profile comprises a genome-wide measurement of gene translational levels.

36. The method of claim 35, wherein less than 20% of the genes in the genome are differentially translated by at least two-fold in the first translational profile as compared to the second translational profile.

37. The method of claim 35, wherein less than 5% of the genes in the genome are differentially translated by at least two-fold in the first translational profile as compared to the second translational profile.

38. The method of claim 35, wherein less than 1% of the genes in the genome are differentially translated by at least two-fold in the first translational profile as compared to the second translational profile.

39-40. (canceled)

41. The method of claim 27, wherein the first and/or second translational profiles are generated using ribosomal profiling, polysome microarray, immunoassay, or a combination thereof.

42. (canceled)

43. The method of claim 27, wherein the biological sample comprises a human cell.

44-45. (canceled)

46. The method of claim 43, wherein the cell is a cancer cell from prostate cancer, breast cancer, bladder cancer, lung cancer, renal cell carcinoma, endometrial cancer, melanoma, ovarian cancer, thyroid cancer, or brain cancer.

47-49. (canceled)

50. A method of identifying an agent that inhibits an oncogenic signaling pathway in a biological sample, the method comprising:
(a) contacting the biological sample with an agent;
(b) determining a first translational profile for the contacted biological sample, wherein the first translational profile comprises a measurement of gene translational levels for a substantial portion of the genome;
(c) comparing the first translational profile to a second translational profile comprising a measurement of gene translation levels for the substantial portion of the genome in a control sample that has not been contacted with the agent;
(d) identifying in the first translational profile a plurality of genes having decreased translational levels as compared to the translational levels of the plurality of genes in the second translational profile; and
(e) determining whether, for the plurality of genes identified in step (d), there is a common consensus sequence and/or regulatory element in the untranslated regions (UTRs) of the genes that is shared by at least 10% of the plurality of genes identified in step (d);
wherein a decrease in the translational levels of at least 10% of the genes sharing the common consensus sequence and/or UTR regulatory element in the first translational profile as compared to the second translational profile identifies the agent as an inhibitor of an oncogenic signaling pathway.

51. The method of claim 50, wherein the oncogenic signaling pathway is the mammalian target of rapamycin (mTOR) pathway, the PI3K pathway, the AKT pathway, the Ras pathway, the Myc pathway, the Wnt pathway, or the BRAF pathway.

52. The method of claim 50, wherein the first and/or second translational profile comprises a measurement of gene translational levels for at least 500 genes, at least 5000 genes, or a genome-wide measurement of gene translational levels.

53-54. (canceled)

55. The method of claim 50, wherein the biological molecule is a human cell.

56-57. (canceled)

58. The method of claim 55, wherein the cell is a cancer cell from prostate cancer, breast cancer, bladder cancer, lung cancer, renal cell carcinoma, endometrial cancer, melanoma, ovarian cancer, thyroid cancer, or brain cancer.

59. A method of identifying a drug candidate molecule, the method comprising:
(a) contacting a biological sample with the drug candidate molecule;
(b) determining a translational profile for the contacted biological sample, wherein the translational profile comprises translational levels for a plurality of genes; and
(c) comparing the first translational profile to a second translational profile comprising translational levels for the plurality of genes in a control sample that has not been contacted with the drug candidate molecule,
wherein the drug candidate molecule is identified as suitable for use in a therapeutic intervention when one or more genes of a biological pathway is differentially translated in the first translational profile as compared to the second translational profile, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway.

60. The method of claim 59, wherein the one or more genes have a 5′ terminal oligopyrimidine tract (5′ TOP) and/or a pyrimidine-rich translational element (PRTE).

61. The method of claim 59, wherein the one or more genes are selected from the group consisting of SEQ ID NOs:1-144.

62. The method of claim 59, wherein one or more genes from each of at least two or at least three of the biological pathways is differentially translated in the first translational profile as compared to the second translational profile.

63-64. (canceled)

65. The method of claim 59, wherein the translational profile comprises a genome-wide measurement of gene translational levels.

66. The method of claim 65, wherein less than 20% of the genes in the genome are differentially translated in the first translational profile as compared to the second translational profile.

67. The method of claim 65, wherein less than 5% of the genes in the genome are differentially translated by at least two-fold in the first translational profile as compared to the second translational profile.

68. The method of claim 65, wherein less than 1% of the genes in the genome are differentially translated by at least two-fold in the first translational profile as compared to the second translational profile.

69. The method of claim 59, further comprising comparing the translational profile for the contacted biological sample with a control translational profile for a second biological sample that has been contacted with a known therapeutic agent.

70. The method of claim 69, wherein the known therapeutic agent is a known inhibitor of an oncogenic pathway.

71. The method of claim 70, wherein the known therapeutic agent is a known inhibitor of the mTOR pathway.

72. A method of identifying a subject as a candidate for treatment with an mTOR inhibitor, the method comprising:
(a) determining a first translational profile in a sample from the subject, wherein the first translational profile comprises translational levels for one or more genes having a 5′ terminal oligopyrimidine tract (5′ TOP) and/or a pyrimidine-rich translational element (PRTE); and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
wherein a translational level of the one or more genes in the first translational profile that is at least as high as the translational level of the one or more genes in the second translational profile identifies the subject as a candidate for treatment with the mTOR inhibitor.

73. The method of claim 72, wherein the one or more genes are selected from the genes listed in Table 1, Table 2, and/or Table 3.

74. The method of claim 72, wherein the one or more genes are cell invasion and/or metastasis genes.

75. The method of claim 72, wherein the one or more genes are selected from Y-box binding protein 1 (YB1), vimentin, metastasis associated 1 (MTA1), and CD44.

76. A method of identifying a subject as a candidate for treatment with an mTOR inhibitor, the method comprising:
(a) determining a first translational profile in a sample from the subject, wherein the first translational profile comprises translational levels for one or more genes selected from the group consisting of SEQ ID NOs:1-144; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
wherein a translational level of the one or more genes in the first translational profile that is at least as high as the translational level of the one or more genes in the second translational profile identifies the subject as a candidate for treatment with the mTOR inhibitor.

77. A method of identifying a subject as a candidate for treatment with an mTOR inhibitor, the method comprising:
(a) determining a first translational profile in a sample from the subject, wherein the translational profile comprises translational levels for one or more genes of a biological pathway, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
wherein a translational level of the one or more genes that is at least as high as the translational level of the one or more genes in the second translational profile identifies the subject as a candidate for treatment with an mTOR inhibitor.

78. The method of claim 77, wherein the translational level of one or more genes from each of at least two or at least three of the biological pathways is at least as high in the first translational profile as in the second translational profile.

79-80. (canceled)

81. The method of claim 72, wherein the first and/or second translational profile comprises a measurement of gene translational levels for at least 500 genes.

82. The method of claim 72, wherein the first and second translational profiles are differential profiles from before and after administration of the mTOR inhibitor.

83. The method of claim 72, wherein the subject has a cancer.

84. The method of claim 83, wherein the cancer is prostate cancer, breast cancer, bladder cancer, lung cancer, renal cell carcinoma, endometrial cancer, melanoma, ovarian cancer, thyroid cancer, or brain cancer.

85. (canceled)

86. A method of identifying a subject as a candidate for treatment with a therapeutic agent, the method comprising:
(a) determining a first translational profile in a sample from the subject, wherein the translational profile comprises translational levels for one or more genes of a biological pathway, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the therapeutic agent prior to administration of the therapeutic agent to the known responder;
wherein a translational level of the one or more genes that is at least as high as the translational level of the one or more genes in the second translational profile identifies the subject as a candidate for treatment with the therapeutic agent.

87. The method of claim 86, wherein the translational level of one or more genes from each of at least two or at least three of the biological pathways is at least as high in the first translational profile as in the second translational profile.

88. (canceled)

89. The method of claim 86, wherein the first and second translational profiles are differential profiles from before and after administration of the therapeutic agent.

90. The method of claim 86, wherein the subject has a disease.

91. The method of claim 90, wherein the disease is cancer.

92. (canceled)

93. A method of treating a subject having a cancer, the method comprising:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes having a 5′ terminal oligopyrimidine tract (5′ TOP) and/or a pyrimidine-rich translational element (PRTE); and wherein the control sample is from a known responder to the mTOR inhibitor prior to administration of the mTOR inhibitor to the known responder;
thereby treating the cancer in the subject.

94. The method of claim 93, wherein the one or more genes are selected from the genes listed in Table 1, Table 2, and/or Table 3.

95. The method of claim 93, wherein the one or more genes are cell invasion and/or metastasis genes.

96. The method of claim 93, wherein the one or more genes are selected from Y-box binding protein 1 (YB1), vimentin, metastasis associated 1 (MTA1), and CD44.

97. A method of treating a subject having a cancer, the method comprising:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes selected from the group consisting of SEQ ID NOs:1-144; and wherein the control sample is from a known responder to the mTOR inhibitor prior to administration of the mTOR inhibitor to the known responder;
thereby treating the cancer in the subject.

98. A method of treating a subject having a cancer, the method comprising:
administering an mTOR inhibitor to a subject that has been selected as having a first translational profile comprising a translational level of one or more genes that is at least as high as the translational level of the one or more genes in a second translational profile from a control subject;
wherein the first and second translational profiles comprise translational levels for one or more genes of a biological pathway selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the mTOR inhibitor prior to administration of the mTOR inhibitor to the known responder;
thereby treating the cancer in the subject.

99. The method of claim 98, wherein the translational level of one or more genes from each of at least two or at least three of the biological pathways is at least as high in the first translational profile as in the second translational profile.

100. (canceled)

101. The method of claim 93, wherein the first and/or second translational profile comprises a measurement of gene translational levels for at least 500 genes.

102. The method of claim 93, wherein the first and second translational profiles are differential profiles from before and after administration of the mTOR inhibitor.

103. The method of claim 93, wherein the cancer is prostate cancer, breast cancer, bladder cancer, lung cancer, renal cell carcinoma, endometrial cancer, melanoma, ovarian cancer, thyroid cancer, or brain cancer.

104. (canceled)

105. The method of claim 93, further comprising: monitoring the translational levels of the one or more genes in the subject subsequent to administering the mTOR inhibitor.

106. A method of treating a subject in need thereof, the method comprising:
administering a therapeutic agent to a subject that has been selected as having a first translational profile comprising a translational level of one or more genes that is at least as high as the translational level of the one or more genes in a second translational profile;
wherein the first and second translational profiles comprise translational levels for one or more genes of a biological pathway selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the therapeutic agent prior to administration of the therapeutic agent to the known responder;
thereby treating the subject.

107. The method of claim 106, wherein the translational level of one or more genes from each of at least two or at least three of the biological pathways is at least as high in the first translational profile as in the second translational profile.

108. (canceled)

109. The method of claim 106, wherein the first and second translational profiles are differential profiles from before and after administration of the therapeutic agent.

110. The method of claim 106, wherein the subject in need of treatment has a disease.

111. The method of claim 110, wherein the disease is cancer.

112. (canceled)

113. A method of identifying an agent for normalizing a translational profile in a subject in need thereof, the method comprising:
(a) determining a first translational profile for a first biological sample from the subject, wherein the first translational profile comprises translational levels for a plurality of genes;
(b) comparing the first translational profile to a second translational profile comprising translational levels for the plurality of genes, wherein the second translational profile is from a control sample, wherein the control sample is from a non-diseased subject;
(c) identifying one or more genes of a biological pathway as differentially translated in the first translational profile as compared to the second translational profile, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and DNA methylation pathway;
(d) contacting a second biological sample from the subject with an agent;
(e) determining a third translational profile for the second biological sample, wherein the third translational profile comprises translational levels for the one or more genes identified as differentially translated in the first translational profile as compared to the second translational profile; and
(f) comparing the translational levels for the one or more genes in the third translational profile to the translational levels for the one or more genes in the first and second translational profiles;
wherein a translational level for the one or more genes in the third translational profile that is closer to the translational level for the one or more genes in the second translational profile than to the translational level for the one or more genes in the first translational profile identifies the agent as an agent for normalizing the translational profile in the subject.

114. A method of normalizing a translational profile in a subject in need thereof, the method comprising:
administering to the subject an agent that has been selected as an agent that normalizes the translational profile in the subject, wherein the agent is selected by:
(a) determining a first translational profile for a first biological sample from the subject, wherein the first translational profile comprises translational levels for a plurality of genes;
(b) comparing the first translational profile to a second translational profile comprising translational levels for the plurality of genes, wherein the second translational profile is from a control sample, wherein the control sample is from a non-diseased subject;
(c) identifying one or more genes of a biological pathway as differentially translated in the first translational profile as compared to the second translational profile, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and DNA methylation pathway;
(d) contacting a second biological sample from the subject with the agent;
(e) determining a third translational profile for the second biological sample, wherein the third translational profile comprises translational levels for the one or more genes identified as differentially translated in the first translational profile as compared to the second translational profile; and
(f) comparing the translational levels for the one or more genes in the third translational profile to the translational levels for the one or more genes in the first and second translational profiles; wherein a translational level for the one or more genes in the third translational profile that is closer to the translational level for the one or more genes in the second translational profile than to the translational level for the one or more genes in the first translational profile identifies the agent as an agent for normalizing the translational profile in the subject;
thereby normalizing the translational profile in the subject.

115. The method of claim 113, wherein one or more genes from each of at least two or at least three of the biological pathways is differentially translated in the first translational profile as compared to the second translational profile.

116-117. (canceled)

118. The method of claim 113, wherein the first, second, and/or third translational profiles comprise a genome-wide measurement of gene translational levels.

119. The method of claim 113, wherein the agent is a peptide, protein, inhibitory RNA, or small organic molecule.


SEQUENCE LISTING (234 sequences)

SEQ ID NO: 1 - DNA, 3163 bp, Homo sapiens
  eukaryotic translation elongation factor 2 (EEF2, EF-2, EF2), polypeptidyl-tRNA translocase, SCA26
ctcttccgccgtcgtcgccgccatcctcggcgcgactcgcttctttcggttctacctgggagaatccaccgccatccgccaccatggtgaacttcacggtagaccagatccgcgccatcatggacaagaaggccaacatccgcaacatgtctgtcatcgcccacgtggaccatggcaagtccacgctgacagactccctggtgtgcaaggcgggcatcatcgcctcggcccgggccggggagacacgcttcactgatacccggaaggacgagcaggagcgttgcatcaccatcaagtcaactgccatctccctcttctacgagctctcggagaatgacttgaacttcatcaagcagagcaaggacggtgccggcttcctcatcaacctcattgactcccccgggcatgtcgacttctcctcggaggtgactgctgccctccgagtcaccgatggcgcattggtggtggtggactgcgtgtcaggcgtgtgcgtgcagacggagacagtgctgcggcaggccattgccgagcgcatcaagcctgtgctgatgatgaacaagatggaccgcgccctgctggagctgcagctggagcccgaggagctctaccagactttccagcgcatcgtggagaacgtgaacgtcatcatctccacctacggcgagggcgagagcggccccatgggcaacatcatgatcgatcctgtcctcggtaccgtgggctttgggtctggcctccacgggtgggccttcaccctgaagcagtttgccgagatgtatgtggccaagttcgccgccaagggggagggccagttggggcctgccgagcgggccaagaaagtagaggacatgatgaagaagctgtggggtgacaggtactttgacccagccaacggcaagttcagcaagtcagccaccagccccgaagggaagaagctgccacgcaccttctgccagctgatcctggaccccatcttcaaggtgtttgatgcgatcatgaatttcaagaaagaggagacagcaaaactgatagagaaactggacatcaaactggacagcgaggacaaggacaaagaaggcaaacccctgctgaaggctgtgatgcgccgctggctgcctgccggagacgccttgttgcagatgatcaccatccacctgccctcccctgtgacggcccagaagtaccgctgcgagctcctgtacgaggggcccccggacgacgaggctgccatgggcattaaaagctgtgaccccaaaggccctcttatgatgtatatttccaaaatggtgccaacctccgacaaaggtcggttctacgcctttggacgagtcttctcggggctggtctccactggcctgaaggtcaggatcatggggcccaactatacccctgggaagaaggaggacctctacctgaagccaatccagagaacaatcttgatgatgggccgctacgtggagcccatcgaggatgtgccttgtgggaacattgtgggcctcgtgggcgtggaccagttcctggtgaagacgggcaccatcaccaccttcgagcacgcgcacaacatgcgggtgatgaagttcagcgtcagccctgttgtcagagtggccgtggaggccaagaacccggctgacctgcccaagctggtggaggggctgaagcggctggccaagtccgaccccatggtgcagtgcatcatcgaggagtcgggagagcatatcatcgcgggcgccggcgagctgcacctggagatctgcctgaaggacctggaggaggaccacgcctgcatccccatcaagaaatctgacccggtcgtctcgtaccgcgagacggtcagtgaagagtcgaacgtgctctgcctctccaagtcccccaacaagcacaaccggctgtacatgaaggcgcggcccttccccgacggcctggccgaggacatcgataaaggcgaggtgtccgcccgtcaggagctcaagcagcgggcgcgctacctggccgagaagtacgagtgggacgtggctgaggcccgcaagatctggtgctttgggcccgacggcaccggccccaacatcctcaccgacatcaccaagggtgtgcagtacctcaacgagatcaaggacagtgtggtggccggcttccagtgggccaccaaggagggcgcactgtgtgaggagaacatgcggggtgtgcgcttcgacgtccacgacgtcaccctgcacgccgacgccatccaccgcggagggggccagatcatccccacagcacggcgctgcctctatgccagtgtgctgaccgcccagccacgcctcatggagcccatctaccttgtggagatccagtgtccagagcaggtggtcggtggcatctacggggttttgaacaggaagcggggccacgtgttcgaggagtcccaggtggccggcacccccatgtttgtggtcaaggcctatctgcccgtcaacgagtcctttggcttcaccgctgacctgaggtccaacacgggcggccaggcgttcccccagtgtgtgtttgaccactggcagatcctgcccggagaccccttcgacaacagcagccgccccagccaggtggtggcggagacccgcaagcgcaagggcctgaaagaaggcatccctgccctggacaacttcctggacaaattgtaggcggcccttcctgcagcgcctgccgccccggggactcgcagcacccacagcaccacgtcctcgaattctcagacgacacctggagactgtcccgacacagcgacgctcccctgagaggtttctggggcccgctgcgtgccatcactcaaccataacacttgatgccgtttctttcaatatttatttccagagtccggaggcagcagacacgccctcttagtagggacttaatgggccggtcggggagggggaggcgggatgggacacccaacactttttccatttcttcagagggaaactcagatgtccaaactaattttaacaaacgcattaagaggtttatttgggtacatggcccgcagtggcttttgccccagaaaggggaaaggaacacgcgggtagatgatttctagcaggcaggaagtcctgtgcggtgtcaccatgagcacctccagctgtactagtgccattggaataataaatttgataaggtggtgaaaaaa

SEQ ID NO: 2 - DNA, 532 bp, Homo sapiens
  40S ribosomal protein S12, S12
ctctttccctgccgccgccgagtcgcgcggaggcggaggcttgggtgcgttcaagattcaacttcacccgtaacccaccgccatggccgaggaaggcattgctgctggaggtgtaatggacgttaatactgctttacaagaggttctgaagactgccctcatccacgatggcctagcacgtggaattcgcgaagctgccaaagccttagacaagcgccaagcccatctttgtgtgcttgcatccaactgtgatgagcctatgtatgtcaagttggtggaggccctttgtgctgaacaccaaatcaacctaattaaggttgatgacaacaagaaactaggagaatgggtaggcctttgtaaaattgacagagaggggaaaccccgtaaagtggttggttgcagttgtgtagtagttaaggactatggcaaggagtctcaggccaaggatgtcattgaagagtatttcaaatgcaagaaatgaagaaataaatctttggctcacaaaaaaaaaaaaaaaaaaaaaaaaaaaaaa

SEQ ID NO: 3 - DNA, 674 bp, Homo sapiens
  60S ribosomal protein L12
atctggcttgtccgcgcgatttccggcctctcggctttcggctcggaggaggccaaggtgcaacttccttcggtcgtcccgaatccgggttcatccgacaccagccgcctccaccatgccgccgaagttcgaccccaacgagatcaaagtcgtatacctgaggtgcaccggaggtgaagtcggtgccacttctgccctggcccccaagatcggccccctgggtctgtctccaaaaaaagttggtgatgacattgccaaggcaacgggtgactggaagggcctgaggattacagtgaaactgaccattcagaacagacaggcccagattgaggtggtgccttctgcctctgccctgatcatcaaagccctcaaggaaccaccaagagacagaaagaaacagaaaaacattaaacacagtgggaatatcacttttgatgagattgtcaacattgctcgacagatgcggcaccgatccttagccagagaactctctggaaccattaaagagatcctggggactgcccagtcagtgggctgtaatgttgatggccgccatcctcatgacatcatcgatgacatcaacagtggtgctgtggaatgcccagccagttaagcacaaaggaaaacatttcaataaaggatcatttgacaactggtggaaaaaaaaaaaaaaa

SEQ ID NO: 4 - DNA, 962 bp, Homo sapiens
  40S ribosomal protein S2, S2, LLREP3, protein LLRep3, OK/KNS-cl.6
cttcttttccgacaaaacaccaaatggcggatgacgccggtgcagcgggggggcccgggggccctggtggccctgggatggggaaccgcggtggcttccgcggaggtttcggcagtggcatccggggccggggtcgcggccgtggacggggccggggccgaggccgcggagctcgcggaggcaaggccgaggataaggagtggatgcccgtcaccaagttgggccgcttggtcaaggacatgaagatcaagtccctggaggagatctatctcttctccctgcctattaaggaatcagagatcattgatttcttcctgggggcctctctcaaggatgaggttttgaagattatgccagtgcagaagcagacccgtgccggccagcgcaccaggttcaaggcatttgttgctatcggggactacaatggccacgtcggtctgggtgttaagtgctccaaggaggtggccaccgccatccgtggggccatcatcctggccaagctctccatcgtccccgtgcgcagaggctactgggggaacaagatcggcaagccccacactgtcccttgcaaggtgacaggccgctgcggctctgtgctggtacgcctcatccctgcacccaggggcactggcatcgtctccgcacctgtgcctaagaagctgctcatgatggctggtatcgatgactgctacacctcagcccggggctgcactgccaccctgggcaacttcgccaaggccacctttgatgccatttctaagacctacagctacctgacccccgacctctggaaggagactgtattcaccaagtctccctatcaggagttcactgaccacctcgtcaagacccacaccagagtctccgtgcagcggactcaggctccagctgtggctacaacatagggtttttatacaagaaaaataaagtgaattaagcgtgaaaaaaaaaaaaaaaaaaaa

SEQ ID NO: 5 - DNA, 1196 bp, Homo sapiens
  60S ribosomal protein L13a, L13A, transcript variant 1, tissue specific transplantation antigen 1 (TSTA1), 23 kDa highly basic protein
cacttctgccgcccctgtttcaagggataagaaaccctgcgacaaaacctcctccttttccaagcggctgccgaagatggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaaa

SEQ ID NO: 6 - DNA, 671 bp, Homo sapiens
  60S ribosomal protein L18a, L18A, ribosomal protein L18a-like protein
ggtagtgaaggcctggtgaacggctgcgcgacagaggacacttccttttgcgggtggcggcgaacgcggagagcacgccatgaaggcctcgggcacgctacgagagtacaaggtagtgggtcgctgcctgcccacccccaaatgccacacgccgcccctctaccgcatgcgaatctttgcgcctaatcatgtcgtcgccaagtcccgcttctggtactttgtatctcagttaaagaagatgaagaagtcttcaggggagattgtctactgtgggcaggtgtttgagaagtcccccctgcgggtgaagaacttcgggatctggctgcgctatgactcccggagcggcacccacaacatgtaccgggaataccgggacctgaccaccgcaggcgctgtcacccagtgctaccgagacatgggtgcccggcaccgcgcccgagcccactccattcagatcatgaaggtggaggagatcgcggccagcaagtgccgccggccggctgtcaagcagttccacgactccaagatcaagttcccgctgccccaccgggtcctgcgccgtcagcacaagccacgcttcaccaccaagaggcccaacaccttcttctaggtgcagggccctcgtccgggtgtgccccaaataaactcaggaacgccccggtgctcgccgc

SEQ ID NO: 7 - DNA, 3528 bp, Homo sapiens
  eukaryotic translation elongation factor 1 alpha 1 (EEF1A1, EE1A1, EEF-1, EEF1A), cervical cancer suppressor 3 (CCS3), leukocyte receptor cluster member 7, prostate tumor- inducing protein 1 (PTI1), glucocorticoid receptor AF-1 specific elongation factor (GRAF-1EF)
ctttttcgcaacgggtttgccgccagaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgtgttgcatagaaagcttcatgttgctaaaccaatgttaagtgaatctttggaaacaaaatgtttccaaattactgggatgtgcatgttgaaacgtgggttaaaatgactgggcagtgaaagttgactatttgccatgacataagaaataagtgtagtggctagtgtacaccctatgagtggaagggtccattttgaagtcagtggagtaagctttatgccagtttgatggtttcacaagttctattgagtgctattcagaataggaacaaggttctaatagaaaaagatggcaatttgaagtagctataaaattagactaatctacattgcttttctcctgcagagtctaataccttttatgctttgataattagcagtttgtctacttggtcactaggaatgaaactacatggtaataggcttaacaggtgtaatagcccacttactcctgaatctttaagcatttgtgcatttgaaaaatgcttttcgcgatcttcctgctgggattacaggcatgagccactgtgcctgacctcccatatgtaaaagtgtctaaaggtttttttttggttataaaaggaaaatttttgcttaagtttgaaggataggtaaaattaaaggacatgctttctgtttgtgtgatggtttttaaaaattttttttaagatggagttcttgttgcccaggctagaatgcaatggcaaaatctcactgcaatctcctcctcctgggttcaagcaattctcctacttcagcctcccaagtagctgggattacaggcatgtgctaatttggtgtttttaatagagatgaggtttttccatgttggtcaggctggtctcaaactcctgaccttaggtgatcgcctcggcctcctaaagtgctggaattacaggcatgagccaccatgcctggccaggacatgtgttcttaaggacatgctaagcaggagttaaagcagcccaagagataaggcctcttaaagtgactggcaatgtgtattgctcaagattcaaaggtacttgaattggccatagacaagtctgtaatgaagtgttatcgttttccctcatctgagtctgaattagataaaatgccttcccatcagccagtgctctgaggtatcaagtctaaattgaactagagatttttgtccttagtttctttgctatctaatgtttacacaagtaaatagtctaagatttgctggatgacagaaaaaacaggtaaggcctttaatagatggccaatagatgccctgataatgaaagttgacacctgtaagatttaccagtagagaattcttgacatgcaaggaagcaagatttaactgaaaaattgttcccactggaagcaggaatgagtcagtttacttgcatatactgagattgagattaacttcctgtgaaacccagtgtcttagacaactgtggcttgagcaccacctgctggtattcattacaaacttgctcactacaataaatgaattttaagctttaaaaaaaaaaaaaaaaaa

SEQ ID NO: 8 - DNA, 4439 bp, Homo sapiens
  60S ribosomal protein L28 transcript variant 1, L28
ctctttccgtctcaggtcgccgctgcgaagggagccgccgccatgtctgcgcatctgcaatggatggtcgtgcggaactgctccagtttcctgatcaagaggaataagcagacctacagcactgagcccaataacttgaaggcccgcaattccttccgctacaacggactgattcaccgcaagactgtgggcgtggagccggcagccgacggcaaaggtgtcgtggtggtcattaagcggagatccggccagcggaagcctgccacctcctatgtgcggaccaccatcaacaagaatgctcgcgccacgctcagcagcatcagacacatgatccgcaagaacaagtaccgccccgacctgcgcatggtgagctggggtttggggatcaggcttggggagactggccagtgctgtggggaagggcctcccactactggttgcaatatgggctggagagggatggattcttgctttcagcctactccccacacccagcattggcctagggggcggcttgtggagtgtatgggctgagccttgctctgctcccccgcccccaggcagccatccgcagggccagcgccatcctgcgcagccagaagcctgtgatggtgaagaggaagcggacccgccccaccaagagctcctgagccccctgcccccagagcaataaagtcagctggctttctcacctgcctcgactgggcctccctttttgaaacgctctggggagctctggccctgtgtgttgtcattcaggccatgtcatcaaaactctgcatgtcaccttgtccatctggaggtgatgtcaatggctggccatgcaggaggggtggggtagctgccttgtccctggtgagggcaagggtcactgtcttcacagaaaaagtttgctgacttgtgattgagacctactgtcccattgtgaggtggcctgaagaatcccagctggggcagtggcttccattcagaagaagaaaggccttttctagcccagaagggtgcaggctgagggctgggccctgggccctggtgctgtagcacggtttggggacttggggtgttcccaagacctgggggacgacagacatcacgggaggaagatgagatgacttttgcatccagggagtgggtgcagccacatttggaggggatgggctttacttgatgcaacctcatctctgagatgggcaacttggtgggtggtggcttataactgtaagggagatggcagccccagggtacagccagcaggcattgagcagccttagcattgtccccctactcccgtcctccaggtgtccccatccctcccctgtctctttgagctggctcttgtcacttaggtctcatctcagtggccgctcctgggccaccctgtcacccaagctttcctgattgcccagccctcttgtttcctttggcctgtttgctccctagtgtttattacagcttgtgaggccaggagtttgagaccatcctaggcaacataatgagacaccgtctctaaaataaaattagctgggtgtggtggtgcaccgcctgtggtcccagctcctcagaggttgagtagaggctgaggtgagcggagcacttgagccaagagtatgaggctgcagtgagcccatgagccccaccactacactccagcctggaagacaccatgacacacagtgaggcctggatggggaaagagtcctgctgttgatcctcacatgtttcctgggcacctaactctgtcagccactgccagggaccaaggatccagcatccatggcacccctggttcctgccatcctggggtacccgattcaaagaaggactctgctccctgtctgagaccacccccggctctgactgagagtaaggggactgtcagggcctcgacttgccattggttggggtcgtacggggctgggagccctgcgttttgaggcagaccactgcccttccgacctcagtcctgtctgctccagtcttgcccagctcgaaggagagcagatctgaccacttgccagcccctgtctgctgtgaattaccatttcctttgtccttcccttagttgggtctattagctcagattgagaggtgttgccttaaaactgagttgggtgacttggtacctgctcaggaccccccgcactgtcccaatcccactcaggcccacctccagctggcctcactccgctggtgacttcgtacctgctcaggagcccccactgtcccagtcccactcaggcccatctctggctggcctcactgcgctgggactccgccttcataaggagagctcactgctcacgttagtagatggccccttctcgtgaggcctctcccctggcacctgcttcagttgtcctccacagcactgatttgcagcccacaagctggcaggtttatctgtctcatgtttgtcttgtgctggtgggcaaggggtttgtctagcacaccagcatataatgagatgcttgatgaatggtgcatattgaatgtataaagcccaccggtcctgagagtttgctcactggagactttctggagatggagtctcgctctgttgcccaggctggcgagtgcaatggcgcgatcttggctcactgcagcctccacctcctgggttcaagcgattctcctgcctcagcctcccgagtagctgggattacaggtgggtgtcaccacacccagctcagtattgtatttttagcagagatggggtttcaccattttgcccaggctggtttggaactcctgacttcaaattacccacctgcctcagcctcccaaagtgctggcattacaggcgctcgaggctttctgatgtggctgctgctgctcagaaggccttgtccttaaccacctccttgcctgccctggaggcttgtgcctctaggccccaccccctgtggagtcctgctggctttctccatccctatctgaatcctccctgctgtgtggcctcccctggtctcatccgtaacacagcccagcttagtgggcctctgttcctgcgggtggccagcctgtctgtgtggctgggctggggaggccacgtctggtatctgaatgctatcggtgggttggggtggaggaaccaggagagggctggagggagggagatggtctcagccccacagagtttggagtcctcagtgtgctgagcaaacgtggagacaccatttccctcctctagacctcatcttggagagagagatgttggatggggccatctattccagctttattcacacaaatcatgtctgttggcctggaaattggaaaaccagttaaaccaaaaacatgatattaagaaaacaggcaggctcaccatagtaaaaatgctgaaagccaaagacaaaattgggagaacaaaagaaaagcgtcttgtcacatacagaaggtccctgataaagttagtagctgccctcatcagaaaccaggcccaggcagtggggacacatccagagtgctgaaagaacctcccccaggtcatcctatccccaagagtgatgcccggcagcattcccagctcagggctaatggttcacggaagccaggaatcaaactgcctgggttccagtcccagctctgccagttatgcccagctgtggggacttgggcagctcgtttagtagcaccgtgcctcagtttcccatatgtaaaaggccattttgagtgcctttcacagccctgcataaggcaggtgtctcagtgttcactgctgtctctccagctcttagtccagtagctgcatggtgagtgagcgtagggcgcaccctggaaggctgccaagcccaaagttgtgcagagcgctggggactccagactccccacagcagcagagactcgggactgaggcatcctctgttcacaggacatgctggcatctactgggtcagggctctgctgctcggtggctgtgcaaccttgggcaagttcctcaacctctctgtgtcttcgtaccctcatctgtaacatgcgtgtcgatagaccctactactcagggttgatgagaagattaaatgtgcaaaacctgcttgactgtgcccacaaatcctgattgtaggaataaattaatgactttttataaatattttgatcagatggactcatgatcacagatgtcttcacatgcctatgactaatttgtacacaaactaatgctcgtgtttcccaagcacctggaagacatgccagatccatgtgcagtaatgcctggtggctccaggtctgccccgccgtcctgtggggctgtgagctttcccagcctcctgcccgtgtttgtgaatatcattctgtcctcagctgcatttccagcccaggctgtttggcgctgcccaggaatggtatcaattcccctgtttctcttgtagccagttactagaataaaatcatctactttaaaaaaaaaaaaaaaaaaaaaaaaaaaaaaaaaaaaaaaa

SEQ ID NO: 9 - DNA, 401 bp, Homo sapiens
  40S ribosomal protein S28, S28
ctctccgccagaccgccgccgcgccgccatcatggacaccagccgtgtgcagcctatcaagctggccagggtcaccaaggtcctgggcaggaccggttctcagggacagtgcacgcaggtgcgcgtggaattcatggacgacacgagccgatccatcatccgcaatgtaaaaggccccgtgcgcgagggcgacgtgctcacccttttggagtcagagcgagaagcccggaggttgcgctgagcttggctgctcgctgggtcttggatgtcgggttcgaccacttggccgatgggaatggtctgtcacagtctgctccttttttttgtccgccacacgtaactgagatgctcctttaaataaagcgtttgtgtttcaagttaaaaaaaaaaaaaaaaaaaaa

SEQ ID NO: 10 - DNA, 361 bp, Homo sapiens
  40S ribosomal protein S27, S27, metallopan- stimulin 1, metallopanstimulin 1 (MPS-1, MPS1)
ctttccggcggtgacgacctacgcacacgagaacatgcctctcgcaaaggatctccttcatccctctccagaagaggagaagaggaaacacaagaagaaacgcctggtgcagagccccaattcctacttcatggatgtgaaatgcccaggatgctataaaatcaccacggtctttagccatgcacaaacggtagttttgtgtgttggctgctccactgtcctctgccagcctacaggaggaaaagcaaggcttacagaaggatgttccttcaggaggaagcagcactaaaagcactctgagtcaagatgagtgggaaaccatctcaataaacacattttggataaatcctgaaaaaaaaaa

SEQ ID NO: 11 - DNA, 918 bp, Homo sapiens
  60S ribosomal protein L34 transcript variant 1, L34, leukemia-associated protein
cttttttcttcctcttccggggacgttgtctgcaggcactcagaatggtccagcgtttgacataccgacgtaggctttcctacaatacagcctctaacaaaactaggctgtcccgaacccctggtaatagaattgtttacctttataccaagaaggttgggaaagcaccaaaatctgcatgtggtgtgtgcccaggcagacttcgaggggttcgtgctgtaagacctaaagttcttatgagattgtccaaaacaaagaaacatgtcagcagggcctatggtggttccatgtgtgctaaatgtgttcgtgacaggatcaagcgtgctttccttatcgaggagcagaaaatcgttgtgaaagtgttgaaggcacaagcacagagtcagaaagctaaataaaaaaatgaaacttttttgagtaataaaaatgaaaagacgctgtccaatagaaaaagttggtgtgctggagctacctcacctcagcttgagagagccagttgtgtgcatctctttccagttttgcatccagtgacgtctgcttggcatcttgagattgttatggtgagagtatttacacctcagcaaatgctgcaaaatcctgttttcccccagagagctggaggttaaatactaccagcacatccctagatactactcaagttacagtatatgatcactaatatagtatgctcttggtaccaggagctctgatatatatctggtacatgtttgataatgacttgattgttattataagtacttattaatacttcgattctgtaaagagtttagggtttgattttataaaatccaaaatgagccttttattgaatccagttctctatgtgaccagttctctgtatgaatggaagggaaaagaattaaaaatcttgcaaaggggaaaaaaaaaaaaaaaaaa

SEQ ID NO: 12 - DNA, 4906 bp, Homo sapiens
  60S ribosomal protein L27a, L27A
aacgaggcccttagggtcggcttaggcggttccctgaccaaggcgccagaaaagggcctggctcaagcaagcacgggcggcgtgcagtacagcacacctagccccgattcttcaacagttctcgccctccgagcctagcacaacgagcctcaccgaaaccgtacaccgccaccaggacactccgtgatgggggatcaccaccctcagaaagaggaagcgactagcaggcgcgcaatcccgcgagaccaggaggccccgcccgaagcccggcctctgtgaccggaagtgaggcgttttgccccgcccccgtggccgatacctcgcgagacttggcgaaggccttcctttttcgtctgggctgccaacatgccatccagactgaggaagacccggaaacttaggggccacgtgagccacggccacggccgcataggcaagcaccggaagcaccccggcggccgcggtaatgctggtggtctgcatcaccaccggatcaacttcgacaaataccacccaggctactttgggaaagttggtatgaagcattaccacttaaagaggaaccagagcttctgcccaactgtcaaccttgacaaattgtggactttggtcagtgaacagacacgggtgaatgctgctaaaaacaagactggggctgctcccatcattgatgtggtgcgatcgggctactacaaagttctgggaaagggaaagctcccaaagcagcctgtcatcgtgaaggccaaattcttcagcagaagagctgaggagaagattaagagtgttgggggggcctgtgtcctggtggcttgaagccacatggagggagtttcattaaatgctaactactttttccttgtggtgtgagtgtaggttcttcagtggcacctctacatcctgtgtgcattgggagcccaggttctagtacttagggtatgaagacatggggtcctctcctgacttccctcaaatatatggtaaacgtaagaccaacacagacgttggccagttaaacatttctgtttataaagtcagaataatacctgttgatcactgaaaggcctgcatgtattgtactctgaattttacagtgaatgagagaatgtaccctaattgttcaacagggctcaaaaggaaagattccattttgatgggtcacattctaaagaggggcagtgtgataggaatgagatggtcctttaggacttaagttctcagcccaaggtttttccacgtggccccctcatcttttttttttttttaaacggagtctctcttgccaggctggagtgcagtggcacgatctcggctcactgcagcctccgcctcccaggttaagcgattctcctgcctcagcttcctgactaactgggattacaggcgcccaccaccatgcccagctaatttttgtattttcagtagagatggggtttcaccatgttggccatgctggtctctaactcctaacctcaagtgatctgcccacatcggcctccaaaagttctgggattatagtgtgagccactgcgcccggccatggctccttaatcttgatccaaattattgttacatccagaatgtgatgaatcaaaatctcgagatgggggtccagcaatctgaaatttcagtatgccagggcttttctgtatgtcaaagtgggtttgaaatagttaatttttcttctagtctgaaatgtatcgggaaaatttggaaatcctgaaggctggaaattgaaataagtttttctaggatttgtgtctcttgctattggaaaactgatggtgaccaattcatgtttacaaataagatcctcatagatctcggtaaattataatttgctacagttttatggttcttcctgtgattttgagctttttttgacccaaaataatacagtctaaaactatagacaaataagatggcacttagactcctgggttttagttagtggaggtttccttagtgcactgtggggtcataataagccgagaaccatggctgtctatgggacacatctgtcaggacaacctttagaggatgttggggatcaaatagaaggcacagagaagcactgaattggcttacataagaataggctagaattacaagtagtgaaacctcgattcagctggacaattttaaacaaatgtatcatttggcttgtatcttctgttgtgctggagaagttagaaataagggctctccagaccagcctgaccaacctggagaaaccttgtctctactaaatacacaaaattagccaggcgtggtggcacatgcctgtaatcccagctactttggaggctgagccaggagaatctccaggaggcggaggttgctgtgagccgagatcgtgccattgcactccagcttgggcaacaagagtgaaactctgtccaccccccccaaaaaaagtaagggctctccattagggcccatagaggacttgtaatatggaacctgaatccaaggatcccacaataagtggtcagtagttcatgatgaattaaaagactcaatatttggtcttcacccaatacctgtgtgacttttagtcctaatttcctcatctttaaaatttcagtgaaagtgcctacctgaggattgtgtagattaaaatggaaaccgtgcacttaattttttgttttgttttgagacggagtctcgctctgtcgcccaggctggagtgcagtggtgcgatctcagatcactgcaagctccgcctcctaggttcagaccattctcctgcctcagcttcccaagtagctgggactacaggcgcccgccactgcgcccggctaattttttgcatttttagtagagacagggtttcaccgtgttagccaggatggtctcgatctcctgatctgcccgcctcagcctcccaaagtgctgggattacaggcatgagccaccgcgcccggcccaggcacttaatttttgtgtttgacttagtaacttaagtgcaaactattacgggagcagatggagtcaattggccttcatgtgattgtcagtgggaaattggtccaagcagagggaatactggttcaggaaactggtttgggaaggttaggcaaacgggaagtgctatggtggagagaaagattactctggccgggctgtaaaggacggctacaatgggaggctgaaggcagaaccaagaaaatgggagtgagtatggaaaaggtacgattcagacggcataatggacgggacttggagactgaattgtagtgggccgaccacaaaatgataaggcatggaaggaagtagagtttggggggaaggatccctagtcccttaatggctaccttcttccccaggagttgttaggccatccgatcccctggcctgggaaagaaacactgatttcgttgctggcttgttcactcaccagaagctacagctactaacagttctaaaaactgtttcatgtgatgaggaacagacgaaaatagttttgagccctaagtccgccgattccagtgctttcttgaacccgcatttactaaaatattttcatgactgccaagctttgaatagcctgctgtgttcatggaggctcatactggcgatctctagtggctggctaaagcttgaattgcaaaagatctaatttctggtctaatgtatatatgccttaaatatagttgcgttcaaacgtgggagctgcaggtgcaacttgattttatgacaaatggctgccacataatttgcacaagcagtgctcgtcaagggcagctaaatcaggcgagctttcaatcaaaataaatgtactactaaaccctacttagcggctaactagcccaagagcagacagcccacggacggactgcaagtcggaagcgcgggcggaagctgtgcagcgcccacctggtggctccatcggccgcgttcatcagtcagcacgacccgacctcagtggcgtcctcacaacacagaccggaccttgggtcttaccccggcacctgagaaccacttccggtgagtagcttctacttccggagacgatgactcccccgcgtcccagaccggaagaagcccggcggagaccggcctcgctcggccacttccggcaagggcggagccggccagtggtgcgcgagcgcagataactcccctggagaggcgggatgttcaactccacccctggtccttgggcggccgtgggtccccttcgaagcggaggaatggccaacctcgccgcacttcgagcccctttagggtgcgtttaagaacagtgggcgtggcctttacgtaaatcttcgagatgggaacctccagaatttgtctcaattgtctaaaaggtaatgagcgtcagcgacattcaagggcactttgggctaaaaaagaaagtgcttgtacacggatggaaatattctagaagaacataaaaggaatttcctcttaggaggttagggaaatgagcacgaagtatgttttggtgcagttttttgttcaacccaatgcgtattttcatattgagaggcaatataaatggagcgaaagtatcttgagaaaaaaaaaaaaactaccagaacttgccgttgctgaaaagtaatattttctctttcgagagttttcatggccttttaaattacacccccacctccacaggcaaataaatttgttttggaatgcataccacatcatctggctctagaaacgtattttgtgtagctcccctagcaagaatataggttaaagcgtaaatttaattcctggctctattttacatcccaatttttattttcctctcattcccactttacgttgtttcaaataacctagtttgtgtatccctgtaagtcattttggtataaagtaggttataagtgtacatgcgaaaagatgtttttaacaaaaatgtaactgaaaaaaaaaaaaaaaaaaaaaaaaaaaa

SEQ ID NO: 13 - DNA, 2335 bp, Homo sapiens
  60S ribosomal protein L10 transcript variant 1, L10, laminin receptor homolog, tumor suppressor QM (QM), Wilms tumor-related protein, AUTSX5, DXS648, DXS648E, NOV
gggctacgcccgggcgcaagcgccaagagcggctgcgtctatggtcatgacgtctgacagagcgtccacccgtcttcgacaggactctatggttcttacgcgcgcagacagaccgcctatataagccatgcgcaggcggaggagcgcctctttcccttcggtgtgccactgaagatcctggtgtcgccatgggccgccgccccgcccgttgttaccggtattgtaagaacaagccgtacccaaagtctcgcttctgccgaggtgtccctgatgccaagattcgcatttttgacctggggcggaaaaaggcaaaagtggatgagtttccgctttgtggccacatggtgtcagatgaatatgagcagctgtcctctgaagccctggaggctgcccgaatttgtgccaataagtacatggtaaaaagttgtggcaaagatggcttccatatccgggtgcggctccaccccttccacgtcatccgcatcaacaagatgttgtcctgtgctggggctgacaggctccaaacaggcatgcgaggtgcctttggaaagccccagggcactgtggccagggttcacattggccaagttatcatgtccatccgcaccaagctgcagaacaaggagcatgtgattgaggccctgcgcagggccaagttcaagtttcctggccgccagaagatccacatctcaaagaagtggggcttcaccaagttcaatgctgatgaatttgaagacatggtggctgaaaagcggctcatcccagatggctgtggggtcaagtacatccccaatcgtggccctctggacaagtggcgggccctgcactcatgagggcttccaatgtgctgcccccctcttaatactcaccaataaattctacttcctgtccacctatgtctttgtatctacattcttgacggggaaggaacttcctctgggaacctttgggtcattgccctttcacttcagaaacaggttgacaactcagccctgctcatgaggcagcaaaccctgcaaagggctgggactggtggccttatgtcagttgtctactctggagcttgacttggacctccccaggtcctaggcagtaggttgaaaaacactgaagtgcttttcatgaagcacagctgcagcaaagccttgcaatcccaggctggggtcagcctacagttgtgttgcttattacaacacatgcggaccaagaggggcttgtgggctagaggctgaccagcagcgtttatttagcaagggtaggtgtgcatcacattgggcttgttctcacccatctggtttggccattcctccttggtgggaatcatccaggtactgctgaggtcacctgcgatttgccccatttcctatctctagcaacctcctgggccccatgcccccaccccttctagaacctgcattcccagggccttcaccacctgaccaaaggtctaggctaacctttggtcatttgtaacaagacctcggaacagacacgtgtgtggcatggtttggcctggggatcttagatgtctgacctgaactattgtagaacagcgctggcttttgggggagcagcaaaaatgagaggagtgctaggtgggtggcctgagcatctgtatccagggacaggactccaaaggcttttggtcccagagctggggtatgttggccccagcccccagcctgtggctcccaaaaggcctctggttttttgtaatctcagtttacagccatttcttaggtttttaattacctttattttattttgccaaacatacctgggaataccttttattttttttttaccttggggtgatggttccaaaccataaatgtgattatagttaacacatgacccttctagcgtcccagccagtgtttttcctgacctctgttctttggagaggaggatggaagggaggggtccggcacgctgctggcattttgctgtgtcctgcagcccctttccgggacacctgggttcacacagctttttagcttacataactggtgcagattttctgtgtggagatgttgccttgaccagccttggctggactttaccaggcatgcagaagcctgtaccaacacagactacagcacccaggaggtgcgagtgtggctgctcagcggttataacaggcctgactgcattgttcaccggattataatgagccaaaatgtttcccggtgtttgctggtttcagggaaggagtttgatatagcagattaaccaccctccttgtagctattggggcttaatggtttcctggtgattcttaccaatccacaataaacatggcccattggcatatctgcaaaaaaaaaaaaaaaaa

SEQ ID NO: 14 - DNA, 2381 bp, Homo sapiens
  eukaryotic translation elongation factor 1 delta (guanine nucleotide exchange protein) (EEF1D, EF-1D, EF1D, EF-1-delta) transcript variant 3, antigen NY-CO-4, FP1047
gacaccactggccgcgaagcgcagggggggcgcgcgcgctgccgcccgttcccagcacttgtgcagactcctccggggaagagcggcctccgcggtcatctccaacggaattctgcctttgaagtgtcggggcacggcgcgtcgagggtcctggcggccaggcggggcgtgtgcaagggtcgcgtcccccccccgggcccccggcccgtggctcttggtagagcccagtgcttcatttcccgtgcgcggcccgggcggccctccctttcatcagtcttcccgcgtccgccgattcctcctccttggtcgccgcgtccttggctggcgtgaggccaaagcaaaatgaggagcgggaaggcctcctgcaccctggagaccgtgtgggaagacaagcacaagtatgaggaggccgagcggcgcttctacgaacacgaggccacacaggcggccgcctccgcccagcagctgccagccgaggggccagccatgaatgggcccggccaggacgaccctgaggacgctgatgaggcggaagcccctgacggcggcagcaggcgtgatcccaggaagagccaggacagcaggaagcccctgcagaaaaagaggaagcgctcccccaagagcgggctcggccccgcggacctggccctcctgggcctctcggccgaacgcgtgtggctggacaagtcacttttcgaccaggcagagagctcctaccgccagaagctggcagatgtggctgcccaggcagcctggcctcctgccttggccccttggggtctctgcacccatggaaaccaggtggcctgccaccacgtgacctgggggatctgggtcaacaagtcctccttcgaccaggctgagcgggccttcgtggagtggtctcaggccctgttgctggcccccgagggcagccgcaggcaggggactcccaacacaggccagcaggtggccgtccccgacctggcccaccagcccagcccaccggtcaatggccagcccccgctgggcagcctgcaggcactggttcgggaggtgtggctggagaagccccggtatgatgcagccgagaggggcttctacgaggccctgtttgacggccatcccccagggaaggtgcgcctgcaagagcgagccggcctggccgagggtgcccggcggggccgcagagaccggcggggccgcaacatcttagggaacaagcgggccgggctgcgacgggccgatggggaggccccctctgccttgccctactgttacttcctgcagaaggatgcagaggccccctggctcagcaagcctgcctacgacagcgccgagtgccgccaccacgctgccgaggccctgcgggtggcctggtgcctcgaagctgcctccctgtctcaccgacccggtcctcggtctggcctgtccgtgtccagcctgagacccaacagaaaaatggctacaaacttcctagcacatgagaagatctggttcgacaagttcaaatatgacgacgcagaaaggagattctacgagcagatgaacgggcctgtggcaggtgcctcccgccaggagaacggcgccagcgtgatcctccgtgacattgcgagagccagagagaacatccagaaatccctggctggaagctcaggccccggggcctccagcggcaccagcggagaccacggtgagctcgtcgtccggattgccagtctggaagtggagaaccagagtctgcgtggcgtggtacaggagctgcagcaggccatctccaagctggaggcccggctgaacgtgctggagaagagctcgcctggccaccgggccacggccccacagacccagcacgtatctcccatgcgccaagtggagcccccagccaagaagccagccacaccagcagaggatgacgaggatgatgacattgacctgtttggcagtgacaatgaggaggaggacaaggaggcggcacagctgcgggaggagcggctacggcagtacgcggagaagaaggccaagaagcctgcactggtggccaagtcctccatcctgctggatgtcaagccttgggatgatgagacggacatggcccagctggaggcctgtgtgcgctctatccagctggacgggctggtctggggggcttccaagctggtgcccgtgggctacggtatccggaagctacagattcagtgtgtggtggaggacgacaaggtggggacagacttgctggaggaggagatcaccaagtttgaggagcacgtgcagagtgtcgatatcgcagctttcaacaagatctgaagcctgagtgtgtgtacgtgcgcgcgtgcgtgaggccctgccacgattaaagactgagaccggccctctggctccgtcctggtcatttcctgctc

SEQ ID NO: 15 - DNA, 1523 bp, Homo sapiens
  glioma tumor suppressor candidate region gene 2 (GLTSCR2), protein interacting with carboxyl terminus 1 (PICT-1, PICT1), p60
tggctgagttcttcctttgacaagatggcggcaggaggcagtggcgttggtgggaagcgcagctcgaaaagcgatgccgattctggtttcctggggctgcggcccacttcggtggacccagcgctgaggcggcggcggcgaggcccaagaaataagaagcggggctggcggcggcttgctcaggagccgctggggctggaggttgaccagttcctggaagacgtgcggctacaggagcgcacgagcggtggcttgttgtcagaggccccaaatgaaaaactcttcttcgtggacactggctccaaggaaaaagggctgacaaagaagagaaccaaagtccagaagaagtcactgcttctcaagaaaccccttcgggttgacctcatcctcgagaacacatccaaagtccctgcccccaaagacgtcctcgcccaccaggtccccaacgccaagaagctcaggcggaaggagcagctatgggagaagctggccaagcagggcgagctgccccgggaggtgcgcagggcccaggcccggctcctcaacccttctgcaacaagggccaagcccgggccccaggacaccgtagagcggcccttctacgacctctgggcctcagacaaccccctggacaggccgttggttggccaggatgagtttttcctggagcagaccaagaagaaaggagtgaagcggccagcacgcctgcacaccaagccgtcccaggcacccgccgtggaggtggcgcctgccggagcttcctacaatccatcctttgaagaccaccagaccctgctctcagcggcccacgaggtggagttgcagcggcagaaggaggcggagaagctggagcggcagctggccctgcccgccacggagcaggccgccacccaggagtccacattccaggagctgtgcgaggggctgctggaggagtcggatggtgagggggagccaggccagggcgaggggccggaggctggggatgccgaggtctgtcccacgcccgcccgcctggccaccacagagaagaagacggagcagcagcggcggcgggagaaggctgtgcacaggctgcgggtacagcaggccgcgttgcgggccgcccggctccggcaccaggagctgttccggctgcgcgggatcaaggcccaggtggccctgaggctggcggagctggcgcggcggcagaggcggcggcaggcgcggcgggaggctgaggctgacaagccccgaaggctggggcggctcaagtaccaggcacctgacatcgacgtgcagctgagctcggagctgacagactcgctcaggaccctgaagcccgagggcaacatccttcgagaccggttcaagagcttccagaggaggaatatgatcgagcctcgagagagagccaagttcaaacgcaagtacaaggtgaagctggtggagaagcgggcgttccgtgagatccagttgtagctgccatcagatgccggagactcgcccttcaataaaaaatctcttctagctgatcagtggga

SEQ ID NO: 16 - DNA, 1348 bp, Homo sapiens
  60S ribosomal protein L3 transcript variant 1, L3, HIV-1 TAR RNA-binding protein B (TARBP-B), ASC-1
ccagatttggctttatatagcggacccgtaaggccgaccggcctctaccggcgggatttgatggcgtgatgtctcacagaaagttctccgctcccagacatgggtccctcggcttcctgcctcggaagcgcagcagcaggcatcgtgggaaggtgaagagcttccctaaggatgacccgtccaagccggtccacctcacagccttcctgggatacaaggctggcatgactcacatcgtgcgggaagtcgacaggccgggatccaaggtgaacaagaaggaggtggtggaggctgtgaccattgtagagacaccacccatggtggttgtgggcattgtgggctacgtggaaacccctcgaggcctccggaccttcaagactgtctttgctgagcacatcagtgatgaatgcaagaggcgtttctataagaattggcataaatctaagaagaaggcctttaccaagtactgcaagaaatggcaggatgaggatggcaagaagcagctggagaaggacttcagcagcatgaagaagtactgccaagtcatccgtgtcattgcccacacccagatgcgcctgcttcctctgcgccagaagaaggcccacctgatggagatccaggtgaacggaggcactgtggccgagaagctggactgggcccgcgagaggcttgagcagcaggtacctgtgaaccaagtgtttgggcaggatgagatgatcgacgtcatcggggtgaccaagggcaaaggctacaaaggggtcaccagtcgttggcacaccaagaagctgccccgcaagacccaccgaggcctgcgcaaggtggcctgtattggggcatggcatcctgctcgtgtagccttctctgtggcacgcgctgggcagaaaggctaccatcaccgcactgagatcaacaagaagatttataagattggccagggctaccttatcaaggacggcaagctgatcaagaacaatgcctccactgactatgacctatctgacaagagcatcaaccctctgggtggctttgtccactatggtgaagtgaccaatgactttgtcatgctgaaaggctgtgtggtgggaaccaagaagcgggtgctcaccctccgcaagtccttgctggtgcagacgaagcggcgggctctggagaagattgaccttaagttcattgacaccacctccaagtttggccatggccgcttccagaccatggaggagaagaaagcattcatgggaccactgaagaaagaccgaattgcaaaggaagaaggagcttaatgccaggaacagattttgcagttggtggggtctcaataaaagttattttccactgacaaaaaaaaaaa

SEQ ID NO: 17 - DNA, 2869 bp, Homo sapiens
  poly(A) binding protein, cytoplasmic 1 (PABPC1, PAB1, PABP1, PABPL1), poly(A) binding protein, cytoplasmic 2 (PABPC2), polyadenylate-binding protein 1 (PABP)
cccttctccccggcggttagtgctgagagtgcggagtgtgtgctccgggctcggaacacacatttattattaaaaaatccaaaaaaaatctaaaaaaatcttttaaaaaaccccaaaaaaatttacaaaaaatccgcgtctcccccgccggagacttttattttttttcttcctcttttataaaataacccggtgaagcagccgagaccgacccgcccgcccgcggccccgcagcagctccaagaaggaaccaagagaccgaggccttcccgctgcccggacccgacaccgccaccctcgctccccgccggcagccggcagccagcggcagtggatcgaccccgttctgcggccgttgagtagttttcaattccggttgatttttgtccctctgcgcttgctccccgctcccctccccccggctccggcccccagccccggcactcgctctcctcctctcacggaaaggtcgcggcctgtggccctgcgggcagccgtgccgagatgaaccccagtgcccccagctaccccatggcctcgctctacgtgggggacctccaccccgacgtgaccgaggcgatgctctacgagaagttcagcccggccgggcccatcctctccatccgggtctgcagggacatgatcacccgccgctccttgggctacgcgtatgtgaacttccagcagccggcggacgcggagcgtgctttggacaccatgaattttgatgttataaagggcaagccagtacgcatcatgtggtctcagcgtgatccatcacttcgcaaaagtggagtaggcaacatattcattaaaaatctggacaaatccattgataataaagcactgtatgatacattttctgcttttggtaacatcctttcatgtaaggtggtttgtgatgaaaatggttccaagggctatggatttgtacactttgagacgcaggaagcagctgaaagagctattgaaaaaatgaatggaatgctcctaaatgatcgcaaagtatttgttggacgatttaagtctcgtaaagaacgagaagctgaacttggagctagggcaaaagaattcaccaatgtttacatcaagaattttggagaagacatggatgatgagcgccttaaggatctctttggcaagtttgggcctgccttaagtgtgaaagtaatgactgatgaaagtggaaaatccaaaggatttggatttgtaagctttgaaaggcatgaagatgcacagaaagctgtggatgagatgaacggaaaggagctcaatggaaaacaaatttatgttggtcgagctcagaaaaaggtggaacggcagacggaacttaagcgcaaatttgaacagatgaaacaagataggatcaccagataccagggtgttaatctttatgtgaaaaatcttgatgatggtattgatgatgaacgtctccggaaagagttttctccatttggtacaatcactagtgcaaaggttatgatggagggtggtcgcagcaaagggtttggttttgtatgtttctcctccccagaagaagccactaaagcagttacagaaatgaacggtagaattgtggccacaaagccattgtatgtagctttagctcagcgcaaagaagagcgccaggctcacctcactaaccagtatatgcagagaatggcaagtgtacgagctgttcccaaccctgtaatcaacccctaccagccagcacctccttcaggttacttcatggcagctatcccacagactcagaaccgtgctgcatactatcctcctagccaaattgctcaactaagaccaagtcctcgctggactgctcagggtgccagacctcatccattccaaaatatgcccggtgctatccgcccagctgctcctagaccaccatttagtactatgagaccagcttcttcacaggttccacgagtcatgtcaacacagcgtgttgctaacacatcaacacagacaatgggtccacgtcctgcagctgcagccgctgcagctactcctgctgtccgcaccgttccacagtataaatatgctgcaggagttcgcaatcctcagcaacatcttaatgcacagccacaagttacaatgcaacagcctgctgttcatgtacaaggtcaggaacctttgactgcttccatgttggcatctgcccctcctcaagagcaaaagcaaatgttgggtgaacggctgtttcctcttattcaagccatgcaccctactcttgctggtaaaatcactggcatgttgttggagattgataattcagaacttcttcatatgctcgagtctccagagtcactccgttctaaggttgatgaagctgtagctgtactacaagcccaccaagctaaagaggctgcccagaaagcagttaacagtgccaccggtgttccaactgtttaaaattgatcagggaccatgaaaagaaacttgtgcttcaccgaagaaaaatatctaaacatcgaaaaacttaaatattatggaaaaaaaacattgcaaaatataaaataaataaaaaaaggaaaggaaactttgaaccttatgtaccgagcaaatgccaggtctagcaaacataatgctagtcctagattacttattgatttaaaaacaaaaaaacacaaaaaaatagtaaaatataaaaacaaattaatgttttatagaccctgggaaaaagaattttcagcaaagtacaaaaatttaaagcattcctttctttaattttgtaattctttactgtggaatagctcagaatgtcagttctgttttaagtaacagaattgataactgagcaaggaaacgtaatttggattataaaattcttgctttaataaaaattccttaaacagtgaaaaaaaaa

SEQ ID NO: 18 - DNA, 434 bp, Homo sapiens
  60S ribosomal protein L37a, L37A
acttccgctcgtccgcctaataccgcgcctgcgcaccgcgtctcttcctttctgggctcggacctaggtcgcggcgacatggccaaacgtaccaagaaagtcgggatcgtcggtaaatacgggacccgctatggggcctccctccggaaaatggtgaagaaaattgaaatcagccagcacgccaagtacacttgctctttctgtggcaaaaccaagatgaagagacgagctgtggggatctggcactgtggttcctgcatgaagacagtggctggcggtgcctggacgtacaataccacttccgctgtcacggtaaagtccgccatcagaagactgaaggagttgaaagaccagtagacgctcctctactctttgagacatcactggcctataataaatgggttaatttatgtaacaaaaaaaaaaaaaaaaaa

SEQ ID NO: 19 - DNA, 755 bp, Homo sapiens
  40S ribosomal protein S5, S5
ctcttcctgtctgtaccagggcggcgcgtggtctacgccgagtgacagagacgctcaggctgtgttctcaggatgaccgagtgggagacagcagcaccagcggtggcagagaccccagacatcaagctctttgggaagtggagcaccgatgatgtgcagatcaatgacatttccctgcaggattacattgcagtgaaggagaagtatgccaagtacctgcctcacagtgcagggcggtatgccgccaaacgcttccgcaaagctcagtgtcccattgtggagcgcctcactaactccatgatgatgcacggccgcaacaacggcaagaagctcatgactgtgcgcatcgtcaagcatgccttcgagatcatacacctgctcacaggcgagaaccctctgcaggtcctggtgaacgccatcatcaacagtggtccccgggaggactccacacgcattgggcgcgccgggactgtgagacgacaggctgtggatgtgtcccccctgcgccgtgtgaaccaggccatctggctgctgtgcacaggcgctcgtgaggctgccttccggaacattaagaccattgctgagtgcctggcagatgagctcatcaatgctgccaagggctcctcgaactcctatgccattaagaagaaggacgagctggagcgtgtggccaagtccaaccgctgattttcccagctgctgcccaataaacctgtctgccctttggggcagtcccagccaaaaaaaaaaaaaaa

SEQ ID NO: 20 - DNA, 582 bp, Homo sapiens
  60S ribosomal protein L21, L21
tttcctttcggccggaaccgccatcttccagtaattcgccaaaatgacgaacacaaagggaaagaggagaggcacccgatatatgttctctaggccttttagaaaacatggagttgttcctttggccacatatatgcgaatctataagaaaggtgatattgtagacatcaagggaatgggtactgttcaaaaaggaatgccccacaagtgttaccatggcaaaactggaagagtctacaatgttacccagcatgctgttggcattgttgtaaacaaacaagttaagggcaagattcttgccaagagaattaatgtgcgtattgagcacattaagcactctaagagccgagatagcttcctgaaacgtgtgaaggaaaatgatcagaaaaagaaagaagccaaagagaaaggtacctgggttcaactaaagcgccagcctgctccacccagagaagcacactttgtgagaaccaatgggaaggagcctgagctgctggaacctattccctatgaattcatggcataataggtgttaaaaaaaaaaataaaggacctctgggctacaaaaaaaaaaaaaaaaaa

SEQ ID NO: 21 - DNA, 488 bp, Homo sapiens
  40S ribosomal protein S15a transcript variant 1, S15a
ctctttccgccatctttccgcgccgccacaatggtgcgcatgaatgtcctggcagatgctctcaagagtatcaacaatgccgaaaagagaggcaaacgccaggtgcttattaggccgtgctccaaagtcatcgtccggtttctcactgtgatgatgaagcatggttacattggcgaatttgaaatcattgatgaccacagagctgggaaaattgttgtgaacctcacaggcaggctaaacaagtgtggggtgatcagccccagatttgacgtgcaactcaaagacctggaaaaatggcagaataatctgcttccatcccgccagtttggtttcattgtactgacaacctcagctggcatcatggaccatgaagaagcaagacgaaaacacacaggagggaaaatcctgggattctttttctagggatgtaatacatatatttacaaataaaatgcctcatggactctggtgcttccaaaaaaaaaaaa

SEQ ID NO: 22 - DNA, 1125 bp, Homo sapiens
  guanine nucleotide binding protein (G protein), beta polypeptide 2-like 1 (GNB2L1), cell proliferation-inducing gene 21 protein (PIG21), receptor of activated protein kinase C 1 (RACK1), lung cancer oncogene 7 (HLC-7), H12.3
ctctctttcactgcaaggcggcggcaggagaggttgtggtgctagtttctctaagccatccagtgccatcctcgtcgctgcagcgacacacgctctcgccgccgccatgactgagcagatgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccacagcgtgctctgcggggtcactcccactttgttagtgatgtggttatctcctcagatggccagtttgccctctcaggctcctgggatggaaccctgcgcctctgggatctcacaacgggcaccaccacgaggcgatttgtgggccataccaaggatgtgctgagtgtggccttctcctctgacaaccggcagattgtctctggatctcgagataaaaccatcaagctatggaataccctgggtgtgtgcaaatacactgtccaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattggccacacaggctatctgaacacggtgactgtctctccagatggatccctctgtgcttctggaggcaaggatggccaggccatgttatgggatctcaacgaaggcaaacacctttacacgctagatggtggggacatcatcaacgccctgtgcttcagccctaaccgctactggctgtgtgctgccacaggccccagcatcaagatctgggatttagagggaaagatcattgtagatgaactgaagcaagaagttatcagtaccagcagcaaggcagaaccaccccagtgcacctccctggcctggtctgctgatggccagactctgtttgctggctacacggacaacctggtgcgagtgtggcaggtgaccattggcacacgctagaagtttatggcagagctttacaaataaaaaaaaaactggcttttctgacaaaaaaaaaaaaaaaa

SEQ ID NO: 23 - DNA, 644 bp, Homo sapiens
  60S ribosomal protein L11 transcript variant 1, L11, cell growth-inhibiting protein 34 (GIG34), CLL-associated antigen KW-12, DBA7
aaggccctcggccggaagctccgctttctcttcctgctctccatcatggcgcaggatcaaggtgaaaaggagaaccccatgcgggaacttcgcatccgcaaactctgtctcaacatctgtgttggggagagtggagacagactgacgcgagcagccaaggtgttggagcagctcacagggcagacccctgtgttttccaaagctagatacactgtcagatcctttggcatccggagaaatgaaaagattgctgtccactgcacagttcgaggggccaaggcagaagaaatcttggagaagggtctaaaggtgcgggagtatgagttaagaaaaaacaacttctcagatactggaaactttggttttgggatccaggaacacatcgatctgggtatcaaatatgacccaagcattggtatctacggcctggacttctatgtggtgctgggtaggccaggtttcagcatcgcagacaagaagcgcaggacaggctgcattggggccaaacacagaatcagcaaagaggaggccatgcgctggttccagcagaagtatgatgggatcatccttcctggcaaataaattcccgtttctatccaaaagagcaataaaaagttttcagtgaaatgtgcaaaaaaaaaaaa

SEQ ID NO: 24 - DNA, 1013 bp, Homo sapiens
  40S ribosomal protein S20 transcript variant 1, S20
atatttcctgttccggggcgtgtgggacccggatgcaagcgtgctatataagcgttgctcaagtcccacccctttctttttgaggaagacgcggtcgtaagggctgaggatttttggtccgcacgctcctgctcctgactcaccgctgttcgctctcgccgaggaacaagtcggtcaggaagcccgcgcgcaacagccatggcttttaaggataccggaaaaacacccgtggagccggaggtggcaattcaccgaattcgaatcaccctaacaagccgcaacgtaaaatccttggaaaaggtgtgtgctgacttgataagaggcgcaaaagaaaagaatctcaaagtgaaaggaccagttcgaatgcctaccaagactttgagaatcactacaagaaaaactccttgtggtgaaggttctaagacgtgggatcgtttccagatgagaattcacaagcgactcattgacttgcacagtccttctgagattgttaagcagattacttccatcagtattgagccaggagttgagttgatcgaatctacagatgcagaacccatggatacagagggccagcagtacactttgagaagtgtattcgaatccccggggacatgtcctttttaactgcattctcctccgccaaaaaagtgaccaagcagagtctttctctgtcacccaggctggagtgcaatggcgtgatctcagctcactgcaacctctgcctcctgggttcaagtgattctcgtgtctcagcctcctgagtagctgagactacaggtgtgcaccagtgttcccagctgatttttgtattttatgtagagatggggttatgccattttggccaggctagtctcgaactcctgagctcaggtgatacacacacctcagcaaatcttttaaattatacattctgtgatatttccttgactttcttatccagcacttgtattgattatttttcattttgataatgttgggtttttaaaaactcctttatgatggaaaatttc

SEQ ID NO: 25 - DNA, 890 bp, Homo sapiens
  60S ribosomal protein L7a, L7A, surfeit locus protein 3, surfeit 3 (SURF3), thyroid hormone receptor uncoupling protein (TRUP), PLA-X polypeptide
tttcctttctctctcctcccgccgcccaagatgccgaaaggaaagaaggccaagggaaagaaggtggctccggccccagctgtcgtgaagaagcaggaggctaagaaagtggtgaatcccctgtttgagaaaaggcctaagaattttggcattggacaggacatccagcccaaaagagacctcacccgctttgtgaaatggccccgctatatcaggttgcagcggcagagagccatcctctataagcggctgaaagtgcctcctgcgattaaccagttcacccaggccctggaccgccaaacagctactcagctgcttaagctggcccacaagtacagaccagagacaaagcaagagaagaagcagagactgttggcccgggccgagaagaaggctgctggcaaaggggacgtcccaacgaagagaccacctgtccttcgagcaggagttaacaccgtcaccaccttggtggagaacaagaaagctcagctggtggtgattgcacacgacgtggatcccatcgagctggttgtcttcttgcctgccctgtgtcgtaaaatgggggtcccttactgcattatcaagggaaaggcaagactgggacgtctagtccacaggaagacctgcaccactgtcgccttcacacaggtgaactcggaagacaaaggcgctttggctaagctggtggaagctatcaggaccaattacaatgacagatacgatgagatccgccgtcactggggtggcaatgtcctgggtcctaagtctgtggctcgtatcgccaagctcgaaaaggcaaaggctaaagaacttgccactaaactgggttaaatgtacactgttgagttttctgtacataaaaataattgaaataatacaaattttccttc

SEQ ID NO: 26 - DNA, 872 bp, Homo sapiens
  40S ribosomal protein S19, S19, Diamond- Blackfan anemia (DBA, DBA1)
gtactttcgccatcatagtattctccaccactgttccttccagccacgaacgacgcaaacgaagccaagttcccccagctccgaacaggagctctctatcctctctctattacactccgggagaaggaaacgcgggaggaaacccaggcctccacgcgcgaccccttggccctcccctttacctctccacccctcactagacaccctcccctctaggcggggacgaactttcgccctgagagaggcggagcctcagcgtctaccctcgctctcgcgagctttcggaactctcgcgagaccctacgcccgacttgtgcgcccgggaaaccccgtcgttccctttcccctggctggcagcgcggaggccgcacgatgcctggagttactgtaaaagacgtgaaccagcaggagttcgtcagagctctggcagccttcctcaaaaagtccgggaagctgaaagtccccgaatgggtggataccgtcaagctggccaagcacaaagagcttgctccctacgatgagaactggttctacacgcgagctgcttccacagcgcggcacctgtacctccggggtggcgctggggttggctccatgaccaagatctatgggggacgtcagagaaacggcgtcatgcccagccacttcagccgaggctccaagagtgtggcccgccgggtcctccaagccctggaggggctgaaaatggtggaaaaggaccaagatggcggccgcaaactgacacctcagggacaaagagatctggacagaatcgccggacaggtggcagctgccaacaagaagcattagaacaaaccatgctgggttaataaattgcctcattcgtaaaaaaaaaaaaaaaaaaaaaaaaa

SEQ ID NO: 27 - DNA, 418 bp, Homo sapiens
  40S ribosomal protein S21, S21, 8.2 kDa differentiation factor
cttgcccgccgatatctctgccgggtgactagctgcttcctttctctctcgcgcgcggtgtggtggcagcaggcgcagcccagcctcgaaatgcagaacgacgccggcgagttcgtggacctgtacgtgccgcggaaatgctccgctagcaatcgcatcatcggtgccaaggaccacgcatccatccagatgaacgtggccgaggttgacaaggtcacaggcaggtttaatggccagtttaaaacttatgctatctgcggggccattcgtaggatgggtgagtcagatgattccattctccgattggccaaggccgatggcatcgtctcaaagaacttttgactggagagaatcacagatgtggaatatttgtcataaataaataatgaaaacctaaaaaaaaaaaaaaaaaaaaaaa

SEQ ID NO: 28 - DNA, 1289 bp, Homo sapiens
  60S acidic ribosomal protein P0, LP0, P0, ribosomal protein L10E, L10E, acidic ribosomal phosphoprotein P0, PRLP0, RPP0
gtctgacgggcgatggcgcagccaatagacaggagcgctatccgcggtttctgattggctactttgttcgcattataaaaggcacgcgcgggcgcgaggcccttctctcgccaggcgtcctcgtggaaggcccgggaccgcgggatgggtgtcggcgtgaccaggcctgagctccctgtctctcctcagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttctttaaaaagtaaaaaaaaaaaaaaaaaaaaaaaaa

SEQ ID NO: 29 - DNA, 753 bp, Homo sapiens
  40S ribosomal protein S9, S9
ctctttctcagtgaccgggtggtttgcttaggcgcagacggggaagcggagccaacatgccagtggcccggagctgggtttgtcgcaaaacttatgtgaccccgcggagacccttcgagaaatctcgtctcgaccaagagctgaagctgatcggcgagtatgggctccggaacaaacgtgaggtctggagggtcaaatttaccctggccaagatccgcaaggccgcccgggaactgctgacgcttgatgagaaggacccacggcgtctgttcgaaggcaacgccctgctgcggcggctggtccgcattggggtgctggatgagggcaagatgaagctggattacatcctgggcctgaagatagaggatttcttagagagacgcctgcagacccaggtcttcaagctgggcttggccaagtccatccaccacgctcgcgtgctgatccgccagcgccatatcagggtccgcaagcaggtggtgaacatcccgtccttcattgtccgcctggattcccagaagcacatcgacttctctctgcgctctccctacgggggtggccgcccgggccgcgtgaagaggaagaatgccaagaagggccagggtggggctggggctggagacgacgaggaggaggattaagtccacctgtccctcctgggctgctggattgtctcgttttcctgccaaataaacaggatcagcgctttacaaaaaaaaaaaaaaaaaaaaaaaaaaaaaaaaaaaaaaaaaa

SEQ ID NO: 30 - DNA, 2108 bp, Homo sapiens
  40S ribosomal protein S3 transcript variant 1, S3, IMR-90 ribosomal protein S3
atactcacttccgcccgcgagccacttcctttcctttcagcggagcgcggcggcaagatggcagtgcaaatatccaagaagaggaagtttgtcgctgatggcatcttcaaagctgaactgaatgagtttcttactcgggagctggctgaagatggctactctggagttgaggtgcgagttacaccaaccaggacagaaatcattatcttagccaccagaacacagaatgttcttggtgagaagggccggcggattcgggaactgactgctgtagttcagaagaggtttggctttccagagggcagtgtagagctttatgctgaaaaggtggccactagaggtctgtgtgccattgcccaggcagagtctctgcgttacaaactcctaggagggcttgctgtgcggagggcctgctatggtgtgctgcggttcatcatggagagtggggccaaaggctgcgaggttgtggtgtctgggaaactccgaggacagagggctaaatccatgaagtttgtggatggcctgatgatccacagcggagaccctgttaactactacgttgacactgctgtgcgccacgtgttgctcagacagggtgtgctgggcatcaaggtgaagatcatgctgccctgggacccaactggtaagattggccctaagaagcccctgcctgaccacgtgagcattgtggaacccaaagatgagatactgcccaccacccccatctcagaacagaagggtgggaagccagagccgcctgccatgccccagccagtccccacagcataacagggtctccttggcagctgtattctggagtctggatgttgctctctaaagacctttaataaaattttgtacaaagacacaaggtctgactagactgttcagtattcagactgaggggcatgttggcctctggagcattacatatcttcttggttttaaccatacttgtggtatttgcaagggccagaacagtaagacccaagcagagccaaccagagaaataatatttgtgtgatagagaaggctgatagcaagcaaggcagcaccttgattcgttgtcctgtagttcaggattgtaggtttagaagagggatatgtttgagtttttcctatgcataaggcgatccacgttgcacatagaaagtgaatataaatggccattatattttgtgtcatgctgtgctctaagtgttctttacatatgtactcgttaatcaacctctctaaagtgtaaaggaaatttgcttgcaccactgaaggcacataaggctcagaagtaaatttgcctaagcagtataaagctatcattagaatccacattcctaagttgtgttctcttaggggatcatggaaccagtcattggtactacaggctattatgttctggagaactgtgaagaacatttaaattgtctctgattttatctatcaatgttttgaagtattttctaccagtgtctgtacttcacaagaaattcggcactattttttcaggcaaaactagtgagggacaggttggcttgaaaatcatgagactgttgttaaatcagatgctggttgatcacagaggggacttccagggaaagctgttatcaggtggctgcttcctggtgatgcagcctggctgatgagataaccctggctccacagatggcttagcaggtgctgtgatgatttggttttcttctcaattagactgagctgcacatggtgtttatattgcttggcacatggtaagggcttaatatttgaggtaattatgtagggcgtacactgacaagtatctgacccccccttcctttttgactcataaattggtcatcttaaccatttaagtgtacacttctatagtgacagagttagccctctgtccaagggatttgcatctgtggattcaaccaactttgggtcaaaaataatcaaaaaggatggttgtgtgtgtattgaacatgtagacttatttttcttattttcaaaatactatattttcttgtcacttattttcttgtacactgcagttgtaacagctatgtagcatgtacattaggtattaaaagtaatccagtgaagattgaaagtctaaaaaaaaaaaaaaaaaa

SEQ ID NO: 31 - DNA, 6668 bp, Homo sapiens
  cartilage associated protein precursor (CRTAP, CASP), leprecan-like 3 (LEPREL3), OI7
aggctggcgtccccgccccgaaagcactgggcccgccgcgtcgcaccgtcctctttcctttccttctccctccccttttcccttccttcgtcccttccttccttcctttcgccgggcgcgatggagccggggcgccggggggccgcggcgctgctagcgctgctgtgcgtggcctgcgcgctgcgcgccgggcgcgcccaatacgaacgctacagcttccgcagcttcccacgggacgagctgatgccgctcgagtcggcctaccggcacgcgctggacaagtacagcggcgagcactgggccgagagcgtgggctacctggagatcagcctgcggctgcaccgcttgctgcgcgacagcgaggccttctgccaccgcaactgcagcgccgcgccgcagcccgagcccgccgccggcctcgccagctatcccgagctgcgcctcttcgggggcctgctgcgccgcgcgcactgcctcaagcgctgcaagcagggcctgccagccttccgccagtcccagcccagccgcgaggtgctggcggacttccagcgccgcgagccctacaagttcctgcagttcgcttacttcaaggcaaataatctccccaaagccatcgccgctgctcacacctttctactgaagcatcctgatgacgaaatgatgaagaggaacatggcatattataagagcctgcctggtgccgaggactacattaaagacctggaaaccaagtcatatgaaagcctgttcatccgagcagtgcgggcatacaacggtgagaactggagaacatccatcacagacatggagctggcccttcccgacttcttcaaagccttttacgagtgtctcgcagcctgcgagggttccagggagatcaaggacttcaaggatttctacctttccatagcagatcattatgtagaagttctggaatgcaaaatacagtgtgaagagaacctcaccccagttataggaggctatccggttgagaaatttgtggctaccatgtatcattacttgcagtttgcctattataagttgaacgacctgaagaatgcagccccctgtgcagtcagctatctgctctttgatcagaatgacaaggtcatgcagcagaacctggtgtattaccagtaccacagggacacttggggcctctcggatgagcacttccagcccagacctgaagcagttcagttctttaatgtgaccacactccagaaggagctgtatgactttgctaaggaaaatataatggatgatgatgagggagaagttgtggaatatgtggatgacctcttggaactggaggagaccagctagcccacagcaaccaaagagacttcctcttggcgttcaggaaacacagattctttgtccttttcccaacagcccaggctgttgatacctcagagccttctctttactctccaaagtgaaagggaagcccccgtctctctaactgcatgtcatcaggggtgagcctgcctttcctatcttcacacctgccacctcatgttcacacctatctttctcaccttttttttgagatggagtctcgctctcttgcccaggctggagtgcaatggcacgttctcagctcactgcaacctccgcctcttgggttcaagcaattctgctgcatcagcctcccgagtacctgggattacaggcatgtgccaccacgcccggctaattttgtatttttagtagagacggggttttgccatgttggccaggctggtctcgaactcttgacttcagatgatccatctgccttggcctcccacagtgctgggattacaggcgtgagccaccatgcccggcctctttctcacctttacacctgtcttcttatcctcacatctgttttcacaccttcatccctgtcttcctcatgttcacacttgtcttccccatgttcatagctgcctttcttaccattttggtttgaagggcagtcttctctggcttgtttttttgtttttcccagaaaatcagtattattttttaaataagaaaaacattcctagaagatgataattgtgaaaacctcctttggcttatttgcttttccagattttagtctcctttctccccatccgggaaagatggtggaagacataggctaaatttctccagcctcacaatggtcttcacttggtctgacttgtaccaattctagcacccactgaaaaacaagttgagtagagagtgtagagtgcagaaatgtggcttttgccccactttgcatctccaaaattacaacggttggccgatcccatttgaggacaatgcttagttataagtctccgagttggaaaaggaagaaagccagagctgtctagtttcattcattctttcagtaaatatttattgagtacctactgtgtgctaggcattgacctgggaactagagatacttcacagaataacagggaaagttccctgtgctcatggagcttacattctacagggagaaagagatagccaatacataggaataaatatatacaaggtatcatgtagtgataattgctgtggagaaaaataaagcaggggagggagtaagaaatcctggagatgaggctgcagttttaaatggggcctcactgggaatgtgacgttgagcagagacgttagggaagtggatcctggacaaggcattccaggcagaggaacaggatgtgcactgccccaaagtgagaacttgctctacgtggtcaggaaagagcagggagaccaagcagagtcgtgggcaggggtagaatggaaggagaggcggctggggaggacaggtggtggagggccttggcttctgctaagtgagatgggaaccactggagggtttgaacagaggagtgccttgattgatttatattttgcaagggtcattctagctgccatattgtgaaaaactttagtggacaagggcagaaggaagagggaagacctgttaggaagctactgcaaggttccaggcttgggcctgggccacagcaacagcagtggtcaaatatctagatttattttgaaaagagccaataggatttgctgagagtttgaatgtggagtgtaagagaaggaagagttaatgatgacattaaggtttttggcctgaatagcaggaaagatggagttaccagttactgaaatagggaaggatgggctgggtaagtatggaatttggtgcaaagcaggctgtctgtggttggaatgggaggttctggctgcaaatcaaagtggagagttctctcaggtcaggtctgcagcagagctcgagacagggatctgaatgcacttggtttattgttgggggtgctctcagaaggaacctgtgaaagcctttatcagtcatttattggctgtgagaagttctctgggagtgtgggtacatttgaaggcaagtgacttcagttgagggcaagtctctggaaaagaggctgtaggcatctggcagctaccatgcatggtagtgtgttgggggtgggggtcctgggcactggctgtgtgaagggatctggcagggcaccacagcgccccctactgaaccatcagcatgtcagtggcatttaaagccatgcagctggaggggccactgagattgtctctgagtattactgagaagcaacagaaaagagccatggatggagcccttgggctctctgggaaatgggaaatcagccaaaggactgagaaggagttaccttaaggtcagagaaaaccaagagagtgtggtgttctggaagctgagctttctttattcaacctcattcccttctccaaataagccacttgtgtagttgggcccctccagggttgaaggcaagaggagaaaggcacagcgtttgggaaacaagacttttcctgcaatagcctgggaaggaataaaaggatagagtgtttgggtttttgtgtaatggtggttaattggggtggaacactcacacgttgtgctttttctgggcttcccttatcccccagaacactctaccaacctcggggaactcgggcacatccttctgtttctccttcagctctatcctgctttcctcatcccttctgacaccacgtcctcactcacctgcacaagaatccctgcatcaggttctcctttgagggtacccacccaggacagtcccctaccacttctgtcttgggctgaagttgcccacgtccacaaaatctgtactcccagcgggggtgtttggcccgaggagtcagtgttattactggtggatgcaccgtgtccacagcagcccccaatcccagcgatgcgtcagatcttacgtggcttcctgctgggggagatggccttcacccacgggatgccgggttctcctttctttcctcaccccaacctttactccaccagagaaacttccttttgaactcagtggggaagagggtgatgagacaggactagaaagtagtgggggacccagcgagtggacgccctgctccgggattcctgagtctgtaaatagtgtgcccagcagctgtgaactccccttatagcctcaggctgcagtgtccttcccagctgtgtgagaaaatgaaagccgacgtccacagggacccaggcagggttgggtgttgtgactcactccacctctgtgccctgcagaggtactgttgggtccttgtcttgtgagcctggggtgagctctctgtacatgttgttgttccacgtatgggttgacttggcatgctggggggtcctcgttcactctctgaagttggcctcctttcactggggattgaaaagcacctccacccctaccctagtgatgtcccctgaggacccgggtgatagtacagtcaatattgtcagtactttgctttgattgaaggctgtagagctgagttaccaaaatttctatttcaaaggaaaccaaaccttaaaaaaaaaaaacaaaaactgggctgggtcttccaaacctaccatgaaaccctggtgtgcaggctgcactcaatgacctcaacccaacacctccctgagtgtgcttcttggaagagcctagaagattcctggatggagaccccattggttcagcctcaagtctggcccgtcttcgaaaaaacaaacacatttgtaagctttgtgggagcttccaggcctgctctaagatgccttgcttgtcctttgacccatcagcatggagctcagtggttgctgtttggttctgcaggctggtggggaggccgcccatcgtggtggggcatctgtccagccccattgccactcagggcatccaaacaggaggcacccgctgggaagggtctaaagatactccttgtggccactgctactgttcacacttgacttgtggagaagcgaagggctgaggggaggtttgtgtacacccatgtatttaaaagtgactgactgactgaaatgagcacataccgacatatgcaacatactaataccttcctgattttcgagactttctaattactacaactaacctgttgtgctcacctctggaattcagaaagagagccactgcgagcactgaccacaagggctgccttaggaaggaaatgtgtgctttcaggagttcttattaggggaattttaagagcacaaaaattttattgaacccaccttagtgacaaacagaaaatgatttgttagattgtcagttggaaggggtttattatgactgctggtgaaataaacagtgaccagtttctgccaacatttatggaaataacgtttctaggttttaaatgtgagccgtaaactgaagctacttcagttaaaaaaaaaaaaatcaatacttaaactgtagggaaaaggtggattggtgccagagaaaacattatttaatagtgtcagaacatggaactgcaacacagtttgtagcaaggcactgagaaaaacccacaactaatccttcatcgcagctgtctgaactcttgatcatctgccatcccccaaacagtgacttctttttttctggtgactccaggcctgaatgacctagtgtggagagtttaaactatgtacaagggaggaaagaaaaaaaggaaaggaaccttaagtaagcctcagccaaaggttttatgcattggacttcctgtgcctgcccccaggggcaagactgatccccatgcctgtgcccatgacatctccctgaaagaggacaccatgacagcccggctttgccttgactgacccactgctaccccagaaataagaatcaagagcagctattgttatccttagagtgttttccgtctagggccggctcgtgaacagccacatatccttgcacctgacactgtccccacacaaatagctggctttcgttgcttgttgaatgaatgagtgagttggctctatatccccttggagctggccggtaagatattagtgcctcattttacaagaagagaataggaaggaattaagcaatttggccaacagatacaagatagattccagagttttatctcccactttagggtggcagccagtaggccaaactccaaagaccgttgctgatgtctttttctgcctcccctctttgggttagtgtggtatgtacaagctcactcttgttgaaaattagaaaatagttgaaaacaaaaggtttttgtttttctttttaaatcacattaaatgttttacattgcttaaaaaaaaaaaaaaaaaa

SEQ ID NO: 32 - DNA, 967 bp, Homo sapiens
  hypothetical protein LOC80097, family with sequence similarity 128, member B (FAM128B), mitotic-spindle organizing protein 2B (MZT2B), mitotic-spindle organizing protein associated with a ring of gamma-tubulin 2B (MOZART2B)
tttcttgaaagaggcatttaccgagcgcccaatgtatgcctggcactgggctgggtgctgccacctaagcgagcacgaccaatgcagtctatcagggaggcccagatcgccaagcagcggacccctgcggtccgccatgccactcccggctcctagagcgccgctcagcacaccgtgagcgcccaataactgttgggcttcaatgacgccgcggaggcggccccgtccccgcgctcccgcccctcccgccagggcagcccgggaggccagacgttgacgctgcagggagagggtggtgggcgcagccgctagggggcgcggcggggcggagcgcacctttccgcgggccgcggggatggcggcgcagggcgtagggcctgggccggggtcggcggcgcccccggggctggaggcggcccggcagaagctggcgctgcggcggaagaaggtgctgagcaccgaggagatggagctgtacgagctggcgcaggcggcgggcggcgctatcgaccccgacgtgttcaagatcctggtggacctgctgaagctgaacgtggcccccctcgccgtcttccagatgctcaagtccatgtgtgccgggcagaggctagcgagcgagccccaggaccctgcggccgtgtctctgcccacgtcgagcgtgcccgagacccgagggagaaacaaaggcagcgctgccctcgggggagcattggccctggcggaacgcagcagccgcgaaggatccagccagaggatgccacgccagcccagcgctaccaggctgcccaaggggggcgggcctgggaagagccctacacggggcagcacctaggatggggcagagacttgttgcatctttgtccccagcaaaggctacatgttacctccttcaattgataataaacctttctgagatgcagagggtccaggtcaaaaaaaaaaaaaaaaaaaaaaaaaaaaaaaaaaa

SEQ ID NO: 33 - DNA, 1787 bp, Homo sapiens
  60S ribosomal protein L32 transcript variant 3, L32, PP9932
gacctcctgggatcgcatctggagagtgcctagtattctgccagcttcggaaagggagggaaagcaagcctggcagaggcacccattccattcccagcttgctccgtagctggcgattggaagacactctgcgacagtgttcagtccctgggcaggaaagcctccttccaggattcttcctcacctggggccgcttcttccccaaaaggcatcatggccgccctcagaccccttgtgaagcccaagatcgtcaaaaagagaaccaagaagttcatccggcaccagtcagaccgatatgtcaaaattaagcgtaactggcggaaacccagaggcattgacaacagggttcgtagaagattcaagggccagatcttgatgcccaacattggttatggaagcaacaaaaaaacaaagcacatgctgcccagtggcttccggaagttcctggtccacaacgtcaaggagctggaagtgctgctgatgtgcaacaaatcttactgtgccgagatcgctcacaatgtttcctccaagaaccgcaaagccatcgtggaaagagctgcccaactggccatcagagtcaccaaccccaatgccaggctgcgcagtgaagaaaatgagtaggcagctcatgtgcacgttttctgtttaaataaatgtaaaaactgccatctggcatcttccttccttgattttaagtcttcagcttcttggccaacttagtttgccacagagattgttcttttgcttaagcccctttggaatctcccatttggaggggatttgtaaaggacactcagtccttgaacaggggaatgtggcctcaagtgcacagactagccttagtcatctccagttgaggctgggtatgaggggtacagacttggccctcacaccaggtaggttctgagacacttgaagaagcttgtggctcccaagccacaagtagtcattcttagccttgcttttgtaaagttaggtgacaagttattccatgtgatgcttgtgagaattgagaaaatatgcatggaaatatccagatgaatttcttacacagattcttacgggatgcctaaattgcatcctgtaacttctgtccaaaaagaacaggatgatgtacaaattgctcttccaggtaatccaccacggttaactggaaaagcactttcagtctcctataaccctcccaccagctgctgcttcaggtataatgttacagcagtttgccaaggcggggacctaactggtgacaattgagcctcttgactggtactcagaatttagtgacacgtggtcctgattttttttggagacggggtcttgctctcacccaggctgggagtgcagtggcacactgactacagccttgacctccccaggctcaggtgatcttcccacctcagccttccaagtagctgggactacagatgcacacctccaaacctgggtagtttttgaagtttttttgtagaggtggtctagccatgttgcctaggctcccgaactcctgagctcaagcaatcctgcttcagcctcccaaagtactgggattacaggcatcttctgtagtatataggtcatgagggatatgggatgtggtacttatgagacagaaatgcttacaggatgtttttctgtaaccatcctggtcaacttagcagaaatgctgcgctgggtataataaagcttttctacttctagtctagacaggaatcttacagattgtctcctgttcaaaacctagtcataaatatttataatgcaaactggtcaaaaaaaaaaaaaaaaaa

SEQ ID NO: 34 - DNA, 1552 bp, Homo sapiens
  eukaryotic translation elongation factor 1 gamma (EEF1G, EF1G, EF-1-gamma; eEF-1B gamma), translation elongation factor eEF-1 gamma chain, pancreatic tumor-related protein, GIG35, PRO1608
aactgtcgtacgtatgtcttctgtttcgtcctcgctttccggctgctgtttctccacggctctcctctttccccctcccttctctcccgggcggcttactttgcggcagcgccgagaaccccaccccctttctttgcggaatcaccatggcggctgggaccctgtacacgtatcctgaaaactggagggccttcaaggctctcatcgctgctcagtacagcggggctcaggtccgcgtgctctccgcaccaccccacttccattttggccaaaccaaccgcacccctgaatttctccgcaaatttcctgccggcaaggtcccagcatttgagggtgatgatggattctgtgtgtttgagagcaacgccattgcctactatgtgagcaatgaggagctgcggggaagtactccagaggcagcagcccaggtggtgcagtgggtgagctttgctgattccgatatagtgcccccagccagtacctgggtgttccccaccttgggcatcatgcaccacaacaaacaggccactgagaatgcaaaggaggaagtgaggcgaattctggggctgctggatgcttacttgaagacgaggacttttctggtgggcgaacgagtgacattggctgacatcacagttgtctgcaccctgttgtggctctataagcaggttctagagccttctttccgccaggcctttcccaataccaaccgctggttcctcacctgcattaaccagccccagttccgggctgtcttgggcgaagtgaaactgtgtgagaagatggcccagtttgatgctaaaaagtttgcagagacccaacctaaaaaggacacaccacggaaagagaagggttcacgggaagagaagcagaagccccaggctgagcggaaggaggagaaaaaggcggctgcccctgctcctgaggaggagatggatgaatgtgagcaggcgctggctgctgagcccaaggccaaggaccccttcgctcacctgcccaagagtacctttgtgttggatgaatttaagcgcaagtactccaatgaggacacactctctgtggcactgccatatttctgggagcactttgataaggacggctggtccctgtggtactcagagtatcgcttccctgaagaactcactcagaccttcatgagctgcaatctcatcactggaatgttccagcgactggacaagctgaggaagaatgccttcgccagtgtcatcctttttggaaccaacaatagcagctccatttctggagtctgggtcttccgaggccaggagcttgcctttccgctgagtccagattggcaggtggactacgagtcatacacatggcggaaactggatcctggcagcgaggagacccagacgctggttcgagagtacttttcctgggagggggccttccagcatgtgggcaaagccttcaatcagggcaagatcttcaagtgaacatctcttgccatcacctagctgcctgcacctgcccttcagggagatgggggtcattaaaggaaactgaacattgaaaaaaaaaaaaaaaa

SEQ ID NO: 35 - DNA, 1978 bp, Homo sapiens
  60S ribosomal protein L22-like 1
cgctctcagcgcgtgacgcagcacgctttgatataaatgcagaccgcgcggccgtagcttcctctctgctctcgcggccgactcgcaagatggcgccgcagaaagacaggaagcccaagaggtcaacctggaggtttaatttggaccttactcatccagtagaagatggaatttttgattctggaaattttgagcaatttctacgggagaaggttaaagtcaatggcaaaactggaaatctcgggaatgttgttcacattgaacgcttcaagaataaaatcacagttgtttctgagaaacagttctctaaaaggtatttgaaataccttaccaagaaataccttaagaagaacaatcttcgtgattggcttcgagtggttgcatctgacaaggagacctacgaacttcgttacttccagattagtcaagatgaagatgaatcagagtcggaggactaggcaaaggctccccttacagggctttgcttattaataaaataaatgaagtatacatgagaaataccaagaaattggcttttagtttatcagtgaataaaaaatattatactcttgaacttttgtctcatttttttgagtatgctgtttatatgattttgatttccctctgataactatcaacagtatttaaatagcttatagctggtataattttttcccacgatttccaaaatcttttatgtactcaggtaaaagtagcgttatataggaaatcttttttttagacactctcgttctgtcacccaggctggagtgcagtgactcagcttcctaaatagctggaattacaggtgtgagccaccatgcccggctaattttttgtacttttagtagagtagggtttggccatgttggccaggctggtttcaaactcctgacctcaagtgatctacccacctcggcttcccaaagtgctgattatagctgtgaaccaccatgcccggccaggaaatcttactgtagaacaattttttatatagctgtataaaatgtatatgattgtcttgacagtctcaaatactgtttttaatagcttgtaaatgtaatctcaagtgcttagaacagttcttacatataagttgctctgtagtttgctcttatagttagcccaaagactctgggtgtgaggcctgctgtaaaccaatgttaaactgcttattagaaagccctaaccacctgctttgtaggcaccagaaactcaaaaccaaatctcaactcagctacagaatctactgtggtccttgtctgaaaaaattagttcactcggttggaatcttgtctcagagcatcctcatctctttctcaaaagcccctaccccaacaccggcgtgttggttgtctattgaaacttacaagtggatggaccctttctcccgaataaactggcctttgaaagctctaatcgaaatggtttggcaaaatccatactgcaggagattagggaggacaagaatgatgtgcctttttgtactgctgagcctgatggtggtgccactacttcaggtacttagatgagtcttgatgctaatagaattgtgtcgccaaacatatctggacagttacaacctaatctatgcattaattggtttgggaattgcttgaaattattgtttaattcaatgttttaattcgttttcctaaaaatttaagtgcccccatcatcgtgcaatacctcagtgcagcaactccttgattcttggatgactgaacttcctaacttggctctgccccattgttcccatttttcatgtttttcacaaatagttaaccaggtacctactactgtgcaccgctgcagagcattgaggatgtatgtgatgagtaaaaacacccagcctgctctgctgtgttagtattatgacggaaactgatcaaatcacatgtgaacaaatttactgctacaaaagggagggcttaataaaaggaatttcatctgggaaggcaaaaaaaaaaaaaaaaaa

SEQ ID NO: 36 - DNA, 1561 bp, Homo sapiens
  Y box binding protein 1 (YB1, YB-1, YBX1), nuclease sensitive element binding protein 1 (NSEP1, NSEP-1), DNA-binding protein B (DBPB), CCAAT-binding transcription factor I subunit A (CBF-A), enhancer factor I subunit A (EFI-A), BP-8, CSDA2, CSDB, MDR-NF1
gggcttatcccgcctgtcccgccattctcgctagttcgatcggtagcgggagcggagagcggaccccagagagccctgagcagccccaccgccgccgccggcctagttaccatcacaccccgggaggagccgcagctgccgcagccggccccagtcaccatcaccgcaaccatgagcagcgaggccgagacccagcagccgcccgccgccccccccgccgcccccgccctcagcgccgccgacaccaagcccggcactacgggcagcggcgcagggagcggtggcccgggcggcctcacatcggcggcgcctgccggcggggacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgcggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaaggccaggcccaacaacgccggccctaccgcaggcgaaggttcccaccttactacatgcggagaccctatgggcgtcgaccacagtattccaaccctcctgtgcagggagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcgtccgctcccgaggctgagcagggcggggctgagtaaatgccggcttaccatctctaccatcatccggtttagtcatccaacaagaagaaatatgaaattccagcaataagaaatgaacaaaagattggagctgaagacctaaagtgcttgctttttgcccgttgaccagataaatagaactatctgcattatctatgcagcatggggtttttattatttttacctaaagacgtctctttttggtaataacaaacgtgttttttaaaaaagcctggtttttctcaatacgcctttaaaggtttttaaattgtttcatatctggtcaagttgagatttttaagaacttcatttttaatttgtaataaaagtttacaacttgattttttcaaaaaagtcaacaaactgcaagcacctgttaataaaggtcttaaataataaaaaaaaaaaaaaa

SEQ ID NO: 37 - DNA, 4041 bp, Homo sapiens
  eukaryotic translation initiation factor 4B (EIF4B, EIF-4B), PRO1843
ggccacatgtcgcgcatgtcttcccgtcggacggcgtgccacctcgccgcgcagctttacgaacctagagcagcgccgccccgcctcctgtctccgtcctcacctccccgccccctcccagcttcgcgtctcctagctcgacgcgcccgctataatcacgtgattgcctcatccgggtcttttgcgttctctttccctctcccaacatggcggcctcagcaaaaaagaagaataagaaggggaagactatctccctaacagactttctggctgaggatgggggtactggtggaggaagcacctatgtttccaaaccagtcagctgggctgatgaaacggatgacctggaaggagatgtttcgaccacttggcacagtaacgatgacgatgtgtatagggcgcctccaattgaccgttccatccttcccactgctccacgggctgctcgggaacccaatatcgaccggagccgtcttcccaaatcgccaccctacactgcttttctaggaaacctaccctatgatgttacagaagagtcaattaaggaattctttcgaggattaaatatcagtgcagtgcgtttaccacgtgaacccagcaatccagagaggttgaaaggttttggttatgctgaatttgaggacctggattccctgctcagtgccctgagtctcaatgaagagtctctaggtaacaggagaattcgagtggacgttgctgatcaagcacaggataaagacagggatgatcgttcttttggccgtgatagaaatcgggattctgacaaaacagatacagactggagggctcgtcctgctacagacagctttgatgactacccacctagaagaggtgatgatagctttggagacaagtatcgagatcgttatgattcagaccggtatcgggatgggtatcgggatgggtatcgggatggcccacgccgggatatggatcgatatggtggccgggatcgctatgatgaccgaggcagcagagactatgatagaggctatgattcccggataggcagtggcagaagagcatttggcagtgggtatcgcagggatgatgactacagaggaggcggggaccgctatgaagaccgatatgacagacgggatgatcggtcgtggagctccagagatgattactctcgggatgattataggcgtgatgatagaggtcccccccaaagacccaaactgaatctaaagcctcggagtactcctaaggaagatgattcctctgctagtacctcccagtccactcgagctgcttctatctttggaggggcaaagcctgttgacacagctgctagagaaagagaagtagaagaacggctacagaaggaacaagagaagttgcagcgtcagctggatgagccaaaactagaacgacggcctcgggagagacacccaagctggcgaagtgaagaaactcaggaacgggaacggtcgaggacaggaagtgagtcatcacaaactgggacctccaccacatctagcagaaatgcacgaaggagagagagtgagaagtctctagaaaatgaaacactcaataaggaggaagattgccactctccaacttctaaacctcccaaacctgatcagcccctaaaggtaatgccagcccctccaccaaaggagaatgcttgggtgaagcgaagttctaaccctcctgctcgatctcagagctcagacacagagcagcagtcccctacaagtggtgggggaaaagtagctccagctcaaccatctgaggaaggaccaggaaggaaagatgaaaataaagtagatgggatgaatgccccaaaaggccaaactgggaactctagccgtggtccaggagacggagggaacagagaccactggaaggagtcagataggaaagatggcaaaaaggatcaagactccagatctgcacctgagccaaagaaacctgaggaaaatccagcttccaagttcagttctgcaagcaagtatgctgctctctctgttgatggtgaagatgaaaatgagggagaagattatgccgaatagacctctacatcctgtgcttttctcctagtttctctccaccctggaacattcgagagcaaatcaaaacctctatccagacaagacaaaataaaactcaccatctcctgaagacctttcttacctttttttaaaaacaaaaaatgaaattattttgcatgctgctgcagcctttaaagtattgaagtaactggagaattgccaatacagccagagagaaagggactacagctttttagaggaaaagttgtggtgcgttatgtcaccatgcagttgccagtgtgattagtgcctaggggtctccatttagcagaaatggtaatgacagtgatataatgcctggaacctggttgggcagtaggggagggaggtagaaggaaaagtgtgagatttctaccttttagtttttatcctattgtggcatatatgaattctcaaacattatctgaataaattttccactcttggaaaggtagatttagcctcaagttgttctagtctccaggaggctgccagcccctcctcttatttaattctgagttttgggggccagcctagagggaattccttttttttttttaaccccccaggggggtagttgggagtgagactataggccataaagaatgggactgcattggaccaaaataaatgggaaaatcgtggtttgaaaagaagcttttgggaagtgatgagtcattttgcaccaggtaataggggaaaattgtgtgacctccagcaaacacatgaatggttatttcctggagccggaagcacttgggggtcgtggtaattcccagtgttttctgtgtcctagttttaccctttctaaacactgtcctttttgaaagttttgaatatatccacattctattgaaaccttgaaactaaaaatttagactcttatcatcatcttaagttcttcatgctactcttaacctcccaaaaagcagtatctaagtcacatacatgatgtcttgggcattttctcagccatggagaactctgaaaggaagaatcgctgcttttctcaagcaaatcggtttcttgatgtcttttggttctccttgcctgctcctgatgcttggaccccttttattgatcagagtgctctagaataatggatggtcttggatgatggataaatagggacagggacagttaaattgggagcctttcttacaaccttgatgggatttttccccccaagtttccttctccactgaaatgccacactaatgcttgttggattcatgaggtggccagaccaatgtgttgttttgttgttgtttttttaagcttcccttgagagaataaatggtaatggagagaactatttaacaaggtcctggtttctcttgcaacacagtagctaaacttgcctgcttttatatgcatttttgtagggatcagcttggtagacagtattagcggagaaacaccttgatcttggtttgcaagcccttctcccatcagtcctagattaggccctgttcagccatgcaggggtgttggtttatgcgtgctgcagcagtgggcataatgaatataatttacccagtggacaaaggtgtgtaccaagtgaatttaaataattggtgtggattggccagtagctaagaagtgggcttttaaagagtattgaagattgaaagggtttttctttcttttttaaaaaagaaaaacaaactattgattgtagataatgaaaagctagggtttgccctcttcatgtctactctccttccaaatagttatatccaaaactgtttttccctctcccctaccttgtcccccctattaaaatagaaacagggattgattaatgtcccgctcctgaatacatgtaaaatttgtacaaaaatatcttctatgaaaatgatttgtaatctgtagacttattacctgggagatgtcttgatgtaaaatcccatcctttgggttgtgggttttttgttttctccaaataaatctgatctttaaagttcaaaaaaaaaaaaaa

SEQ ID NO: 38 - DNA, 511 bp, Homo sapiens
  60S acidic ribosomal protein P2, LP2, P2, RPP2, D11S2243E, ribosomal protein, large, P2, acidic ribosomal phosphoprotein P2, renal carcinoma antigen NY-REN-44
ggtttaaccccgcctcttgcgtcggcgccttccttttcctccctgtcgccaccgaggtcgcacgcgtgagacttctccgccgcctccgccgcagacgccgccgcgatgcgctacgtcgcctcctacctgctggctgccctagggggcaactcctcccccagcgccaaggacatcaagaagatcttggacagcgtgggtatcgaggcggacgacgaccggctcaacaaggttatcagtgagctgaatggaaaaaacattgaagacgtcattgcccagggtattggcaagcttgccagtgtacctgctggtggggctgtagccgtctctgctgccccaggctctgcagcccctgctgctggttctgcccctgctgcagcagaggagaagaaagatgagaagaaggaggagtctgaagagtcagatgatgacatgggatttggcctttttgattaaattcctgctcccctgcaaataaagcctttttacacatctcaaaaaaaaaaaaaaaaaa

SEQ ID NO: 39 - DNA, 603 bp, Homo sapiens
  40S ribosomal protein S16, S16
gaaaagcggccagggtggcccctagctttccttttccggttgcggcgccgcgcggtgaggttgtctagtccacgctcggagccatgccgtccaagggcccgctgcagtctgtgcaggtcttcggacgcaagaagacagcgacagctgtggcgcactgcaaacgcggcaatggtctcatcaaggtgaacgggcggcccctggagatgattgagccgcgcacgctacagtacaagctgctggagccagttctgcttctcggcaaggagcgatttgctggtgtagacatccgtgtccgtgtaaagggtggtggtcacgtggcccagatttatgctatccgtcagtccatctccaaagccctggtggcctattaccagaaatatgtggatgaggcttccaagaaggagatcaaagacatcctcatccagtatgaccggaccctgctggtagctgaccctcgtcgctgcgagtccaaaaagtttggaggccctggtgcccgcgctcgctaccagaaatcctaccgataagcccatcgtgactcaaaactcacttgtataataaacagtttttgagggattttaaagtttcaagaaaaaaaaaaaaaaa

SEQ ID NO: 40 - DNA, 2151 bp, Homo sapiens
  vimentin (VIM), epididymis luminal protein 113 (HEL113), CTRCT30
gcctctccaaaggctgcagaagtttcttgctaacaaaaagtccgcacattcgagcaaagacaggctttagcgagttattaaaaacttaggggcgctcttgtcccccacagggcccgaccgcacacagcaaggcgatggcccagctgtaagttggtagcactgagaactagcagcgcgcgcggagcccgctgagacttgaatcaatctggtctaacggtttcccctaaaccgctaggagccctcaatcggcgggacagcagggcgcgtcctctgccactctcgctccgaggtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaactcaaaaaaaaaaaaaaa

SEQ ID NO: 41 - DNA, 1787 bp, Homo sapiens
  guanidinoacetate N-methyltransferase transcript variant 2 (GAMT), epididymis secretory protein Li 20 (HEL-S-20), Cmisc_feature2, PIG2, TP53I2
gggcctgttggacccgcccccggcccacaagcccctgcagggagcgggcccgggcggcgcgcgatcgaggtcgggtcgccgtccagcctgcagcatgagcgcccccagcgcgacccccatcttcgcgcccggcgagaactgcagccccgcgtggggggcggcgcccgcggcctacgacgcagcggacacgcacctgcgcatcctgggcaagccggtgatggagcgctgggagaccccctatatgcacgcgctggccgccgccgcctcctccaaagggggccgggtcctggaggtgggctttggcatggccatcgcagcgtcaaaggtgcaggaggcgcccattgatgagcattggatcatcgagtgcaatgacggcgtcttccagcggctccgggactgggccccacggcagacacacaaggtcatccccttgaaaggcctgtgggaggatgtggcacccaccctgcctgacggtcactttgatgggatcctgtacgacacgtacccactctcggaggagacctggcacacacaccagttcaacttcatcaagaaccacgcctttcgcctgctgaagccggggggcgtcctcacctactgcaacctcacctcctggggggagctgatgaagtccaagtactcagacatcaccatcatgtttgaggtgcgcccacctgaagttccccatgggtctccaggaagtgaccttggatgggggtgggaaggggctgctggagccaccttgctacctggggagggtcccttcctgaccccctgggtgggctggactgtgctggttcatttagaaatcaaagtcctttgcctggcgcagtggctgccaggagcagtggctcaggtctataatcccagcactgtggaaggccgaggtgggcagattgcttgagcctaggagttcaagaccagcctgggctacagagcaagacctcatctttcctaaaaaaaaaaaatacaaaaaactagctgggcgtggtggcgtgtgcctgtactcccagctacttgggaggctgaggtaggaggattgcttgggccccaggggcagaggttgcagtgagcccagatcgaaccactgcactccagcctgggtaacagagccagaccctgtctcaagaaaaaagaaagaaagaaagaaagaaagaaaatttaaaaaaaaaatggaaatcaggctgggcgtggtggctcacacctgtgatcccggcacttcggaaggccaaggcgggtggatcacttgagcccaggagtttgagcctagcctggccaacatggcagaatcccatctctactaaaaatacaaaagttagccaggcgtggtggcgggcgcctgtaattccagctactcgggaggctgaggcatgagaatcacttgaacatgggaggcggaggttgctgtgagccgagattgcaccgttgtactccagcctgggcaacagaccgagactccatctcaacaaaacaaaaacaaaaaaaatggaaatcaaagtccctcaccagctcaacaacctcccatggctccccagtgccctgtggttcagcctaagccctgcgtgttcccctccctccagctgcctccacctggctgtctttgctggttcagcagtgcttgtctcgctgttccctctgcctggtggaggcggcctatctgagaagggctctatgtggttgccctgggctgctgttgtgagagccggctgggtgcctgtggcccctggggcagcttttcttccaaaatgggaactagtggcctgtgttctaaaaaaaaaaaaaaaaaa

SEQ ID NO: 42 - DNA, 2331 bp, Homo sapiens
  heat shock 70kDa protein 8 transcript variant 1 (HSPA8), lipopolysaccharide-associated protein 1 (LAP1), epididymis secretory sperm binding protein Li 72p (HEL-S-72p), N-myristoyltransferase inhibitor protein 71 (NIP71)
ccttctggaaggttctaagatagggtataagaggcagggtggcgggcggaaaccggtctcattgaactcgcctgcagctcttgggttttttgtggcttccttcgttattggagccaggcctacaccccagcaaccatgtccaagggacctgcagttggtattgatcttggcaccacctactcttgtgtgggtgttttccagcacggaaaagtcgagataattgccaatgatcagggaaaccgaaccactccaagctatgtcgcctttacggacactgaacggttgatcggtgatgccgcaaagaatcaagttgcaatgaaccccaccaacacagtttttgatgccaaacgtctgattggacgcagatttgatgatgctgttgtccagtctgatatgaaacattggccctttatggtggtgaatgatgctggcaggcccaaggtccaagtagaatacaagggagagaccaaaagcttctatccagaggaggtgtcttctatggttctgacaaagatgaaggaaattgcagaagcctaccttgggaagactgttaccaatgctgtggtcacagtgccagcttactttaatgactctcagcgtcaggctaccaaagatgctggaactattgctggtctcaatgtacttagaattattaatgagccaactgctgctgctattgcttacggcttagacaaaaaggttggagcagaaagaaacgtgctcatctttgacctgggaggtggcacttttgatgtgtcaatcctcactattgaggatggaatctttgaggtcaagtctacagctggagacacccacttgggtggagaagattttgacaaccgaatggtcaaccattttattgctgagtttaagcgcaagcataagaaggacatcagtgagaacaagagagctgtaagacgcctccgtactgcttgtgaacgtgctaagcgtaccctctcttccagcacccaggccagtattgagatcgattctctctatgaaggaatcgacttctatacctccattacccgtgcccgatttgaagaactgaatgctgacctgttccgtggcaccctggacccagtagagaaagcccttcgagatgccaaactagacaagtcacagattcatgatattgtcctggttggtggttctactcgtatccccaagattcagaagcttctccaagacttcttcaatggaaaagaactgaataagagcatcaaccctgatgaagctgttgcttatggtgcagctgtccaggcagccatcttgtctggagacaagtctgagaatgttcaagatttgctgctcttggatgtcactcctctttcccttggtattgaaactgctggtggagtcatgactgtcctcatcaagcgtaataccaccattcctaccaagcagacacagaccttcactacctattctgacaaccagcctggtgtgcttattcaggtttatgaaggcgagcgtgccatgacaaaggataacaacctgcttggcaagtttgaactcacaggcatacctcctgcaccccgaggtgttcctcagattgaagtcacttttgacattgatgccaatggtatactcaatgtctctgctgtggacaagagtacgggaaaagagaacaagattactatcactaatgacaagggccgtttgagcaaggaagacattgaacgtatggtccaggaagctgagaagtacaaagctgaagatgagaagcagagggacaaggtgtcatccaagaattcacttgagtcctatgccttcaacatgaaagcaactgttgaagatgagaaacttcaaggcaagattaacgatgaggacaaacagaagattctggacaagtgtaatgaaattatcaactggcttgataagaatcagactgctgagaaggaagaatttgaacatcaacagaaagagctggagaaagtttgcaaccccatcatcaccaagctgtaccagagtgcaggaggcatgccaggaggaatgcctgggggatttcctggtggtggagctcctccctctggtggtgcttcctcagggcccaccattgaagaggttgattaagccaaccaagtgtagatgtagcattgttccacacatttaaaacatttgaaggacctaaattcgtagcaaattctgtggcagttttaaaaagttaagctgctatagtaagttactgggcattctcaatacttgaatatggaacatatgcacaggggaaggaaataacattgcactttataaacactgtattgtaagtggaaaatgcaatgtcttaaataaaactatttaaaattggcaccataaaaaaaaaaaaaa

SEQ ID NO: 43 - DNA, 401 bp, Homo sapiens
  60S ribosomal protein L39, L39, RPL39P42, RPL39_23_1806
cttccgccagcttccctcctcttcctttctccgccatcgtggtgtgttcttgactccgctgctcgccatgtcttctcacaagactttcaggattaagcgattcctggccaagaaacaaaagcaaaatcgtcccattccccagtggattcggatgaaaactggaaataaaatcaggtacaactccaaaaggagacattggagaagaaccaagctgggtctataaggaattgcacatgagatggcacacatatttatgctgtctgaaggtcacgatcatgttaccatatcaagctgaaaatgtcaccactatctggagatttcgacgtgttttcctctctgaatctgttatgaacacgttggttggctggattcagtaataaatatgtaaggcctttcttttt

SEQ ID NO: 44 - DNA, 2211 bp, Homo sapiens
  adenosylhomocysteinase transcript variant 1 (AHCY, adoHcyase), S-adenosylhomocysteine hydrolase, S-adenosyl-L- homocysteine hydrolase (SAHH)
atgaatatgcaagtgcgaggaagatatttaaaggcgtcggcgccacgcgcatatccctgctcggcgctgcccgcccagttcctgttcccagactgaggcccagcccccttcgcccgtttccatcacgagtgccgccagcatgtctgacaaactgccctacaaagtcgccgacatcggcctggctgcctggggacgcaaggccctggacattgctgagaacgagatgccgggcctgatgcgtatgcgggagcggtactcggcctccaagccactgaagggcgcccgcatcgctggctgcctgcacatgaccgtggagacggccgtcctcattgagaccctcgtcaccctgggtgctgaggtgcagtggtccagctgcaacatcttctccacccaggaccatgcggcggctgccattgccaaggctggcattccggtgtatgcctggaagggcgaaacggacgaggagtacctgtggtgcattgagcagaccctgtacttcaaggacgggcccctcaacatgattctggacgacgggggcgacctcaccaacctcatccacaccaagtacccgcagcttctgccaggcatccgaggcatctctgaggagaccacgactggggtccacaacctctacaagatgatggccaatgggatcctcaaggtgcctgccatcaatgtcaatgactccgtcaccaagagcaagtttgacaacctctatggctgccgggagtccctcatagatggcatcaagcgggccacagatgtgatgattgccggcaaggtagcggtggtagcaggctatggtgatgtgggcaagggctgtgcccaggccctgcggggtttcggagcccgcgtcatcatcaccgagattgaccccatcaacgcactgcaggctgccatggagggctatgaggtgaccaccatggatgaggcctgtcaggagggcaacatctttgtcaccaccacaggctgtattgacatcatccttggccggcactttgagcagatgaaggatgatgccattgtgtgtaacattggacactttgacgtggagatcgatgtcaagtggctcaacgagaacgccgtggagaaggtgaacatcaagccgcaggtggaccggtatcggttgaagaatgggcgccgcatcatcctgctggccgagggtcggctggtcaacctgggttgtgccatgggccaccccagcttcgtgatgagtaactccttcaccaaccaggtgatggcgcagatcgagctgtggacccatccagacaagtaccccgttggggttcatttcctgcccaagaagctggatgaggcagtggctgaagcccacctgggcaagctgaatgtgaagttgaccaagctaactgagaagcaagcccagtacctgggcatgtcctgtgatggccccttcaagccggatcactaccgctactgagagccaggtctgcgtttcaccctccagctgctgtccttgcccaggccccacctctcctccctaagagctaatggcaccaactttgtgattggtttgtcagtgtcccccatcgactctctggggctgatcacttagtttttggcctctgctgcagccgtcatactgttccaaatgtggcagcgggaacagagtaccctcttcaagccccggtcatgatggaggtcccagccacagggaaccatgagctcagtggtcttggaacagctcactaagtcagtccttccttagcctggaagtcagtagtggagtcacaaagcccatgtgttttgccatctaggccttcacctggtctgtggacttatacctgtgtgcttggtttacaggtccagtggttcttcagcccatgacagatgagaaggggctatattgaagggcaaagaggaactgttgtttgaattttcctgagagcctggcttagtgctgggccttctcttaaacctcattacaatgaggttagtacttttagtccctgttttacaggggttagaatagactgttaaggggcaactgagaaagaacagagaagtgacagctaggggttgagaggggccagaaaaacatgaatgcaggcagatttcgtgaaatctgccaccactttataaccagatggttcctttcacaaccctgggtcaaaaagagaataatttggcctataatgttaaaagaaagcaggaaggtgggtaaataaaaatcttggtgcctggaagaaaaaa

SEQ ID NO: 45 - DNA, 2022 bp, Homo sapiens
  eukaryotic translation elongation factor 1 alpha 2, eukaryotic elongation factor 1 A-2 (EEF1A2, EF-1-alpha-2, eEF1A-2, EEF1AL), elongation factor-1 alpha (EF1A), statin S1 (HS1, STN, STNL)
gccccgcccccgcccgcggcgcgtttctcccccgcctcccgcgtccgtctttgcagcccgcgcctcccgcatcgcctcgcgtccccgtggcgcccgcccgcgcgcgtccgcgccccgccccctcccgcgcggttccgcattggcgtgctgcagggcgcggtgcactgcgccgccaccgtcaataggtggaccccctcccggagataaaaccgccggcgccggcgccgccagtccctctggctgagacctcggctccggaatcactgcagcccccctcgccctgagccagagcaccccgggtcccgccagcccctcacactcccagcaaaatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgaagcgcgggcgcccgcggcgcgaccctccccggcggcgccgcgctccgaaccccggcccggcccccgccccgcccccgccccgcgcgccgctccggcgccccgcacccccgccaggcgcatgtctgcacctccgcttgccagaggccctcggtcagcgactggatgctcgccatcaaggtccagtggaagttcttcaagaggaaaggcgcccccgccccaggcttccgcgcccagcgctcgccacgctcagtgcccgttttaccaataaactgagcgaccccagaaaaaaaaaaaaaaaaaa

SEQ ID NO: 46 - DNA, 719 bp, Homo sapiens
  60S ribosomal protein L10a, L10A, neural precursor cell expressed developmentally down-regulated protein 6 (NEDD6), Csa-19
agtctcttttccggttagcgcggcgtgagaagccatgagcagcaaagtctctcgcgacaccctgtacgaggcggtgcgggaagtcctgcacgggaaccagcgcaagcgccgcaagttcctggagacggtggagttgcagatcagcttgaagaactatgatccccagaaggacaagcgcttctcgggcaccgtcaggcttaagtccactccccgccctaagttctctgtgtgtgtcctgggggaccagcagcactgtgacgaggctaaggccgtggatatcccccacatggacatcgaggcgctgaaaaaactcaacaagaataaaaaactggtcaagaagctggccaagaagtatgatgcgtttttggcctcagagtctctgatcaagcagattccacgaatcctcggcccaggtttaaataaggcaggaaagttcccttccctgctcacacacaacgaaaacatggtggccaaagtggatgaggtgaagtccacaatcaagttccaaatgaagaaggtgttatgtctggctgtagctgttggtcacgtgaagatgacagacgatgagcttgtgtataacattcacctggctgtcaacttcttggtgtcattgctcaagaaaaactggcagaatgtccgggccttatatatcaagagcaccatgggcaagccccagcgcctatattaaggcacatttgaataaattctattaccagttc

SEQ ID NO: 47 - DNA, 3230 bp, Homo sapiens
  poly(A) binding protein, cytoplasmic 4 (inducible form) transcript variant 1, inducible poly(A)-binding protein, poly(A)-binding protein 4, polyadenylate-binding protein 4 (PABPC4, PABP-4, iPABP), activated-platelet protein 1 (APP-1, APP1)
ttcgaccagcagcgcccggggcgggcgggtataaatggagcggtggctccctcggccgcctctctccgccccgggtcgctgccgcctccgccgctttcgggcttcgcagcctgaggaaaaaaagagaaaaagataaaaaaaatctgaaaacgcttcaaaatcctgaaaaaaaaaaaggaaaagaaaaaacgaatcctcggagaacccgcggggaagtcactttcgtacgcttccggcctgccccgcgcccgccgccgcagcgcttggcgtccgtcggtctccgtccgtcggtccgggggtgagccgcccgcccggcccgccgtgccctccccccgctcgggccccgagccccgcgccccgcgcctgccccggcgcaccacgtgtccgtgctgcccttcgccgcccgcccggggctcgccgagtcggcgcccacaaagatttggtttccctctgccccggcggttgtaatcttaaaccgccggagcccgaggcctatatttatagagaaacgcgtgtccccgaggccgccgtgggcagcgtccggtcgcctcttaaaggatttttacccttcggaaggggattccccgtttaatttttttcctactttgattttttgaaatttggagcttcgcaccaggaccgcggagaagtgcaaagtcgcggggagggccgtattgtgcggagagccttttgtctgcggtgctgcggccgtgggagccggcccccgcctcccgtttccgtcccgtctccaagcccgccgactccagctcgtcctcgccgcgccggtgccacctgtgagccgcggcgcgggcccgggctccgaaggcgcccctttgtcctgcggcgggcccgataagaagtcctcctggcggggctcggggtggtggggggcggggagatgaacgctgcggccagcagctaccccatggcctccctgtacgtgggcgacctgcattcggacgtcaccgaggccatgctgtacgaaaagttcagccccgcggggcctgtgctgtccatccgggtctgccgcgatatgatcacccgccgctccctgggctatgcctacgtcaacttccagcagccggccgacgctgagcgggctttggacaccatgaactttgatgtgattaagggaaagccaatccgcatcatgtggtctcagagggatccctctttgagaaaatctggtgtgggaaacgtcttcatcaagaacctggacaaatctatagataacaaggcactttatgatactttttctgcttttggaaacatactgtcctgcaaggtggtgtgtgatgagaacggctctaagggttatgcctttgtccacttcgagacccaagaggctgccgacaaggccatcgagaagatgaatggcatgctcctcaatgaccgcaaagtatttgtgggcagattcaagtctcgcaaagagcgggaagctgagcttggagccaaagccaaggaattcaccaatgtttatatcaaaaactttggggaagaggtggatgatgagagtctgaaagagctattcagtcagtttggtaagaccctaagtgtcaaggtgatgagagatcccaatgggaaatccaaaggctttggctttgtgagttacgaaaaacacgaggatgccaataaggctgtggaagagatgaatggaaaagaaataagtggtaaaatcatatttgtaggccgtgcacaaaagaaagtagaacggcaggcagagttaaaacggaaatttgaacagttgaaacaggagagaattagtcgatatcagggggtgaatctctacattaagaacttggatgacactattgatgatgagaaattaaggaaagaattttctccttttggatcaattaccagtgctaaggtaatgctggaggatggaagaagcaaagggtttggcttcgtctgcttctcatctcctgaagaagcaaccaaagcagtcactgagatgaatggacgcattgtgggctccaagccactatatgttgccctggcccagaggaaggaagagagaaaggctcacctgaccaaccagtatatgcaacgagtggctggaatgagagcacttcctgccaatgccatcttaaatcagttccagcctgcagcgggtggctactttgtgccagcagtcccacaggctcagggaaggcctccatattatacacctaaccagttagcacagatgaggcctaatccacgctggcagcaaggtgggagacctcaaggcttccaaggaatgccaagtgctatacgccagtctgggcctcgtccaactcttcgccatctggctccaactggtaatgctccggcctctcgtggcctccctactaccactcagagagtcgggtctgagtgcccggaccgcttggctatggactttggtggggctggtgccgcccagcaagggctgactgacagctgccagtctggaggcgttcccacagctgtgcagaacttagcgccacgcgctgctgttgctgctgctgctccccgggctgttgccccctacaaatacgcctccagtgtccgcagccctcatcctgccatacagcctctgcaggcaccccagcctgcggtccatgtgcaggggcaggagccactgactgcctccatgctggctgcagcacccccccaggaacagaagcagatgctgggagaacgcttgttcccactcatccaaacaatgcattcaaatctggctgggaagatcacgggaatgctgctggagatagacaactctgagctgctgcacatgttagagtcccccgagtctctccgctccaaggtggatgaagctgtagcagttctacaggctcatcatgccaagaaagaagctgcccagaaggtgggcgctgttgctgctgctacctcttagacaaggaaaaaccgattcaaaagccaaataaccccttatggaattcaactcaaggtttgaagacttcctagcttgtcctatggacctcaacaccaaggattacaaattgcaaatttaataggtcattttgtatcaaaaggtcaattatgaagcacctagaatttttcaattatacgaatatgttctttgggttctgctgtggcccagacagtgttaactttttttttattgtgggttttgattttttcccccagaaattggttttatttgatgtacccaagtcttacgtttcccaataaagaaaaaaaatctccataaaactgaaaaaaaaaaaaaaaaaaaaaaaaaa

SEQ ID NO: 48 - DNA, 956 bp, Homo sapiens
  40S ribosomal protein S4, X-linked, ribosomal protein S4X, S4X, single copy abundant mRNA protein (SCAR), cell cycle gene 2 (CCG2), DXS306, SCR10
gggcggagcagctgaaaatccggcgcgcgcagtctccagccccaatttctacgcgcaccggaagacggaggtcctctttccttgcctaacgcagccatggctcgtggtcccaagaagcatctgaagcgggtggcagctccaaagcattggatgctggataaattgaccggtgtgtttgctcctcgtccatccaccggtccccacaagttgagagagtgtctccccctcatcattttcctgaggaacagacttaagtatgccctgacaggagatgaagtaaagaagatttgcatgcagcggttcattaaaatcgatggcaaggtccgaactgatataacctaccctgctggattcatggatgtcatcagcattgacaagacgggagagaatttccgtctgatctatgacaccaagggtcgctttgctgtacatcgtattacacctgaggaggccaagtacaagttgtgcaaagtgagaaagatctttgtgggcacaaaaggaatccctcatctggtgactcatgatgcccgcaccatccgctaccccgatcccctcatcaaggtgaatgataccattcagattgatttggagactggcaagattactgatttcatcaagttcgacactggtaacctgtgtatggtgactggaggtgctaacctaggaagaattggtgtgatcaccaacagagagaggcaccctggatcttttgacgtggttcacgtgaaagatgccaatggcaacagctttgccactcgactttccaacatttttgttattggcaagggcaacaaaccatggatttctcttccccgaggaaagggtatccgcctcaccattgctgaagagagagacaaaagactggcggccaaacagagcagtgggtgaaatgggtccctgggtgacatgtcagatctttgtacgtaattaaaaatattgtggcaggattaatagca

SEQ ID NO: 49 - DNA, 1129 bp, Homo sapiens
  60S ribosomal protein L31 transcript variant 2, L31
tggcgacccggaagttgtacttgcaactgcggctttccttctcccacaatccttcgcgctcttcctttccaacttggacgctgcagaatggctcccgcaaagaagggtggcgagaagaaaaagggccgttctgccatcaacgaagtggtaacccgagaatacaccatcaacattcacaagcgcatccatggagtgggcttcaagaagcgtgcacctcgggcactcaaagagattcggaaatttgccatgaaggagatgggaactccagatgtgcgcattgacaccaggctcaacaaagctgtctgggccaaaggaataaggaatgtgccataccgaatccgtgtgcggctgtccagaaaacgtaatgaggatgaagattcaccaaataagctatatactttggttacctatgtacctgttaccactttcaaaatttctgtgctaaacagtgttacagtcgccaagagcccataaagggagccctcctggaagtggatgaggccttgggtctcggctcttcattgcttcctgagctgcagcagatgcctttacaaccaagctcaccgaggacgtctgtctcccatattaccctggcagagggccaggcctgttctacacggccggggtttcaacaaggtactgatgtcttctgcccttgcctcttcgacaggcaagtaataagacttaagtgaagagaattctttaggcacacaaattcacatttgatgtaatctcattatacttcctgatctgtgattgaaaactttcatttcgtaactagtatgtctgtcccacctttaaaaagtttttcattatgaaagtaagtatttgttagaattaagtctatttaaatgaaaaaaacttagatatgagtctgcatggcctcaggaaaatgatgttttaaaatagagattttaggttgtctgcactctagcttttttgtcgttttcttaaggcttttttaactgcatcaaaaattcagatacgaaacatacactaaaaaataatacatcatatcttaatttccactgaacttgatttaaattcagagttacacagtatgaatatcacaatcagatatgttcaaaaaggtctgaacaattgattttctgaaaccatgaaggactac

SEQ ID NO: 50 - DNA, 512 bp, Homo sapiens
  60S acidic ribosomal protein P1 transcript variant 1, LP1, P1, RPP1, ribosomal protein, large, P1, acidic ribosomal phosphoprotein P1
cctttcctcagctgccgccaaggtgctcggtccttccgaggaagctaaggctgcgttggggtgaggccctcacttcatccggcgactagcaccgcgtccggcagcgccagccctacactcgcccgcgccatggcctctgtctccgagctcgcctgcatctactcggccctcattctgcacgacgatgaggtgacagtcacggaggataagatcaatgccctcattaaagcagccggtgtaaatgttgagcctttttggcctggcttgtttgcaaaggccctggccaacgtcaacattgggagcctcatctgcaatgtaggggccggtggacctgctccagcagctggtgctgcaccagcaggaggtcctgccccctccactgctgctgctccagctgaggagaagaaagtggaagcaaagaaagaagaatccgaggagtctgatgatgacatgggctttggtctttttgactaaacctcttttataacatgttcaataaaaagctgaacttt

SEQ ID NO: 51 - DNA, 646 bp, Homo sapiens
  40S ribosomal protein S11, S11
tctccttacgtcacttcctctccagcccctgcgtaatcgataaggaaacccggacgctgctgcccctttctttttttcaggcggccgggaagatggcggacattcagactgagcgtgcctaccaaaagcagccgaccatctttcaaaacaagaagagggtcctgctgggagaaactggcaaggagaagctcccgcggtactacaagaacatcggtctgggcttcaagacacccaaggaggctattgagggcacctacattgacaagaaatgccccttcactggtaatgtgtccattcgagggcggatcctctctggcgtggtgaccaagatgaagatgcagaggaccattgtcatccgccgagactatctgcactacatccgcaagtacaaccgcttcgagaagcgccacaagaacatgtctgtacacctgtccccctgcttcagggacgtccagatcggtgacatcgtcacagtgggcgagtgccggcctctgagcaagacagtgcgcttcaacgtgctcaaggtcaccaaggctgccggcaccaagaagcagttccagaagttctgaggctggacatcggcccgctccccacaatgaaataaagttattttctcattcccaggccagacttgggatcttccgcg

SEQ ID NO: 52 - DNA, 602 bp, Homo sapiens
  60S ribosomal protein L26, L26, DBA11
ataggtctcgcgagatctttggtaaacttacagaaccggaagcagcgtgtagttctcttcccttttgcggccatcaccgaagcgggagcggccaaaatgaagtttaatccctttgtgacttccgaccgaagcaagaatcgcaaaaggcatttcaatgcaccttcccacattcgaaggaagattatgtcttcccctctttccaaagagctgagacagaagtacaacgtgcgatccatgcccatccgaaaggatgatgaagttcaggttgtacgtggacactataaaggtcagcaaattggcaaagtagtccaggtttacaggaagaaatatgttatctacattgaacgggtgcagcgggaaaaggctaatggcacaactgtccacgtaggcattcaccccagcaaggtggttatcactaggctaaaactggacaaagaccgcaaaaagatcctcgaacggaaagccaaatctcgccaagtaggaaaggaaaagggcaaatacaaggaagaaaccattgagaagatgcaggaataaagtaatcttatatacaagctttgattaaaacttgaaacaaagagcctgaaaaaaaaaaaaaaaaaaaa

SEQ ID NO: 53 - DNA, 939 bp, Homo sapiens
  60S ribosomal protein L14 transcript variant 1, L14
cttctcgcctaacgccgccaacatggtgagtcttactgttgcgggctccggggccgtcgaccatgccgctcgacctccacctccgctgggaagctgaggcgccaaacggctcccagagggtcccgggaagcgcatggtgttcaggcgcttcgtggaggttggccgggtggcctatgtctcctttggacctcatgccggaaaattggtcgcgattgtagatgttattgatcagaacagggctttggtcgatggaccttgcactcaagtgaggagacaggccatgcctttcaagtgcatgcagctcactgatttcatcctcaagtttccgcacagtgcccaccagaagtatgtccgacaagcctggcagaaggcagacatcaatacaaaatgggcagccacacgatgggccaagaagattgaagccagagaaaggaaagccaagatgacagattttgatcgttttaaagttatgaaggcaaagaaaatgaggaacagaataatcaagaatgaagttaagaagcttcaaaaggcagctctcctgaaagcttctcccaaaaaagcacctggtactaagggtactgctgctgctgctgctgctgctgctgctgctaaagttccagcaaaaaagatcaccgccgcgagtaaaaaggctccagcccagaaggttcctgcccagaaagccacaggccagaaagcagcgcctgctccaaaagctcagaagggtcaaaaagctccagcccagaaagcacctgctccaaaggcatctggcaagaaagcataagtggcaatcataaaaagtaataaaggttctttttgacctgttgacaaatgtatttaagcctttggatttaaagcctgttgaggctggagttaggaggcagattgatagtaggattataataaacattaaataatcagttcaaaaaaaaaaaaaaaaaa

SEQ ID NO: 54 - DNA, 1586 bp, Homo sapiens
  60S ribosomal protein L37, L37, L37a, G1.16
gcgccccgcaggaagtgcttccctgggcggaagcttctgagcgtgatatagcggaagtgccttctcttccggtctttctggtctcggccgcagaagcgagatgacgaagggaacgtcatcgtttggaaagcgtcgcaataagacgcacacgttgtgccgccgctgtggctctaaggcctaccaccttcagaagtcgacctgtggcaaatgtggctaccctgccaagcgcaagagaaagtataactggagtgccaaggctaaaagacgaaataccaccggaactggtcgaatgaggcacctaaaaattgtataccgcagattcaggcatggattccgtgaaggaacaacacctaaacccaagagggcagctgttgcagcatccagttcatcttaagaatgtcaacgattagtcatgcaataaatgttctggttttaaaaaatacatatctggttttggtaaggtatttttaatcaattaggcttgtagtatcagtgaaatactgtaggtttagggactgggctagcttcatatcagatttacttgttaagtgactgttttggaatgtttacttttggactgggtttgtaacacggttaaaggcaatgagaaacaagcagaattccaggagtccttgaagcagagggcactggaagacaatatagcagattaaaatagcacagctcatgtggcataggtgggtattttagatgtttgagtaaatttgaaagagtatgatgtttaaattacctttagcaacatgttcatctgctatgctgtcatgactagggggatgattattagtcacatagagcttgggagtaccactggaaacgtatgggtaggagtttaggtggcttctgtttttcaaaagatgatcttatcctagtatctgtaatgctcacttggcacacctgacttgtgggctgtgtgtaaggtggctagctaagtgaaaaaagcctgctaggtgtgagtcaacttaagaatatgtaaataggtttgagaaaaagtagggcttgggtgcaagtaaagattgagcaggaaataaaggaaaatcaagtataatccctgagatttgtagactaaaggcaatgatgtgggactacttggtcgaatttttttagccctcaacttggtaattgggtgtttctgtgttaaagcactgaaacttgctgtcgtgccttcctagttttcgtggtttattgacagggttgggggttttttttgtttttttaaaatgaagggacaaagtcaactggactgctgagtgagagggcaggggcagttgaagggaacatgaattgctggaacagctacataaaatagtgatgtagccaagtcatgctatttaaattataattctccactgtgtttagaataacatctgaggttcttaacctggccttggaagggtatcacttttacttgtaacctggaatggctttataatgtgctagctaattgctactctcatcttgtattttaactcctaatttacccttcaggtctcagcttcagaacattcacttataaagaaaccctgctgattaaatctctcttgggcttcctcccaaaaaaaaaaaaaaaa

SEQ ID NO: 55 - DNA, 866 bp, Homo sapiens
  60S ribosomal protein L7, L7, humL7-1
tcctctttttccggctggaaccatggagggtgtagaagagaagaagaaggaggttcctgctgtgccagaaacccttaagaaaaagcgaaggaatttcgcagagctgaagatcaagcgcctgagaaagaagtttgcccaaaagatgcttcgaaaggcaaggaggaagcttatctatgaaaaagcaaagcactatcacaaggaatataggcagatgtacagaactgaaattcgaatggcgaggatggcaagaaaagctggcaacttctatgtacctgcagaacccaaattggcgtttgtcatcagaatcagaggtatcaatggagtgagcccaaaggttcgaaaggtgttgcagcttcttcgccttcgtcaaatcttcaatggaacctttgtgaagctcaacaaggcttcgattaacatgctgaggattgtagagccatatattgcatgggggtaccccaatctgaagtcagtaaatgaactaatctacaagcgtggttatggcaaaatcaataagaagcgaattgctttgacagataacgctttgattgctcgatctcttggtaaatacggcatcatctgcatggaggatttgattcatgagatctatactgttggaaaacgcttcaaagaggcaaataacttcctgtggcccttcaaattgtcttctccacgaggtggaatgaagaaaaagaccacccattttgtagaaggtggagatgctggcaacagggaggaccagatcaacaggcttattagaagaatgaactaaggtgtctaccatgattatttttctaagctggttggttaataaacagtacctgctctcaaattgaaataaaaaaaaaaaaaaaaaaaaaaaaaaaaaa

SEQ ID NO: 56 - DNA, 1941 bp, Homo sapiens
  heterogeneous nuclear ribonucleoprotein A1 transcript variant 2 (HNRNPA1, hnRNP A1, hnRNP-A1, HNRPA1), heterogeneous nuclear ribonucleoprotein B2 protein, hnRNP A1-like 3 (HNRPA1L3), IBMPFD3, single-strand DNA-binding protein UP1, helix-destabilizing protein
gagagggcgaaggtaggctggcagatacgttcgtcagcttgctcctttctgcccgtggacgccgccgaagaagcatcgttaaagtctctcttcaccctgccgtcatgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggtggttatggaggaggcggccctggttactctggaggaagcagaggctatggaagtggtggacagggttatggaaaccagggcagtggctatggcgggagtggcagctatgacagctataacaacggaggcggaggcggctttggcggtggtagtggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttagcaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaagggttttaatgtagatttttttttttgcaccccatgctgttgattgctaaatgtaacagtctgatcgtgacgctgaataaatgtcttttttttaatgtgctgtgtaaagttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgtaatcaataaacgatttaattctcttgaaaaaaaaaaaaaaaaaa

SEQ ID NO: 57 - DNA, 705 bp, Homo sapiens
  40S ribosomal protein S8, S8, OK/SW-cl.83
ctctttccagccagcgccgagcgatgggcatctctcgggacaactggcacaagcgccgcaaaaccgggggcaagagaaagccctaccacaagaagcggaagtatgagttggggcgcccagctgccaacaccaagattggcccccgccgcatccacacagtccgtgtgcggggaggtaacaagaaataccgtgccctgaggttggacgtggggaatttctcctggggctcagagtgttgtactcgtaaaacaaggatcatcgatgttgtctacaatgcatctaataacgagctggttcgtaccaagaccctggtgaagaattgcatcgtgctcatcgacagcacaccgtaccgacagtggtacgagtcccactatgcgctgcccctgggccgcaagaagggagccaagctgactcctgaggaagaagagattttaaacaaaaaacgatctaaaaaaattcagaagaaatatgatgaaaggaaaaagaatgccaaaatcagcagtctcctggaggagcagttccagcagggcaagcttcttgcgtgcatcgcttcaaggccgggacagtgtggccgagcagatggctatgtgctagagggcaaagagttggagttctatcttaggaaaatcaaggcccgcaaaggcaaataaatccttgttttgtcttcacccatgtaataaaggtgtttattgttttgttcccaca

SEQ ID NO: 58 - DNA, 1401 bp, Homo sapiens
  glyceraldehyde-3-phosphate dehydrogenase transcript variant 1 (GAPDH, G3PD; GAPD), peptidyl-cysteine S-nitrosylase GAPDH, aging-associated gene 9 protein, epididymis secretory sperm binding protein Li 162eP (HEL-S-162eP)
ggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaaaaaaaaaaaaaaaaaaa

SEQ ID NO: 59 - DNA, 903 bp, Homo sapiens
  60S ribosomal protein L8 transcript variant 1, L8
agataaggccgctcgctgacgccgtgtttcctctttcggccgcgctggtgaacaggacccgtcgccatgggccgtgtgatccgtggacagaggaagggcgccgggtctgtgttccgcgcgcacgtgaagcaccgtaaaggcgctgcgcgcctgcgcgccgtggatttcgctgagcggcacggctacatcaagggcatcgtcaaggacatcatccacgacccgggccgcggcgcgcccctcgccaaggtggtcttccgggatccgtatcggtttaagaagcggacggagctgttcattgccgccgagggcattcacacgggccagtttgtgtattgcggcaagaaggcccagctcaacattggcaatgtgctccctgtgggcaccatgcctgagggtacaatcgtgtgctgcctggaggagaagcctggagaccgtggcaagctggcccgggcatcagggaactatgccaccgttatctcccacaaccctgagaccaagaagacccgtgtgaagctgccctccggctccaagaaggttatctcctcagccaacagagctgtggttggtgtggtggctggaggtggccgaattgacaaacccatcttgaaggctggccgggcgtaccacaaatataaggcaaagaggaactgctggccacgagtacggggtgtggccatgaatcctgtggagcatccttttggaggtggcaaccaccagcacatcggcaagccctccaccatccgcagagatgcccctgctggccgcaaagtgggtctcattgctgcccgccggactggacgtctccggggaaccaagactgtgcaggagaaagagaactagtgctgagggcctcaataaagtttgtgtttatgccaaaaaaaaaaaaaaaaaaaaaaaaaaaaa

SEQ ID NO: 60 - DNA, 737 bp, Homo sapiens
  60S ribosomal protein L29, L29, ribosomal protein YL43 homologue, heparin/heparan sulfate-interacting protein, heparin/heparan sulfate-binding protein, HP/HS-interacting protein, cell surface heparin-binding protein (HIP), HUMRPL29, RPL29P10; RPL29_3_370
ttccggcgttgttgaccctatttcccgtgctgcaccgcagcccctttctcttccggttctaggcgcttcgggagccgcggcttatggtgcagacatggccaagtccaagaaccacaccacacacaaccagtcccgaaaatggcacagaaatggtatcaagaaaccccgatcacaaagatacgaatctcttaagggggtggaccccaagttcctgaggaacatgcgctttgccaagaagcacaacaaaaagggcctaaagaagatgcaggccaacaatgccaaggccatgagtgcacgtgccgaggctatcaaggccctcgtaaagcccaaggaggttaagcccaagatcccaaagggtgtcagccgcaagctcgatcgacttgcctacattgcccaccccaagcttgggaagcgtgctcgtgcccgtattgccaaggggctcaggctgtgccggccaaaggccaaggccaaggccaaggccaaggatcaaaccaaggcccaggctgcagccccagcttcagttccagctcaggctcccaaacgtacccaggcccctacaaaggcttcagagtagatatctctgccaacatgaggacagaaggactggtgcgaccccccacccccgcccctgggctaccatctgcatggggctggggtcctcctgtgctatttgtacaaataaacctgaggcaggaaaaaaaaaaaaaaaaaaaaaaaaaaaaaaaaaaaaaaaaaaa

SEQ ID NO: 61 - DNA, 950 bp, Homo sapiens
  40S ribosomal protein S3a transcript variant 1, S3A, v-fos transformation effector protein 1, fte-1, FTE1, MFTL
tagacggcgcgccccgcccccgtacgcctaagttctcgcgcgactcccacttccgcccttttggctctctgaccagcaccatggcggttggcaagaacaagcgccttacgaaaggcggcaaaaagggagccaagaagaaagtggttgatccattttctaagaaagattggtatgatgtgaaagcacctgctatgttcaatataagaaatattggaaagacgctcgtcaccaggacccaaggaaccaaaattgcatctgatggtctcaagggtcgtgtgtttgaagtgagtcttgctgatttgcagaatgatgaagttgcatttagaaaattcaagctgattactgaagatgttcagggtaaaaactgcctgactaacttccatggcatggatcttacccgtgacaaaatgtgttccatggtcaaaaaatggcagacaatgattgaagctcacgttgatgtcaagactaccgatggttacttgcttcgtctgttctgtgttggttttactaaaaaacgcaacaatcagatacggaagacctcttatgctcagcaccaacaggtccgccaaatccggaagaagatgatggaaatcatgacccgagaggtgcagacaaatgacttgaaagaagtggtcaataaattgattccagacagcattggaaaagacatagaaaaggcttgccaatctatttatcctctccatgatgtcttcgttagaaaagtaaaaatgctgaagaagcccaagtttgaattgggaaagctcatggagcttcatggtgaaggcagtagttctggaaaagccactggggacgagacaggtgctaaagttgaacgagctgatggatatgaaccaccagtccaagaatctgtttaaagttcagacttcaaatagtggcaaataaaaagtgctatttgtgatggtttgcttctgaaaaaaaaaaaaaaaaaa

SEQ ID NO: 62 - DNA, 893 bp, Homo sapiens
  60S ribosomal protein L18 transcript variant 1, L18
gaaagctatcgagaacacggcctgggtagggccagagaggcccccgacgtgctggcccttccctcttggacgttgcgcttgttcctgcgctctatgctctctgccgttatcgcccggctagtcagtcgtccaactcaccacagagaagtccggatcgtggtagagcgccgcgtcgcacccatgtgacgtcacggcggcgccactcgcttgaggctttccccgcccaccccagcccgttctctctttccggacctggccgagcaggaggcgccatcatgggagtggacatccgccataacaaggaccgaaaggttcggcgcaaggagcccaagagccaggatatctacctgaggctgttggtcaagttatacaggtttctggccagaagaaccaactccacattcaaccaggttgtgttgaagaggttgtttatgagtcgcaccaaccggccgcctctgtccctttcccggatgatccggaagatgaagcttcctggccgggaaaacaagacggccgtggttgtggggaccataactgatgatgtgcgggttcaggaggtacccaaactgaaggtatgtgcactgcgcgtgaccagccgggcccgcagccgcatcctcagggcagggggcaagatcctcactttcgaccagctggccctggactcccctaagggctgtggcactgtcctgctctccggtcctcgcaagggccgagaggtgtaccggcatttcggcaaggccccaggaaccccgcacagccacaccaaaccctacgtccgctccaagggccggaagttcgagcgtgccagaggccgacgggccagccgaggctacaaaaactaaccctggatcctactctcttattaaaaagatttttgctgacagtgcaaaaaa

SEQ ID NO: 63 - DNA, 557 bp, Homo sapiens
  60S ribosomal protein L36 transcript variant 2, L36
gtttcccgcactgccggtatccgccgccatccggactcccgggtcctctgtgcaggttggaggatggttggttgtggcgagcgaggctgaaggagccgggacgcggggctctgggcctcgggaactgagccggtactcacctccgccccttctccccgtcgctgtccgcagccatggccctacgctaccctatggccgtgggcctcaacaagggccacaaagtgaccaagaacgtgagcaagcccaggcacagccgacgccgcgggcgtctgaccaaacacaccaagttcgtgcgggacatgattcgggaggtgtgtggctttgccccgtacgagcggcgcgccatggagttactgaaggtctccaaggacaaacgggccctcaaatttatcaagaaaagggtggggacgcacatccgcgccaagaggaagcgggaggagctgagcaacgtactggccgccatgaggaaagctgctgccaagaaagactgagcccctcccctgccctctccctgaaataaagaacagcttgacagaaaaaaaaaaaaaaaaaaaaaa

SEQ ID NO: 64 - DNA, 7343 bp, Homo sapiens
  agrin precursor, agrin proteoglycan (AGRN)
cccgtccccggcgcggcccgcgcgctcctccgccgcctctcgcctgcgccatggccggccggtcccacccgggcccgctgcggccgctgctgccgctccttgtggtggccgcgtgcgtcctgcccggagccggcgggacatgcccggagcgcgcgctggagcggcgcgaggaggaggcgaacgtggtgctcaccgggacggtggaggagatcctcaacgtggacccggtgcagcacacgtactcctgcaaggttcgggtctggcggtacttgaagggcaaagacctggtggcccgggagagcctgctggacggcggcaacaaggtggtgatcagcggctttggagaccccctcatctgtgacaaccaggtgtccactggggacaccaggatcttctttgtgaaccctgcacccccatacctgtggccagcccacaagaacgagctgatgctcaactccagcctcatgcggatcaccctgcggaacctggaggaggtggagttctgtgtggaagataaacccgggacccacttcactccagtgcctccgacgcctcctgatgcgtgccggggaatgctgtgcggcttcggcgccgtgtgcgagcccaacgcggaggggccgggccgggcgtcctgcgtctgcaagaagagcccgtgccccagcgtggtggcgcctgtgtgtgggtcggacgcctccacctacagcaacgaatgcgagctgcagcgggcgcagtgcagccagcagcgccgcatccgcctgctcagccgcgggccgtgcggctcgcgggacccctgctccaacgtgacctgcagcttcggcagcacctgtgcgcgctcggccgacgggctgacggcctcgtgcctgtgccccgcgacctgccgtggcgcccccgaggggaccgtctgcggcagcgacggcgccgactaccccggcgagtgccagctcctgcgccgcgcctgcgcccgccaggagaatgtcttcaagaagttcgacggcccttgtgacccctgtcagggcgccctccctgacccgagccgcagctgccgtgtgaacccgcgcacgcggcgccctgagatgctcctacggcccgagagctgccctgcccggcaggcgccagtgtgtggggacgacggagtcacctacgaaaacgactgtgtcatgggccgatcgggggccgcccggggtctcctcctgcagaaagtgcgctccggccagtgccagggtcgagaccagtgcccggagccctgccggttcaatgccgtgtgcctgtcccgccgtggccgtccccgctgctcctgcgaccgcgtcacctgtgacggggcctacaggcccgtgtgtgcccaggacgggcgcacgtatgacagtgattgctggcggcagcaggctgagtgccggcagcagcgtgccatccccagcaagcaccagggcccgtgtgaccaggccccgtccccatgcctcggggtgcagtgtgcatttggggcgacgtgtgctgtgaagaacgggcaggcagcgtgtgaatgcctgcaggcgtgctcgagcctctacgatcctgtgtgcggcagcgacggcgtcacatacggcagcgcgtgcgagctggaggccacggcctgtaccctcgggcgggagatccaggtggcgcgcaaaggaccctgtgaccgctgcgggcagtgccgctttggagccctgtgcgaggccgagaccgggcgctgcgtgtgcccctctgaatgcgtggctttggcccagcccgtgtgtggctccgacgggcacacgtaccccagcgagtgcatgctgcacgtgcacgcctgcacacaccagatcagcctgcacgtggcctcagctggaccctgtgagacctgtggagatgccgtgtgtgcttttggggctgtgtgctccgcagggcagtgtgtgtgtccccggtgtgagcaccccccgcccggccccgtgtgtggcagcgacggtgtcacctacggcagtgcctgcgagctacgggaagccgcctgcctccagcagacacagatcgaggaggcccgggcagggccgtgcgagcaggccgagtgcggttccggaggctctggctctggggaggacggtgactgtgagcaggagctgtgccggcagcgcggtggcatctgggacgaggactcggaggacgggccgtgtgtctgtgacttcagctgccagagtgtcccaggcagcccggtgtgcggctcagatggggtcacctacagcaccgagtgtgagctgaagaaggccaggtgtgagtcacagcgagggctctacgtagcggcccagggagcctgccgaggccccaccttcgccccgctgccgcctgtggcccccttacactgtgcccagacgccctacggctgctgccaggacaatatcaccgcagcccggggcgtgggcctggctggctgccccagtgcctgccagtgcaacccccatggctcttacggcggcacctgtgacccagccacaggccagtgctcctgccgcccaggtgtggggggcctcaggtgtgaccgctgtgagcctggcttctggaactttcgaggcatcgtcaccgatggccggagtggctgtacaccctgcagctgtgatccccaaggcgccgtgcgggatgactgtgagcagatgacggggctgtgctcgtgtaagcccggggtggctggacccaagtgtgggcagtgtccagacggccgtgccctgggccccgcgggctgtgaagctgacgcttctgcgcctgcgacctgtgcggagatgcgctgtgagttcggtgcgcggtgcgtggaggagtctggctcagcccactgtgtctgcccgatgctcacctgtccagaggccaacgctaccaaggtctgtgggtcagatggagtcacatacggcaacgagtgtcagctgaagaccatcgcctgccgccagggcctgcaaatctctatccagagcctgggcccgtgccaggaggctgttgctcccagcactcacccgacatctgcctccgtgactgtgaccaccccagggctcctcctgagccaggcactgccggccccccccggcgccctccccctggctcccagcagtaccgcacacagccagaccacccctccgccctcatcacgacctcggaccactgccagcgtccccaggaccaccgtgtggcccgtgctgacggtgccccccacggcaccctcccctgcacccagcctggtggcgtccgcctttggtgaatctggcagcactgatggaagcagcgatgaggaactgagcggggaccaggaggccagtgggggtggctctggggggctcgagcccttggagggcagcagcgtggccacccctgggccacctgtcgagagggcttcctgctacaactccgcgttgggctgctgctctgatgggaagacgccctcgctggacgcagagggctccaactgccccgccaccaaggtgttccagggcgtcctggagctggagggcgtcgagggccaggagctgttctacacgcccgagatggctgaccccaagtcagaactgttcggggagacagccaggagcattgagagcaccctggacgacctcttccggaattcagacgtcaagaaggattttcggagtgtccgcttgcgggacctggggcccggcaaatccgtccgcgccattgtggatgtgcactttgaccccaccacagccttcagggcacccgacgtggcccgggccctgctccggcagatccaggtgtccaggcgccggtccttgggggtgaggcggccgctgcaggagcacgtgcgatttatggactttgactggtttcctgcgtttatcacgggggccacgtcaggagccattgctgcgggagccacggccagagccaccactgcatcgcgcctgccgtcctctgctgtgacccctcgggccccgcaccccagtcacacaagccagcccgttgccaagaccacggcagcccccaccacacgtcggccccccaccactgcccccagccgtgtgcccggacgtcggcccccggccccccagcagcctccaaagccctgtgactcacagccctgcttccacggggggacctgccaggactgggcattgggcgggggcttcacctgcagctgcccggcaggcaggggaggcgccgtctgtgagaaggtgcttggcgcccctgtgccggccttcgagggccgctccttcctggccttccccactctccgcgcctaccacacgctgcgcctggcactggaattccgggcgctggagcctcaggggctgctgctgtacaatggcaacgcccggggcaaggacttcctggcattggcgctgctagatggccgcgtgcagctcaggtttgacacaggttcggggccggcggtgctgaccagtgccgtgccggtagagccgggccagtggcaccgcctggagctgtcccggcactggcgccggggcaccctctcggtggatggtgagacccctgttctgggcgagagtcccagtggcaccgacggcctcaacctggacacagacctctttgtgggcggcgtacccgaggaccaggctgccgtggcgctggagcggaccttcgtgggcgccggcctgagggggtgcatccgtttgctggacgtcaacaaccagcgcctggagcttggcattgggccgggggctgccacccgaggctctggcgtgggcgagtgcggggaccacccctgcctgcccaacccctgccatggcggggccccatgccagaacctggaggctggaaggttccattgccagtgcccgcccggccgcgtcggaccaacctgtgccgatgagaagagcccctgccagcccaacccctgccatggggcggcgccctgccgtgtgctgcccgagggtggtgctcagtgcgagtgccccctggggcgtgagggcaccttctgccagacagcctcggggcaggacggctctgggcccttcctggctgacttcaacggcttctcccacctggagctgagaggcctgcacacctttgcacgggacctgggggagaagatggcgctggaggtcgtgttcctggcacgaggccccagcggcctcctgctctacaacgggcagaagacggacggcaagggggacttcgtgtcgctggcactgcgggaccgccgcctggagttccgctacgacctgggcaagggggcagcggtcatcaggagcagggagccagtcaccctgggagcctggaccagggtctcactggagcgaaacggccgcaagggtgccctgcgtgtgggcgacggcccccgtgtgttgggggagtccccggttccgcacaccgtcctcaacctgaaggagccgctctacgtagggggcgctcccgacttcagcaagctggcccgtgctgctgccgtgtcctctggcttcgacggtgccatccagctggtctccctcggaggccgccagctgctgaccccggagcacgtgctgcggcaggtggacgtcacgtcctttgcaggtcacccctgcacccgggcctcaggccacccctgcctcaatggggcctcctgcgtcccgagggaggctgcctatgtgtgcctgtgtcccgggggattctcaggaccgcactgcgagaaggggctggtggagaagtcagcgggggacgtggataccttggcctttgacgggcggacctttgtcgagtacctcaacgctgtgaccgagagcgagaaggcactgcagagcaaccactttgaactgagcctgcgcactgaggccacgcaggggctggtgctctggagtggcaaggccacggagcgggcagactatgtggcactggccattgtggacgggcacctgcaactgagctacaacctgggctcccagcccgtggtgctgcgttccaccgtgcccgtcaacaccaaccgctggttgcgggtcgtggcacatagggagcagagggaaggttccctgcaggtgggcaatgaggcccctgtgaccggctcctccccgctgggcgccacgcagctggacactgatggagccctgtggcttgggggcctgccggagctgcccgtgggcccagcactgcccaaggcctacggcacaggctttgtgggctgcttgcgggacgtggtggtgggccggcacccgctgcacctgctggaggacgccgtcaccaagccagagctgcggccctgccccaccccatgagctggcaccagagccccgcgcccgctgtaattattttctatttttgtaaacttgttgctttttgatatgattttcttgcctgagtgttggccggagggactgctggcccggcctcccttccgtccaggcagccgtgctgcagacagacctagtgccgagggatggacaggcgaggtggcagcgtggagggctcggcgtggatggcagcctcaggacacacacccctgcctcaaggtgctgagcccccgccttgcactgcgcctgccccacggtgtccccgccgggaagcagccccggctcctgaatcaccctcgctccgtcaggcgggactcgtgtcccagagaggaaggggctgctgaggtctgatggggcccttcctccgggtgaccccacagggcctttccaagcccccatttgagctgctccttcctgtgtgtgctctgggccctgcctcggcctcctgcgccaatactgtgacttccaaacaatgttactgctgggcacagctctgcgttgctcccgtgctgcctgcgccagccccaggctgctgaggagcagaggccagaccagggccgatctgggtgtcctgaccctcagctggccctgcccagccaccctggacgtgaccgtatccctctgccacaccccaggccctgcgaggggctatcgagaggagctcactgtgggatggggttgacctctgccgcctgcctgggtatctgggcctggccatggctgtgttcttcatgtgttgattttatttgacccctggagtggtgggtctcatctttcccatctcgcctgagagcggctgagggctgcctcactgcaaatcctccccacagcgtcagtgaaagtcgtccttgtctcagaatgaccaggggccagccagtgtctgaccaaggtcaaggggcaggtgcagaggtggcagggatggctccgaagccagaaatgccttaaactgcaacgtcccgtcccttccccacccccatcccatccccacccccagccccagcccagtcctcctaggagcaggacccgatgaagcgggcggcggtggggctgggtgccgtgttactaactctagtatgtttctgtgtcaatcgctgtgaaataaagtctgaaaactttaaaagcaaaaaaaaaaaaaaaaaa

SEQ ID NO: 65 - DNA, 829 bp, Homo sapiens
  tumor protein, translationally-controlled 1 transcript variant 2 (TPT1), fortilin, translationally-controlled tumor protein (TCTP), histamine-releasing factor (HRF), p02, p23
ccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgttaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcatttgagag

SEQ ID NO: 66 - DNA, 881 bp, Homo sapiens
  60S ribosomal protein L36a transcript variant 1, L36A, ribosomal protein L44, L44, RPL44, L44-like ribosomal protein (L44L), cell growth-inhibiting gene 15 protein, cell migration-inducing gene 6 protein (MIG6)
gagtcctctcagccgcccgagggcgctgcgctgagccttacactctatgattgctcctaccgactcccatgaggaagtgcgatcgggaacctcctatatacttccgtttgcctcgcggtttctttctttccgcgccgatagcgctcacgcaagcatggttaacgtccctaaaacccgccggactttctgtaagaagtgtggcaagcaccaaccccataaagtgacacagtacaagaagggcaaggattctctgtacgcccagggaaagcggcgttatgacaggaagcagagtggctatggtgggcaaactaagccgattttccggaaaaaggctaaaactacaaagaagattgtgctaaggcttgagtgcgttgagcccaactgcagatctaagagaatgctggctattaaaagatgcaagcattttgaactgggaggagataagaagagaaagggccaagtgatccagttctaagtgtcatcttttattatgaagacaataaaatcttgagtttatgttcacttcatttgtttgctgttcatcttttgggagggaataagctagagccatcaatacaattccgcttgtggggaaatttatgcctcttactggtactacttgttttgcattgaagctgactggttgagttcacatcatatgttgcaattttctaatttggcacttcaatcactaggggccttatgaggcagtttgtcattatgcaatggttattggttatcatgtgagtagacacatttcaggctaatagggagaagtcagtaacacattcatagtgaatatgagatgtctttgctaagagttaagtgtcagatctttgttataacagttaatttaataaagaattttggcattgttcttc

SEQ ID NO: 67 - DNA, 1351 bp, Homo sapiens
  adenine nucleotide translocator 2 (fibroblast) (ANT2), solute carrier family 25 (mitochondrial carrier, adenine nucleotide translocator) member 5 (SLC25A5), ADP,ATP carrier protein 2 (AAC2), fibroblast isoform, 2F1, T2, T3
agctccggctccccctatataaatcggccatttgcttcgctccgccccgcagcgccggagtcaaagccggttcccggcccagtcccgtcctgcagcagtctgcctcctctttcaacatgacagatgccgctgtgtccttcgccaaggacttcctggcaggtggagtggccgcagccatctccaagacggcggtagcgcccatcgagcgggtcaagctgctgctgcaggtgcagcatgccagcaagcagatcactgcagataagcaatacaaaggcattatagactgcgtggtccgtattcccaaggagcagggagttctgtccttctggcgcggtaacctggccaatgtcatcagatacttccccacccaggctcttaacttcgccttcaaagataaatacaagcagatcttcctgggtggtgtggacaagagaacccagttttggctctactttgcagggaatctggcatcgggtggtgccgcaggggccacatccctgtgttttgtgtaccctcttgattttgcccgtacccgtctagcagctgatgtgggtaaagctggagctgaaagggaattccgaggcctcggtgactgcctggttaagatctacaaatctgatgggattaagggcctgtaccaaggctttaacgtgtctgtgcagggtattatcatctaccgagccgcctacttcggtatctatgacactgcaaagggaatgcttccggatcccaagaacactcacatcgtcatcagctggatgatcgcacagactgtcactgctgttgccgggttgacttcctatccatttgacactgttcgccgccgcatgatgatgcagtcagggcgcaaaggaactgacatcatgtacacaggcacgcttgactgctggcggaagattgctcgtgatgaaggaggcaaagcttttttcaagggtgcatggtccaatgttctcagaggcatgggtggtgcttttgtgcttgtcttgtatgatgaaatcaagaagtacacataagttatttcctaggatttttccccctgtgaacaggcatgttgtattatataacatatcttgagcattcttgacagactcctggctgtcagtttctcagtggcaactatttactggttgaaaatgggaagcaataatattcatctgaccagttttctcttaaagccatttccatgatgatgatgatgggactcaattgtattttttatttcagtcactcctgataaataacaaatttggagaaataaaaatatctaaaataaattttgtctgcagtatattttcatataaaaatgcatatttgagtgctacattcgaataaatactacctttttagtgaa

SEQ ID NO: 68 - DNA, 2179 bp, Homo sapiens
  transketolase transcript variant 1 (TKT, TK, TKT1), epididymis luminal protein 107 (HEL107), Wernicke-Korsakoff syndrome
gatccgagccccgcctcctccccctgccccgcctctcccatccccgccccgccccgcccggcgacttaacgcgcccccgccccgcgcccggcctcggcagccgcctgtcgccgcgggagcagccgctatctctgtgtgtccgcgtgtgcgcccggtccccgcctgccgcaccatggagagctaccacaagcctgaccagcagaagctgcaggccttgaaggacacggccaaccgcctacgtatcagctccatccaggccaccactgcggcgggctctggccaccccacgtcatgctgcagcgccgcagagatcatggctgtcctctttttccacaccatgcgctacaagtcccaggacccccggaatccgcacaatgaccgctttgtgctctccaagggccatgcagctcccatcctctacgcggtctgggctgaagctggtttcctggccgaggcggagctgctgaacctgaggaagatcagctccgacttggacgggcacccggtcccgaaacaagctttcaccgacgtggccactggctccctgggccagggcctcggggccgcttgtgggatggcctacaccggcaaatacttcgacaaggccagctaccgagtctattgcttgctgggagacggggagctgtcagagggctctgtatgggaggccatggccttcgccagcatctataagctggacaaccttgtggccattctagacatcaatcgcctgggccagagtgacccggccccactgcagcaccagatggacatctaccagaagcggtgcgaggccttcggttggcatgccatcatcgtggatggacacagcgtggaggagctgtgcaaggcctttggccaggccaagcaccagccaacagccatcattgccaagaccttcaagggccgagggatcacgggggtagaagataaggagtcttggcatgggaagcccctccccaaaaacatggctgagcagatcatccaggagatctacagccagatccagagcaaaaagaagatcctggcaacccctccacaggaggacgcaccctcagtggacattgccaacatccgcatgcccagcctgcccagctacaaagttggggacaagatagccacccgcaaggcctacgggcaggcactggccaagctgggccatgccagtgaccgcatcatcgccctggatggggacaccaaaaattccaccttctcggagatcttcaaaaaggagcacccggaccgcttcatcgagtgctacattgctgagcagaacatggtgagcatcgcggtgggctgtgccacccgcaacaggacggtgcccttctgcagcacttttgcagccttcttcacgcgggcctttgaccagattcgcatggccgccatctccgagagcaacatcaacctctgcggctcccactgcggcgtttccatcggggaagacgggccctcccagatggccctagaagatctggctatgtttcggtcagtccccacatcaactgtcttttacccaagtgatggcgttgctacagagaaggcagtggaactagccgccaatacaaagggtatctgcttcatccggaccagccgcccagaaaatgccatcatctataacaacaatgaggacttccaggtcggacaagccaaggtggtcctgaagagcaaggatgaccaggtgaccgttatcggggctggggtgaccctgcacgaggccttggccgctgccgaactgctgaagaaagaaaagatcaacatccgcgtgctggaccccttcaccatcaagcccctggacagaaaactcattctcgacagcgctcgtgccaccaagggcaggatcctcaccgtggaggaccattattatgaaggtggcattggtgaggctgtgtccagtgcagtagtgggcgagcctggcatcactgtcacccacctggcagttaaccgggtaccaagaagtgggaagccggctgagctgctgaagatgtttggtatcgacagggatgccattgcacaagctgtgaggggcctcatcaccaaggcctagggcgggtatgaagtgtggggcgggggtctatacattcctgagattctgggaaaggtgctcaaagatgtactgagaggaggggtaaatatatgttttgagaaaaatgaattggccctgaaaaaaaaaaaaaaaaaa

SEQ ID NO: 69 - DNA, 2617 bp, Homo sapiens
  lipase maturation factor 2 (LMF2), transmembrane protein 153 (TMEM153), transmembrane protein 112B (TMEM112B)
gggccctgctctagcgggccgcgtagcggacatggcgggctcccggctcccgcggcagctcttcctccagggcgtggcggccgtcttcatgtttgctttcgcttccctctacacgcaaatcccaggcctgtatggccccgagggcatcctacctgcaaggaggacgctgcggcctcagggcaaggggcgctggcagcagctgtgggagaccccgacgctgctgtgggaagcgccgagactggggctggacacggcccagggcctggagctgctgagcctgctgggtgcactagtggccctgggagccctgctgctgagcccactgcgccaccctgtcatctacttgctgctttgggccgcctacctgtcagcctgccaggtgggccaggtgttcctttatttccagtgggactccctgctgctagagactggcttcctggccgtgctggtggccccgctgaggccagcctcccaccgcaaggaggccccccagggcaggcaggcaggggccctgccccacgaagacctccccttctggctggtgcgatggctgctgttccgcctcatgttcgcctcaggcgtggtcaagctgaccagccgctgccctgcgtggtgggggctcactgccctcacctaccactacgagacccagtgcctgcccacgcccgccgcctggttcgcacaccacctgccggtctggctgcacaagctcagcgtggtggccaccttcctaattgagatcgctgtgccgcccctgttcttcgcccccattcgacgcctgcgcttggctgctttctactcgcaggtgctgctgcaggtcctgattatcatcaccggcaactacaacttcttcaacctgatgacgctggtgcttaccactgcgctgctggacgaccagcacctggctgctgagcctggccacggcagccgcaagaagacggccacctcctggcccaaggccctgctggccaccctgtcgctgctgctggaactagccgtctacgggcttctggcctatggcactgtgcactactttggcctggaggttgactggcagcagcgcaccatccactccagaaccactttcaccttccaccagttttctcagtggctgaagacactgacgctgcccactgtgtggctgggtgtggcctccctggtctgggagctgctgagtgccctgtggaggtggacccaggtgcggggctggctacggaagctcagtgctgtagtccaactgtcccttgtgggcactgcgaccgtggccttgttcctgattagcctggtgccgtactcctacgtggagcccgggacccacgggcgcctctggaccggggcccaccgcctgtttggtgccgtggagcacctacagctggccaactcctacggcctcttccgccgcatgactgggcttggtggacggcctgaggtggtgctggagggcagttacgacggccaccactggacggagatcgagttcatgtacaagcctgggaacctgagccggccgcccccggttgtggtgccccaccagccacgcctggactggcagatgtggtttgcagccctgggcccacacacgcacagcccgtggttcacaagcctggtcttgcgcctgctgcagggcaaggagccagtgatccgccttgtccagagccaagtggccaggtatcccttccacaagcagccgcccacctacgtccgagcccagcgctacaagtactggttctcccagcctggggagcagggccagtggtggcggcgccagtgggtggaggagttcttcccatccgtgtccctgggggaccccacgctggagacgctgctcaggcagtttggactacaggagaaaagcccacctcgcacccgcagcgccaacagcaccctggcccaggccctccactggactcgctctcagctgtctcccctggaggcccccgccctgctctgggggctcctcatggccgtgggggctgtcagatttgtgcaagccctgctagcaccctgttctctccggtcctccccgctggcaccagtcagcggggagaagcgcaggccagcctcccagaaagactccggagctgcctccgaacaggccaccgcagcccccaacccctgctccagtagttcgaggaccacccggcgaaagaagtagctgtgttctcccagctgcacgtcctgagagggccaggtcgccgggagtgctctggcctccggcaggacaggacccagccactgtgccttagctgaccctgcagggccaggcacaggttggggggctgcccctggggtttgcagggtgctgcattgagggctccaggccccacccccacgccagccatgcccctccccaggactcccactattgcctctgtgattggcccaggaggaaaacacgaccaagctcaagacccttcccctgccctgggctgtgggggtctgagtctagagcccccaaccctaggccccgtgccagaggggaagaggctgactcccaggggaagaggggaagcactgtcatcttccacgtcatcttcacaccagcccatcctgccctttagatctgggcaccaataaaggcgtcttttgtgcttggctgaaaaaaaaaaaaaaaaaa

SEQ ID NO: 70 - DNA, 4499 bp, Homo sapiens
  60S ribosomal protein L13 transcript variant 1, L13, breast basic conserved protein 1 (BBC1), D16S444E, D16S44E, OK/SW-cl.46
ccagagtgcattgcggggccgcttcctttccgctcggctgttttcctgcgcaggagccgcagggccgtaggcagccatggcgcccagccggaatggcatggtcttgaagccccacttccacaaggactggcagcggcgcgtggccacgtggttcaaccagccggcccgtaagatccgcagacgtaaggcccggcaagccaaggcgcgccgcatcgccccgcgccccgcgtcgggtcccatccggcccatcgtgcgctgccccacggttcggtaccacacgaaggtgcgcgccggccgcggcttcagcctggaggagctcagggtggccggcattcacaagaaggtggcccggaccatcggcatttctgtggatccgaggaggcggaacaagtccacggagtccctgcaggccaacgtgcagcggctgaaggagtaccgctccaaactcatcctcttccccaggaagccctcggcccccaagaagggagacagttctgctgaagaactgaaactggccacccagctgaccggaccggtcatgcccgtccggaacgtctataagaaggagaaagctcgagtcatcactgaggaagagaagaatttcaaagccttcgctagtctccgtatggcccgtgccaacgcccggctcttcggcatacgggcaaaaagagccaaggaagccgcagaacaggatgttgaaaagaaaaaataaagccctcctggggacttggaatcagtcggcagtcatgctgggtctccacgtggtgtgtttcgtgggaacaactgggcctgggatggggcttcactgctgtgacttcctcctgccaggggatttggggctttcttgaaagacagtccaagccctggataatgctttactttctgtgttgaagcactgttggttgtttggttagtgactgatgtaaaacggttttcttgtggggaggttacagaggctgacttcagagtggacttgtgttttttctttttaaagaggcaaggttgggctggtgctcacagctgtaatcccagcactttgaggttggctgggagttcaagaccagcctggccaacatgtcagaactactaaaaataaagaaatcagccatgcttggtgctgcacacttgtagttgcagctcctgggaggcagaggtgagggatcacttaacccaggaggcagaggctgcactgagccaggatcacgccactgcactctagcctgggcaacagtgagactgtctcaaaaaaaaaaaaagagacagggtcttcggcacccaggctggagtacagtgccacaatcatggctcactgcagtcttgaactcatggcctcaagcagtcctccctcagcctcccaagtagaggggtttataggcacgagaccctgcacccaacctagagttgccttttttaagcaaagcagtttctagttaatgtagcatcttggactttggggcgtcattcttaagcttgttgtgcccggtaaccatggtcctcttgctctgattaacccttccttcaatgggcttcttcacccagacaccaaggtatgagatggccctgccaagtgtcggcctctcctgttaaacaaaaacattctaaagccattgttcttgcttcatggacaagaggcagccagagagagtgccagggtgccctggtctgagctggcatccccatgtcttctgtgtccgagggcagcatggtttctcgtgcagtgctcagacacagcctgccctagtcctaccagctcacagcagcacctgctctccttggcagctatggccatgacaaccccagagaagcagcttcagggaccgagtcagattctgttttgtctacatgcctctgccgggtgccggtattgaggcacccagggagctgttactggcgtggaaataggtgatgctgctacctctgctgctgcactcacagccacacttgatacacgatgacaccttgcttgtttggaaacatctaaacatctagtagatgacttgcaggctgttggctaccagtttcctgtctgaggtgtatatgttaacttcgtgatcagtttgtatgtttgggactcttgtcctatgtaaagttaaggtgggccgggtgcagtggctcacgcctgtaatcctaacactgggaggccgaggcgggtggatcacctgatggtgaaacctcatctctactgaaaatacaaaaattagctgagtggtgacacacgcctgtaatcccagctacttggtaggcttgaacccaggaggcagagattgcagtgagccgagctgcaccactgtgctccagcctgggtgacagcgagactcagtctcaaaaaaagttgtacaaggtggatggttggaagcttgagcctaggctcgaatccctctcacgtgagagggcctgaagatttctggtggattccaacctggctgaagactggccgtggggggtgcaggggtctccagcgctctgccctccagcctgcttcctccctgcccacaccgcactaggggaagggcctttcctgctgcctgcggggccgcacctggagtaggtaatgccatgtggtgacgtgaatggagcagaggtctgtgccccatcacaccgccttgctgtttttactgtgggacaaaagcactctgatctgcgtgttccgggggccctcctaccagccgacttgacgggaagtcagggttcaggtatcatctgtgcacctggggcggggtagtctgcactgaacctgccagagtcccctcctcatttcactgaaagtcacagtctccagggctgtgttgctaaccttacgttctctccgtttgcttaatctattaagagccctaacaggagaggatgggctttctctgttgtctggggccctgctgttggccggtgctcttagcaagaggtcatttttctaggttgcgctgggacattgtgagtttggtgagggtcatggatgtgggctgggctgggctgggctgggccgggctgcctgctgcctgctgctcccctacctgaaatgcagctagtgcggctctgcccttcctggggctgaggaaggcttctgcaggatagctggggggctgggcaggtgggtgaggcagcctccctgctgacactcagtccttgtagctggagcaagatctcctgatccaggtacgggcctgtctgctccaagaaagactctgccaccagatgcaaaggggccctttgttttaacttagtccctggggaccgcctgattcagcacctgtcggcccaggataccccgctggtggggacaagtgcctgagtgtgggccgtgcccgagtgtggccatccctgagtggggccgtcctgactaggaagtggcttttcagttgtgatgtgtgggcctgacctagggggcgctgtggaacccgggctggaaccagccctctgtgccaggccgcagacaggttccgccggccctgaggggcagctgccatggcgtgggtcactgggagctgagaggaagggcccccaccgcacctcaggcaaagcggctctgggaacaccttgatttcgtccatgtgagccgtcccagggagggcagccaagctgtgaagcctgagaaactgacctgtgtgccacgagcttgtggtctgctgcccggtggaggaagtgcaggtgcgcccaggctcctcattccgttttgcaggattccttcggggtgtgagcatttcctattcagcctgtcgcccccggggagcacgggctggctctgtggtgcccgtggccttttgtagaagcgttggttttacggcaggttcatctctggggcagcctcccacagtgggtggggctttgccagcagtgcccacgggggtcatggggccaggcgcgctccggcgcctgcagaactgatcggggatagtctcaggaggcgctagtcacgtgccccggtgatcggggatagtctcagaaggcgctagtctcctgccccggtgatcggggatagtctcaggaggcacgagtcgcctgcctcggtgatgcaccgtttctcacaccggctgctctggcccgagctaaaggggaagacgtgtgcggataggagctgcacacaattttcctccatgtattgtttattttgctttttcttttggctagacattaggaatttcagttttcccaagttgtatttttccttttctattttaaaattatcatgcagggctgggtgaggtcgctcacgcctatagtctcaaaactttgggaggctgaggggggaggatggcatgagcccaggagtttaaggctgcagtgagccgagatcgctccactgtcctccagcctgcatgacagagcgagaccctatctcaggaaaaaaaaaaacaaaactattatgcagtagtttcgaccctggaagacgagtgtgcatctttgagttgtaacacgtgtacctcgcccatccaggcgtagtttcatttggaatctggttatcctgtagttgctttgttaaaaatatatgtaattgcaaatcattt

SEQ ID NO: 71 - DNA, 1494 bp, Homo sapiens
  cathepsin H (CTSH), pro-cathepsin H preproprotein, cathepsin B3, cathepsin BA, N-benzoylarginine-beta- naphthylamide hydrolase, aleurain, ACC-4, ACC-5, CPSB
ccacgctcgtgccgctccccccccgcgctcccagttgacgctctgggccgccacctccgcggaccctgagcgcaagagccaagccgccagcgctgcgatgtgggccacgctgccgctgctctgcgccggggcctggctcctgggagtccccgtctgcggtgccgccgaactgtgcgtgaactccttagagaagtttcacttcaagtcatggatgtctaagcaccgtaagacctacagtacggaggagtaccaccacaggctgcagacgtttgccagcaactggaggaagataaacgcccacaacaatgggaaccacacatttaaaatggcactgaaccaattttcagacatgagctttgctgaaataaaacacaagtatctctggtcagagcctcagaattgctcagccaccaaaagtaactaccttcgaggtactggtccctacccaccttccgtggactggcggaaaaaaggaaattttgtctcacctgtgaaaaatcagggtgcctgcggcagttgctggactttctccaccactggggccctggagtctgcgatcgccatcgcaaccggaaagatgctgtccttggcggaacagcagctggtggactgcgcccaggacttcaataatcacggctgccaagggggtctccccagccaggctttcgagtatatcctgtacaacaaggggatcatgggtgaagacacctacccctaccagggcaaggatggttattgcaagttccaacctggaaaggccatcggctttgtcaaggatgtagccaacatcacaatctatgacgaggaagcgatggtggaggctgtggccctctacaaccctgtgagctttgcctttgaggtgactcaggacttcatgatgtatagaacgggcatctactccagtacttcctgccataaaactccagataaagtaaaccatgcagtactggctgttgggtatggagaaaaaaatgggatcccttactggatcgtgaaaaactcttggggtccccagtggggaatgaacgggtacttcctcatcgagcgcggaaagaacatgtgtggcctggctgcctgcgcctcctaccccatccctctggtgtgagccgtggcagccgcagcgcagactggcggagaaggagaggaacgggcagcctgggcctgggtggaaatcctgccctggaggaagttgtggggagatccactgggacccccaacattctgccctcacctctgtgcccagcctggaaacctacagacaaggaggagttccaccatgagctcacccgtgtctatgacgcaaagatcaccagccatgtgccttagtgtccttcttaacagactcaaaccacatggaccacgaatattctttctgtccagaagggctactttccacatatagagctccagggactgtcttttctgtattcgctgttcaataaacattgagtgagcacctccccagatggagcatgctggtcctggaaaaaaaaa

SEQ ID NO: 72 - DNA, 5604 bp, Homo sapiens
  family with sequence similarity 83, member H, FAM83H variant 1 (FAM83H), AI3
gggcggcggtcggctcctgctgcccctgtgccgagaccccgcgcacctggccaggcccctggccccaacatggcccgtcgctctcagagctcctcgcagggggacaacccactggcacccgggtacctgccgcctcactacaaagagtactaccgcctggcggtggatgcactggccgagggtggctcggaggcctacagccgcttcctcgctaccgagggggcaccagacttcctgtgccctgaagagctggaacatgtgagccgacaccttcggcctccgcagtatgttacccgagagccacctgaaggcagccttctcgacgtggacatggatggctcctcgggtacatactggccagtgaactcagaccaggccgtgcctgagcttgatttgggctggcctctgaccttcggcttccagggcaccgaggtgaccaccttggtgcagccaccgccccccgacagccccagtatcaaggatgaggcccgcaggatgatccgttccgcccagcaggtggtggccgtggtgatggacatgttcactgatgtggacctgctcagcgaagtgctggaggccgcggcccgtcgggtcccagtctacatcctgctggatgagatgaacgcgcagcacttcctggacatggccgacaagtgccgtgtcaacctgcaccacgtggatttcctgcgcgtacggactgtggcgggccccacctactactgccgcactgggaagtccttcaagggccacgtcaaggagaagttcctgctggtggactgtgccgtggtgatgagtgggagctacagcttcatgtggtcctttgagaagatccaccgcagcctggcgcacgtgttccaaggagagctggtctccagcttcgacgaggagttccgcatcctcttcgcgcagtccgagccgcttgtgccctcggccgcggccctggcccgcatggacgcctatgccctggctccgtatgccggggccgggcctctcgtgggcgtccctggggtcggggcgccaacccccttctccttccctaaacgagcgcacctcctgttcccgccaccccgggaagagggcctgggcttcccctccttcctcgacccggaccgccacttcctgtcggccttccgccgggaggagccgccgcggatgccggggggcgcgctggaaccgcacgcggggctgcggccgctctcgcggcgcctggaggccgaggccgggccggctggggagctcgcgggcgcgcggggcttcttccaggcgcggcacctggagatggacgccttcaagcggcacagcttcgcgaccgagggcgcgggcgccgtggagaacttcgcggccgcgcggcaggtgtcgcggcagacgttcctcagccacggcgacgacttccgcttccagaccagccacttccaccgtgaccagctctaccagcagcagtaccagtgggacccgcagctcacgccggcgcgcccgcaaggcctgttcgagaagcttcgcgggggccgcgcgggtttcgcggacccggatgacttcaccctgggcgccgggccccgcttcccggagctcggacccgacgggcaccagcggctggactacgtgccgtccagcgcgtcccgcgaggtgcgccacggctcggaccccgccttcgcgcccggaccccgcggcctggagcccagcggagccccgcgccccaacctgacccagcgcttcccatgccaggccgcggcgaggccgggcccagaccccgctcccgaggcggagccggagcgcaggggcgggcccgaggggcgggcagggctgcggcgctggcgtttggcctcctacttgagcggctgccacggcgaggatgggggcgacgacggcctaccggcgcccatggaagcggaggcttacgaagacgacgtgctggctcccgggggccgggcacctgccggcgacctgctcccctcggccttccgcgtcccagcagccttccccaccaaggtcccggtgccaggcccgggcagcggcggcaacggcccagagcgcgagggcccggaggagcctggcctggccaagcaggactcattccgctcgcgcctgaaccccctggtccagcgcagctccaggctgcgctcctcgctcatcttcagcacgtcacaggccgagggcgcggccggggctgcggcggccactgagaaggtgcagctgctgcacaaggagcagacggtcagcgagacgctggggcccggcggagaggccgtgcgctccgcggcttccaccaaggtggcggagctgctggagaagtacaagggcccagcccgtgatcccggcggcggcgcgggcgccatcaccgttgccagccacagcaaggccgtcgtgtcccaggcgtggcgggaagaggtggcggccccaggtgccgtggggggcgagcgccgcagcctcgagagctgcctgctggacctgcgcgactcctttgcacagcagctgcaccaggaggcggagcggcagccgggagccgcgtcgctcaccgcggcgcagctgctcgacacactgggccggagcggctccgaccgcctgccttcccgcttcctctctgcccagagccactcaacgtccccgcaagggctggacagccctctgccgctggaagggtccggagcgcaccaggtgctccataatgagtcaaaagggagccccacctcggcttaccctgagcggaaggggagccccacgcctgggttttccactcgaagaggaagtccaactacaggatttatcgagcagaaggggagccccacctcagcctaccccgagcgcaggggtagtccggtgccccccgtgccggagcgcaggagcagtccggtgccccccgtgccggagcgcaggggcagcctcacccttaccatctccggggagtccccgaaggccgggcccgcggaggaggggccgagcggccccatggaagtcctgcgcaaaggctccttgcgtcttaggcagctgctgagccccaagggcgagcggcgcatggaggatgagggtggcttcccagtgccgcaggagaacggccaacccgagagcccgcggcgtctgtcactgggccagggtgacagcacggaggctgccacagaagagcggggtccgcgggcgcgcctgtcctcagccacggccaacgccttgtacagcagcaaccttcgggatgacacgaaggccattctggagcagatcagtgcccacggccagaagcaccgtgcggtccctgccccgagccccggcccgacccacaacagccccgagctaggccgtccaccggctgctggcgtcctggccccagatatgtccgacaaggacaagtgttcagccatcttccgctcggacagcttggggacccagggccggctgagccgcacgctgccagccagcgcggaggagcgcgatcggctgctgcgccgcatggagagcatgcgcaaggagaagcgcgtgtacagccgcttcgaggtcttctgcaagaaagaggaggccagcagccctggggcaggggaaggccccgcggaggagggcaccagggacagcaaggtgggcaagttcgtgcccaagatcctgggcacgttcaaaagcaagaagtgagtcttctggcctggcaacccaggccagggtgcccgcatcgctgccccggtcatccagaagccccgcggaacagagagccctgctcatgtgcttgagcagcggctgtcaggccacggccgcttggggcttggctgagtgcgccagacctcggctccactggaggctcacctggcagctgccgtctctgccccctggcctccccaacgctggggctgcacccctcgccaccagtgcctttctcccctcagcaccttcatctctgcaccgtcagccttgcgtggcgcagcgtctggctccgccatctctttgtgcctcagtcccccccgccccctttatttttttgagacctagggctggagtgcagttgagcggtctgggctcactgcaacctctgcctcccgggttccagcgattctcctgcctcagcctcctgagtagctgggattacagatgtatgctaccacgcccaggtagtttttgtatttttagtagagacagggtttcactatgttggccaggctggtctccaactcctggcctcaaatgatcagcccgcttcagcctcccaaagtggggggattacaggcgtgagccttgcaccccgctaagtcccctatcctcttgcaagggtctcgcctctgtgcctcaattcctcattctctgggcccttctcctcctcagggcctcctgttctcagggcctcccccctccccgctccctccctctctcaaggtctcctccttccctcccccccccgtctcccccctcccccgcctgggcttcacttcctttcctacttggattctcctgctcgctgcctcccagcatcttttttggaggcccgtctcttgctgtggggaagactgggctggctgcgggcagtttgcaaggggtgggtggggcgggggggggagctggaccagaagatgccccttggagtggcaaggaagctggacagggcaggcctctggggacgggacacagggaagcccgaaggggcgccttggccaggtctgccatctcctccagcgaggctctggccagcactgggtgagagtggggagggggcactggcctttgcagcacagtaaaacatggtccagacaacctgtggccccggcctcatgagcaccccctgcacaggcccggcccaagccaggcgctagaagggctggttgtggagtgcttatccttgacaggtatggggccaggtgagggcaggggacaaggtgcagctgaggccgagcccaactaggtcctgggcacccctgcaggtgggagtggtccttgtcctcctggtatccagcagacacccccctctccccaccagccccattctcaggtcctttcctctttgtcaccaacaccaagaatctgtccagggttcttggcttatcttttatctcttttcactcctagagaggaattgcaattgactcagaatgacacattttggcaccacgtgtgtagaaagcccccactgttagatgatagcctcgtgaaattcatgtttctgtattctcctatttcttttcaaaaactaattttttttttagtgtaataaatcctaagagggaactgatttaagaaacaaggccgccaaacaaaggcagcagttccgactccagcagctgggaaaggaaggaaagtgaccccactttcactcctgcacagcccactggttaccaaaaccaccgtgcaagtcgggatgacagcagggacttctggccaggtgggaaaggtgcctggaagcgggatgcgcctgtgcgtctcttggccatgatgttcttgtgggcatgttattcttggtgctgcctggggtgttgctgagcggacaggctctccagctggagtccatggagaggccagaggctggcggccctgcctgggccttcggagcctcctgcctgcaccctccacctcttctaaaccatgatgtggcacattttggtgttaataaaacacaacacacaaagtaaaaaaaaaaaaaaaaaa

SEQ ID NO: 73 - DNA, 735 bp, Homo sapiens
  40S ribosomal protein S29 transcript variant 2, S29
cttttacctcgttgcactgctgagagcaagatgggtcaccagcagctgtactggagccacccgcgaaaattcggccagggttctcgctcttgtcgtgtctgttcaaaccggcacggtctgatccggaaatatggcctcaatatgtgccgccagtgtttccgtcagtacgcgaaggatatcggtttcattaagaaagacctgagctgtcttccttggcactgcctatggaggtgacacccatctcctccatcatggccatcctgagaccgctcgcgaagcccaagatcatcaaaaagagcaccaagttcactgggaaccagtcagactgatatgtcaaaattaagggtaactggtggaaacacagaggtattaacaacagggttcatagaaggtttgagggccagatctatgcccaacattggttatgggagaaacaaaaagacaaagcacatactgcccagtggcttctggaagttcctggtccacaacgttaaggagctggaagtactgctggtgagcaacaaatcttactgtgttgagatcactcatgatgtttcttccaagaactgcaaagccatcttggaaagagcagcccaggtggtcatcagagtcaccaatgccaatgccagcctgcacagtgcagaaagtgaatagacagtgaatgtgtttgttttattggggtttaaataaaaccaataaaactgtaaaaacaaaaacaacaaaaaaaaaaaaaaa

SEQ ID NO: 74 - DNA, 594 bp, Homo sapiens
  60S ribosomal protein L23, L23, ribosomal protein L17, rpL17
ggccacgtgaggagggtgggcggggcgttaaagttcatatcccagtgtcctttgaatcgacttccttttttcttttttccggcgttcaagatgtcgaagcgaggacgtggtgggtcctctggtgcgaaattccggatttccttgggtcttccggtaggagctgtaatcaattgtgctgacaacacaggagccaaaaacctgtatatcatctccgtgaaggggatcaagggacggctgaacagacttcccgctgctggtgtgggtgacatggtgatggccacagtcaagaaaggcaaaccagagctcagaaaaaaggtacatccagcagtggtcattcgacaacgaaagtcataccgtagaaaagatggcgtgtttctttattttgaagataatgcaggagtcatagtgaacaataaaggcgagatgaaaggttctgccattacaggaccagtagcaaaggagtgtgcagacttgtggccccggattgcatccaatgctggcagcattgcatgattctccagtatatttgtaaaaaataaaaaaaaaaactaaacccattaaaaagtatttgtttgcaaaaaaaaaaaaaaaaaa

SEQ ID NO: 75 - DNA, 514 bp, Homo sapiens
  40S ribosomal protein S25, S25
cttcctttttgtccgacatcttgacgaggctgcggtgtctgctgctattctccgagcttcgcaatgccgcctaaggacgacaagaagaagaaggacgctggaaagtcggccaagaaagacaaagacccagtgaacaaatccgggggcaaggccaaaaagaagaagtggtccaaaggcaaagttcgggacaagctcaataacttagtcttgtttgacaaagctacctatgataaactctgtaaggaagttcccaactataaacttataaccccagctgtggtctctgagagactgaagattcgaggctccctggccagggcagcccttcaggagctccttagtaaaggacttatcaaactggtttcaaagcacagagctcaagtaatttacaccagaaataccaagggtggagatgctccagctgctggtgaagatgcatgaataggtccaaccagctgtacatttggaaaaataaaactttattaaatcaaaaaaaaaaaaaaaaaaaaaaaaa

SEQ ID NO: 76 - DNA, 1794 bp, Homo sapiens
  tubulin, beta 3 class III transcript variant 1 (TUBB3), class III beta-tubulin, tubulin beta-4 chain (TUBB4), beta-4, CDCBM, CDCBM1, CFEOM3A
gacatcagccgatgcgaagggcggggccgcggctataagagcgcgcggccgcggtccccgaccctcagcagccagcccggcccgcccgcgcccgtccgcagccgcccgccagacgcgcccagtatgagggagatcgtgcacatccaggccggccagtgcggcaaccagatcggggccaagttctgggaagtcatcagtgatgagcatggcatcgaccccagcggcaactacgtgggcgactcggacttgcagctggagcggatcagcgtctactacaacgaggcctcttctcacaagtacgtgcctcgagccattctggtggacctggaacccggaaccatggacagtgtccgctcaggggcctttggacatctcttcaggcctgacaatttcatctttggtcagagtggggccggcaacaactgggccaagggtcactacacggagggggcggagctggtggattcggtcctggatgtggtgcggaaggagtgtgaaaactgcgactgcctgcagggcttccagctgacccactcgctggggggcggcacgggctccggcatgggcacgttgctcatcagcaaggtgcgtgaggagtatcccgaccgcatcatgaacaccttcagcgtcgtgccctcacccaaggtgtcagacacggtggtggagccctacaacgccacgctgtccatccaccagctggtggagaacacggatgagacctactgcatcgacaacgaggcgctctacgacatctgcttccgcaccctcaagctggccacgcccacctacggggacctcaaccacctggtatcggccaccatgagcggagtcaccacctccttgcgcttcccgggccagctcaacgctgacctgcgcaagctggccgtcaacatggtgcccttcccgcgcctgcacttcttcatgcccggcttcgcccccctcacagcccggggcagccagcagtaccgggccctgaccgtgcccgagctcacccagcagatgttcgatgccaagaacatgatggccgcctgcgacccgcgccacggccgctacctgacggtggccaccgtgttccggggccgcatgtccatgaaggaggtggacgagcagatgctggccatccagagcaagaacagcagctacttcgtggagtggatccccaacaacgtgaaggtggccgtgtgtgacatcccgccccgcggcctcaagatgtcctccaccttcatcgggaacagcacggccatccaggagctgttcaagcgcatctccgagcagttcacggccatgttccggcgcaaggccttcctgcactggtacacgggcgagggcatggacgagatggagttcaccgaggccgagagcaacatgaacgacctggtgtccgagtaccagcagtaccaggacgccacggccgaggaagagggcgagatgtacgaagacgacgaggaggagtcggaggcccagggccccaagtgaagctgctcgcagctggagtgagaggcaggtggcggccggggccgaagccagcagtgtctaaacccccggagccatcttgctgccgacaccctgctttcccctcgccctagggctcccttgccgccctcctgcagtatttatggcctcgtcctccccacctaggccacgtgtgagctgctcctgtctctgtcttattgcagctccaggcctgacgttttacggttttgttttttactggtttgtgtttatattttcggggatacttaataaatctattgctgtcagatacccttaaaaaaaaaaaaaaaaaaaaaaaaa

SEQ ID NO: 77 - DNA, 660 bp, Homo sapiens
  40S ribosomal protein S10 transcript variant 2, S10, DBA9
ggcggggggcgggtccacgccagcccggaagagacgcagcaccgcgcatgctccttcctttccagccccggtaccggaccctgcagccgcagagatgttgatgcctaagaagaaccggattgccatttatgaactcctttttaaggagggagtcatggtggccaagaaggatgtccacatgcctaagcacccggagctggcagacaagaatgtgcccaaccttcatgtcatgaaggccatgcagtctctcaagtcccgaggctacgtgaaggaacagtttgcctggagacatttctactggtaccttaccaatgagggtatccagtatctccgtgattaccttcatctgcccccggagattgtgcctgccaccctacgccgtagccgtccagagactggcaggcctcggcctaaaggtctggagggtgagcgacctgcgagactcacaagaggggaagctgacagagatacctacagacggagtgctgtgccacctggtgccgacaagaaagccgaggctggggctgggtcagcaaccgaattccagtttagaggcggatttggtcgtggacgtggtcagccacctcagtaaaattggagaggattcttttgcattgaataaacttacagccaaaaaaccttaaaaaaaaaaaaaaaaaa

SEQ ID NO: 78 - DNA, 8481 bp, Homo sapiens
  fatty acid synthase (FASN, FAS), short chain dehydrogenase/reductase family 27X, member 1 (SDR27X1), OA-519
gagagacggcagcggccccggcctccctctccgccgcgcttcagcctcccgctccgccgcgctccagcctcgctctccgccgcccgcaccgccgcccgcgccctcaccagagcagccatggaggaggtggtgattgccggcatgtccgggaagctgccagagtcggagaacttgcaggagttctgggacaacctcatcggcggtgtggacatggtcacggacgatgaccgtcgctggaaggcggggctctacggcctgccccggcggtccggcaagctgaaggacctgtctaggtttgatgcctccttcttcggagtccaccccaagcaggcacacacgatggaccctcagctgcggctgctgctggaagtcacctatgaagccatcgtggacggaggcatcaacccagattcactccgaggaacacacactggcgtctgggtgggcgtgagcggctctgagacctcggaggccctgagccgagaccccgagacactcgtgggctacagcatggtgggctgccagcgagcgatgatggccaaccggctctccttcttcttcgacttcagagggcccagcatcgcactggacacagcctgctcctccagcctgatggccctgcagaacgcctaccaggccatccacagcgggcagtgccctgccgccatcgtggggggcatcaatgtcctgctgaagcccaacacctccgtgcagttcttgaggctggggatgctcagccccgagggcacctgcaaggccttcgacacagcggggaatgggtactgccgctcggagggtgtggtggccgtcctgctgaccaagaagtccctggcccggcgggtgtacgccaccatcctgaacgccggcaccaatacagatggcttcaaggagcaaggcgtgaccttcccctcaggggatatccaggagcagctcatccgctcgttgtaccagtcggccggagtggcccctgagtcatttgaatacatcgaagcccacggcacaggcaccaaggtgggcgacccccaggagctgaatggcatcacccgagccctgtgcgccacccgccaggagccgctgctcatcggctccaccaagtccaacatggggcacccggagccagcctcggggctggcagccctggccaaggtgctgctgtccctggagcacgggctctgggcccccaacctgcacttccatagccccaaccctgagatcccagcgctgttggatgggcggctgcaggtggtggaccagcccctgcccgtccgtggcggcaacgtgggcatcaactcctttggcttcgggggctccaacgtgcacatcatcctgaggcccaacacgcagccgccccccgcacccgccccacatgccaccctgccccgtctgctgcgggccagcggacgcacccctgaggccgtgcagaagctgctggagcagggcctccggcacagccaggacctggctttcctgagcatgctgaacgacatcgcggctgtccccgccaccgccatgcccttccgtggctacgctgtgctgggtggtgagcgcggtggcccagaggtgcagcaggtgcccgctggcgagcgcccgctctggttcatctgctctgggatgggcacacagtggcgcgggatggggctgagcctcatgcgcctggaccgcttccgagattccatcctacgctccgatgaggctgtgaagccattcggcctgaaggtgtcacagctgctgctgagcacagacgagagcacctttgatgacatcgtccattcgtttgtgagcctgactgccatccagataggcctcatagacctgctgagctgcatggggctgaggccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccgggcgtggtgcccgcctgccacaactccaaggacacagtcaccatctcgggacctcaggccccggtgtttgagttcgtggagcagctgaggaaggagggtgtgtttgccaaggaggtgcggaccggcggtatggccttccactcctacttcatggaggccatcgcacccccactgctgcaggagctcaagaaggtgatccgggagccgaagccacgttcagcccgctggctcagcacctctatccccgaggcccagtggcacagcagcctggcacgcacgtcctccgccgagtacaatgtcaacaacctggtgagccctgtgctgttccaggaggccctgtggcacgtgcctgagcacgcggtggtgctggagatcgcgccccacgccctgctgcaggctgtcctgaagcgtggcctgaagccgagctgcaccatcatccccctgatgaagaaggatcacagggacaacctggagttcttcctggccggcatcggcaggctgcacctctcaggcatcgacgccaaccccaatgccttgttcccacctgtggagttcccagctccccgaggaactcccctcatctccccactcatcaagtgggaccacagcctggcctgggacgtgccggccgccgaggacttccccaacggttcaggttccccctcagccgccatctacaacatcgacaccagctccgagtctcctgaccactacctggtggaccacaccctcgacggtcgcgtcctcttccccgccactggctacctgagcatagtgtggaagacgctggcccgcgccctgggcctgggcgtcgagcagctgcctgtggtgtttgaggatgtggtgctgcaccaggccaccatcctgcccaagactgggacagtgtccctggaggtacggctcctggaggcctcccgtgccttcgaggtgtcagagaacggcaacctggtagtgagtgggaaggtgtaccagtgggatgaccctgaccccaggctcttcgaccacccggaaagccccacccccaaccccacggagcccctcttcctggcccaggctgaagtttacaaggagctgcgtctgcgtggctacgactacggccctcatttccagggcatcctggaggccagcctggaaggtgactcggggaggctgctgtggaaggataactgggtgagcttcatggacaccatgctgcagatgtccatcctgggctcggccaagcacggcctgtacctgcccacccgtgtcaccgccatccacatcgaccctgccacccacaggcagaagctgtacacactgcaggacaaggcccaagtggctgacgtggtggtgagcaggtggctgagggtcacagtggccggaggcgtccacatctccgggctccacactgagtcggccccgcggcggcagcaggagcagcaggtgcccatcctggagaagttttgcttcactccccacacggaggaggggtgcctgtctgagcgcgctgccctgcaggaggagctgcaactgtgcaaggggctggtgcaggcactgcagaccaaggtgacccagcaggggctgaagatggtggtgcccggactggatggggcccagatcccccgggacccctcacagcaggaactgccccggctgttgtcggctgcctgcaggcttcagctcaacgggaacctgcagctggagctggcgcaggtgctggcccaggagaggcccaagctgccagaggaccctctgctcagcggcctcctggactccccggcactcaaggcctgcctggacactgccgtggagaacatgcccagcctgaagatgaaggtggtggaggtgctggctggccacggtcacctgtattcccgcatcccaggcctgctcagcccccatcccctgctgcagctgagctacacggccaccgaccgccacccccaggccctggaggctgcccaggccgagctgcagcagcacgacgttgcccagggccagtgggatcccgcagaccctgcccccagcgccctgggcagcgccgacctcctggtgtgcaactgtgctgtggctgccctcggggacccggcctcagctctcagcaacatggtggctgccctgagagaagggggctttctgctcctgcacacactgctccgggggcaccccctcggggacatcgtggccttcctcacctccactgagccgcagtatggccagggcatcctgagccaggacgcgtgggagagcctcttctccagggtgtcgctgcgcctggtgggcctgaagaagtccttctacggctccacgctcttcctgtgccgccggcccaccccgcaggacagccccatcttcctgccggtggacgataccagcttccgctgggtggagtctctgaagggcatcctggctgacgaagactcttcccggcctgtgtggctgaaggccatcaactgtgccacctcgggcgtggtgggcttggtgaactgtctccgccgagagcccggcgggaaccgcctccggtgtgtgctgctctccaacctcagcagcacctcccacgtcccggaggtggacccgggctccgcagaactgcagaaggtgttgcagggagacctggtgatgaacgtctaccgcgacggggcctggggggctttccgccacttcctgctggaggaggacaagcctgaggagccgacggcacatgcctttgtgagcaccctcacccggggggacctgtcctccatccgctgggtctgctcctcgctgcgccatgcccagcccacctgccctggcgcccagctctgcacggtctactacgcctccctcaacttccgcgacatcatgctggccactggcaagctgtcccctgatgccatcccagggaagtggacctcccaggacagcctgctaggtatggagttctcgggccgagacgccagcggcaagcgtgtgatgggactggtgcctgccaagggcctggccacctctgtcctgctgtcaccggacttcctctgggatgtgccttccaactggacgctggaggaggcggcctcggtgcctgtcgtctacagcacggcctactacgcgctggtggtgcgtgggcgggtgcgccccggggagacgctgctcatccactcgggctcgggcggcgtgggccaggccgccatcgccatcgccctcagtctgggctgccgcgtcttcaccaccgtggggtcggctgagaagcgggcgtacctccaggccaggttcccccagctcgacagcaccagcttcgccaactcccgggacacatccttcgagcagcatgtgctgtggcacacgggcgggaagggcgttgacctggtcttgaactccttggcggaagagaagctgcaggccagcgtgaggtgcttggctacgcacggtcgcttcctggaaattggcaaattcgacctttctcagaaccacccgctcggcatggctatcttcctgaagaacgtgacattccacggggtcctactggatgcgttcttcaacgagagcagtgctgactggcgggaggtgtgggcgcttgtgcaggccggcatccgggatggggtggtacggcccctcaagtgcacggtgttccatggggcccaggtggaggacgccttccgctacatggcccaagggaagcacattggcaaagtcgtcgtgcaggtgcttgcggaggagccggaggcagtgctgaagggggccaaacccaagctgatgtcggccatctccaagaccttctgcccggcccacaagagctacatcatcgctggtggtctgggtggcttcggcctggagttggcgcagtggctgatacagcgtggggtgcagaagctcgtgttgacttctcgctccgggatccggacaggctaccaggccaagcaggtccgccggtggaggcgccagggcgtacaggtgcaggtgtccaccagcaacatcagctcactggagggggcccggggcctcattgccgaggcggcgcagcttgggcccgtgggcggcgtcttcaacctggccgtggtcttgagagatggcttgctggagaaccagaccccagagttcttccaggacgtctgcaagcccaagtacagcggcaccctgaacctggacagggtgacccgagaggcgtgccctgagctggactactttgtggtcttctcctctgtgagctgcgggcgtggcaatgcgggacagagcaactacggctttgccaattccgccatggagcgtatctgtgagaaacgccggcacgaaggcctcccaggcctggccgtgcagtggggcgccatcggcgacgtgggcattttggtggagacgatgagcaccaacgacacgatcgtcagtggcacgctgccccagcgcatggcgtcctgcctggaggtgctggacctcttcctgaaccagccccacatggtcctgagcagctttgtgctggctgagaaggctgcggcctatagggacagggacagccagcgggacctggtggaggccgtggcacacatcctgggcatccgcgacttggctgctgtcaacctggacagctcactggcggacctgggcctggactcgctcatgagcgtggaggtgcgccagacgctggagcgtgagctcaacctggtgctgtccgtgcgcgaggtgcggcaactcacgctccggaaactgcaggagctgtcctcaaaggcggatgaggccagcgagctggcatgccccacgcccaaggaggatggtctggcccagcagcagactcagctgaacctgcgctccctgctggtgaacccggagggccccaccctgatgcggctcaactccgtgcagagctcggagcggcccctgttcctggtgcacccaatcgagggctccaccaccgtgttccacagcctggcctcccggctcagcatccccacctatggcctgcagtgcacccgagctgcgccccttgacagcatccacagcctggctgcctactacatcgactgcatcaggcaggtgcagcccgagggcccctaccgcgtggccggctactcctacggggcctgcgtggcctttgaaatgtgctcccagctgcaggcccagcagagcccagcccccacccacaacagcctcttcctgttcgacggctcgcccacctacgtactggcctacacccagagctaccgggcaaagctgaccccaggctgtgaggctgaggctgagacggaggccatatgcttcttcgtgcagcagttcacggacatggagcacaacagggtgctggaggcgctgctgccgctgaagggcctagaggagcgtgtggcagccgccgtggacctgatcatcaagagccaccagggcctggaccgccaggagctgagctttgcggcccggtccttctactacaagctgcgtgccgctgagcagtacacacccaaggccaagtaccatggcaacgtgatgctactgcgcgccaagacgggtggcgcctacggcgaggacctgggcgcggactacaacctctcccaggtatgcgacgggaaagtatccgtccacgtcatcgagggtgaccaccgcacgctgctggagggcagcggcctggagtccatcatcagcatcatccacagctccctggctgagccacgcgtgagcgtgcgggagggctaggcccgtgcccccgcctgccaccggaggtcactccaccatccccaccccaccccaccccacccccgccatgcaacgggattgaagggtcctgccggtgggaccctgtccggcccagtgccactgccccccgaggctgctagatgtaggtgttaggcatgtcccacccacccgccgcctcccacggcacctcggggacaccagagctgccgacttggagactcctggtctgtgaagagccggtggtgcccgtgcccgcaggaactgggctgggcctcgtgcgcccgtggggtctgcgcttggtctttctgtgcttggatttgcatatttattgcattgctggtagagacccccaggcctgtccaccctgccaagactcctcaggcagcgtgtgggtcccgcactctgcccccatttccccgatgtcccctgcgggcgcgggcagccacccaagcctgctggctgcggccccctctcggccaggcattggctcagcccgctgagtggggggtcgtgggccagtccccgaggagctgggcccctgcacaggcacacagggcccggccacacccagcggccccccgcacagccacccgtggggtgctgcccttatgcccggcgccgggcaccaactccatgtttggtgtttgtctgtgtttgtttttcaagaaatgattcaaattgctgcttggattttgaaatttactgtaactgtcagtgtacacgtctggaccccgtttcatttttacaccaatttggtaaaaatgctgctctcagcctcccacaattaaaccgcatgtgatctccaaaaaaaaaaaaaaaaaaaaaaaa

SEQ ID NO: 79 - DNA, 985 bp, Homo sapiens
  60S ribosomal protein L17 transcript variant 1, L17, ribosomal protein L23, RPL23, gene encoding putative NFkB activating protein, PD-1
cctgcctcctcagatctcgtttcttcggctacgaatctcgcgagaagtcaagttctcatgagttctcccaaaatccaccgctcttcctctttccctaagcagcctgagggttgactggattggtgaggcccgtgtggctacttctgtggaagcagtgctgtagttactggaagataaaagggaaagcaagcccttggtgggggaaagtatggctgcgatgatggcatttcttaggacacctttggattaataatgaaaacaactactctctgagcagctgttcgaatcatctgatatttatactgaatgagttactgtaagtacgtattgacagaattacactgtactttcctctaggtgatctgtgaaaatggttcgctattcacttgacccggagaaccccacgaaatcatgcaaatcaagaggttccaatcttcgtgttcactttaagaacactcgtgaaactgctcaggccatcaagggtatgcatatacgaaaagccacgaagtatctgaaagatgtcactttacagaaacagtgtgtaccattccgacgttacaatggtggagttggcaggtgtgcgcaggccaagcaatggggctggacacaaggtcggtggcccaaaaagagtgctgaatttttgctgcacatgcttaaaaacgcagagagtaatgctgaacttaagggtttagatgtagattctctggtcattgagcatatccaagtgaacaaagcacctaagatgcgccgccggacctacagagctcatggtcggattaacccatacatgagctctccctgccacattgagatgatccttacggaaaaggaacagattgttcctaaaccagaagaggaggttgcccagaagaaaaagatatcccagaagaaactgaagaaacaaaaacttatggcacgggagtaaattcagcattaaaataaatgtaattaaaaggaaaagaaaaaaaaaaaaaaaaaaaaaaaa

SEQ ID NO: 80 - DNA, 3486 bp, Homo sapiens
  eukaryotic translation initiation factor 2, subunit 3 gamma, 52kDa (EIF2S3, eIF-2gA, EIF2gamma), eukaryotic translation initiation factor 2G (EIF2G), eukaryotic translation initiation factor 2 subunit gamma X (eIF-2gX, eIF-2-gamma X)
tttccttcctcttttggcaacatggcgggcggagaagctggagtgactctagggcagccgcatctttcgcgtcaggatctcaccaccttggatgttaccaagttgacgccactttcacacgaagttatcagcagacaagccacaattaacataggtacaattggtcatgtagctcatgggaaatccacagtcgtcaaagctatttctggagttcatactgtcaggttcaaaaatgaactagaaagaaatattacaatcaagcttggatatgctaatgctaagatttataagcttgatgacccaagttgccctcggccagaatgttatagatcttgtgggagcagtacacctgacgagtttcctacggacattccagggaccaaagggaacttcaaattagtcagacatgtttcctttgttgactgtcctggccacgatattttgatggctactatgctgaacggtgcagcagtgatggatgcagctcttctgttgatagctggtaatgaatcttgccctcagcctcagacatcggaacacctggctgctatagagatcatgaaactgaagcatattttgattctacaaaataaaattgatttggtaaaagaaagtcaggctaaagaacaatacgagcagatccttgcatttgtccaaggtacagtagcagagggagctcccattattccaatttcagctcagctgaaatacaatattgaagttgtttgtgagtacatagtaaagaaaattccagtacccccaagagactttacttcagagccccggcttattgttattagatcttttgatgtcaacaaacctggctgtgaagttgatgaccttaagggaggtgtagctggtggtagtatcctaaaaggagtattaaaggtgggccaggagatagaagtaagacctggtattgtttccaaagatagtgaaggaaaactcatgtgtaaaccaatcttttccaaaattgtatcactttttgcggagcataatgatctgcaatatgctgctccaggcggtcttattggagttggaacaaaaattgaccccactttgtgccgggctgacagaatggtggggcaagtacttggtgcagtcggagctttacctgagatattcacagaattggaaatttcctatttcctgcttagacggcttctaggtgtacgcactgaaggagacaagaaagcagcaaaggttcaaaagctgtctaagaatgaagtgctcatggtgaacataggatccctgtcaacaggagggagagttagtgctgtcaaggccgatttgggtaaaattgttttgaccaatccagtgtgcacagaggtaggagaaaaaattgcccttagccgaagagttgaaaaacactggcgtttaattggttggggtcagataagaagaggagtgacaatcaagccaacagtagatgatgactgaagaataccagttaaataatacattcggatggatttggaagttggaattcctcttaacaaccaaggggtttattttcaaagcaatattggggaattgatttcacagttcgttaccttagtaggtaacggtaaggttattctctttttttttttttttttttttggttatgaaaacttagggactaaaattaatataaaaattggcataatgttggattgaatctacattttggcagaagttaaacattcccacataatgtcaaaattatacatcatgcagttctgtttttttgtttgttttattttgttttgtttttgagtctggctctgtcacccaggctggagtgcagtggcgtgatctgcaacctctgccccccgggttcaagcgattctcctgcctcagcctcccgagtagctgagattacaggtgcgcgccaccacacttggctaatttttgtattattagtagagacggggtttcagcatgttggctaggccggtctctcctgacctcagggtgatcagcccacctcggcctcacaaagtgctgggattacaggcgtgagccaccttgcccagcccacatcatacagtttgaaatgaaactttgccacaaccagcctttgctgtagcacacacatatatcactgaacctgtttgaaataaagttttttttctttttcatgattcgtctttgagtacctccaggctgaaagactgttgtaccagtaaaaacttaaaggcacaaattctccttgaagaccttctcccttttatgtggccccatattttatgttgctttatctttgaaattttgcatgaaaaggaaatgaatggattcgaatgaaattgtcctttagagcatgattacttgttcccatggacaaatatttttctccccttgctcttcctggcctgaaacacgggaaaccagagtcaaaagttatctccctctccctgtgatgccttgagatttttttctgcgttgtttaatgcctgaaatccaagtcttcctccatgggaaaatactgttataccaaataattctagatgagtaacaaagatctttttaggccttcattttatgttttttcttaactgttatattatgattgtgacatagattatactactactaatttttggatgtttcaaaaggtcaagaagtaaaagatgttagaaagcaatgagtgagtccttttgatttttaacttattccccatgtccctatacttcgtgtgcttttcctttttttttttgagacggaggctcactccgtcacctaggctagagtacagtggcacgatcttggctcgctgcaacctctgcctccctgattcaagtgattctcgtgcttcagcctcccatgtagctgatattacaggcacttgccaccatacccggctaatttttgtatttttagtagagatggggtttcaccatgttgcccaggctggtcttgaactcctaacctcaggtgatccgcccgcctctgccttccaaagtgctgggattacaggcgtgagccactctgcccggcttatttttctttatgtttttgcttcgtaagaggttctgttgagcagtgatttgcaactcttgctgacgttgctggggaagctttaaaaaaaaaaaagatgccccacagagattctgattttaattgttctgatttaattggcttggagtagaattcaggcattgatatctttaaaaactccccagtgttgagaaacaaatttagagagttgagaagtaggtatattaaattacagaatcttactgagttttggtagactgataatacaatttgctttgcttttcttaaatttgcattgagatgggatttgaagcatattgtgctcttgtgaatgttgaagttgcattgtagaagtttagaagctctggctatgggttgcctaaattgatgttttgaggaagcatattaatgttataaacttcgctgactttgaaggttgtgttgtagcatgaggaacacaaataaaacaattctaaatcaaactaaaaaaaaaaaaaaaaaaaaaaa

SEQ ID NO: 81 - DNA, 571 bp, Homo sapiens
  60S ribosomal protein L30, L30
agttccggctctgccgtgaagagctttgcattgtgggaagtctttcctttctcgttccccggccatcttagcggctgctgttggttgggggccgtcccgctcctaaggcaggaagatggtggccgcaaagaagacgaaaaagtcgctggagtcgatcaactctaggctccaactcgttatgaaaagtgggaagtacgtcctggggtacaagcagactctgaagatgatcagacaaggcaaagcgaaattggtcattctcgctaacaactgcccagctttgaggaaatctgaaatagagtactatgctatgttggctaaaactggtgtccatcactacagtggcaataatattgaactgggcacagcatgcggaaaatactacagagtgtgcacactggctatcattgatccaggtgactctgacatcattagaagcatgccagaacagactggtgaaaagtaaaccttttcacctacaaaatttcacctgcaaaccttaaacctgcaaaattttcctttaataaaatttgcttgttttaaaaacattgtatctaaaaaaaaaaaaaaaaaa

SEQ ID NO: 82 - DNA, 2004 bp, Homo sapiens
  actin, gamma 1, transcript variant 2 (ACTG1, ACT, ACTG), cytoskeletal gamma-actin, deafness, autosomal dominant 20 (DFNA20), deafness, autosomal dominant 26 (DFNA26), BRWS2
gcgcgccgccgggccgcgcgggcgcgccgcttccgcttaaataacggcgggggaggccgcggtcggtctcagtcgccgctgccagctctcgcactctgttcttccgccgctccgccgtcgcgtttctctgccggtcgcaatggaagaagagatcgccgcgctggtcattgacaatggctccggcatgtgcaaagctggttttgctggggacgacgctccccgagccgtgtttccttccatcgtcgggcgccccagacaccagggcgtcatggtgggcatgggccagaaggactcctacgtgggcgacgaggcccagagcaagcgtggcatcctgaccctgaagtaccccattgagcatggcatcgtcaccaactgggacgacatggagaagatctggcaccacaccttctacaacgagctgcgcgtggccccggaggagcacccagtgctgctgaccgaggcccccctgaaccccaaggccaacagagagaagatgactcagattatgtttgagaccttcaacaccccggccatgtacgtggccatccaggccgtgctgtccctctacgcctctgggcgcaccactggcattgtcatggactctggagacggggtcacccacacggtgcccatctacgagggctacgccctcccccacgccatcctgcgtctggacctggctggccgggacctgaccgactacctcatgaagatcctcactgagcgaggctacagcttcaccaccacggccgagcgggaaatcgtgcgcgacatcaaggagaagctgtgctacgtcgccctggacttcgagcaggagatggccaccgccgcatcctcctcttctctggagaagagctacgagctgcccgatggccaggtcatcaccattggcaatgagcggttccggtgtccggaggcgctgttccagccttccttcctgggtatggaatcttgcggcatccacgagaccaccttcaactccatcatgaagtgtgacgtggacatccgcaaagacctgtacgccaacacggtgctgtcgggcggcaccaccatgtacccgggcattgccgacaggatgcagaaggagatcaccgccctggcgcccagcaccatgaagatcaagatcatcgcacccccagagcgcaagtactcggtgtggatcggtggctccatcctggcctcactgtccaccttccagcagatgtggattagcaagcaggagtacgacgagtcgggcccctccatcgtccaccgcaaatgcttctaaacggactcagcagatgcgtagcatttgctgcatgggttaattgagaatagaaatttgcccctggcaaatgcacacacctcatgctagcctcacgaaactggaataagccttcgaaaagaaattgtccttgaagcttgtatctgatatcagcactggattgtagaacttgttgctgattttgaccttgtattgaagttaactgttccccttggtatttgtttaataccctgtacatatctttgagttcaacctttagtacgtgtggcttggtcacttcgtggctaaggtaagaacgtgcttgtggaagacaagtctgtggcttggtgagtctgtgtggccagcagcctctgatctgtgcagggtattaacgtgtcagggctgagtgttctgggatttctctagaggctggcaagaaccagttgttttgtcttgcgggtctgtcagggttggaaagtccaagccgtaggacccagtttcctttcttagctgatgtctttggccagaacaccgtgggctgttacttgctttgagttggaagcggtttgcatttacgcctgtaaatgtattcattcttaatttatgtaaggttttttttgtacgcaattctcgattctttgaagagatgacaacaaattttggttttctactgttatgtgagaacattaggccccagcaacacgtcattgtgtaaggaaaaataaaagtgctgccgtaaccaaaaaaaaaaaaaaaaaaaa

SEQ ID NO: 83 - DNA, 3455 bp, Homo sapiens
  collagen, type VI, alpha 2 transcript variant 2C2 (COL6A2), collagen VI, alpha-2 polypeptide, collagen alpha-2(VI) chain, collagen VI alpha-2 C-terminal globular domain, PP3610
gcttactcggcgcccgcgcctcgggccgtcgggagcggagcctcctcgggaccaggacttcagggccacaggtgctgccaagatgctccagggcacctgctccgtgctcctgctctggggaatcctgggggccatccaggcccagcagcaggaggtcatctcgccggacactaccgagagaaacaacaactgcccagagaagaccgactgccccatccacgtgtacttcgtgctggacacctcggagagcgtcaccatgcagtcccccacggacatcctgctcttccacatgaagcagttcgtgccgcagttcatcagccagctgcagaacgagttctacctggaccaggtggcgctgagctggcgctacggcggcctgcacttctctgaccaggtggaggtgttcagcccaccgggcagcgaccgggcctccttcatcaagaacctgcagggcatcagctccttccgccgcggcaccttcaccgactgcgcgctggccaacatgacggagcagatccggcaggaccgcagcaagggcaccgtccacttcgccgtggtcatcaccgacggccacgtcaccggcagcccctgcgggggcatcaagctgcaggccgagcgggcccgcgaggagggcatccggctcttcgccgtggcccccaaccagaacctgaaggagcagggcctgcgggacatcgccagcacgccgcacgagctctaccgcaacgactacgccaccatgctgcccgactccaccgagatcgaccaggacaccatcaaccgcatcatcaaggtcatgaaacacgaagcctacggagagtgctacaaggtgagctgcctggaaatccctgggccctctggccccaagggctaccgtggacagaagggtgccaagggcaacatgggtgagccgggagagcctggccagaagggaagacagggagacccgggcatcgaaggccccattggattcccaggacccaagggcgttcctggcttcaaaggagagaagggtgaatttggagccgacggtcgcaagggggcccctggcctggctggcaagaacgggaccgatggacagaagggcaagctggggcgcatcggacctcctggctgcaagggagaccctggaaaccggggccccgacggttacccgggggaagcagggagtccaggggagcgaggagaccaaggcggcaagggggaccctggccgcccaggacgcagagggcccccgggagaaatcggggccaagggaagcaaggggtatcaaggcaacagtggagccccaggaagtcctggtgtgaaaggagccaagggcgggcctgggccccgcggacccaaaggcgagccggggcgcaggggagaccccggcaccaagggcagcccaggcagcgatggccccaagggggagaagggggaccctggccctgaggggccccgcggcctggctggagaggttggcaacaaaggagccaagggagaccgaggcttgcctggacccagaggcccccagggagctcttggggagcccggaaagcagggatctcggggagaccccggtgatgcaggaccccgtggagactcaggacagccaggccccaagggagaccccggcaggcctggattcagctacccaggaccccgaggagcacccggagaaaaaggcgagcccggcccacgcggccccgagggaggccgaggcgactttggcttgaaaggagaacctgggaggaaaggagagaaaggagagcctgcggatcctggtccccctggtgagccaggccctcgggggccaagaggagtcccaggacccgagggtgagcccggcccccctggagaccccggtctcacggagtgtgacgtcatgacctacgtgagggagacctgcgggtgctgcgactgtgagaagcgctgtggcgccctggacgtggtcttcgtcatcgacagctccgagagcattgggtacaccaacttcacactggagaagaacttcgtcatcaacgtggtcaacaggctgggtgccatcgctaaggaccccaagtccgagacagggacgcgtgtgggcgtggtgcagtacagccacgagggcacctttgaggccatccagctggacgacgaacgtatcgactccctgtcgagcttcaaggaggctgtcaagaacctcgagtggattgcgggcggcacctggacaccctcagccctcaagtttgcctacgaccgcctcatcaaggagagccggcgccagaagacacgtgtgtttgcggtggtcatcacggacgggcgccacgaccctcgggacgatgacctcaacttgcgggcgctgtgcgaccgcgacgtcacagtgacggccatcggcatcggggacatgttccacgagaagcacgagagtgaaaacctctactccatcgcctgcgacaagccacagcaggtgcgcaacatgacgctgttctccgacctggtcgctgagaagttcatcgatgacatggaggacgtcctctgcccggaccctcagatcgtgtgcccagaccttccctgccaaacagagctgtccgtggcacagtgcacgcagcggcccgtggacatcgtcttcctgctggacggctccgagcggctgggtgagcagaacttccacaaggcccggcgcttcgtggagcaggtggcgcggcggctgacgctggcccggagggacgacgaccctctcaacgcacgcgtggcgctgctgcagtttggtggccccggcgagcagcaggtggccttcccgctgagccacaacctcacggccatccacgaggcgctggagaccacacaatacctgaactccttctcgcacgtgggcgcaggcgtggtgcacgccatcaatgccatcgtgcgcagcccgcgtggcggggcccggaggcacgcagagctgtccttcgtgttcctcacggacggcgtcacgggcaacgacagtctgcacgagtcggcgcactccatgcgcaagcagaacgtggtacccaccgtgctggccttgggcagcgacgtggacatggacgtgctcaccacgctcagcctgggtgaccgcgccgccgtgttccacgagaaggactatgacagcctggcgcaacccggcttcttcgaccgcttcatccgctggatctgctagcgccgccgcccgggccccgcagtcgagggtcgtgagcccaccccgtccatggtgctaagcgggcccgggtcccacacggccagcaccgctgctcactcggacgacgccctgggcctgcacctctccagctcctcccacggggtccccgtagccccggcccccgcccagccccaggtctccccaggccctccgcaggctgcccggcctccctccccctgcagccatcccaaggctcctgacctacctggcccctgagctctggagcaagccctgacccaataaaggctttgaacccataaaaaaaaaaaaaaaa

SEQ ID NO: 84 - DNA, 2801 bp, Homo sapiens
  ubiquitin-60S ribosomal protein L40 precursor transcript variant 2 (UBA52), RPL40, ubiquitin-52 amino acid fusion protein, ubiquitin carboxyl extension protein 52 (CEP52, HUBCEP52)
gctccgtgcgcaagcgctttcggcggcgattaggtggtttccggttccgctatcttctttttcttcagcgaggcggccgagctgacgcaaacatgcagatctttgtgaagaccctcactggcaaaaccatcacccttgaggtcgagcccagtgacaccattgagaatgtcaaagccaaaattcaagacaaggagggtatcccacctgaccagcagcgtctgatatttgccggcaaacagctggaggatggccgcactctctcagactacaacatccagaaagagtccaccctgcacctggtgttgcgcctgcgaggtggcattattgagccttctctccgccagcttgcccagaaatacaactgcgacaagatgatctgccgcaagtgctatgctcgccttcaccctcgtgctgtcaactgccgcaagaagaagtgtggtcacaccaacaacctgcgtcccaagaagaaggtcaaataaggtggttctttccttgaagggcagcctcctgcccaggccccgtggccctggagcctcaataaagtgtccctttcattgactggagcagcaattggtgtcctcatggctgatctgtccagggaggtggctgaagagtgggcatctcccttagggactctactcagcactccattctgtgccacctgtggggtcttctgtcctagattctgtcacatcggcattggtccctgccctatgcccctgactctggatttgtcatctgtaaaactggagtaaaaacctcagtcgtgtaattggtgggactgaggatcagttttgtcattgctgggatcctgtcaggcactttgaggtgtccctcaggccttggccctgaagtgtctaggtgtgtggagatgggtagaaaattaggtacacccaatggtgtagaacgttgattctcaaatttttttattttatacaaatggggtctcactatgttgtccaggctggtcttgaactcctgggctcaagccatccgcccatctcagcccctcaaagtgttgggattacaagcaagaactgccatgcctgacccagttctcagttttttgtttgtttgtttgtttgtttgttttgagacggagtcttgctctgtcgcccaggctggagtgcagtggcgcagtctcggcttactacaacctctgcctccggggttcacatccttctcctgcctcagcctcccgagtagctgggactacaggtgcccgccacaactcctggctaattttttgtatttttagtagagacggggtttcactgggttagccaggttggtctcgatctcctgaccttgtgatccattcgccttggcctcccagaatgctggtattacaggcgtgagccagcacgcctggcccagttactcagttttgaatctgaggccgtgacatcactcatggtctgcagtcagtgctctgcccctgagctgtaccctctcctatgataatcactcttaagaagggcaacccttggtgttttccccttaaggtcacccaggctggaatgcagtggtgtggtcatggctccctgtaccctggaactcaggcttgggtgatcctctctcctttgcctccgaagtagccaggactacaggtgtgcacccaccaccacactcagataattgctttggtgtttttaaagcttgtaatgatcagtaggctgaggtgggcaaatcataaggtcaagagttttttagatggggtgagcacagaccaattcctgttttatttactgatttaaaattttgagacagtctcactgtcacccaggttggggtgcagtggtaggatcatagcttgctgcagccttgatctcccaggatcttgcctcagcctcccgagtagctgggactgcatgcttgtgccaccacactcggttaatattttgtagagatggggtcttgctatgttgcccaggctggcttcaaactcctgaacttaaaagcctcctgtttagttttggttttttatcactttttttttttttttttgagatggagccttgctcccatcgtgcaggctggagtgcggtggcgcagtctcggctcactgcagcttctgcctctcgggttcaagcgattctcctttctcagcctcttgagtagctggaattaccagtgtgcgccaccaccaccacgcctggctagtttttctgtttttagtagagacagggttttgctatgttggccaggctggtcttgaactactgacctcttgtgatctacctgtcttggccttccaaagtgctaggattacaagcgtaagccacagcgcctggccttgctacatttttttttttttttttttttttacagacatggtctcgctatgttgcccagaatggttttgcactgggtccaagcagttctgccgcagcctcccaaagtgctgggattacaggggtgaggcaccttgctggcccctgttttgattagggtgcagtgctggtgaagccggtgcacgaggccagtgatgcatcctaatgaggggtggagttggcgggacttcctgggccagtttggggactttcacaaaagacccccatgactcagggttttgagttcttaactgatcgaatgaaggattcaaaattaaccactccaaggggggattgaaggaagaaccactcttaatggacaaaaagaaagaaaggggagggagtaacagggatatgagctctagccgcccaagctagcaatggcaacccttctgggtccccttccagcatgtggaagctttcctttcgcttcattcaataaacagctgctgctcaaaaaaaaaaaaa

SEQ ID NO: 85 - DNA, 1331 bp, Homo sapiens
  alkB, alkylation repair homolog 7 (E. coli), alkylated DNA repair protein alkB homolog 7, alpha-ketoglutarate- dependent dioxygenase alkB homolog 7, mitochondrial (ALKBH7), spermatogenesis associated 11 (SPATA11), spermatogenesis cell proliferation-related protein
gactccataaccgtggccttggccccagtccccctgacttccggacttcagaccagatactgcccatatccccttatgaagtcttggccaggcaacccctagggtgtacgttttctaatgattaaagaggcggtgctaagctgcagacggacttgcgactcagccactggtgtaagtcaggcgggaggtggcgcccaataagctcaagagaggaggcgggttctggaaaaaggccaatagcctgtgaaggcgagtctagcagcaaccaatagctatgagcgagaggcgggactctgagggaagtcaatcgctgccgcaggtaccgccaatggcttttggcgggggcgttccccaaccctgccctctctcatgaccccgctccgggattatggccgggactgggctgctggcgctgcggacgctgccagggcccagctgggtgcgaggctcgggcccttccgtgctgagccgcctgcaggacgcggccgtggtgcggcctggcttcctgagcacggcagaggaggagacgctgagccgagaactggagcccgagctgcgccgccgccgctacgaatacgatcactgggacgcggccatccacggcttccgagagacagagaagtcgcgctggtcagaagccagccgggccatcctgcagcgcgtgcaggcggccgcctttggccccggccagaccctgctctcctccgtgcacgtgctggacctggaagcccgcggctacatcaagccccacgtggacagcatcaagttctgcggggccaccatcgccggcctgtctctcctgtctcccagcgttatgcggctggtgcacacccaggagccgggggagtggctggaactcttgctggagccgggctccctctacatccttaggggctcagcccgttatgacttctcccatgagatccttcgggatgaagagtccttctttggggaacgccggattccccggggccggcgcatctccgtgatctgccgctccctccctgagggcatggggccaggggagtctggacagccgcccccagcctgctgacccccagctttctacagacaccagatttgtgaataaagttggggaatggacagcctaactgggacattgcagtggctgcttgctggggccgggatttgcaggggaacccaggatggcactggcccatagggagctccaggtgtggctggctggacacatggtcaaagtcacaaggccgggagagtggtgtcctttattgcactcactgctggtcgccccagcccactcccctcctcgttgtctctgcatccaggtctccaataaataagtcagccga

SEQ ID NO: 86 - DNA, 1035 bp, Homo sapiens
  60S ribosomal protein L5, L5, DBA6, MSTP030
ggcccttttcccaccccctagcgccgctgggcctgcaggtctctgtcgagcagcggacgccggtctctgttccgcaggatggggtttgttaaagttgttaagaataaggcctactttaagagataccaagtgaaatttagaagacgacgagagggtaaaactgattattatgctcggaaacgcttggtgatacaagataaaaataaatacaacacacccaaatacaggatgatagttcgtgtgacaaacagagatatcatttgtcagattgcttatgcccgtatagagggggatatgatagtctgcgcagcgtatgcacacgaactgccaaaatatggtgtgaaggttggcctgacaaattatgctgcagcatattgtactggcctgctgctggcccgcaggcttctcaataggtttggcatggacaagatctatgaaggccaagtggaggtgactggtgatgaatacaatgtggaaagcattgatggtcagccaggtgccttcacctgctatttggatgcaggccttgccagaactaccactggcaataaagtttttggtgccctgaagggagctgtggatggaggcttgtctatccctcacagtaccaaacgattccctggttatgattctgaaagcaaggaatttaatgcagaagtacatcggaagcacatcatgggccagaatgttgcagattacatgcgctacttaatggaagaagatgaagatgcttacaagaaacagttctctcaatacataaagaacagcgtaactccagacatgatggaggagatgtataagaaagctcatgctgctatacgagagaatccagtctatgaaaagaagcccaagaaagaagttaaaaagaagaggtggaaccgtcccaaaatgtcccttgctcagaagaaggatcgggtagctcaaaagaaggcaagcttcctcagagctcaggagcgggctgctgagagctaaacccagcaattttctatgattttttcagatatagataataaacttatgaacagcaactaaaaa

SEQ ID NO: 87 - DNA, 1043 bp, Homo sapiens
  6-phosphogluconolactonase (PGLS, 6PGL)
ctagcgacggccgtagggagcgcttcctcctccccgccgccgccctcgccatggccgcgccggccccgggcctcatctcggtgttctcgagttcccaggagctgggtgcggcgctagcgcagctggtggcccagcgcgcagcatgctgcctggcaggggcccgcgcccgtttcgcgctcggcctgtcgggcgggagcctcgtctcgatgctagcccgcgagctacccgccgccgtcgcccctgccgggccagctagcttagcgcgctggacgctgggcttctgcgacgagcgcctcgtgcccttcgatcacgccgagagcacgtacggcctctaccggacgcatcttctctccagactgccgatcccagaaagccaggtgatcaccattaaccccgagctgcctgtggaggaggcggctgaggactacgccaagaagctgagacaggcattccaaggggactccatcccggttttcgacctgctgatcctgggggtgggccccgatggtcacacctgctcactcttcccagaccaccccctcctacaggagcgggagaagattgtggctcccatcagtgactccccgaagccaccgccacagcgtgtgaccctcacactacctgtcctgaatgcagcacgaactgtcatctttgtggcaactggagaaggcaaggcagctgttctgaagcgcattttggaggaccaggaggaaaacccgctgcccgccgccctggtccagccccacaccgggaaactgtgctggttcttggacgaggcggccgcccgcctcctgaccgtgcccttcgagaagcattccactttgtagctggccagagggacgccgcagctgggaccaggcacgcggcccatggggctgggcccctgctggccgccactctccgggctctcctttcaaaaagccacgtcgtgctgctgctggaagccaacagcctccggccagcagccctacccggggctcaacacacaggctgtggctctggacatccggatattaaaaggagcgttgctggaaaaaaaaaaa

SEQ ID NO: 88 - DNA, 1976 bp, Homo sapiens
  cold shock domain protein A transcript variant 1 (CSDA),cold-shock domain containing A1 (CSDA1), Y box binding protein 3 (YBX3), single-strand DNA-binding protein NF-GMB, DNA-binding protein A (DBPA), ZO-1-associated nucleic acid-binding protein (ZONAB)
agaaaaacttgtgcggggccatttttggggcagtaagatcgagcgaggagcccaagagcgagcgcgcagcacgaagctcgagccgcctccgccgcgcgaccccacctcggccgccgccgcctgcgccgcgagatccgccccggcctccccgagagcgagccccggccgccgcgaccaccagccgcgctaaccgccgaccaaccgccaccgaggcgcctgagcgagagcagaggaggaggaggcatgagtgaggcgggcgaggccaccaccaccaccaccaccaccctcccgcaggctccgacggaggcggccgccgcggctccccaggaccccgcgcccaagagcccggtgggcagcggtgcgccccaggccgcggccccggcgcccgccgcccacgtcgcaggaaaccccggtggggacgcggcccccgcagccacgggcaccgcggccgccgcctctttagccaccgccgccggcagcgaagacgcggagaaaaaagttctcgccaccaaagtccttggcactgtcaaatggttcaacgtcagaaatggatatggatttataaatcgaaatgacaccaaagaagatgtatttgtacatcagactgccatcaagaagaataacccacggaaatatctgcgcagtgtaggagatggagaaactgtagagtttgatgtggttgaaggagagaagggtgcagaagctgccaatgtgactggcccggatggagttcctgtggaagggagtcgttacgctgcagatcggcgccgttacagacgtggctactatggaaggcgccgtggccctccccggaattacgctggggaggaggaggaggaagggagcggcagcagtgaaggatttgacccccctgccactgataggcagttctctggggcccggaatcagctgcgccgcccccagtatcgccctcagtaccggcagcggcggttcccgccttaccacgtgggacagacctttgaccgtcgctcacgggtcttaccccatcccaacagaatacaggctggtgagattggagagatgaaggatggagtcccagagggagcacaacttcagggaccggttcatcgaaatccaacttaccgcccaaggtaccgtagcaggggacctcctcgcccacgacctgccccagcagttggagaggctgaagataaagaaaatcagcaagccaccagtggtccaaaccagccgtctgttcgccgtggataccggcgtccctacaattaccggcgtcgcccgcgtcctcctaacgctccttcacaagatggcaaagaggccaaggcaggtgaagcaccaactgagaaccctgctccacccacccagcagagcagtgctgagtaacaccaggctcctcaggcaccttcaccatcggcaggtgacctaaagaattaatgaccattcagaaataaagcaaaaagcaggccacaaccttaaccaacaccaaagaaacatccaagcaataaagtggaagactaaccaagatttggacattggaatgtttactgttattctttaagaaacaactacaaaaagaaaatgtcaacaaatttttccagcaagctgagaacctgggaattcctgcacggaagacaagagagtagcctctccagtttcagcaaccgctaggtttctattttttttcctggtttttactgttttggtaatatatatattgaaacaagaaatattaataccacatggggagaaccccaaccaaagaaatctgaaatatatagtaaatgcttttttttccgtttttgttcattttggatgctggtgctaaacctccaagtgtcatgatttaaaaaaaaaaaaaatttatgtccttcttatttatttctaggatgaggggaggataacatttttgctttcttatgtgactctctttgaaaatgtgcagtaagaaattcctcaaaaataaaatttttacccttcagaggacagaatgtttaaaa

SEQ ID NO: 89 - DNA, 972 bp, Homo sapiens
  60S ribosomal protein L6, L6, neoplasm-related protein C140, tax-responsive enhancer element-binding protein 107 (TAXREB107, TXREB1), DNA-binding protein TAXREB107, SHUJUN-2
gattgcttatagaccggaagccgggaccttaattctctttcccatcttgcaagatggcgggtgaaaaagttgagaagccagatactaaagagaagaaacccgaagccaagaaggttgatgctggtggcaaggtgaaaaagggtaacctcaaagctaaaaagcccaagaaggggaagccccattgcagccgcaaccctgtccttgtcagaggaattggcaggtattcccgatctgccatgtattccagaaaggccatgtacaagaggaagtactcagccgctaaatccaaggttgaaaagaaaaagaaggagaaggttctcgcaactgttacaaaaccagttggtggtgacaagaacggcggtacccgggtggttaaacttcgcaaaatgcctagatattatcctactgaagatgtgcctcgaaagctgttgagccacggcaaaaaacccttcagtcagcacgtgagaaaactgcgagccagcattacccccgggaccattctgatcatcctcactggacgccacaggggcaagagggtggttttcctgaagcagctggctagtggcttattacttgtgactggacctctggtcctcaatcgagttcctctacgaagaacacaccagaaatttgtcattgccacttcaaccaaaatcgatatcagcaatgtaaaaatcccaaaacatcttactgatgcttacttcaagaagaagaagctgcggaagcccagacaccaggaaggtgagatcttcgacacagaaaaagagaaatatgagattacggagcagcgcaagattgatcagaaagctgtggactcacaaattttaccaaaaatcaaagctattcctcagctccagggctacctgcgatctgtgtttgctctgacgaatggaatttatcctcacaaattggtgttctaaatgtcttaagaacctaattaaatagctgactacaaaaaaaaaaaaaaaaaaa

SEQ ID NO: 90 - DNA, 2820 bp, Homo sapiens
  40S ribosomal protein S24 transcript variant d, S24, DBA3
aggcatcggcgcggtcagcctcgtggcgcgcccacgcccccacgccggctcttcccggggtccttccgtgcgcgttgatatgattggccggcgaatcgtggttctcttttcctccttggctgtctgaagatagatcgccatcatgaacgacaccgtaactatccgcactagaaagttcatgaccaaccgactacttcagaggaaacaaatggtcattgatgtccttcaccccgggaaggcgacagtgcctaagacagaaattcgggaaaaactagccaaaatgtacaagaccacaccggatgtcatctttgtatttggattcagaactcattttggtggtggcaagacaactggctttggcatgatttatgattccctggattatgcaaagaaaaatgaacccaaacatagacttgcaagacatggcctgtatgagaagaaaaagacctcaagaaagcaacgaaaggaacgcaagaacagaatgaagaaagtcagggggactgcaaaggccaatgttggtgctggcaaaaagatgagggaattggggcttggagtgcaagcattgggaagaatttcccaggaagagagatgcacagatgtgaagaactcgaaggcaagagaaagccggggggttgtgtggcaggtagaagtgccaggaccgtggagcgtgtggacatgtggcagattgcggaggggctgtggcaagtatttacaggtggccgttacctggaggaagactgaaaacagagaacagtgctgccaggcgtgccttttggagagggcactggtcagaaacggagccttcatgtcgcctgcctcacctgctcctgctggttctccccaccctgtggacggtgacttggtcctccacttgccagaagccttgtcagcaaccttgactctgtcaccccacatccaggccatcaacaagtcttttggccctttctttgaaatccaccaggaatccagctgcttctccccaccttcctgcctctctggtctgggccactaatgtcactgccatggccgccttgctgcatttctgaggatgcttcatctctccaccttcttctccactcagcagccagcagggcactgtggaaatcggagtcacatgagctggcacctctgttcagaaccctccagggctccacatctctctcacccaaatgccaaagacctccccacgcccccacaatcccccacgacctggccactggcctcccaccaccttccagctccagcggctcctaccacatttaaggctttccttcctagttttaatttttcctcgtcagcagttgattttattattttcttgtttattggtattttcccactagaaatgaagctgcgtgaagttagagatttttttttttggtctgtgttcctaattagctcattgctatacccctggcgcccagaacaatgccttggacacagtacgcagtagactaaataaatacttgttgaatgactgactgacggaatgacggctgtgtggggagtggattgggtcgtgaggcagaggctgcggtggaaactcaggcaggaggtgatggtggttcttggggctgcggaatgccaagtttagaagctcttcctctgctgtggcacatgaaccggtcactcgagaaggcttttagatttactttgcctaatcccctcttagtgcatgtggggaaactgaggtacacaaaaggaattccccaccaagttaggggcagaacctagcccccttgtctcccagatggatatcttcttttttttttgagacggagtcttgctctgttgcccaggctggagtgcagtggtaccatcttggctcactgcaacctctgcttcccaggttcaagcgattctcctgcctcagcctcctgagtgtctgcgattacaggtgcacacaaccacgcctggctaatttttgtatttttagtagagacggggtttcaccgtgttggtcagggtgacctcaaactcctgacctcatgatccacccagctcagcctcccaacgtgctgggattacaggcatgagccaccgtgcctggctggacatcttgttattaaagcttcttctctctttgtaggggagggggagatgcctctggtggagaagaccagtgtggcagtgactgtgtctgttagtgaacctggtggctggttgagggtctgtcgtggtgactgaggacacatacaaagtgcttttctcagtggtcaccttggtgttggtgaataagggtcagaagatggctcctgtcctagggcactgccagtcggtttggaagctgaaatgcctgcttagcagtttgaggaaacacagaccttggaggatcttctggttgcctcttcaagaattcattctattccccttctgctccccaaatttgcttttcttggggtgggtcttggttggcctaagccaagaaagtatggcatctactccttccatagcaatagctcaggaataggcagtgacccagacctgaaccaatcagtgcatggaattacccctggccaaagtggttgattgaggctgggtgcaagcagagttgtgagaaggctcccatttggtggttggagagatcgcacttgctccagaggtcataatgtgcagatctgaggcttggaactgctgcagacattttgctaccacaagtgaagccaccctgacgacacagttgacaatttggagcagggcagagctgagagaacagcagggaaacagccagagtcttgctcaagcctccctgaagtatctatacccctggactctagttatgggggctaataaatgttatatactgtttaaggtaaaaaa

SEQ ID NO: 91 - DNA, 2099 bp, Homo sapiens
  60S ribosomal protein L22 proprotein, L22, Epstein-Barr virus small RNA-associated protein, EBER-associated protein (EAP), heparin-binding protein 15 (HBP15, HBp15, HBP15/L22)
gcgtctgcgtagttcgctcacctccctttctaactccgctgccgccatggctcctgtgaaaaagcttgtggtgaaggggggcaaaaaaaagaagcaagttctgaagttcactcttgattgcacccaccctgtagaagatggaatcatggatgctgccaattttgagcagtttttgcaagaaaggatcaaagtgaacggaaaagctgggaaccttggtggaggggtggtgaccatcgaaaggagcaagagcaagatcaccgtgacatccgaggtgcctttctccaaaaggtatttgaaatatctcaccaaaaaatatttgaagaagaataatctacgtgactggttgcgcgtagttgctaacagcaaagagagttacgaattacgttacttccagattaaccaggacgaagaagaggaggaagacgaggattaaatttcatttatctggaaaattttgtatgagttcttgaataaaacttgggaaccaaaatggtggtttatccttgtatctctgcagtgtggattgaacagaaaattggaaatcatagtcaaagggcttcccttggttcgccactcatttatttgtaacttgacttctttttttttctgcttaaaaatttcaattctcgtggtaataccagagtagaaggagagggtgactttaccgaactgacagccattggggaggcagatgcgggtgtggaggtgtgggctgaaggtagtgactgtttgattttaaaaagtgtgactgtcagttgtatctgttgcttttctcaatgattcagggatacaaatgggcttctctcattcattaaaagaaaacgcgacatctttctaagattctctgtgggaaaatgactgtcaataaaatgcgggtttctgggccattcgtcttactttcattttttgattacaaatttctcttgacgcacacaattatgtctgctaatcctcttcttcctagagagagaaactgtgctccttcagtgttgctgccataaaggggtttggggaatcgattgtaaaagtcccaggttctaaattaactaaatgtgtacagaaatgaacgtgtaagtaatgtttctacaggtctttgcaacaaactgtcactttcgtctccagcagagggagctgtaggaatagtgcttccagatgtggtctcccgtgtggggcccagcaatgggggcccctgatgccaagagctctggaggttcttgaaagaggggacacgaaggaggagtgactgggaagcctcccatgccaaggaggtgggaggtgccctggaaatagctgcctcatgccacttaggccatgactggatttaatgtcagtggtgtgccacagtgcagaggctagacaactgaaaggggctaccaaggctgggaaaaaaatgcaattgttgctgtgagtgactttgaaagactctggtgccttgtggtgcccttctgaaattcaaacagtaatgcaaaagtgtctgcattagaatttacggtgtctaaaattcatgtttttaaaagagcttgcctacagatggtttccacacttgaaattgtgccctgcgagttgcatagctggaagttcaatgctcagtcctaccttggctcccattaaacatttggtgctctgtggattgagttgaacgtgttgaggctttgcaatttcacttgtgttaaaggctctggcatttttccatttctatgcaaatttctttgaagcagaattgcttgcatatttcttctctgccgtcacagaaagcagagtttctttcaaacttcactgaggcatcagttgctctttggcaatgtcccttaaccatgattattaactaagtttgtggcttgagtttacaaattctacttgttgcattgatgttcccatgtagtaagtcatttttagtttggttgtgaaaaaaccctgggctgaagttggcatttcagttaaaagaaaaaaagaaactagtcccagatttgaaaacttgtaataaaattgaaactcactggttttctatgtctttttgaactcttgtaatcgagttttgatcatattttctattaaagtggctaacacctggctactcttactgtaaaaaaaaaaaaaaa

SEQ ID NO: 92 - DNA, 3499 bp, Homo sapiens
  adaptor-related protein complex 2, alpha 1 subunit transcript variant 1 (AP2A1), clathrin-associated/ assembly/adaptor protein, large, alpha 1 (CLAPA1), alpha1-adaptin, alpha-adaptin A, AP-2 complex subunit alpha-1, 100 kDa coated vesicle protein A
cggctcagagctccggaccgcgggcggaggggaggggcagggggcggtgccacggcctgccagcccgcccgcccgcccgccagccagccctccccgcggccggctcggctccttggcgctgcctggggtcctttccgcccggtccccgcttgccagcccccgctgctctgtgccctgtccggccaggcctggagccgacaccaccgccatcatgccggccgtgtccaagggcgatgggatgcgggggctcgcggtgttcatctccgacatccggaactgtaagagcaaagaggcggaaattaagagaatcaacaaggaactggccaacatccgctccaagttcaaaggagacaaagccttggatggctacagtaagaaaaaatatgtgtgtaaactgcttttcatcttcctgcttggccatgacattgactttgggcacatggaggctgtgaatctgttgagttccaataaatacacagagaagcaaataggttacctgttcatttctgtgctggtgaactcgaactcggagctgatccgcctcatcaacaacgccatcaagaatgacctggccagccgcaaccccaccttcatgtgcctggccctgcactgcatcgccaacgtgggcagccgggagatgggcgaggcctttgccgctgacatcccccgcatcctggtggccggggacagcatggacagtgtcaagcagagtgcggccctgtgcctccttcgactgtacaaggcctcgcctgacctggtgcccatgggcgagtggacggcgcgtgtggtacacctgctcaatgaccagcacatgggtgtggtcacggccgccgtcagcctcatcacctgtctctgcaagaagaacccagatgacttcaagacgtgcgtctctctggctgtgtcgcgcctgagccggatcgtctcctctgcctccaccgacctccaggactacacctactacttcgtcccagcaccctggctctcggtgaagctcctgcggctgctgcagtgctacccgcctccagaggatgcggctgtgaaggggcggctggtggaatgtctggagactgtgctcaacaaggcccaggagccccccaaatccaagaaggtgcagcattccaacgccaagaacgccatcctcttcgagaccatcagcctcatcatccactatgacagtgagcccaacctcctggttcgggcctgcaaccagctgggccagttcctgcagcaccgggagaccaacctgcgctacctggccctggagagcatgtgcacgctggccagctccgagttctcccatgaagccgtcaagacgcacattgacaccgtcatcaatgccctcaagacggagcgggacgtcagcgtgcggcagcgggcggctgacctcctctacgccatgtgtgaccggagcaatgccaagcagatcgtgtcggagatgctgcggtacctggagacggcagactacgccatccgcgaggagatcgtcctgaaggtggccatcctggccgagaagtacgccgtggactacagctggtacgtggacaccatcctcaacctcatccgcattgcgggcgactacgtgagtgaggaggtgtggtaccgtgtgctacagatcgtcaccaaccgtgatgacgtccagggctatgccgccaagaccgtctttgaggcgctccaggcccctgcctgtcacgagaacatggtgaaggttggcggctacatccttggggagtttgggaacctgattgctggggacccccgctccagccccccagtgcagttctccctgctccactccaagttccatctgtgcagcgtggccacgcgggcgctgctgctgtccacctacatcaagttcatcaacctcttccccgagaccaaggccaccatccagggcgtcctgcgggccggctcccagctgcgcaatgctgacgtggagctgcagcagcgagccgtggagtacctcaccctcagctcagtggccagcaccgacgtcctggccacggtgctggaggagatgccgcccttccccgagcgcgagtcgtccatcctggccaagctgaaacgcaagaaggggccaggggccggcagcgccctggacgatggccggagggaccccagcagcaacgacatcaacgggggcatggagcccacccccagcactgtgtcgacgccctcgccctccgccgacctcctggggctgcgggcagcccctcccccggcagcacccccggcttctgcaggagcagggaaccttctggtggacgtcttcgatggcccggccgcccagcccagcctggggcccacccccgaggaggccttcctcagcgagctggagccgcctgcccccgagagccccatggctttgctggctgacccagctccagctgctgacccaggtcctgaggacatcggccctcccattccggaagccgatgagttgctgaataagtttgtgtgtaagaacaacggggtcctgttcgagaaccagctgctgcagatcggagtcaagtcagagttccgacagaacctgggccgcatgtatctcttctatggcaacaagacctcggtgcagttccagaatttctcacccactgtggttcacccgggagacctccagactcagctggctgtgcagaccaagcgcgtggcggcgcaggtggacggcggcgcgcaggtgcagcaggtgctcaatatcgagtgcctgcgggacttcctgacgcccccgctgctgtccgtgcgcttccggtacggtggcgccccccaggccctcaccctgaagctcccagtgaccatcaacaagttcttccagcccaccgagatggcggcccaggatttcttccagcgctggaagcagctgagcctccctcaacaggaggcgcagaaaatcttcaaagccaaccaccccatggacgcagaagttactaaggccaagcttctggggtttggctctgctctcctggacaatgtggaccccaaccctgagaacttcgtgggggcggggatcatccagactaaagccctgcaggtgggctgtctgcttcggctggagcccaatgcccaggcccagatgtaccggctgaccctgcgcaccagcaaggagcccgtctcccgtcacctgtgtgagctgctggcacagcagttctgagccctggactctgccccgggggatgtggccggcactgggcagccccttggactgaggcagttttggtggatgggggacctccactggtgacagagaagacaccagggtttgggggatgcctgggactttcctccggccttttgtatttttatttttgttcatctgctgctgtttacattctggggggttagggggagtccccctccctccctttcccccccaagcacagaggggagaggggccagggaagtggatgtctcctcccctcccaccccaccctgttgtagcccctcctaccccctccccatccaggggctgtgtattattgtgagcgaataaacagagagacgctaa

SEQ ID NO: 93 - DNA, 5125 bp, Homo sapiens
  nucleosome assembly protein 1-like 1 transcript variant 2 (NAP1L1, NAP1, NAP1L), NAP-1-related protein, HSP22-like protein interacting protein
aaaagatatggtggggtgcttaacagaggaggttagacaccggcgggaaccagaggagcccaagcgcggcgcctgggcctcggggctgcaggagtcctcggtgggggtatggaggtcgccggggaaggaggacggttcagttgctaggcaacccggcctggacccgcctctcgctcgcgttgctgggagactacaaggccgggaggagggcggcgaaagggccctacgtgctgacgctaattgtatatgagcgcgagcggcgggctcttgggtcttttttagcgccatctgctcgcggcgccgcctcctgctcctcccgctgctgctgccgctgccgccctgagtcactgcctgcgcagctccggccgcctggctccccatactagtcgccgatatttggagttcttacaacatggcagacattgacaacaaagaacagtctgaacttgatcaagatttggatgatgttgaagaagtagaagaagaggaaactggtgaagaaacaaaactcaaagcacgtcagctaactgttcagatgatgcaaaatcctcagattcttgcagcccttcaagaaagacttgatggtctggtagaaacaccaacaggatacattgaaagcctgcctagggtagttaaaagacgagtgaatgctctcaaaaacctgcaagttaaatgtgcacagatagaagccaaattctatgaggaagttcacgatcttgaaaggaagtatgctgttctctatcagcctctatttgataagcgatttgaaattattaatgcaatttatgaacctacggaagaagaatgtgaatggaaaccagatgaagaagatgagatttcggaggaattgaaagaaaaggccaagattgaagatgagaaaaaagatgaagaaaaagaagaccccaaaggaattcctgaattttggttaactgtttttaagaatgttgacttgctcagtgatatggttcaggaacacgatgaacctattctgaagcacttgaaagatattaaagtgaagttctcagatgctggccagcctatgagttttgtcttagaatttcactttgaacccaatgaatattttacaaatgaagtgctgacaaagacatacaggatgaggtcagaaccagatgattctgatcccttttcttttgatggaccagaaattatgggttgtacagggtgccagatagattggaaaaaaggaaagaatgtcactttgaaaactattaagaagaagcagaaacacaagggacgtgggacagttcgtactgtgactaaaacagtttccaatgactctttctttaacttttttgcccctcctgaagttcctgagagtggagatctggatgatgatgctgaagctatccttgctgcagacttcgaaattggtcactttttacgtgagcgtataatcccaagatcagtgttatattttactggagaagctattgaagatgatgatgatgattatgatgaagaaggtgaagaagcggatgaggaaggggaagaagaaggagatgaggaaaatgatccagactatgacccaaagaaggatcaaaacccagcagagtgcaagcagcagtgaagcaggatgtatgtggccttgaggataacctgcactgtaatagcctaaacacaactcttatttacttacagccttatgtttttgtattttcttggtagactaggtaatttttttttaaaggacaggaaactgatattttaaagaccaatttgttctacctagcattttaactagtttttctgccagctatgttgaatgcacaaattctgtcacgcatgttcattcattgctacataatttggttcttctggaatatttttatgtagctcttggagtacagctatgaaaattaacaactgttaaaggaaataccttttttttttttttgtaattttttccttgaagaaccaaagtattttttcagctggttgttgaatagggttaagtccgcttggattagctgtgcctttcattactttgttacagaaatgcagtgacttatactaagacaatttattgtttaaaaaaaaaattggcaagacaactatatggttaagaatttccagtatgaccacacccaataactgttattagagtgttaatggattattgtgttttaggtgacatagttaactgtaaagtaacctgactcagtatagttactggtaccacagtgaggtgaataaaacgggattttcagaagttagcctgaatttaactgtatttttaaatttaacctccattaactaagcatcttttctttgtggtagggtctaccttctgcttccctggaaaggatgaatttacatcatttgacaagcctattttcaagttatttgttgtttgtttgcttgtttttgtttttgcagctaaaataaaaatttcaaatacaattttagttcttacaagataatgtcttaattttgtaccaattcaggtagaagtagaggcctaccttgaattaagggttatactcagtttttaacacattgttgaagaaaaggtaccagctttggaacgagatgctatactaataagcaagtgtaaaaaaaaaaaaaaaagaggaagaaaatcttaagtgattgatgctgttttcttttaaaaaaaaaaaaaaaaattcattttctttgggttagagctagagagaaggccccaagcttctatggtttcttctaattcttattgcttaaagtatgagtatgtcacttacccgtgcttctgtttactgtgtaattaaaatgggtagtactgtttacctaactacctcatggatgtgttaaggcatattgagttaaatctcatataatgtttctcaatcttgttaaaagctcaaaattttgggcctatttgtaatgccagtgtgacactaagcattttgttcacaccacgctttgataactaaactggaaaacaaaggtgttaagtacctctgttctggatctgggcagtcagcactctttttagatctttgtgtggctcctatttttatagaagtggagggatgcactatttcacaaggtccaagatttgttttcagatatttttgatgactgtattgtaaatactacagggatagcactatagtattgtagtcatgagacttaaagtggaaataagactatttttgacaaaagatgccattaaatttcagactgtagagccacatttacaatacctcaggctaattactgttaattttggggttgaacttttttttgacagtgagggtggattattggattgtcattagaggaaggtctagatttcctgctcttaataaaattacattgaattgatttttagaggtaatgaaaacttcctttctgagaagttagtgttaaggtcttggaatgtgaacacattgtttgtagtgctatccattcctctcctgagattttaacttactactggaaatccttaaccaattataatagctttttttctttattttcaaaatgatttcctttgctttgattagacactatgtgctttttttttttaaccatagttcatcgaaatgcagctttttctgaacttcaaagatagaatcccatttttaatgaactgaagtagcaaaatcatctttttcattctttaggaaatagctattgccaaagtgaaggtgtagataatacctagtcttgttacataaaggggatgtggtttgcagaagaattttctttataaaattgaagttttaagggacgtcagtgtttatgccatttttccagttccaaaatgattccattccattctagaaatttgaagtatgtaacctgaaatccttaataaaatttggatttaattttataaaatgtactggtgatattttgggtgtttttttttaaatgaatgtatatactttttttttgaagagtggagagtagtgatgtctagagggagctattttgtgctgaggccactatgttctgtaaatatataattttaagagcaacctcacaatccctgctaagtggagtttattatttgaagactaaaatggaattccatagttcctgataggttatattctgggttattattctgagttatctacaaacatttttgagatttgtctttacactctgattgtagtttccagcagcccatgcacactgccaagtaagtctcattttttcctgttagaaatggtgaaatatcatataatcacttataaagaaaactgatatgaaaaaattttagagttgtttgctttatggtcactcaagtagggtaagtgttccacaaattccacaagttgatagtttaacatggatgtctgaaagccacatatataatttcttaggattcttaaattagtaaatctagcttactgaagcagtattagcatcactattttagattgcaaaaataccttaattgtgtggaactggcttgtagagtggtacttaagaaaaatgggattctacctctatttctgttttagcacacttaatcaggaaaggatatattaactttcataaaaatatttttgttgtgtgaataggttaatgatatggtaaggcccctaaaataactgaattaattgtttattgtaattgtaggccattcccattattaaaaataaagacaaaacttgaagtaactgaaaatcttatcgtgctatgtagaaatattgaactaatattcaaatatttgaatgctttggtttcagggattggtttaaaattggagtccttttttatgggttagtcttacaaaaatttaagcctttatatttttgactttaaatcaaaacaaatgttattttaaatgtacagaatagattggtagtgcagaagagtgtaagttcttcataggagctttagaaaagagaaatatgtgctaattcagtttttttttaatctgcactgtacatatatacttggtaattatgagcttgattttgtttttggaaatatgtgttcataatttaggtaatttgctacttaaagcactaagtctctgatacctgaaaagtacatgtaaatggtgatggtgaaataatactgcagttaacttaatagatgtatactggtgatttttgtatgctggattaaaactccagatattaaaatataacctggataaaaagcc

SEQ ID NO: 94 - DNA, 787 bp, Homo sapiens
  40S ribosomal protein S14 transcript variant 2, S14, emetine resistance (EMTB)
ctccgccccctcccactctctctttccggtgtggagtctggagacgacgtgcaggtaggaggcccgggcgcgacaatcggggggcatcctgcggcgaggggaccctgtggggcttgggacgagagacgggggtctttccgtgggaaccgagctaggtgccgggcaagagacgcgcggctggcccacctggatcctggccaactcgggattgagttcgttcctggtctcagaaggcccgttttgctttcagggaggagcttgtgaaaaatggcacctcgaaaggggaaggaaaagaaggaagaacaggtcatcagcctcggacctcaggtggctgaaggagagaatgtatttggtgtctgccatatctttgcatccttcaatgacacttttgtccatgtcactgatctttctggcaaggaaaccatctgccgtgtgactggtgggatgaaggtaaaggcagaccgagatgaatcctcaccatatgctgctatgttggctgcccaggatgtggcccagaggtgcaaggagctgggtatcaccgccctacacatcaaactccgggccacaggaggaaataggaccaagacccctggacctggggcccagtcggccctcagagcccttgcccgctcgggtatgaagatcgggcggattgaggatgtcacccccatcccctctgacagcactcgcaggaaggggggtcgccgtggtcgccgtctgtgaacaagattcctcaaaatattttctgttaataaattgccttcatgtaaactgtttcaaaaaaaaa

SEQ ID NO: 95 - DNA, 978 bp, Homo sapiens
  ethylmalonic encephalopathy 1 (ETHE1), persulfide dioxygenase ETHE1, mitochondrial, sulfur dioxygenase ETHE1, hepatoma subtracted clone one protein (HSCO), YF13H12, protein ETHE1, mitochondrial
agtgccgtagcgcccggctcctgcaggcgctcggcctccgctcattcctgaccccgcagtgggcgcgatggcggaggctgtactgagggtcgcccggcggcagctgagccagcgcggcgggtctggagcccccatcctcctgcggcagatgttcgagcctgtgagctgcaccttcacgtacctgctgggtgacagagagtcccgggaggccgttctgatcgacccagtcctggaaacagcgcctcgggatgcccagctgatcaaggagctggggctgcggctgctctatgctgtgaatacccactgccacgcggaccacattacaggctcggggctgctccgttccctcctccctggctgccagtctgtcatctcccgccttagtggggcccaggctgacttacacattgaggatggagactccatccgcttcgggcgcttcgcgttggagaccagggccagccctggccacaccccaggctgtgtcaccttcgtcctgaatgaccacagcatggccttcactggagatgccctgttgatccgtgggtgtgggcggacagacttccagcaaggctgtgccaagaccttgtaccactcggtccatgaaaagatcttcacacttccaggagactgtctgatctaccctgctcacgattaccatgggttcacagtgtccaccgtggaggaggagaggactctgaaccctcggctcaccctcagctgtgaggagtttgtcaaaatcatgggcaacctgaacttgcctaaacctcagcagatagactttgctgttccagccaacatgcgctgtggggtgcagacacccactgcctgatctcacttctgtcagatgctcccatccactattaatgcactaggtgggaggagagggcggcaatgacactgcacctctcctttcccaccgcattccctggagctccctaaataaaactttttttaacgtgaaaaaaaaaaaaaaaa

SEQ ID NO: 96 - DNA, 1890 bp, Homo sapiens
  cyclin I (CCNI, CYC1, CYI), cyclin ITI
cccgcgagcggacgcggcagcgcctctgtctcgctttttcttatttttcccccctttcccctttctttttttttttttcttttcttttctcccctccccccctttcaccatttcccctcggaggcgctttccccgggcaggggcagagccggtctcaccccccgcctctccccggcccccgccgccctatggcgagagggagccccctcccaacccgggctcgagcggcggcggcctcaggccgggggtcatcatggaactaattcgctgaccgacccagcggccgcagccgtgcgtcccgctcgagcgccagcgcccgcgcccgcgccccccgatccgcttcccctttctccctcctcagttggccgagtcgtcccgcgcgcaccgcctccgcgcgcctatgagaatgaggtggtaacgggcccccggatgaccccgcgtcaccactgtgaggcctacagctctgccggggaggaggaggaggaggaagaggaggagaaggtagctacagcaagctgggtagcaggcagatccaaaggatatcatgaagtttccagggcctttggaaaaccagagattgtctttcctgttggaaaaggcaatcactagggaagcacagatgtggaaagtgaatgtgcggaaaatgccttcaaatcagaatgtttctccatcccagagagatgaagtaattcaatggctggccaaactcaagtaccaattcaacctttacccagaaacatttgctctggctagcagtcttttggataggtttttagctaccgtaaaggctcatccaaaatacttgagttgtattgcaatcagctgttttttcctagctgccaagactgttgaggaagatgagagaattccagtactaaaggtattggcaagagacagtttctgtggatgttcctcatctgaaattttgagaatggagagaattattctggataagttgaattgggatcttcacacagccacaccattggattttcttcatattttccatgccattgcagtgtcaactaggcctcagttacttttcagtttgcccaaattgagcccatctcaacatttggcagtccttaccaagcaactacttcactgtatggcctgcaaccaacttctgcaattcagaggatccatgcttgctctggccatggttagtctggaaatggagaaactcattcctgattggctttctcttacaattgaactgcttcagaaagcacagatggatagctcccagttgatccattgtcgggagcttgtggcacatcacctttctactctgcagtcttccctgcctctgaattccgtttatgtctaccgtcccctcaagcacaccctggtgacctgtgacaaaggagtgttcagattacatccctcctctgtcccaggcccagacttctccaaggacaacagcaagccagaagtgccagtcagaggtacagcagccttttaccatcatctcccagctgccagtgggtgcaagcagacctctactaaacgcaaagtagaggaaatggaagtggatgacttctatgatggaatcaaacggctctataatgaagataatgtctcagaaaatgtgggttctgtgtgtggcactgatttatcaagacaagagggacatgcttccccttgtccacctttgcagcctgtttctgtcatgtagtttcaacaagtgctacctttgagtgtaaactaaggtagactactttgggaatgagaacatgcaaaatcaggaaaggctgtagaaggaaatataccttaacaggctgatttggagtgagccagaaaaaaaaaataaaactctcattatttgtgtggctaattataattcagcgttatttaagcacataaagaccaaaaaaaaaaaaaaaaaaa

SEQ ID NO: 97 - DNA, 2856 bp, Homo sapiens
  metastasis associated 1 transcript variant 1 (MTA1), metastasis-associated protein MTA1 isoform MTA1
gcggccctcccgtccctgcgcggcctcggcggcctcggcggcggcggcggcggcggcggcggcagcagcgcggcccctttaaacgcctgcggcgccccccgcccccgccatcgcgcctccattttcccggccgcccgcgccgagcgccgcgcccgccccgggcccctccgccgccgccggcccggacatggccgccaacatgtacagggtcggagactacgtctactttgagaactcctccagcaacccatacctgatccggagaatcgaggagctcaacaagacggccaatgggaacgtggaggccaaagtggtgtgcttctaccggaggcgggacatctccagcaccctcatcgccctggccgacaagcacgcaaccctgtcagtctgctataaggccggaccgggggcggacaacggcgaggaaggggaaatagaagaggaaatggagaatccggaaatggtggacctgcccgagaaactaaagcaccagctgcggcatcgggagctgttcctctcccggcagctggagtctctgcccgccacgcacatcaggggcaagtgcagcgtcaccctgctcaacgagaccgagtcgctcaagtcctacctggagcgggaggatttcttcttctattctctagtctacgacccacagcagaagaccctgctggcagataaaggagagattcgagtaggaaaccggtaccaggcagacatcaccgacttgttaaaagaaggcgaggaggatggccgagaccagtccaggttggagacccaggtgtgggaggcgcacaacccactcacagacaagcagatcgaccagttcctggtggtggcccgctctgtgggcaccttcgcacgggccctggactgcagcagctccgtccgacagcccagcctgcacatgagcgccgcagctgcctcccgagacatcaccctgttccacgccatggatactctccacaagaacatctacgacatctccaaggccatctcggcgctggtgccgcagggcgggcccgtgctctgcagggacgagatggaggagtggtctgcatcagaggccaaccttttcgaggaagccctggaaaaatatgggaaggatttcacggacattcagcaagattttctcccgtggaagtcgctgaccagcatcattgagtactactacatgtggaagaccaccgacagatacgtgcagcagaaacgcttgaaagcagctgaagctgagagcaagttaaagcaagtttatattcccaactataacaagccaaatccgaaccaaatcagcgtcaacaacgtcaaggccggtgtggtgaacggcacgggggcgccgggccagagccctggggctggccgggcctgcgagagctgttacaccacacagtcttaccagtggtattcttggggtccccctaacatgcagtgtcgtctctgcgcatcttgttggacatattggaagaaatatggtggcttgaaaatgccaacccggttagatggagagaggccaggaccaaaccgcagtaacatgagtccccacggcctcccagcccggagcagcgggagccccaagtttgccatgaagaccaggcaggctttctatctgcacacgacgaagctgacgcggatcgcccggcgcctgtgccgtgagatcctgcgcccgtggcacgctgcgcggcacccctacctgcccatcaacagcgcggccatcaaggccgagtgcacggcgcggctgcccgaagcctcccagagcccgctggtgctgaagcaggcggtacgcaagccgctggaagccgtgcttcggtatcttgagacccacccccgcccccccaagcctgaccccgtgaaaagcgtgtccagcgtgctcagcagcctgacgcccgccaaggtggcccccgtcatcaacaacggctcccccaccatcctgggcaagcgcagctacgagcagcacaacggggtggacggcaacatgaagaagcgcctcttgatgcccagtaggggtctggcaaaccacggacaggccaggcacatgggaccaagccggaacctcctgctcaacgggaagtcctaccccaccaaagtgcgcctgatccgggggggctccctgcccccagtcaagcggcggcggatgaactggatcgacgccccggatgacgtgttctacatggccacagaggagaccaggaagatccgcaagctgctctcatcctcggaaaccaagcgtgctgcccgccggccctacaagcccatcgccctgcgccagagccaggccctgccgccgcggccaccgccacctgcgcccgtcaacgacgagcccatcgtcatcgaggactaggggccgcccccacctgcggccgccccccgcccctcgcccgcccacacggccccttcccagccagcccgccgcccgcccctcagtttggtagtgccccacctcccgccctcacctgcagagaaacgcgctccttggcggacactgggggaggagaggaagaagcgcggctaacttattccgagaatgccgaggagttgtcgtttttagctttgtgtttactttttggctggagcggagatgaggggccaccccgtgcccctgtgctgcggggccttttgcccggaggccgggccctaaggttttgttgtgttctgttgaaggtgccattttaaattttatttttattactttttttgtagatgaacttgagctctgtaacttacacctggaatgttaggatcgtgcggccgcggccggccgagctgcctggcggggttggcccttgtcttttcaagtaattttcatattaaacaaaaacaaagaaaaaaaatcttataaaaaggaaaaaaaaaaaaaaaaaa

SEQ ID NO: 98 - DNA, 1286 bp, Homo sapiens
  eukaryotic translation initiation factor 3, subunit H (EIF3H), eukaryotic translation initiation factor 3, subunit 3 gamma, 40kDa (eIF3-gamma, eIF3-p40), eukaryotic translation initiation factor 3, subunit 2 (beta, 36kD)
ctctttcttcctgtctgcttggaaagatggcgtcccgcaaggaaggtaccggctctactgccacctcttccagctccaccgccggcgcagcagggaaaggcaaaggcaaaggcggctcgggagattcagccgtgaagcaagtgcagatagatggccttgtggtattaaagataatcaaacattatcaagaagaaggacaaggaactgaagttgttcaaggagtgcttttgggtctggttgtagaagatcggcttgaaattaccaactgctttcctttccctcagcacacagaggatgatgctgactttgatgaagtccaatatcagatggaaatgatgcggagccttcgccatgtaaacattgatcatcttcacgtgggctggtatcagtccacatactatggctcattcgttacccgggcactcctggactctcagtttagttaccagcatgccattgaagaatctgtcgttctcatttatgatcccataaaaactgcccaaggatctctctcactaaaggcatacagactgactcctaaactgatggaagtttgtaaagaaaaggatttttcccctgaagcattgaaaaaagcaaatatcacctttgagtacatgtttgaagaagtgccgattgtaattaaaaattcacatctgatcaatgtcctaatgtgggaacttgaaaagaagtcagctgttgcagataaacatgaattgctcagccttgccagcagcaatcatttggggaagaatctacagttgctgatggacagagtggatgaaatgagccaagatatagttaaatacaacacatacatgaggaatactagtaaacaacagcagcagaaacatcagtatcagcagcgtcgccagcaggagaatatgcagcgccagagccgaggagaacccccgctccctgaggaggacctgtccaaactcttcaaaccaccacagccgcctgccaggatggactcgctgctcattgcaggccagataaacacttactgccagaacatcaaggagttcactgcccaaaacttaggcaagctcttcatggcccaggctcttcaagaatacaacaactaagaaaaggaagtttccagaaaagaagttaacatgaactcttgaagtcacaccagggcaactcttggaagaaatatatttgcatattgaaaagcacagaggatttctttagtgtcattgccgattttggctataacagtgtctttctagccataataaaataaaacaaaatcttgactgcttgctcatttgaaaaaaaaaaaa

SEQ ID NO: 99 - DNA, 766 bp, Homo sapiens
  60S ribosomal protein L9 transcript variant 1, L9, NPC-A-16
acgcgatacaagtacgtaatgacgacagacgttctttctttgctgcgtctactgcgagaatgaagactattctcagcaatcagactgtcgacattccagaaaatgtcgacattactctgaagggacgcacagttatcgtgaagggccccagaggaaccctgcggagggacttcaatcacatcaatgtagaactcagccttcttggaaagaaaaaaaagaggctccgggttgacaaatggtggggtaacagaaaggaactggctaccgttcggactatttgtagtcatgtacagaacatgatcaagggtgttacactgggcttccgttacaagatgaggtctgtgtatgctcacttccccatcaacgttgttatccaggagaatgggtctcttgttgaaatccgaaatttcttgggtgaaaaatatatccgcagggttcggatgagaccaggtgttgcttgttcagtatctcaagcccagaaagatgaattaatccttgaaggaaatgacattgagcttgtttcaaattcagcggctttgattcagcaagccacaacagttaaaaacaaggatatcaggaaatttttggatggtatctatgtctctgaaaaaggaactgttcagcaggctgatgaataagatctaagagttacctggctacagaaagaagatgccagatgacacttaagacctacttgtgatatttaaatgatgcaataaaagacctattgatttggaccttcttcttaaaaaaaaaaaaaaaaaaa

SEQ ID NO: 100 - DNA, 3325 bp, Homo sapiens
  40S ribosomal protein S23, S23, homolog of yeast ribosomal protein S28
ggggtccttggctgggcggggcttgctcgcggtggcttgtggctccttcctgcggtgcttctctctttcgctcaggcccgtggcgccgacaggatgggcaagtgtcgtggacttcgtactgctaggaagctccgtagtcaccgacgagaccagaagtggcatgataaacagtataagaaagctcatttgggcacagccctaaaggccaacccttttggaggtgcttctcatgcaaaaggaatcgtgctggaaaaagtaggagttgaagccaaacagccaaattctgccattaggaagtgtgtaagggtccagctgatcaagaatggcaagaaaatcacagcctttgtacccaatgacggttgcttgaactttattgaggaaaatgatgaagttctggttgctggatttggtcgcaaaggtcatgctgttggtgatattcctggagtccgctttaaggttgtcaaagtagccaatgtttctcttttggccctatacaaaggcaagaaggaaagaccaagatcataaatattaatggtgaaaacactgtagtaataaattttcatatgccaaaaaatgtttgtatcttactgtcccctgttctcaccacgaagatcatgttcattaccaccaccacccccccttattttttttatcctaaaccagcaaacgcaggacctgtaccaattttaggagacaataagacagggttgtttcaggattctctagagttaataacatttgtaacctggcacagtttccctcatcctgtggaataagaaaatgggatagatctggaataaatgtgcagtattgtagtattactttaagaactttaagggaacttcaaaaactcactgaaattctagtgagatactttcttttttattcttggtattttccatatcgggtgcaacacttcagttaccaaatttcattgcacatagattatcttaggtacccttggaaatgcacattcttgtatccatcttacaggggcccaagatgataaatagtaaactcaaaattgctccccactctgtttattatttaaaggtgtcaggatctgtgttgtaatgtgtctacattaatgtgtttaggagaatacaggcattggatcatttagttgatggaagtatatgccaggcaagggagataaggtatacgacaagactgatgttttcagtatcttctcatgaggttgtcagagaccttcatgtcttcaaagactagtcagcaaatgaagtggtttagtgtagagacaagattggttgtgttttgataatttaagctaggtattgagtacatgtggattttgctgtccacaaatacttgtttcagagttttcatggatacagtggcatggttgaaatgaagctgtgagccttctgctttaaatctgatgtaagaaactcctgttaacaaatagtaagtatgggttaattagccctttgatcaaagcctagctttacattgtttaggatctttggaaaacaattggtttggttgcccactttccgtaggatcaagagcagaacctttcacatggcacagaagaacccaggttgcgcttcatacctgcatattccagccttagcctgccatttctctccttggcactttgtgctccagcaacactggtctcagttggtcatcctcaaacttgggttccatatccagcctcaggacctctgttcctgttactatggttccttgcatgtcgcctgctcttactaaagagctcgtgtgttttccagcacacttcggtttatctcttgatgatgatgctagtctctccctccgcaagggcggaaaggctgcctgttggtttgtaccagtgtttcctaacgtgtagctgcagtcagtatttggctaagctgttcccaggggctcaacagatgctttcggatgagccttaactgacccaatcctttgtgatgcgggagagattgctaggcctcgctcacctggccagaaccagggaaagaggccgcggttgcagcgcgattccaggccctgggcgtcaggcgcggggtgggcagctctccccgggcggtggggcccttgtgaccgcgaggcggggcgcaccaggaagggagtgggacagcgcgggcgcccagggatgtggcctggttacctgccttctctgatacgtcaagacaccttcaacaatggcttgcagctgtaccctgttggctgcacccaggacgcccttttcactgctaagcagtcctacctgaggcccaggggctgccagattgacccataaataatctccggcgcctcagatccagaagctgctgagcctgatcttagtgccttctcctttctctgtgtggccccccagcccctttccccactgccttgtgtccaaggccctttccttcatgtatccatggaggagagacaaaaatacacatcaataaaataagatagggaatccataaatagacattcagaagtatggccaacggatttatcttaaaaccaatggaggaagaagagtttcaataaatgttgtggacttccatttgtcaaagaccaaaacaaaggaaccccaaccttacatgtaatacaaacttaactcaaaatggatcatatatctaaatgtaaaatggaaagctataaaactgaaaacagactatctttacaacctaggcgtaggtatagtttttagacattacaccaaaagcacatgccgtaaaagaaaaaatagataaattggtggatttcattaaaattaaaaaactttttctctctgaaaaatcctgttaagctgggcgctgtggttcatgcctgtaatcccagcactttgggaggctgagttgggaagaaattaatagcttgaggccaggagttcaagatcatcctgggcagcaaagtcatacactcttgagggaagagagagaccttctcatattgttttatattgttttatactcagtacctgttttaagaaaaaaacaaggaagtgaaatcaaagacaggcagcccggcaccaggcctgaaaccagccctgggcctgcctggcctaaacctagtagttaaaaatcaacttacgacttagaacctgatgttatccgtagattccaagcattgtataaaaaaattgtgaaactccctgttgtgttctgtaccagtgcatgaaacccctgtcacatatcccctagattgctcaatcaatcacgaccctttcatgtgaaatctttagtgttgtgagcccttaaaagggacagaaattgtgcacttgaggagctcagattttaaggctgtagcttgccgatgctcccagctgaataaagcccttccttctacaaaaaaaaaaaaa

SEQ ID NO: 101 - DNA, 829 bp, Homo sapiens
  40S ribosomal protein S6, S6, phosphoprotein NP33
cctcttttccgtggcgcctcggaggcgttcagctgcttcaagatgaagctgaacatctccttcccagccactggctgccagaaactcattgaagtggacgatgaacgcaaacttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactctg

SEQ ID NO: 102 - DNA, 745 bp, Homo sapiens
  60S ribosomal protein S7, S7, DBA8
gcgctgtttccgcctcttgccttcggacgccggattttgacgtgctctcgcgagatttgggtctcttcctaagccggcgctcggcaagttctcccaggagaaagccatgttcagttcgagcgccaagatcgtgaagcccaatggcgagaagccggacgagttcgagtccggcatctcccaggctcttctggagctggagatgaactcggacctcaaggctcagctcagggagctgaatattacggcagctaaggaaattgaagttggtggtggtcggaaagctatcataatctttgttcccgttcctcaactgaaatctttccagaaaatccaagtccggctagtacgcgaattggagaaaaagttcagtgggaagcatgtcgtctttatcgctcagaggagaattctgcctaagccaactcgaaaaagccgtacaaaaaataagcaaaagcgtcccaggagccgtactctgacagctgtgcacgatgccatccttgaggacttggtcttcccaagcgaaattgtgggcaagagaatccgcgtcaaactagatggcagccggctcataaaggttcatttggacaaagcacagcagaacaatgtggaacacaaggttgaaactttttctggtgtctataagaagctcacgggcaaggatgttaattttgaattcccagagtttcaattgtaaacaaaaatgactaaataaaaagtatatattcacagtaaaaaaaaaaaaaaaaaa

SEQ ID NO: 103 - DNA, 748 bp, Homo sapiens
  60S ribosomal protein L19, L19
gcagataatgggaggagccgggcccgagcgagctctttcctttcgctgctgcggccgcagccatgagtatgctcaggcttcagaagaggctcgcctctagtgtcctccgctgtggcaagaagaaggtctggttagaccccaatgagaccaatgaaatcgccaatgccaactcccgtcagcagatccggaagctcatcaaagatgggctgatcatccgcaagcctgtgacggtccattcccgggctcgatgccggaaaaacaccttggcccgccggaagggcaggcacatgggcataggtaagcggaagggtacagccaatgcccgaatgccagagaaggtcacatggatgaggagaatgaggattttgcgccggctgctcagaagataccgtgaatctaagaagatcgatcgccacatgtatcacagcctgtacctgaaggtgaaggggaatgtgttcaaaaacaagcggattctcatggaacacatccacaagctgaaggcagacaaggcccgcaagaagctcctggctgaccaggctgaggcccgcaggtctaagaccaaggaagcacgcaagcgccgtgaagagcgcctccaggccaagaaggaggagatcatcaagactttatccaaggaggaagagaccaagaaataaaacctcccactttgtctgtacatactggcctctgtgattacatagatcagccattaaaataaaacaagccttaatctgcaaaaaaaaaaaaaaaa

SEQ ID NO: 104 - DNA, 1458 bp, Homo sapiens
  60S ribosomal protein L4, L4, ribosomal protein L1
aagcacttccttttcctgtggcagcagccgggctgagaggagcgtggctgtctcctctctccgccatggcgtgtgctcgcccactgatatcggtgtactccgaaaagggggagtcatctggcaaaaatgtcactttgcctgctgtattcaaggctcctattcgaccagatattgtgaactttgttcacaccaacttgcgcaaaaacaacagacagccctatgctgtcagtgaattagcaggtcatcagactagtgctgagtcttggggtactggcagagctgtggctcgaattcccagagttcgaggtggtgggactcaccgctctggccagggtgcttttggaaacatgtgtcgtggaggccgaatgtttgcaccaaccaaaacctggcgccgttggcatcgtagagtgaacacaacccaaaaacgatacgccatctgttctgccctggctgcctcagccctaccagcactggtcatgtctaaaggtcatcgtattgaggaagttcctgaacttcctttggtagttgaagataaagttgaaggctacaagaagaccaaggaagctgttttgctccttaagaaacttaaagcctggaatgatatcaaaaaggtctatgcctctcagcgaatgagagctggcaaaggcaaaatgagaaaccgtcgccgtatccagcgcaggggcccgtgcatcatctataatgaggataatggtatcatcaaggccttcagaaacatccctggaattactctgcttaatgtaagcaagctgaacattttgaagcttgctcctggtgggcatgtgggacgtttctgcatttggactgaaagtgctttccggaagttagatgaattgtacggcacttggcgtaaagccgcttccctcaagagtaactacaatcttcccatgcacaagatgattaatacagatcttagcagaatcttgaaaagcccagagatccaaagagcccttcgagcaccacgcaagaagatccatcgcagagtcctaaagaagaacccactgaaaaacttgagaatcatgttgaagctaaacccatatgcaaagaccatgcgccggaacaccattcttcgccaggccaggaatcacaagctccgggtggataaggcagctgctgcagcagcggcactacaagccaaatcagatgagaaggcggcggttgcaggcaagaagcctgtggtaggtaagaaaggaaagaaggctgctgttggtgttaagaagcagaagaagcctctggtgggaaaaaaggcagcagctaccaagaaaccagcccctgaaaagaagcctgcagagaagaaacctactacagaggagaagaagcctgctgcataaactcttaaatttgattattccataaaggtcaaatcattttggacagcttcttttgaataaagacctgattatacaggcagtgagaaacatgaaaaaaaaaaaaaaaaaa

SEQ ID NO: 105 - DNA, 529 bp, Homo sapiens
  40S ribosomal protein S13, S13
cgctctcctttcgttgcctgatcgccgccatcatgggtcgcatgcatgctcccgggaagggcctgtcccagtcggctttaccctatcgacgcagcgtccccacttggttgaagttgacatctgacgacgtgaaggagcagatttacaaactggccaagaagggccttactccttcacagatcggtgtaatcctgagagattcacatggtgttgcacaagtacgttttgtgacaggcaataaaattttaagaattcttaagtctaagggacttgctcctgatcttcctgaagatctctaccatttaattaagaaagcagttgctgttcgaaagcatcttgagaggaacagaaaggataaggatgctaaattccgtctgattctaatagagagccggattcaccgtttggctcgatattataagaccaagcgagtcctccctcccaattggaaatatgaatcatctacagcctctgccctggtcgcataaatttgtctgtgtactcaagcaataaaatgattgtttaacta

SEQ ID NO: 106 - DNA, 2836 bp, Homo sapiens
  GC-rich sequence DNA-binding factor 1 (GCFC, GCFC1), chromosome 21 open reading frame 66 (C21orf66), PAX3 and PAX7 binding protein 1 transcript variant 2 (PAXBP1), functional spliceosome-associated protein 105 (FSAP105), BM020
ctctctctctttctccaagtattgagagcgcgtgggagcataggcgcatgcgcgctcgtggggtgcgcggtagcaacagaggactcgacccggctggagctccggagagcgcgcgtgcgccgtcacgagctcggcgctgccggggccgcggtgtggaagcgagtattcgaccgccgtgcgggccgcggggatgttccgaaaggcccggcgggtgaacgtgcgcaagcggaacgactccgaagaggaagagcgggaacgcgatgaggagcaggagccgccgccgttgttgccgccgccgggcacgggcgaagaggcgggccccggtggcggcgacagggcccctggcggggagtcgctgctgggcccggggccgtcgccgccttccgcgctgaccccgggcctcggggctgaggccgggggcggcttccccggcggcgcggagcccggcaacgggctgaagccgcgcaagaggcctcgcgagaacaaagaggtgccccgggccagcctgctcagcttccaggacgaggaggaagaaaatgaagaagttttcaaagtgaagaaatcaagttatagcaaaaagatagtaaaattgctcaagaaggaatataaagaagatcttgaaaaatcgaagattaagacagaactcaactcatcagctgaaagtgaacaacctttggacaaaacaggacatgttaaggatacaaatcaagaagatggagttatcatcagtgaacatggtgaagatgaaatggatatggaaagtgaaaaagaggaagaaaagccaaagactggtggagctttttcaaatgctttatcttcattgaatgttcttcgtccaggagaaattccagatgcagcttttatacatgctgcaaggaaaaagcgccaaatggcccgagaattgggagatttcactcctcatgataatgagcctggtaaaggccgccttgttagagaagatgagaatgatgccagtgatgatgaagatgacgatgagaaacgccggatagttttttctgtgaaagaaaagtcacaaagacaaaaaattgctgaggaaataggaattgaggggagtgatgatgatgctttagtaactggagaacaggatgaagagctcagccgatgggaacaggagcagataaggaaaggaattaatatccctcaggttcaagccagtcaacccgcagaagtgaatatgtactaccagaacacttaccagacaatgccttacggctcatcctatggcattccttatagttatacggcctatggatcatcagatgccaaatctcaaaaaacagataatacagtccctttcaaaactcccagtaatgagatgactcccgttactattgatttggtaaagaaacagcttaaagacaggttggactccatgaaagaattgcacaaaacaaatcgacagcagcatgagaaacatctgcaaagccgagtggactctaccagggctattgaaagattagaagggtcttctgggggtattggtgaacggtataaatttttgcaagaaatgcgagggtatgtccaagacttgcttgagtgtttcagtgaaaaggtgccactgattaatgaacttgaatcagcaatacatcagctgtacaaacagcgagcttcccgccttgtccaaagacgacaagatgatattaaagatgaatcttcggagttttcaagccattcaaacaaagctctgatggcaccaaatcttgactcctttggacgcgatcgggcactgtatcaagagcatgcaaaacgtcgcattgcagagcgggaggccaggaggactcgtcgtagacaagccagagaacaaaccggtaagatggcagatcaccttgaaggcctttccagtgatgatgaagaaacttctacagatattactaatttcaatctggaaaaagatcgaatttcaaaagaatccggcaaagtttttgaagatgtccttgaaagtttctattcaattgactgtattaaatcacagtttgaagcatggcgttcaaaatactacacatcctacaaagatgcttacattggcctttgtttgccaaaattattcaaccccctcatacgacttcagctcctcacttggactcctcttgaggcaaaatgtcgtgactttgagaatatgctgtggtttgaatctttgctgttttatggttgtgaagaacgagagcaagaaaaagatgatgtagatgttgccctactacctaccattgtggaaaaggtgattcttcctaaactaacagtgatagctgaaaatatgtgggaccctttttctacaacacagacttcaagaatggtgggaattacactaaaattaatcaatggatatccttcagtagtgaatgcagaaaataaaaatacacaggtatacctaaaggcacttttattgagaatgagaagaactttagatgatgatgtatttatgcccttatatcccaaaaatgtcttagaaaataaaaattctgggccttacttgttttttcaacgacagttttggtcttcagttaaggtcataaaacccccattccagagagggtcctgccccatacccagaaggaaagaatgctgctcagagaggccaagaagaatctggacggacaggccttgtgttgtcttctcttgagcagtagatcatacctttttgtccagtcatgtttctgcatgcctgtacatatttcgttaaacctaaacatagtagacaatttctcctccatctttgggtcttcattctgaaggctgtcatgttacataaaactgtgatcaaataaatttatatgccttttctcctgttaaaaaaaaaaaaaaaaaaaaaaaaaaaaaa

SEQ ID NO: 107 - DNA, 1374 bp, Homo sapiens
  leucine carboxyl methyltransferase 1 transcript variant 1 (LCMT1, LCMT), protein phosphatase methyltransferase 1 (PPMT1), protein-leucine O-methyltransferase,[phosphatase 2A protein]-leucine-carboxy methyltransferase 1, CGI-68
agccggcgtgggcggcgtcactgagccgcgccagctgagccaggtagggccctaccctcttctgttgctttctccctgtggctcgcgccgtcccccgccgcccgtcgaccccgcttccatgtccctggcggacacagctcccaggaacctccacgcccatggccactaggcagagggaatcctctatcacctcctgctgttccacctcgagctgcgacgcagacgacgagggcgtgcgcggcacctgcgaagatgcttccctgtgcaagaggtttgcagtaagcattggctactggcatgacccttacatacagcactttgtgagactgtctaaagagaggaaagcccctgaaatcaacagaggatattttgctcgagtccatggtgtcagtcagcttataaaggcatttctacggaagacagaatgtcattgtcaaattgtcaaccttggggcaggcatggataccaccttctggagattaaaggatgaagatcttctcccaagtaaatattttgaggttgactttccaatgattgtcacgagaaagctgcacagtatcaaatgcaagcctcccctatccagccccattctagaactgcattcagaggacacacttcagatggatggacacatactggattcaaagagatatgccgttattggagcagatctccgagacctgtctgaactggaagagaagctaaagaaatgtaacatgaatacacaattgccaacactcctgatagctgaatgtgtgctggtttacatgactccagagcagtccgcaaacctcctgaagtgggcagccaacagttttgagagagccatgttcataaactacgaacaggtgaacatgggtgatcggtttgggcagatcatgattgaaaacctgcggagacgccagtgtgacctggcgggagtggagacctgcaagtcattagagtcacagaaagaacggctcctgtcgaatgggtgggaaacagcatcggccgtcgacatgatggagttgtacaacaggttacctcgagctgaagtgagcaggatagaatcacttgaattcctggatgaaatggagctgctggagcagctcatgcggcattactgcctttgctgggcaaccaaaggaggaaatgagcttgggctgaaggagataacttattaatctgtcgaaggcttatgccgagccagaagccgaagccacttgccctcctggaggagacctgcaagctccctgagcggtgggcgggcctcgtccgcaggtctcatcccacactcttgagaagccttggtcactacagtggtcgcacatgttcctcttcctgttcctgttgacatgtcgttgtttaaataaatctcacttgccaccagtaaaa

SEQ ID NO: 108 - DNA, 2091 bp, Homo sapiens
  eukaryotic translation initiation factor 3, subunit L transcript variant 1 (EIF3L), eukaryotic translation initiation factor 3 subunit 6-interacting protein (EIF3S6IP), eukaryotic translation initiation factor 3 subunit E-interacting protein (EIF3EIP)
gctgaacttccggcctcaggacgcaggcgcgggccgctcatttcgctctttccggcggtgctcgcaagcgaggcagccatgtcttatcccgctgatgattatgagtctgaggcggcttatgacccctacgcttatcccagcgactatgatatgcacacaggagatccaaagcaggaccttgcttatgaacgtcagtatgaacagcaaacctatcaggtgatccctgaggtgatcaaaaacttcatccagtatttccacaaaactgtctcagatttgattgaccagaaagtgtatgagctacaggccagtcgtgtctccagtgatgtcattgaccagaaggtgtatgagatccaggacatctatgagaacagctggaccaagctgactgaaagattcttcaagaatacaccttggcccgaggctgaagccattgctccacaggttggcaatgatgctgtcttcctgattttatacaaagaattatactacaggcacatatatgccaaagtcagtgggggaccttccttggagcagaggtttgaatcctattacaactactgcaatctcttcaactacattcttaatgccgatggtcctgctccccttgaactacccaaccagtggctctgggatattatcgatgagttcatctaccagtttcagtcattcagtcagtaccgctgtaagactgccaagaagtcagaggaggagattgactttcttcgttccaatcccaaaatctggaatgttcatagtgtcctcaatgtccttcattccctggtagacaaatccaacatcaaccgacagttggaggtatacacaagcggaggtgaccctgagagtgtggctggggagtatgggcggcactccctctacaaaatgcttggttacttcagcctggtcgggcttctccgcctgcactccctgttaggagattactaccaggccatcaaggtgctggagaacatcgaactgaacaagaagagtatgtattcccgtgtgccagagtgccaggtcaccacatactattatgttgggtttgcatatttgatgatgcgtcgttaccaggatgccatccgggtcttcgccaacatcctcctctacatccagaggaccaagagcatgttccagaggaccacgtacaagtatgagatgattaacaagcagaatgagcagatgcatgcgctgctggccattgccctcacgatgtaccccatgcgtattgatgagagcattcacctccagctgcgggagaaatatggggacaagatgttgcgcatgcagaaaggtgacccacaagtctatgaagaacttttcagttactcctgccccaagttcctgtcgcctgtagtgcccaactatgataatgtgcaccccaactaccacaaagagcccttcctgcagcagctgaaggtgttttctgatgaagtacagcagcaggcccagctttcaaccatccgcagcttcctgaagctctacaccaccatgcctgtggccaagctggctggcttcctggacctcacagagcaggagttccggatccagcttcttgtcttcaaacacaagatgaagaacctcgtgtggaccagcggtatctcagccctggatggtgaatttcagtcagcctcagaggttgacttctacattgataaggacatgatccacatcgcggacaccaaggtcgccaggcgttatggggatttcttcatccgtcagatccacaaatttgaggagcttaatcgaaccctgaagaagatgggacagagaccttgatgatattcacacacattcaggaacctgttttgatgtattataggcaggaagtgtttttgctaccgtgaaacctttacctagatcagccatcagcctgtcaactcagttaacaagttaaggaccgaagtgtttcaagtggatctcagtaaaggatctttggagccagatttgtcgtctcattattgtaggagagaatttgtgggttgtggcagtaatacatttcccatgtgtcctgatgctttcaggatacatcagttgttagtgtttaaattgagttatttttattttgtgcttttgagatggagtctcactctgtct

SEQ ID NO: 109 - DNA, 6209 bp, Homo sapiens
  importin 7 (IPO7, Imp7), RAN-binding protein 7 (RANBP7)
gttctatccggggccttggcgcttctctttcctttcgcgccggttgccgctgcggagcgcggcgggtccatgtgcgcagtgagtggcgctattcctggcccagtagcacccgagccccgggtttgaccgagtccgcgctgcgatggaccccaacaccattatcgaggccctgcggggcaccatggacccagccctgcgtgaggccgcggagcgccagctcaatgaagcacacaagtctctgaattttgtctcaacactgctccagattactatgtcggaacagctggatttacctgtgagacaggcaggtgttatctatctgaaaaatatgataacacagtattggcctgatcgagaaacagcaccaggggatatatccccttatactattccagaagaagatcgccattgtattcgagaaaatattgtagaagccattatccattctcctgagctcatcagggtacagcttactacatgcattcatcacatcatcaaacatgattatccaagccgctggactgccattgtggacaaaattggcttttatcttcagtccgataacagtgcttgttggctaggaattcttctttgcctttatcagcttgtgaaaaattatgagtataaaaaaccagaggagcggagtccattggtagcagcaatgcagcattttctgccagttctaaaggatcgttttatccagcttctttctgaccagtctgatcagtctgtcctcatccagaaacagatattcaagatcttctatgctcttgttcagtatacactaccactggaactgataaaccaacagaacctgacagaatggatagaaattttaaagactgttgtgaacagggatgtacctaatgaaacacttcaagttgaagaagatgatcgacctgagttaccatggtggaaatgcaagaagtgggccttacatattttagcaagactttttgaaagatatggaagccctggcaatgtttccaaggagtataatgaatttgctgaagtatttctgaaggcatttgctgttggtgtccagcaagttttattgaaggtgttatatcagtacaaggagaagcaatatatggctcctcgagttttacaacagacattaaattatattaatcaaggagtttctcatgctctcacctggaagaatctgaagccccatatacaaggcattatccaagatgttatttttccattgatgtgctatacagatgctgatgaggaactttggcaagaagacccttacgaatatatacgcatgaagtttgatgtgtttgaagatttcatttctcctaccactgctgcccagacacttttgtttacagcctgtagtaagaggaaagaggtactgcaaaagactatgggattttgttaccagattcttacagaaccaaatgctgaccctcgaaaaaaagatggagccctgcatatgattggctctttagctgaaatacttctgaagaaaaagatctataaagatcagatggaatacatgttgcagaatcatgtattccctctcttcagcagtgaactaggctacatgagagcaagggcttgctgggtacttcactatttttgtgaagtgaagttcaaaagtgatcagaaccttcaaacagccttagagctaacaagaagatgtctgattgatgatagagaaatgcctgtgaaagtggaagctgccattgcccttcaagtattgatcagcaatcaagaaaaagctaaagaatatatcacaccattcatcagacctgtaatgcaggctcttcttcacattataagagaaacagaaaatgatgaccttaccaatgtaattcagaaaatgatctgtgaatatagtgaagaagttactcctattgcagtagaaatgacacaacatttggcaatgacatttaaccaagtaatccagacggggccagatgaagaaggtagtgatgacaaagcagttactgctatgggaattctgaatacaattgatacacttcttagtgtagttgaagatcataaagagataacccaacagcttgagggaatctgcttacaggtcattggtactgttttacaacagcatgtcttagaattctatgaggagatcttctctttagcgcacagtttgacatgtcaacaagtgtctccacagatgtggcagctactaccccttgtatttgaagtctttcagcaagatggctttgattactttacagatatgatgcccctccttcataattatgtaacagttgatacagacacacttctgtctgacaccaagtatcttgaaatgatatacagtatgtgcaaaaaggttcttacaggagttgcaggagaagatgcagagtgtcatgcagcaaaattgttagaggtcatcattctgcagtgcaaagggcgtggcattgaccagtgcattcccttattcgtggaagcagccttagaaagactgacaagagaggttaagacaagtgaacttcgaactatgtgtctgcaagttgcaattgcagctttgtattataatccacacctactactcaataccttagaaaatcttcgcttccctaataatgttgaaccagttacaaatcattttattacacagtggcttaatgatgttgactgtttcttggggcttcatgacagaaagatgtgtgttctcggactctgtgctcttattgatatggaacagataccccaagttttaaatcaggtttctggacagattttgccggcttttatccttttatttaacggattgaaaagagcatatgcctgccatgcagaacatgagaatgacagtgatgatgatgatgaagctgaagatgatgatgaaaccgaggaactggggagtgatgaagatgatattgatgaagatgggcaagaatatttggagattctggctaagcaggctggtgaagatggagatgatgaagattgggaagaagatgatgctgaagagactgctctggaaggctattccacaatcattgatgatgaagataaccctgttgatgagtatcagatatttaaagctatctttcaaactattcaaaatcgtaatcctgtgtggtatcaggcactgactcacggtcttaatgaagaacaaagaaaacagttacaggacatagcaactctggctgatcaaagaagagcagcccatgaatccaaaatgattgagaagcatggaggatacaaattcagtgctccagttgtgccaagttctttcaattttggaggcccagcaccagggatgaattgagttatctctttctttcctgctgtgtgcttgtagtgaagagcttgtgttcctcctagtagtggttccagaactggttcatgttatctattctaaactaataatcaatagatggacaaaagaaacaacaaccccaggagatgggacctgatcatgcaacctggcactggaaaagaaatcagcgggattttgggggtggggggggatgggaggtaccttagagggagtattttctttattttttgaagaaagtaagatcctgactctgaagcttcaaagtgacactgtggaaatctgaaacgaggggatgtcatgaaggcagcttttctttttctgaggaaaaaataggcatgggctacaggactatttaaaatgtctcatttacagtataaaactcaaaggtagatgtaatttttacacctatgagtatttgtccaatttctgtctcttcctcaccattgggtatctattctttatatgtaaataagataaggtcatctgatagccttattcagtcttcatcattttcatcattgttcctatgtagattattggacatttattgtagcactacataactgattataaaaatctgtaaatgaattagcactttcatattgaaacaagcctgctagcctatgtataaaatagcaaaatgtttgctgtttataaaaagatgtaatggggtggggggcaggggtaatttcaagttattaatttaaaaatgaactagcaattttgtacctggtgactttgtggtgcactcacctctgatagtgacttgaattcggtatgtaaaaaggggttagtggtatttcattgctgctaaaaatgacaactccctctgtgtcctgtttttcttaaagctgtcagtgtacaagtgggtatttgaataccagaccttactgtaaaaaataaaaaaggtggtatctagagcatgtaaattggatataaagttctgctcttaaagagttgatctaagagtatggctaaacatctatatatgcaatctattaaaagaacttaattcggctattatgtcttgatttgattgcagttttttcctaattataacaaatttttcctcattggcctgtttttaatcctgtgcctagaaggagtacaaaatgcacactttacaaaattgatatttaacacttacccactcccctttccccatctcttctaccgctcttgttgatcgtggtatctgatcttgactagataggctgaaggcacatggttccctccaaaaaccactattgataccactacaaaaacaagccagcaaaaagatactgtagagaggttggcttgcttccctctcttcctaactgcatgttgaaaaataagccgttattgatcttaaacatcggtcagatgagtcatacattgggttattttttatatacatgtatacacaaaatatttcaaattgaaagcaacatcttaatggattcaaaactattacaagctgttgtctaaaacaggtgagaaaaaaatttataactgtaaaaacaaatgcacatattgatatttaaaatgcgtaattaagaaaacccattgttgttgtgtttttcttgtataccaataattaagccactactgttggcactgtttggttttctattttaacactgaaggagtgaaagtatttcctatatttatgaatttactactaaaatcttggcaaaaaaagaaaaaaattgtctaacgtgtgtgggtgaaaactgttaatcaagtgtttctactcccccccgaaaatcccctgaaagttggacaccaactgtataccctaggttgcttaaagggatttcactattatataaagtcaataaaaatgaagtagttgtatatatgcaacattgtgtacagaggggaaataatgaatagtattaaagaaacattctcgtcttcctttacctttaatcccctaatacctagtctactttttaaattttcagacttcactgctttttgaattcataattctaattttcacattattgttaatggaaaatcatatctaataaaggttttagttattcccatgcacagtatgaaaattctcatttgctgaggttttgtttcaagaaaatgtattggcatgtctttgagaacatgttttattgtctcctgtgtcatataatccaaactaatctccgtttacagactttaacttgaaattagaccttataattaaactatttaaatagtgttcaaatgatagtttctaatgcatcaaatatatacctcagttttcatgatttcctttaacattataatttggtatagatcaagaatcttaacatgtatcagtttctagatgaggctgcaggatttttggaaaactttttgaatgtatttacaatattctcttgtaattagctacatagggacttgtctttttttctttttacatacagcttttcctacagttttattaccctgtaatttttttttagttgtagaagttaattctgattttgtgtggatttcagtatttgtctttgttaatggcacatattagcataaatcacttttgtaaatgtaagctttctttttttttcttgaaaaagcctttctatttatcagtattaaataaaggaagttaatctgtttctctgcaggtaataaaatagtgacacactgtattaagatagtgactgctatactcaactctggaagagactagagtatagagcatgagtggcaaaaccacagcccttgggccatatgctgctattcagtcccagatgtagcccctgaagcaagcataaagaaaaatgaattaaaaattaaattaatatggaaagttaaaaaatggattacattagtatgactaaaccatgtctttggcaaagatctaacacaatgtcttaagtataataggtagtctctgtttgtaaaataaatgacttaaatttaaaacatcaaaaaaaaaaaaaaaaaa

SEQ ID NO: 110 - DNA, 4004 bp, Homo sapiens
  pyruvate carboxylase transcript variant 1, mitochondrial precursor (PC, PCB), pyruvic carboxylase
gtcagtggaggcagcagcggtagaggcggcggcgaggactggcgacggcgaggagatagtgtctgccttctggagagctgaccaaacactaaggatgctgaagttccgaacagtccatgggggcctgaggctcctgggaatccgccgaacctccaccgcccccgctgcctccccaaatgtccggcgcctggagtataagcccatcaagaaagtcatggtggccaacagaggtgagattgccatccgtgtgttccgggcctgcacggagctgggcatccgcaccgtagccatctactctgagcaggacacgggccagatgcaccggcagaaagcagatgaagcctatctcatcggccgcggcctggcccccgtgcaggcctacctgcacatcccagacatcatcaaggtggccaaggagaacaacgtagatgcagtgcaccctggctacgggttcctctctgagcgagcggacttcgcccaggcctgccaggatgcaggggtccggtttattgggccaagcccagaagtggtccgcaagatgggagacaaggtggaggcccgggccatcgccattgctgcgggtgttcccgttgtccctggcacagatgcccccatcacgtccctgcatgaggcccacgagttctccaacacctacggcttccccatcatcttcaaggcggcctatgggggtggagggcgtggcatgagggtggtgcacagctacgaggagctggaggagaattacacccgggcctactcagaggctctggccgcctttgggaatggggcgctgtttgtggagaagttcatcgagaagccacggcacatcgaggtgcagatcttgggggaccagtatgggaacatcctgcacctgtacgagcgagactgctccatccagcggcggcaccagaaggtggtcgagattgcccccgccgcccacctggacccgcagcttcggactcggctcaccagcgactctgtgaaactcgctaaacaggtgggctacgagaacgcaggcaccgtggagttcctggtggacaggcacggcaagcactacttcatcgaggtcaactcccgcctgcaggtggagcacacggtcacagaggagatcaccgacgtagacctggtccatgctcagatccacgtggctgagggcaggagcctacccgacctgggcctgcggcaggagaacatccgcatcaacgggtgtgccatccagtgccgggtcaccaccgaggaccccgcgcgcagcttccagccggacaccggccgcattgaggtgttccggagcggagagggcatgggcatccgcctggataatgcttccgccttccaaggagccgtcatctcgccccactacgactccctgctggtcaaagtcattgcccacggcaaagaccaccccacggccgccaccaagatgagcagggcccttgcggagttccgcgtccgaggtgtgaagaccaacatcgccttcctgcagaatgtgctcaacaaccagcagttcctggcaggcactgtggacacccagttcatcgacgagaacccagagctgttccagctgcggcctgcacagaaccgggcccaaaagctgttgcactacctcggccatgtcatggtaaacggtccaaccaccccgattcccgtcaaggccagccccagccccacggaccccgttgtccctgcagtgcccataggcccgcccccggctggtttcagagacatcctgctgcgagaggggcctgagggctttgctcgagctgtgcggaaccacccggggctgctgctgatggacacgaccttcagggacgcccaccagtcactgctggccactcgtgtgcgcacccacgatctcaaaaagatcgccccctatgttgcccacaacttcagcaagctcttcagcatggagaactggggaggagccacgtttgacgtcgccatgcgcttcctgtatgagtgcccctggcggcggctgcaggagctccgggagctcatccccaacatccctttccagatgctgctgcggggggccaatgctgtgggctacaccaactacccagacaacgtggtcttcaagttctgtgaagtggccaaagagaatggcatggatgtcttccgtgtgtttgactccctcaactacttgcccaacatgctgctgggcatggaggcggcaggaagtgccggaggcgtggtggaggctgccatctcatacacgggcgacgtggccgaccccagccgcaccaagtactcactgcagtactacatgggcttggccgaagagctggtgcgagctggcacccacatcctgtgcatcaaggacatggccgggctgctgaagcccacggcctgcaccatgctggtcagctccctccgggaccgcttccccgacctcccactgcacatccacacccacgacacgtcaggggcaggcgtggcagccatgctggcctgtgcccaggctggagctgatgtggtggatgtggcagctgattccatgtctgggatgacttcacagcccagcatgggggccctggtggcctgtaccagagggactcccctggacacagaggtgcccatggagcgcgtgtttgactacagtgagtactgggagggggctcggggactgtacgcggccttcgactgcacggccaccatgaagtctggcaactcggacgtgtatgaaaatgagatcccagggggccagtacaccaacctgcacttccaggcccacagcatggggcttggctccaagttcaaggaggtcaagaaggcctatgtggaggccaaccagatgctgggcgatctcatcaaggtgacgccctcctccaagatcgtgggggacctggcccagtttatggtgcagaatggattgagccgggcagaggccgaagctcaggcggaagagctgtcctttccccgctccgtggtggagttcctgcagggctacatcggtgtcccccatggggggttccccgaaccctttcgctctaaggtactgaaggacctgccaagggtggaggggcggcctggagcctccctccctcccctggatctgcaggcactggagaaggagctggtagaccggcatggggaggaggtgacgccggaagatgtgctctcagcagctatgtaccccgatgtgtttgcccacttcaaggacttcactgccacctttggccccctggatagcctgaatactcgcctcttcctgcagggacccaagatcgcagaggagtttgaggtggagctggagcggggcaagacgctgcacatcaaagccctggccgtgagcgacctgaaccgggccggccagaggcaggtcttctttgagctcaatgggcagctgcggtccatcttggtcaaggacacccaggccatgaaggagatgcacttccaccccaaggccctaaaggacgtgaagggccagatcggggcgcccatgcctgggaaggtgatagacatcaaagtggtggcaggggccaaggtggccaagggccagcccctgtgtgtgctcagtgccatgaagatggagactgtggtgacctcacccatggagggtactgtccgcaaggttcatgtgaccaaggacatgacactggaaggtgacgacctcatcctggagatcgagtgatcttgccccagaccggcagcctggccatccccaagccttcaacagaagctgtgctgccacggcaggcccaggccagccagtgcccgaggccaggaaggccgggccgtggaggtcctgtccacagctggacaggagagacaccgcctgcggtggttcattcctttcagccatcgtcctttcctccggcggacagctgcttacatgttcatctcttgccaaataagggtcccctcctcactggagactacaagtggtgggtcaggtggtcctaggacccaggggaggtttaggggtcctatctcctgggggaaggggagatctaagatgtcccaggtcctgggaagtttactcaataaagctggctttcccctgc

SEQ ID NO: 111 - DNA, 894 bp, Homo sapiens
  ubiquitin-40S ribosomal protein S27a precursor, S27A, ubiquitin carboxyl extension protein 80 (CEP80, UBCEP80), ubiquitin C (UBC), epididymis luminal protein 112 (HEL112), UBCEP1
ggcgttcttccttttcgatccgccatctgcggtgggtgtctgcacttcggctgctctcgggttagcaccctatggtgccttctcttgtgatccctgacctaacctgtctcttccttttcctcaacctcaggtggagccgccaccaaaatgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaactgtatgagttaataaaagacatgaactaacatttattgttgggttttattgcagtaaaaagaatggtttttaagcaccaaattgatggtcacaccatttccttttagtagtgctactgctatcgctgtgtgaatgttgcctctggggattatgtgacccagtggttctgtatacctgccaggtgccaaccacttgtaaaggtcttgatattttcaattcttagactacctatactttggcagaagttatatttaatgtaagttgtctaaatataa

SEQ ID NO: 112 - DNA, 2282 bp, Homo sapiens
  secreted and transmembrane protein 1 precursor (SECTM1), type 1a transmembrane protein, K12
aaacagccccgcggggaacgcgcgccgcacgcgagctggacccgcccaggcacgaactctcctggaaaaatgctcccggcggctttcctgcttcctttagcgtgaaccgcgggtgcggtgcctcccgtgaaaataataaattcaccgtcacgcttgttgtgaacgcgggtggttcccgaaacttggaggcttcccgtaaacccagctccttcctcatctgggaggtgggtcccgcgcgggtccgccgcctcctccctggcccctccctctcgtgtctttcattttcctggggctccggggcgcggagaagctgcatcccagaggagcgcgtccaggagcggacccgggagtgtttcaagagccagtgacaaggaccaggggcccaagtcccaccagccatgcagacctgccccctggcattccctggccacgtttcccaggcccttgggaccctcctgtttttggctgcctccttgagtgctcagaatgaaggctgggacagccccatctgcacagagggggtagtctctgtgtcttggggcgagaacaccgtcatgtcctgcaacatctccaacgccttctcccatgtcaacatcaagctgcgtgcccacgggcaggagagcgccatcttcaatgaggtggctccaggctacttctcccgggacggctggcagctccaggttcagggaggcgtggcacagctggtgatcaaaggcgcccgggactcccatgctgggctgtacatgtggcacctcgtgggacaccagagaaataacagacaagtcacgctggaggtttcaggtgcagaaccccagtccgcccccgacactgggttctggcctgtgccagcggtggtcactgctgtcttcatcctcttggtcgctctggtcatgttcgcctggtacaggtgccgctgttcccagcaacgccgggagaagaagttcttcctcctagaaccccagatgaaggtcgcagccctcagagcgggagcccagcagggcctgagcagagcctccgctgaactgtggaccccagactccgagcccaccccaaggccgctggcactggtgttcaaaccctcaccacttggagccctggagctgctgtccccccaacccttgtttccatatgccgcagacccatagccgcctgcaaggcagagaggacacaggagagccagccctgagtgccgaccttgggtggcggggcctgggtctctcgtcccacccggagggcacagacaccggcttgcttggcaggctgggcctctgtgtcacccactcctgggtgcgtgcagacccttcccctccaccccccaggtcttccaagctctgcttcctcagtttccaaaatggaaccacctcacctccgcagcacccgacttaccaggacgcatgcccctccctctgccctcatcaaacccacagacccggactccctttctgccaccccaggctggtccggccccaggtgtggggtccgctctctccactcccagggctccgcgcccaagtgagggggcccctgccggagcctcagacacactccagttcagggctgtggggggccttggccacatacctgtcccttggctatgagcaggctttgggggcccttccgcggcagccccgggggccgaggtagggtcgggggcttagaggctgggatggctcctggccccaccgccagggggcagcgcaggccgggctgggaggcggcggcggcggctcgggctggggggtcaggtggacgccgccctccggggctggacgcgcatccctcagtccctcggccacccgggggtcgctccctcgtgcccaccgcacctgccgagcctctttggacccagatctgttcatgcttttgtcttcgtcactgcggcggggccctttgatgtcttcatctgtatggggtggaaaaatcaccgggaatcccccttcagttctttgaaaaagttccatgactcgaatatctgaaatgaagaaaacaaaccgactcacaaacctccaagtagctccaaatgcaatttttaaaatggaaaacaaaaatctgaaagaaacgtctttagtggctttaagccccaaaacgtccctaaggcgtcctcgagatgaagacgggggggagcccccagccaggtggagaccccgcagggacgcggcggcgcccggtgaccgaggcctcgcacagccggccgccctgagggtcgggccggagccagggtccaagaggggcgcgtttgtgtctcgggttaaaataaggttccgtccgcgtgctgggtcaga

SEQ ID NO: 113 - DNA, 593 bp, Homo sapiens
  60S ribosomal protein L41 transcript variant 2, L41, homologue of yeast ribosomal protein YL41, HG12 protein
acccggcgctccattaaatagccgtagacggaacttcgcctttctctcggccttagcgccatttttttgggtgagtgttttttggttcctgcgttgggattccgtgtacaatccatagacatctgacctcggcacttagcatcatcacagcaaactaactgtagcctttctctctttccctgtagaaacctctgcgccatgagagccaagtggaggaagaagcgaatgcgcaggctgaagcgcaaaagaagaaagatgaggcagaggtccaagtaaaccgctagcttgttgcaccgtggaggccacaggagcagaaacatggaatgccagacgctggggatgctggtacaagttgtgggactgcatgctactgtctagagcttgtctcaatggatctagaacttcatcgccctctgatcgccgatcacctctgagacccaccttgctcataaacaaaatgcccatgttggtcctctgccctggacctgtgacattctggactatttctgtgtttatttgtggccgagtgtaacaaccatataataaatcacctcttccgctgttttagctgaagaattaaatcaaaaaaaaaa

SEQ ID NO: 114 - DNA, 5675 bp, Homo sapiens
  tuberous sclerosis 2 transcript variant 1 (TSC2), tuberin, LAM, TSC4
ccggcggcgtcccggggccaggggggtgcgcctttctccgcgtcggggcggcccggagcgcggtggcgcggcgcgggaggggttttctggtgcgtcctggtccaccatggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgactacatggagccagagagaggctctgagaagaagaccagcggccccctttctcctcccacagggcctcctggcccggcgcctgcaggccccgccgtgcggctggggtccgtgccctactccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtcttttgatgacacccccgagaaggacagcttcagggcccggagtactagtctcaacgagagacccaagagtctgaggatagccagaccccccaaacaaggcttgaataactctccacccgtgaaagaattcaaggagagctctgcagccgaggccttccggtgccgcagcatcagtgtgtctgaacatgtggtccgcagcaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtgtgaggccggggccctccctcctgcactggccttggacggtattgcctgtcagtgaaataaataaagtcctgaccccagtgcacagacatagaggcacagattgcaaaaaaaaaaaaaaaaaaaaaaaaaaaaaaaaaaaaaaaaaaaa

SEQ ID NO: 115 - DNA, 5436 bp, Homo sapiens
  collagen, type XVIII, alpha 1 transcript variant 3 (COL18A1), collagen alpha-1(XVIII) chain, endostatin, antiangiogenic agent, multi-functional protein MFP, KNO, KNO1, KS
ctcccagcggcggcggctgcagcgcggcggtccgagcgggtgcaccgcggcggaggaggcagcatcccgcggcgctgacggtcctggggagagcatggcgccgaggtgcccctggccatggccgcggcggcggcgcctcctggacgtgctcgcgcccctggtcctgctgctcggggtccgcgcggcctccgcggagccagagcgcatcagcgaggaggtggggctgctgcagctccttggggaccccccgccccagcaggtcacccagacggatgaccccgacgtcgggctggcctacgtctttgggccagatgccaacagtggccaagtggcccggtaccacttccccagcctcttcttccgtgacttctcactgctgttccacatccggccagccacagagggcccaggggtgctgttcgccatcacggactcggcgcaggccatggtcttgctgggcgtgaagctctctggggtgcaggacgggcaccaggacatctccctgctctacacagaaccaggtgcaggccagacccacacagccgccagcttccggctccccgccttcgtcggccagtggacacacttagccctcagtgtggcaggtggctttgtggccctctacgtggactgtgaggagttccagagaatgccgcttgctcggtcctcacggggcctggagctggagcctggcgccgggctcttcgtggctcaggcggggggagcggaccctgacaagttccagggggtgatcgctgagctgaaggtgcgcagggacccccaggtgagccccatgcactgcctggacgaggaaggcgatgactcagatggggcatccggagactctggcagcgggctcggggacgcccgggagcttctcagggaggagacgggcgcggccctaaaacccaggctccccgcgccaccccccgtcaccacgccacccttggctggaggcagcagcacggaagattccagaagtgaagaagtcgaggagcagaccacggtggcttcgttaggagctcagacacttcctggctcagattctgtctccacgtgggacgggagtgtccggacccctgggggccgcgtgaaagagggcggcctgaaggggcagaaaggggagccaggtgttccgggcccacctggccgggcaggccccccaggatccccatgcctacctggtcccccgggtctcccgtgcccagtgagtcccctgggtcctgcaggcccagcgttgcaaactgtccccggaccacaaggacccccagggcctccggggagggacggcacccctggaagggacggcgagccgggcgaccccggtgaagacggaaagccgggcgacaccgggccacaaggcttccccgggactccaggggacgtaggtcccaagggcgacaagggagaccctggggttggagagagagggcccccaggaccccaagggcctccagggcccccaggaccctccttcagacacgacaagctgaccttcattgacatggagggatctggcttcgggggcgatctggaggccctgcggggtcctcgaggcttccctggacctcccggaccccccggtgtcccaggcctgcccggcgagccaggccgctttggggtgaacagctccgacgtcccaggacccgccggccttcctggtgtgcctgggcgcgagggtccccccgggtttcctggcctcccgggacccccaggccctccgggaagagaggggcccccaggaaggactgggcagaaaggcagcctgggtgaagcaggcgccccaggacataaggggagcaagggagcccccggtcctgctggtgctcgtggggagagcggcctggcaggagcccccggacctgctggaccaccaggcccccctgggccccctgggcccccaggaccaggactccccgctggatttgatgacatggaaggctccggggggcccttctggtcaacagcccgaagcgctgatgggccacagggacctcccggcctgccgggacttaagggggatcctggcgtgcctgggctgccgggggcgaagggagaagttggagcagatggagtccccgggttccccggcctccctggcagagagggcattgctgggccccaggggccaaagggagacagaggcagccggggagaaaagggagatccagggaaggacggagtcgggcagccgggcctccctggcccccccggacccccgggacctgtggtctacgtgtcggagcaggacggatccgtcctgagcgtgccgggacctgagggccggccgggtttcgcaggctttcccggacctgcaggacccaagggcaacctgggctctaagggcgaacgaggctccccgggacccaagggtgagaagggtgaaccgggcagcatcttcagccccgacggcggtgccctgggccctgcccagaaaggagccaagggagagccgggcttccgaggacccccgggtccatacggacggccggggtacaagggagagattggctttcctggacggccgggtcgccccgggatgaacggattgaaaggagagaaaggggagccgggagatgccagccttggatttggcatgaggggaatgcccggccccccaggacctccagggcccccaggccctccagggactcctgtttacgacagcaatgtgtttgctgagtccagccgccccgggcctccaggattgccagggaatcagggccctccaggacccaagggcgccaaaggagaagtgggcccccccggaccaccagggcagtttccgtttgactttcttcagttggaggctgaaatgaagggggagaagggagaccgaggtgatgcaggacagaaaggcgaaaggggggagcccgggggcggcggtttcttcggctccagcctgcccggcccccccggccccccaggccccccaggcccacgtggctaccctgggattccaggtcccaagggagagagcatccggggccagcccggcccacctggacctcagggaccccccggcatcggctacgaggggcgccagggccctcccggccccccaggccccccagggcccccttcatttcctggccctcacaggcagactatcagcgttcccggccctccgggcccccctgggccccctgggccccctggaaccatgggcgcctcctcaggggtgaggctctgggctacacgccaggccatgctgggccaggtgcacgaggttcccgagggctggctcatcttcgtggccgagcaggaggagctctacgtccgcgtgcagaacgggttccggaaggtccagctggaggcccggacaccactcccacgagggacggacaatgaagtggccgccttgcagccccccgtggtgcagctgcacgacagcaacccctacccgcggcgggagcacccccaccccaccgcgcggccctggcgggcagatgacatcctggccagcccccctcgcctgcccgagccccagccctaccccggagccccgcaccacagctcctacgtgcacctgcggccggcgcgacccacaagcccacccgcccacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtagccaccgcctggatgcggatggccggagaggaccggcggctcggaggaagcccccaccgtgggcagggagcggccggccagcccctggccccaggacctggctgccatactttcctgtatagttcacgtttcatgtaatcctcaagaaataaaaggaagccaaagagtgtatttttttaaaagtttaaaacagaagcctgatgctgacattcacctgccccaactctcccctgacctgtgagcccagctgggtcaggcagggtgcagtatcatgccctgtgcaacctcttggcctgatcagaccacggctcgatttctccaggatttcctgctttgggaagccgtgctcgccccagcaggtgctgacttcatctcccacctagcagcaccgttctgtgcacaaaacccagacctgttagcagacaggccccgtgaggcaatgggagctgaggccacactcagcacaaggccatctgggctcctccagggtgtgtgctcgccctgcggtagatgggagggaggctcaggtccctggggctagggggagccccttctgctcagctctgggccattctccacagcaaccccaggctgaagcaggttcccaagctcagaggcgcactgtgacccccagctccggcctgtcctccaacaccaagcacagcagcctggggctggcctcccaaatgagccatgagatgatacatccaaagcagacagctccaccctggccgagtccaagctgggagattcaagggacccatgagttggggtctggcagcctcccatccagggcccccatctcatgcccctggctgggacgtggctcagccagcacttgtccagctgagcgccaggatggaacacggccacatcaaagaggctgaggctggcacaggacatgcggtagccagcacacagggcagtgagggagggctgtcatctgtgcactgcccatggacaggctggctccagatgcagggcagtcattggctgtctcctaggaaacccatatccttaccctccttgggactgaaggggaaccccggggtgcccacaggccgccctgcgggtgaacaaagcagccacgaggtgcaacaaggtcctctgtcagtcacagccacccctgagatccggcaacatcaacccgagtcattcgttctgtggagggacaagtggactcagggcagcgccaggctgaccacagcacagccaacacgcacctgcctcaggactgcgacgaaaccggtggggctggttctgtaattgtgtgtgatgtgaagccaattcagacaggcaaataaaagtgaccttttacactga

SEQ ID NO: 116 - DNA, 3230 bp, Homo sapiens
  calcium binding protein P22 (CHP, CHP1, p22), calcineurin-like EF-hand protein 1, EF-hand calcium-binding domain-containing protein p22, calcineurin B-like protein, SLC9A1 binding protein (SLC9A1BP), Sid470p
accacccctgggttccctcccgggtccgcagtggaaacactgccctctcccttcttgacccctagcccttccttccctccctccttccctcctgtcgccgtctcttctggcgccgctgctcccggaggagctcccggcacggcgatgggttctcgggcctccacgttactgcgggacgaagagctcgaggagatcaagaaggagaccggcttttcccacagtcaaatcactcgcctctacagccggttcaccagcctggacaaaggagagaatgggactctcagccgggaagatttccagaggattccagaacttgccatcaacccactgggggaccggatcatcaatgccttctttccagagggagaggaccaggtaaacttccgtggattcatgcgaactttggctcatttccgccccattgaggataatgaaaagagcaaagatgtgaatggacccgaaccactcaacagccgaagcaacaaactgcactttgcttttcgactatatgatttggataaagatgaaaagatctcccgtgatgagctgttacaggtgctacgcatgatggtcggagtaaatatctcagatgagcagctgggcagcatcgcagacaggaccattcaggaggctgatcaggatggggacagtgccatatctttcacagaatttgttaaggttttggagaaggtggatgtagaacagaaaatgagcatccgatttcttcactaaaggagaccaaactgttccttgcggtctagtatttaagaactggaacttgaaagtcctccttctaccaactccacctccaccccctcattccccttctcccaaagtactactgctgttgcatgacaaccccaaatatgttctgtcaacacaaacctgcctttggtgtataaacagggcattacagaatggtacaccctatatatttctgttcagtatccattcactagttcttcatttataaatatcatcttccccattctgctgctgaatgccacacatccatccagtctgagaaagtgagagaggcaatcatgccaagaacaagccagcaaagctctttcaccagatgtagactgtagccctgctgccttccctccagcgagtctgccagcatgcttcttcatcctttttatatgttctttgcttcctacttccctgtcttccaacatactgttcacttactctggcagtctttctgcttttcattaagcctcaaaatctcctctgttctacttggcaccacaagctatgtcctatatatgtatttctgacttggcaggatagttcaggggtctggcagtttttatttaccttcattattaaatgggcctctgggatgttgcctcttcaggagctttttggtaatcaatacttctctcagaagtatgagaccatcctctgcactctgctctgtcatcaaaggctgctgggtggagataccctttttgaaaggtggccttggtgagaggtatggagccaagtcttctaggttgcttgcccacatcactctatctctggcctctgattctcaactttgtacctgtgtggctcctcttgttagtgcaatgttgactgttgaaaaagcagcagtatgcttacaggtttgcttagtttggggacaccgttaccaccagaatggctgctctgacaatatgcctagggactttctcatggcttttatttaataaggaggctgggcaccctataaagcctcatgcattcacacctttgcagcatggtttatgcctcagtgttatgtgcactggaatgttttccacttcacatttccaagtagaaatattagtgttacggaagtgcctaatatcccagtccaaatttttttttttttttttttttttttttgagacagagtcttgctctgtcacccaggctggagtgcagtggtgcgatcgctcactgcaacctcagcctcctggatttaagtgattctcctgcctcagcctcccaagtagctgggattacaggtgtgcaccaccatgcccggctaattttttgtatttttagtggagacagggtttcaccatgttggccaggctggtctcgaactcctgacctcgtgatccgcctgcctcagcctcccaaagtgctgggattacaggtgtgagccaccacgcctggccccagtccaaaatatttaaagattgtttccttagtgtcttgaagttttgcacaaaattcttttttttgagatggagtctcactctgtcacccaggctggagtgcagtggcgtgatcttggctcactgcaacctctgcctcctgggttcaagcaattctcccacctcagcctcccaagtagctgggattacagacgtgtgccaccatacctgggtaatttttgcatttttagtggagagggagtttcaccatgttggccaggttggtcttgaactcctgacctcaggtgatcctcctgcctcggcctcccaaagtgctgggattacaggcatgagccaccgtgctcagccgcaaaattctttatgaattttacacttggcaaatgttaatgacggaagccatagtctgctcctaatacatgtccaaagcattgactgttgtgtcattagctgcctggttacattagctccctggcttcttgtttagaccactgctaatcccttaaaaacaagaggtctggcactagtagcacaacctaaggtggcattacagatctttgagcgagccacagcaacttttctgccaagtcagcttagtttagacttcagtgaatcaggctattgctatcctaatgtatgtctctatgagtgtatttagccacacatctgcccttggttgactttctgactcattgcttgcttgcttgtttccttgctttggaaaactattgaagattgctaaaaaataccactgcaaagtgatggaaaagggtggagaacaggggagtagccaggctggatggctcaaatataaatgaatgaggaattctttatgaagtatcagtcagattttatgattaagtgatgtaatataggaattatgtaaaagggaagaatgtctgatactgatctattagagaggtactttagaggcttcttgattggcataaagttcctaaggttatagattttccccccttttggctgtatagcaaagtgttttaatccacggttgtgccttattgttccattaaaattgtatcttcgatccatcaataaatacttgtggttgaaacaaaa

SEQ ID NO: 117 - DNA, 4511 bp, Homo sapiens
  phosphofurin acidic cluster sorting protein 1 (PACS1), cytosolic sorting protein PACS-1, MRD17
gcagctcgctggctgctcgcgctcgggcaggcgggctgaggaggctgccgcgcccccgccgccgccgccgcgggggaagcctgggagccagatcggcgtcgcctcggcctccgtaacccccgcctagccgggccatggcggaacgcggaggggcgggcggtggtcccggaggcgccgggggcggcagcggccagcggggatccggggtcgcccagtcccctcagcagccgccgccgcagcagcagcagcagcagccgccgcagcagccgacgccccccaagctggcccaggccacctcgtcgtcctcgtccacctcggcggcggctgcctcctcctcgtcctcgtctacctccacctccatggccgtggcggtggcctcgggctccgcgcctcccggtggcccggggccaggccgcacccccgccccggtgcagatgaacctgtacgccacctgggaggtggaccggagctcgtccagctgcgtgcctaggctattcagcttgaccctgaagaaactcgtcatgctaaaagaaatggacaaagatcttaactcagtggtcatcgctgtgaagctgcagggttcaaaaagaattcttcgctccaacgagatcgtccttccagctagtggactggtggaaacagagctccaattaaccttctcccttcagtaccctcatttccttaagcgagatgccaacaagctgcagatcatgctgcaaaggagaaaacgttacaagaatcggaccatcttgggctataagaccttggccgtgggactcatcaacatggcagaggtgatgcagcatcctaatgaaggcgcactggtgcttggcctacacagcaacgtgaaggatgtctctgtgcctgtggcagaaataaagatctactccctgtccagccaacccattgaccatgaaggaatcaaatccaagctttctgatcgttctcctgatattgacaattattctgaggaagaggaagagagtttctcatcagaacaggaaggcagtgatgatccattgcatgggcaggacttgttctacgaagacgaagatctccggaaagtgaagaagacccggaggaaactaacctcaacctctgccatcacaaggcaacctaacatcaaacagaagtttgtggccctcctgaagcggtttaaagtttcagatgaggtgggctttgggctggagcatgtgtcccgcgagcagatccgggaagtggaagaggacttggatgaattgtatgacagtctggagatgtacaaccccagcgacagtggccctgagatggaggagacagaaagcatcctcagcacgccaaagcccaagctcaagcctttctttgaggggatgtcgcagtccagctcccagacggagattggcagcctcaacagcaaaggcagcctcggaaaagacaccaccagccctatggaattggctgctctagaaaaaattaaatctacttggattaaaaaccaagatgacagcttgactgaaacagacactctggaaatcactgaccaggacatgtttggagatgccagcacgagtctggttgtgccggagaaagtcaaaactcccatgaagtccagtaaaacggatctccagggctctgcctcccccagcaaagtggagggggtgcacacaccccggcagaagaggagcacgcccctgaaggagcggcagctctccaagcccctaagtgagaggaccaacagttccgacagcgagcgctccccagatctgggccacagcacgcagattccaagaaaggtggtgtatgaccagctcaatcagatcctggtgtcagatgcagccctcccagaaaatgtcattctggtgaacaccactgactggcagggccagtatgtggctgagctgctccaggaccagcggaagcctgtggtgtgcacctgctccaccgtggaggtccaggccgtgctgtccgccctgctcacccggatccagcgctactgcaactgcaactcttccatgccgaggccagtgaaggtggctgctgtgggaggccagagctacctgagctccatcctcaggttctttgtcaagtccctggccaacaagacctccgactggcttggctacatgcgcttcctcatcatccccctcggttctcaccctgtggccaaatacttggggtcagtcgacagtaaatacagtagttccttcctggattctggttggagagatctgttcagtcgctcggagccaccagtgtcagagcaactggacgtggcagggcgggtgatgcagtacgtcaacggggcagccacgacacaccagcttcccgtggccgaagccatgctgacttgccggcataagttccctgatgaagactcctatcagaagtttattcccttcattggcgtggtgaaggtgggtctggttgaagactctccctccacagcaggcgatggggacgattctcctgtggtcagccttactgtgccctccacatcaccaccctccagctcgggcctgagccgagacgccacggccacccctccctcctccccatctatgagcagcgccctggccatcgtggggagccctaatagcccatatggggacgtgattggcctccaggtggactactggctgggccaccccggggagcggaggagggaaggcgacaagagggacgccagctcgaagaacaccctcaagagtgtcttccgctcagtgcaggtgtcccgcctgccccatagtggggaggcccagctttctggcaccatggccatgactgtggtcaccaaagaaaagaacaagaaagttcccaccatcttcctgagcaagaaaccccgagaaaaggaggtggattctaagagccaggtcattgaaggcatcagccgcctcatctgctcagccaagcagcagcagactatgctgagagtgtccatcgatggggtcgagtggagtgacatcaagttcttccagctggcagcccagtggcccacccatgtcaagcactttccagtgggactcttcagtggcagcaaggccacctgaggccctgtctcccagccactttccctcctggcactgccaccagcctcaccgcctgcgggcagggggaggccagcaggcccgggcccagcaccccttccctggcaccagggtctgcctctcactcgcccaggtcccgaaggacactgccacagggacgccttccctcccctcccctccagcccacccctgcacagcccctcctccttcccgcttttccccttctccctcctgctccaggcccaaggcgtgttggttttgccttctggtgcccatagtcccctggactgagtcccccaggccttccttcacccgacttccaaactcttccttgtggtatcagtttccttctcggaaatgagaaagctggaatcctggtccccagcaggagagcctagtcctcccccagcccctccagccaccagggtgtcctctaggatgcagctgccagatccactcactctgctgcctccagcaggacccaaggccactttcaactcttatggggttctccacctgccccagagcttctcaagggagggtaagggggcaccctgagcccacaggacccctacttcacagctcacaggggcaggaggcagctcccctgcctccaggaccctgttgctatggtgacacagcgtttctaggacagaggggcctcccagtctccccccaccacccgtgcacgacttcctcaccacccccaggttccctgcagatgtcgtgtgtgtcctgagtgtttctttggttctttgcacgccaagtctcttggttgtaccatgtgacacaccctgtgcactggtcgctgtcttcgtggcttccacccttgttaatgatgctcctgcctctgcctcccagcccctcacccagcacagctctgcctggacttggagagatgggaggcagacccccaccaccatacatgctgtctgtggcccctcagacattctgtttcatctcccattcatctccctcctcccaccgtgtcagtttttctgcctttccctgctctgttcttccccctccttaggccccagcctgggcccagacccatcctcccagccaggtttccctccagcaggctccttccctccctgtcacctccctctcaccaacccggggtctgagcccctcattcctgaccgtccgtgttctcaggagtggttgaggacacagggccccagcccagccctctgcaccccccagcccggccatctgcgccccacagcccctttggagcttttctcttgtcctctcactccttcccagaagtttttgcacagaacttcattttgaaagtgtttttctcattctccatacctcccccaagctctcctccagcccttcccagggctcagccctgctgtcctgagcgtctcctgggccagagagaggagatgggggtgggagggactgagttgatgttgggtttttcattcaataaattggtgatttcttaccgactgcaaaaaaaaaaaaaaaaaa

SEQ ID NO: 118 - DNA, 3585 bp, Homo sapiens
  general transcription factor IIIB (TF3B90, TFIIIB90), BRF1, RNA polymerase III transcription initiation factor 90 kDa subunit transcript variant 3, TATA box binding protein (TBP)-associated factor, RNA polymerase III, GTF3B subunit 2
gtggcatggggcactggctttgctgatgaggagaccagacttcagaggccgtgtcacctggcctgggccgcacagctggtgcatgccaagctcgcgtgcagacgaagctccagaggcctggctgtcaggagggttcctccccgaggacacacacgcaacctcctggaccgccaggctttagacttttaaaacttccatatgcaggaaaccccacaggacgcagccagcatctcgggtcatgggtgcacctgtcaccgcagtcctgggctaaacagtgaacaggagctctgtgtctctgtggtcgttacagttgtagtgaatgttccctgggagagtcctcttcagaagatatttgcggtattttcaacaaaggactctccaggatggaggagcaactacaagtcaggaaggaaaaggctgacaacccagttttttaaaaaacgggcaaaggacgtgaacagacaccatgcaatggcccagagtgcacaaatcatcatttctcccaggaatggggtgcccgagcccccaggcggctgaacttgagacttgtcacgtagtgttggtggaatgcagagcagctggagatcacctgctgcaggtgggtgcgtaactccgcacggccaagccgtttggaaagctgtagcatatgcatgagagtccggaaagttccgagagaaagtgtgtggatgcacccgccaaggacacacccaccgaggccacagctgcgatttcatagtgtgggaccccactggcaaagacacgctggctgctgtggccgtggtttctgtcccagccgtctgtttccatagaaaaagacccgtgcctgtatattccacgctttgcgcacctgctggaattcggggagaagaaccacgaggtgtccatgactgccctgaggctcctacagaggatgaagcgggactggatgcacacaggccggcgcccctcgggcctctgcggagcagcgctcctggttgcagccagaatgcatgacttcaggaggactgtgaaggaggtcatcagtgtggtcaaagtgtgtgagtccacgctgcggaagaggctcacggaatttgaagacacccccaccagtcagttgaccattgatgagttcatgaagatcgacctggaggaggagtgcgaccccccctcgtacacagctgggcagaggaagctgcggatgaagcagcttgaacaagtcctgtcaaaaaaactggaggaggttgaaggtgaaatatccagttaccaggatgcaattgagattgaactagaaaacagccggccaaaggccaaggggggcctggccagcctggcaaaagatggctccaccgaggacaccgcgtccagcttgtgtggcgaggaggacacagaggacgaggagctggaagccgcggccagccacctgaacaaagacttataccgggagctccttggtggtgcccccggcagctcggaagcagcaggaagccccgagtggggcggcagacctccggccctggggtccctgctggaccccctccccactgcagccagcctgggcatctcagactccatccgcgaatgcatctcctctcagagcagcgaccccaaagatgcttcaggagacggtgagctggacctcagtggcattgatgacctggagattgacaggtacatcctgaatgagtcggaagcccgcgtgaaggccgagctgtggatgagggagaacgccgagtacctgcgggaacagagggaaaaagaagcaagaatagcgaaagagaaggagctcggcatctacaaggaacacaagcccaagaagtcttgcaagcgacgggagccaattcaggccagtaccgccagggaggccatcgagaagatgctggagcagaagaagatctccagcaagatcaattatagcgtgctccggggcctcagcagcgccggcgggggcagtccgcacagggaggatgcacagcccgagcatagcgccagtgccaggaagctgtcacgaaggaggacgccggccagcagaagtggggctgaccctgtgaccagtgtggggaaaaggttgaggcctctggtgtctacgcagccagcaaagaaggtggccacgggagaggctttgctcccaagctctcccaccctcggagctgagcctgccaggccccaggcggtgctggtggagagcgggcccgtgtcataccacgccgacgaggaggctgacgaggaggagcctgacgaggaggacggggagccctgcgtcagtgccctgcagatgatgggcagcaacgactatggctgtgatggcgatgaggacgacggctactgaagtgtggcctccaggcaggtgatgtcctggcagggggcctcgcgggtctcctcagcatcagacgggcttccaggaccgcagcaggcaggccccagcgccgagactcctggtgacaggtggcacctgtcccacagccctcgtcccatgtggaacttaccattgggattgtgtttctattcagcaagggaaaccggaccaagcgtctgcatgtgtgtgatcagatgtgggccgggtgtgtgcagggctgggtcccgctgcctgccgtcgactcatccaaggaccctccaaggctggcagtgtggtgttgctactattaaggaaacaggcttggggcagccccactgctggtccaagtgtgtggagggctgagtgtgctggccctgtgactcaggaccagctctggagtctccagcccaccctccgcaccgtcccctcctgagcagcactcggcgccagcagcctctgccagagtggaagccagagccctgcaggtgtccggcgcagccgtgggagctgaggatctggcacttgagaggcagcagctccttgaaggtcctctgcctccagctgtggccctgcatccagatacctgcctcgtccgaggcagacacccccacccctgcctcctccagacccccctccccgctgcctgcaccgcctggagcagcatgggggtcagacccctgctccagggccacttgagttgtgggcccaggagccctgcggctgccggcaggtgaactgagtgcccgacagctgagaccggcgcccacccgtcctgagcatagctctgtaggcagtgcgggcatagcctgcatagtgtcctggcgctgggagttccccgtggacagagccagagggcagtggcgctccctgtcagagctggatcaggccccccatcgaggagggagggcagacggaggcccgagagcctccccaggcctcttcgtgggaaggccccagtaccactcgtaggaggtctcagctctggcatggctgccccggatgtggccgagggggcttcaccctgtgtccttaggagggggtggccttgaggcagagccgtgcctcactgacccccaggggcctcatcctccccatggaatgggctgtatgtcctgccccaacttggcccgcagcaggccagacccccctacccccgcccagagctcagtagccagcctgggtcctgccagggcttctcgagggcttgggggaagaatagatttagtaaagcaggaagatctgttgttacttaacagaaaaaaaaaaaaaaaaaa

SEQ ID NO: 119 - DNA, 2355 bp, Homo sapiens
  prostaglandin E synthase 2 transcript variant 1 (PTGES2), chromosome 9 open reading frame 15 (C9orf15), GATE-binding factor 1 (GBF-1, GBF1), gamma-interferon-activated transcriptional element-binding factor 1
ctctcgggcgccgggcggggccatcccggggctgtccgcggagacgcctatgcggtggaggctccgggcttcagctagggcgggggagcccagcagaaggaccgagcagcctggcaccccactttgccatcctctccctggaaatctcggggtcggcgggccggccgcttcgcgtgggcgaaatcagagacacgtggtttccaaggccccttcgggttcgggaaaattttatggttcgggtcacagtaggaagcggacaatgaggcgggagggcagagagaaccgcaacacctggtgccgggtcgggtcgtttccggggctttcagtggccggaagtcgcggcgcctgtactgactctaggaagggctggagttgttttgaatgggcgcccgtaagagaggtgggcaagtacgtgttacagacggccacgccgccctttaggcggtcaaggtggggcgaggagacgttcgcccccctgcagtcggccgggtcactacccaagagcctttggaggcggaagcatggaacggtctgcaaacgttcccgagcgggcctctgcggctctggcgggcgtttcgaacttgggcgccgggcacacgcccagtcccgagagcgctgagggttcccttagcgtcgccctcaccccggccaacccgcggggcgccagagtcctggccctttaaacgccgcgcgtgcctcggcgtcttcgtttcgcgcgcccgcccgcggcgccggcggagcgaacatggacccggctgcgcgggtggtgcgggcgctgtggcctggtgggtgcgccttggcctggaggctgggaggccgcccccagccgctgctacccacgcagagccgggctggcttcgcgggggcggcgggcggcccgagccccgtggctgcagctcgtaaggggagcccgcggctgctgggagctgcggcgctggccctggggggagccctggggctgtaccacacggcgcggtggcacctgcgcgcccaggacctccacgcagagcgctcagccgcgcagctctccctgtccagccgcctgcagctgaccctgtaccagtacaagacgtgtcccttctgcagcaaggtccgagccttcctcgacttccatgccctgccctaccaggtggtggaggtgaaccctgtgcgcagggctgagatcaagttctcctcctacagaaaggtgcccatcctggtggcccaggaaggagaaagctcgcaacaactaaatgactcctctgtcatcatcagcgccctcaagacctacctggtgtcggggcagcccctggaagagatcatcacctactacccagccatgaaggctgtgaacgagcagggcaaggaggtgaccgagttcggcaataagtactggctcatgctcaacgagaaggaggcccagcaagtgtatggtgggaaggaggccaggacggaggagatgaagtggcggcagtgggcggacgactggctggtgcacctgatctcccccaatgtgtaccgcacgcccaccgaggctctggcgtcctttgactacattgtccgcgagggcaagttcggagccgtggagggtgccgtggccaagtacatgggtgcagcggccatgtacctcatcagcaagcgactcaagagcaggcaccgcctccaggacaacgtgcgcgaggacctctatgaggctgctgacaagtgggtggctgctgtgggcaaggaccggcccttcatggggggccagaagccgaatctcgctgatttggcggtgtatggcgtgctgcgtgtgatggaggggctggatgcgttcgatgacctgatgcagcacacgcacatccagccctggtacctgcgggtggagagggccatcaccgaggcctccccagcgcactgaatgtccccgcgcagagcagagggaaggcagcggaagacgccagctgccagggcctggggccactgggccagcgcctggcgatactggttgggggcaggatcattctgccccttgtccacgcacccccaccagccctctcgcttctaacacagggcacctgctggggctcagggatgttagggacgagttccagccctgccactgccctggggcgacccctccctgtccctgcctccctgctctgccgcccctcttcctggaccctcagtggctgtcccatggctacatcctgtgggtgggggccctcgacaggacagcaggacggtttgttttcagtggaatcccatccctgggttcccctggttcccactcttcccaagcctcccgggactgggacatgtttgcaataaaggaaaggtttgtggcgcctgtcatggcaggcatctcatggaaaaaaaaaaaaaaaaaaaaaaaaa

SEQ ID NO: 120 - DNA, 602 bp, Homo sapiens
  hypothetical protein LOC391356, chromosome 2 open reading frame 79 (C2orf79), peptidyl-tRNA hydrolase domain containing 1 (PTRHD1)
ccaagatgcaccggggagtaggtccggcctttcgggtggtcaggaagatggcggcctctggggcggagccgcaggtcctggtacaatacttggtgttacgaaaggatctatcacaagctccgttctcctggccggcgggcgcactggtagcgcaggcttgtcacgcggccaccgcggccttgcacactcaccgcgaccacccgcacacagccgcttacctccaagagctggggcgcatgcgcaaagtggtcctcgaggccccagatgagaccaccctaaaggagctggccgagaccctgcaacagaagaacattgaccacatgctgtggcttgagcaaccagagaatatcgccacttgtattgctctccggccctaccccaaggaagaagtgggccagtatttgaagaagttccgattgttcaagtaactgctgctttgatgtgtttgaatacgcaggccacccattccaaagcatcatgtgttccttgcagtgtcagcttgctcccgtctttcagttgtgacaatttcttgagggttaagcacatgttcatattaaagttgtcattaataaaaaaaaaaaaaaaaaaaaaaaaaaaaaaaa

SEQ ID NO: 121 - DNA, 2487 bp, Homo sapiens
  phosphoglucomutase 1 transcript variant 1 (PGM1), glucose phosphomutase 1, CDG1T, GSD14
cctttcccctcccgccggacctgccaggaggtgggctggcgcggagggagggccctgtcccctgtccctttaaggaggagggccaaacgccggcctagagtgcggcgtagcccccacccgccgtgccctcaccccagagcagctgcagcctcagccggccgcccctccgccagccaagtccgccgctctgacccccggcagcaagtcgccaccatggtgaagatcgtgacagttaagacccaggcgtaccaggaccagaagccgggcacgagcgggctgcggaagcgggtgaaggtgttccagagcagcgccaactacgcggagaacttcatccagagtatcatctccaccgtggagccggcgcagcggcaggaggccacgctggtggtgggcggggacggccggttctacatgaaggaggccatccagctcatcgctcgcatcgctgccgccaacgggatcggtcgcttggttatcggacagaatggaatcctctccacccctgctgtatcctgcatcattagaaaaatcaaagccattggtgggatcattctgacagccagtcacaacccagggggccccaatggagattttggaatcaaattcaatatttctaatggaggtcctgctccagaagcaataactgataaaattttccaaatcagcaagacaattgaagaatatgcagtttgccctgacctgaaagtagaccttggtgttctgggaaagcagcagtttgacttggaaaataagttcaaacccttcacagtggaaattgtggattcggtagaagcttatgctacaatgctgagaagcatctttgatttcagtgcactgaaagaactactttctgggccaaaccgactgaagatccgtattgatgctatgcatggagttgtgggaccgtatgtaaagaagatcctctgtgaagaactcggtgcccctgcgaactcggcagttaactgcgttcctctggaggactttggaggccaccaccctgaccccaacctcacctatgcagctgacctggtggagaccatgaagtcaggagagcatgattttggggctgcctttgatggagatggggatcgaaacatgattctgggcaagcatgggttctttgtgaacccttcagactctgtggctgtcattgctgccaacatcttcagcattccgtatttccagcagactggggtccgcggctttgcacggagcatgcccacgagtggtgctctggaccgggtggctagtgctacaaagattgctttgtatgagaccccaactggctggaagttttttgggaatttgatggacgcgagcaaactgtccctttgtggggaggagagcttcgggaccggttctgaccacatccgtgagaaagatggactgtgggctgtccttgcctggctctccatcctagccacccgcaagcagagtgtggaggacattctcaaagatcattggcaaaagtatggccggaatttcttcaccaggtatgattacgaggaggtggaagctgagggcgcaaacaaaatgatgaaggacttggaggccctgatgtttgatcgctcctttgtggggaagcagttctcagcaaatgacaaagtttacactgtggagaaggccgataactttgaatacagcgacccagtggatggaagcatttcaagaaatcagggcttgcgcctcattttcacagatggttctcgaatcgtcttccgactgagcggcactgggagtgccggggccaccattcggctgtacatcgatagctatgagaaggacgttgccaagattaaccaggacccccaggtcatgttggccccccttatttccattgctctgaaagtgtcccagctgcaggagaggacgggacgcactgcacccactgtcatcacctaagaagacaggcctgatgtggtacgtccctccacccccggacccatccaagtcatctgattgaagagcatgacagaaacaaaatgtattcaccaagcattttaggatttgactttttcactaaccagttgacgagcagtgcatttacaaggcactgccaaacaagatgcccttgggagctgtgagggaaagaggacctgcgggcttagatcaatctcaattccttttcatgccctcctgcattgctgctgcgtgggtatttgtctccttagccatcaggtacagtttacactacaatgtaagctataggtggagcatcagcagtgagtgaggccattcttcatccttaggatgtggcaatgaaatgatggtgcaagttcctttctcttttgtgaatctttccccccatttcctgtttacatgtaacccaacaaaatgcaatttctagtgccttctgtccaatcagttctttcctctgagtgagacgtacttggctacagatttctgccttgttttgcgacattgtcccattcacacagatattttgggataataaaggaaaataagctacaaaaaaaaaaaaaaaaa

SEQ ID NO: 122 - DNA, 2873 bp, Homo sapiens
  solute carrier family 19 (folate transporter), member 1 transcript variant 1 (SLC19A1), reduced folate carrier protein (RFC, REFC, RFC1), folate transporter 1 (FOLT), placental folate transporter, intestinal folate carrier 1 (IFC-1, IFC1)
agccgcgcgcccgccgcgccgccttgtgggcgctgtagtcccggagtccgcgtgcgcggggccgggtccgggagccccagggcagccgccccgccgagtcgcaggcacagcgtcaccttcgtcccctccggagctgcacgtggcctgagcaggatggtgccctccagcccagcggtggagaagcaggtgcccgtggaacctgggcctgaccccgagctccggtcctggcggcacctcgtgtgctacctttgcttctacggcttcatggcgcagatacggccaggggagagcttcatcaccccctacctcctggggcccgacaagaacttcacgcgggagcaggtcacgaacgagatcacgccggtgctgtcgtactcctacctggccgtgctggtgcccgtgttcctgctcaccgactacctgcgctacacgccggtgctgctgctgcaggggctcagcttcgtgtcggtgtggctgctgctgctgctgggccactcggtggcgcacatgcagctcatggagctcttctacagcgtcaccatggccgcgcgcatcgcctattcctcctacatcttctctctcgtgcggcccgcgcgctaccagcgtgtggccggctactcgcgcgctgcggtgctgctgggcgtgttcaccagctccgtgctgggccagctgctggtcactgtgggccgagtctccttctccacgctcaactacatctcgctggccttcctcaccttcagcgtggtcctcgccctcttcctgaagcgccccaagcgcagcctcttcttcaaccgcgacgaccgggggcggtgcgaaacctcggcttcggagctggagcgcatgaatcctggcccaggcgggaagctgggacacgccctgcgggtggcctgtggggactcagtgctggcgcggatgctgcgggagctgggggacagcctgcggcggccgcagctgcgcctgtggtccctctggtgggtcttcaactcggccggctactacctggtggtctactacgtgcacatcctgtggaacgaggtggaccccaccaccaacagtgcgcgggtctacaacggcgcggcagatgctgcctccacgctgctgggcgccatcacgtccttcgccgcgggcttcgtgaagatccgctgggcgcgctggtccaagctgctcatcgcgggcgtcacggccacgcaggcggggctggtcttccttctggcgcacacgcgccacccgagcagcatctggctgtgctatgcggccttcgtgctgttccgcggctcctaccagttcctcgtgcccatcgccacctttcagattgcatcttctctgtctaaagagctctgtgccctggtcttcggggtcaacacgttctttgccaccatcgtcaagaccatcatcactttcattgtctcggacgtgcggggcctgggcctcccggtccgcaagcagttccagttatactccgtgtacttcctgatcctgtccatcatctacttcttgggggccatgctggatggcctgcggcactgccagcggggccaccacccgcggcagcccccggcccagggcctgaggagtgccgcggaggagaaggcagcacaggcactgagcgtgcaggacaagggcctcggaggcctgcagccagcccagagcccgccgctttccccagaagacagcctgggggctgtggggccagcctccctggagcagagacagagcgacccatacctggcccaggccccggccccgcaggcagctgaattcctgagcccagtgacaaccccttccccctgcactctgtgctccgcccaagcctcaggccctgaggctgcagatgagacttgtccccagctggctgtccatcctcctggtgtcagcaagctgggtttgcagtgtcttccaagcgacggtgttcagaatgtgaaccagtgactctcgggcgcccctgtggtaactttgcaggcggccctcagtgcatccccacgacccctgcctcgagggccgcctgccttagcaatgggggcctccgcttatcctgctagcaggccccctaggattccccctgccctgtgccgcactctggcggtggccacagcgtgctggcgacactcagggcagctgcctggccatgctgtccctgcactgtgccccgcgggctttgttgctggaagaggtgggtggtgggcttctgcgtccaccaggcctcactggctcatgccccttggggggcttgagacaaatcctttctgccccccagggctagtgaagtggcctcttggataccagctcaggggacactggccccacaggagttgtgagccctctagggcagggtgggagccgggaccctcaggtgtagctgagctgtgacattgctggtcatccttggtgctcttgcttttttgaaagatgctttttttttttttaactgacgtagaatgaagaactgcatgtggcttctctgtctctgtggaaaagccatctcaggttggcggcagacacattgtcatcagaggggagcagcggctctggtcctcggagctggttcctctctcccaccctaagggcagccctccatggtcctgtctgtccttctgaagtgtgtccatcctgacctgcgggtcctcagctgctcccacacttgtgccagcccggaggggactggtcccggtcaccgcggacgtgctggccttggtatgtgccaggcttgcctgggctgggcagccttgggggggctgcctttgtggtgggcgctggggaagtacgtcccagcggcctcagggtctaaggagcgctagtgccttgcccacaggtgcgggaccatctgatgtgatgtgaatactcttcccacatacattaaacacacttaagtgaga

SEQ ID NO: 123 - DNA, 5748 bp, Homo sapiens
  CD44 antigen transcript variant 1, hematopoietic cell E- and L-selectin ligand (HCELL), chondroitin sulfate proteoglycan 8 (CSPG8), GP90 lymphocyte homing/adhesion receptor, epican, Hermes antigen, extracellular matrix receptor III (ECMR-III)
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 124 - DNA, 579 bp, Homo sapiens
  60S ribosomal protein L24, L24, ribosomal protein L30, epididymis secretory protein Li 310 (HEL-S-310)
tctttcttttcgccatcttttgtctttccgtggagctgtcgccatgaaggtcgagctgtgcagttttagcgggtacaagatctaccccggacacgggaggcgctacgccaggaccgacgggaaggttttccagtttcttaatgcgaaatgcgagtcggctttcctttccaagaggaatcctcggcagataaactggactgtcctctacagaaggaagcacaaaaagggacagtcggaagaaattcaaaagaaaagaacccgccgagcagtcaaattccagagggccattactggtgcatctcttgctgatataatggccaagaggaatcagaaacctgaagttagaaaggctcaacgagaacaagctatcagggctgctaaggaagcaaaaaaggctaagcaagcatctaaaaagactgcaatggctgctgctaaggcacctacaaaggcagcacctaagcaaaagattgtgaagcctgtgaaagtttcagctccccgagttggtggaaaacgctaaactggcagattagatttttaaataaagattggattataactctagaaaaaaaaaaaaaaaaa

SEQ ID NO: 125 - DNA, 3749 bp, Homo sapiens
  nicalin precursor (NCLN), nicastrin-like protein; nicalin homolog (zebrafish), NET59
gcggtgcccacaggacctcagggcgagtgcgggctgccccgcgcggcgcccgcaggaccccggcggctacccatgccgaggtgagtccgcgggagccgccgccgccgccgtcccgtcccagctgccgccccgcgcggccccgccgccggccaggatgctggaggaagcgggcgaggtgctggagaacatgctgaaggcgtcttgtctgccgctcggcttcatcgtcttcctgcccgctgtgctgctgctggtggcgccgccgctgcctgccgccgacgccgcgcacgagttcaccgtgtaccgcatgcagcagtacgacctgcagggccagccctacggcacacggaatgcagtgctgaacacggaggcgcgcacgatggcggcggaggtgctgagccgccgctgcgtgctcatgcggctactggacttctcctacgagcagtaccagaaggccctgcggcagtcggcgggcgccgtggtcatcatcctgcccagggccatggccgccgtgccccaggacgtcgtccggcaattcatggagatcgagccggagatgctggccatggagaccgccgtccccgtgtactttgccgtggaggacgaggccctgctgtctatctacaagcagacccaggctgcctccgcctcccagggctccgcctctgctgctgaagtactgctgcgcacggccactgccaacggcttccagatggtcaccagcggggtacagagcaaggccgtgagtgactggctgattgccagcgtggaggggcggctgacggggctgggcggagaggaccttcccaccatcgtcatcgtggcccactacgacgcctttggagtggccccctggctgtcgctgggcgcggactccaacgggagcggcgtctctgtgctgctggagctggcacgcctcttctcccggctctacacctacaagcgcacgcacgccgcctacaacctcctgttctttgcgtctggaggaggcaagtttaactaccagggaaccaagcgctggctggaagacaacctggaccacacagactccagcctgcttcaggacaatgtggccttcgtgctgtgcctggacaccgtgggccggggcagcagcctgcacctgcacgtgtccaagccgcctcgggagggcaccctgcagcacgccttcctgcgggagctggagacggtggccgcgcaccagttccctgaggtacggttctccatggtgcacaagcggatcaacctggcggaggacgtgctggcctgggagcacgagcgcttcgccatccgccgactgcccgccttcacgctgtcccacctggagagccaccgtgacggccagcgcagcagcatcatggacgtgcggtcccgggtggattctaagaccctgacccgtaacacgaggatcattgcagaggccctgactcgagtcatctacaacctgacagagaaggggacacccccagacatgccggtgttcacagagcagatgcagatccagcaggagcagctggactcggtgatggactggctcaccaaccagccgcgggccgcgcagctggtggacaaggacagcaccttcctcagcacgctggagcaccacctgagccgctacctgaaggacgtgaagcagcaccacgtcaaggctgacaagcgggacccagagtttgtcttctacgaccagctgaagcaagtgatgaatgcgtacagagtcaagccggccgtctttgacctgctcctggctgttggcattgctgcctacctcggcatggcctacgtggctgtccagcacttcagcctcctctacaagaccgtccagaggctgctcgtgaaggccaagacacagtgacacagccacccccacagccggagcccccgccgctccacagtccctggggccgagcacgagtgagtggacactgccccgccgcgggcggccctgcagggacaggggccctctccctccccggcggtggttggaacactgaattacagagcttttttctgttgctctccgagactggggggggattgtttcttcttttccttgtctttgaacttccttggaggagagcttgggagacgtcccggggccaggctacggacttgcggacgagccccccagtcctgggagccggccgccctcggtctggtgtaagcacacatgcacgattaaagaggagacgccgggaccccctgcccgatcgcgcgcggcctccgcccaccgcctcctgccgcaaggggcctggactgcaggcctgacctgctccctgctccgtgtctgtcctaggacgtcccctcccgctccccgatggtggcgtggacatggttatttatctctgctccttcttgcctggaggagggcagtgccagccctggggttctgggattccagccctcctggagccttttgttccccatgtggtctcagtgacccgtccccctgacagtgggctcggggagctgcatcacccagccttccccttctccgactgcagggtctgatgtcatcattgacagcctttgcttcgtgggggcctggcagggcccctgcctccccgacccccgacccactgcaaatccccgttcccctgcactcctcttctcccagcccatccctccggcccctgtgcctctgcggccccagcccagctcccagggccgtcacctgcttggccctggcccagctccctgccctgagtcctgagccagtgcctggtgtttcctgggctcggtactgggcccccaggccatccaggctttgccacggccagttggtcctccctggggaactgggtgcgggtggagtactgggaggcaggaggtggcccggggaggccttgtggctcctcccctcgctcctcgccctgggcctcagcttcctcatcaatagaaaggatgtgttcggggtgggggcgtcaggtgagaacgtttgctgggaaggagaggacttggggcatggcctctggggccacccttcctggaactcagagaggaaggtccgggccctcgggaagccttggacagaaccctccaccccgcagaccaggcgtcgtgtgtgtgtgggagagaaggaggcccgtgttgagctcagggagaccccggtgtgtccgttctttagcaatataacctacccagtgcgtgccgagcaggcttggtggggaagggacttgagctgggcaagtcctggcctggcacccgcagccgtctcccttccgtggcccagggaggtgtttgctgtccgaaggacctgggccggcccatgggagcctggggttctgtccagataggaccagggggtctcactttggccaccagttcttcggccagcacctctgccctccagaacctgcagcctggaggggtgaggggacaaccacccctctttcctccaggttggcaggggaccctcttctcccgtctgccctgcgggttgcccgcctcctccagagacttgcccaagggcccatcaccactggcctctgggcacttgtgctgagactctgggacccaggcagctgccaccttgtcaccatgagagaatttggggagtgcttgcatgctagccagcaggctcctgtctgggtgccacggggccagcattttggagggagcttccttccttccttcctggacaggtcgtcatgatggatgcactgactgaccgtctggggctcaggctggtgtgggatgcagccggccgatgagaaaataaagccatattgaatgatcgccaaaaaaaaaaaaaaaaa

SEQ ID NO: 126 - DNA, 2350 bp, Homo sapiens
  60S ribosomal protein L15 transcript variant 1, L15, DBA12, EC45, RPL10, RPLY10, RPYL10
aaagacagcggctccaccgcggtacgcggccaccggctttggagcctggaccccaacttgcctcctctcgcggagagacagtcgccgacgctcgcttagccgccgagacctcgccgccaactctctcacctctcgagacgcccaggccgctcaggctcgaatcttgcggagcagggggcgggacaatagcggccgcggcgccccactcggcagaactccgccaccaggcgcgatgccggaactacatgtcccatgacgctctgggaggccgcagctttccaccggaaagagggtggctgaggtgggggaggagcccaaaaggcattgtgggagtacagctctttcctttccgtctggcggcagccatcaggtaagccaagatgggtgcatacaagtacatccaggagctatggagaaagaagcagtctgatgtcatgcgctttcttctgagggtccgctgctggcagtaccgccagctctctgctctccacagggctccccgccccacccggcctgataaagcgcgccgactgggctacaaggccaagcaaggttacgttatatataggattcgtgttcgccgtggtggccgaaaacgcccagttcctaagggtgcaacttacggcaagcctgtccatcatggtgttaaccagctaaagtttgctcgaagccttcagtccgttgcagaggagcgagctggacgccactgtggggctctgagagtcctgaattcttactgggttggtgaagattccacatacaaattttttgaggttatcctcattgatccattccataaagctatcagaagaaatcctgacacccagtggatcaccaaaccagtccacaagcacagggagatgcgtgggctgacatctgcaggccgaaagagccgtggccttggaaagggccacaagttccaccacactattggtggctctcgccgggcagcttggagaaggcgcaatactctccagctccaccgttaccgctaatataagtaaagtttgtaaaattcatacttaataaacaatttaggacagtcatgtctgcttacaggtgttatttgtctgttaaaactagtctgcagatgtttcttgaatgctttgtcaaattaagaaagttaaagtgcaataatgtttgaagacaataagtggtggtgtatcttgtttctaataagataaacttttttgtctttgctttatcttattagggagttgtatgtcagtgtataaaacatactgtgtggtataacaggcttaataaattctttaaaaggagagaactgaaactagccctgtagatttgtctggtgcatgtgatgaaacctgcagctttatcggagtgatggcaatgctctgctggtttattttcaagtggctgcgttttttttagtttggcaggtgtagactttttaagttgggctttagaaaatctgggttagcctgaagaaaattgcctcagcctccacagtaccattttaaattcacataaaaggtgaaagctcctggttcagtgccatggcttcatggcattcagtgattagtggtaatggtaaacactggtgtgttttgaagttgaatgtgcgataaaattattagccttaagattggtaagctagcaatgaatgctagggtgggaagctggtgagccagtggccattagataaatacctttcaagtgtgagcttagacgtcaaccctaaaatacttaaccgtaatgctaattgtgatcattatgaatcccttcagtcacattagggggaaagtagttggctataagtacgtcattcttagtccagtcagtcttaaaaacatcttgggttacccactctgtccactcccataggctacagaaaaagtcacaagcgcatggtttccaaccatatgtgttttctgcagttatttctcttgttctggccaaacaaccctaaaaatccttaccattccacaaagttggaccatcacttgtgcacccactttgactatgagtataccaccacattgcatttctgtttgcaccatgtcttccaggagactagactactgttgtccagggtcaatttgagtgtaaagaaaatgtagacaaggaattgcccaattttaaattctgactttgctgacttaatttaaatgctcgttctgaaccaattttctcctatcttctctaggggtttcaaaagactcagttaattgatttccaggaagtactcatagcaagttcataaaagttcttgagacctaaatttcttcacaaaaaaagaaaagatcttaagtcatacattttaattgtgtagaggttgttcaactgaaggaataaatgtctattaaactaaaacaaatggaccttc

SEQ ID NO: 127 - DNA, 2531 bp, Homo sapiens
  cleft lip and palate transmembrane protein 1 transcript variant 2 (CLPTM1)
gaagtcggggacggggcggggctggcggcgggggcggggacccggagcgggaagatggcggcggcgcaggaggcggacggggcccgcagcgccgtggtggcggccgggggaggcagctccggtcaggtgaccagcaatggcagcatcgggagggacccgccagcggagacccagcctcagaacccaccggcccagccggcacccaatgcctggcaggtcatcaaaggtgtgctgtttaggatcttcatcatctgggccatcagcagttggttccgccgagggccggcccctcaggaccaggcgggccccggaggagctccacgcgtcgccagccgcaacctgttccccaaagacactttaatgaacctgcatgtgtacatctcagagcacgagcactttacagacttcaacgccacgtcggcactcttctgggaacagcacgatcttgtgtatggcgactggactagcggcgagaactcagacggctgctacgagcactttgctgagctcgatatcccacagagcgtccagcagaacggctccatctacatccacgtttacttcaccaagagtggcttccacccagacccccggcagaaggccctgtaccgccggcttgccacagtccacatgtcccggatgatcaacaaatacaagcgcagacgatttcagaaaaccaagaacctgctgacaggagagacagaagcggacccagaaatgatcaagagggctgaggactatgggcctgtggaggtgatctcccattggcaccccaacatcaccatcaacatcgtggacgaccacacgccgtgggtgaagggcagtgtgccccctcccctggatcaatatgtgaagttcgacgccgtgagcggtgactactatcccatcatctacttcaatgactactggaacctgcagaaggactactaccccatcaacgagagcctggccagcctgccgctccgcgtctccttctgcccactctcgctttggcgctggcagctctatgctgcccagagcaccaagtcgccctggaacttcctgggtgatgagttgtacgagcagtcagatgaggagcaggactcggtgaaggtggccctgctggagaccaacccctacctgctggcgctcaccatcatcgtgtctatcgttcacagtgtcttcgagttcctggccttcaagaatgatatccagttctggaacagccggcagtccctggagggcctgtccgtgcgctccgtcttcttcggcgttttccagtcattcgtggtcctcctctacatcctggacaacgagaccaacttcgtggtccaggtcagcgtcttcattggggtcctcatcgacctctggaagatcaccaaggtcatggacgtccggctggaccgagagcacagggtggcaggaatcttcccccgcctatccttcaaggacaagtccacgtatatcgagtcctcgaccaaagtgtatgatgatatggcattccggtacctgtcctggatcctcttcccgctcctgggctgctatgccgtctacagtcttctgtacctggagcacaagggctggtactcctgggtgctcagcatgctctacggcttcctgctgaccttcggcttcatcaccatgacgccccagctcttcatcaactacaagctcaagtctgtggcccaccttccctggcgcatgctcacctacaaggccctcaacacattcatcgacgacctgttcgcctttgtcatcaagatgcccgttatgtaccggatcggctgcctgcgggacgatgtggttttcttcatctacctctaccaacggtggatctaccgcgtcgaccccacccgagtcaacgagtttggcatgagtggagaagaccccacagctgccgcccccgtggccgaggttcccacagcagcaggggccctcacgcccacacctgcacccaccacgaccaccgccaccagggaggaggcctccacgtccctgcccaccaagcccacccagggggccagctctgccagcgagccccaggaagcccctccaaagccagcagaggacaagaaaaaggattagtcgagactggtcctcacctgctccggctcctggcgaccactacccctgcgtcccggccccctcgcctcccctccctgtcgccctttccctggacagatcaggccggggcggtgggaggcccgcctcaggtcagggcccagcgtgtgatgtaggggccggggcaggccagggtttgtttgtggaggcgctgtctgtccctctgtccctctgtgtttccagccatctcgccctgccagcccagcaccactgggaatcatggtgaagctgatgcagcgttgccgagggggtgggttgggcgggggtggggccgggcccccctacgggatgcccacggccgttcatcatcttgtccctcgtccccctaccacactccccctcctagaccgccgccctttaacacagtctggatttaataaattcatatgggtgtttaacttaaactcagcactaaaaaaaaaaaaaaaaaa

SEQ ID NO: 128 - DNA, 1710 bp, Homo sapiens
  evolutionarily conserved signaling intermediate in Toll pathway,mitochondrial precursor transcript variant 1 (ECSIT), SITPEC
gatttgctacctccctggagctccctgacccggacgctctctgggccaatatggcagcgcccagcaacaagacagagctggcctggagtccgcggctggccgcgtgagtaggtgattgtctgacaagcagaggcatgagctgggtccaggccaccctactggcccgaggcctctgtagggcctggggaggcacctgcggggccgccctcacaggaacctccatctctcaggtccctcgccggctccctcggggcctccactgcagcgcagctgcccatagctctgaacagtccctggttcccagcccaccggaaccccggcagaggcccaccaaggctctggtgccctttgaggacctgtttgggcaggcgcctggtggggaacgggacaaggcgagcttcctgcagacggtgcagaaatttgcggagcacagcgtgcgtaagcggggccacattgacttcatctacctggccctgcgcaagatgcgggagtatggtgtcgagcgggacctggctgtgtacaaccagctgctcaacatcttccccaaggaggtcttccggcctcgcaacatcatccagcgcatcttcgtccactaccctcggcagcaggagtgtgggattgctgtcctggagcagatggagaaccacggtgtgatgcccaacaaggagacggagttcctgctgattcagatctttggacgcaaaagctaccccatgctcaagttggtgcgcctgaagctgtggttccctcgattcatgaacgtcaaccccttcccagtgccccgggacctgccccaggaccctgtggagctggccatgtttggcctgcggcacatggagcctgaccttagtgccagggtcaccatctaccaggttcctttgcccaaagactcaacaggtgcagcagatcccccccagccccacatcgtaggaatccagagtcccgatcagcaggccgccctggcccgccacaatccagcccggcctgtctttgttgagggccccttctccctgtggctccgcaacaagtgtgtgtattaccacatcctcagagctgacttgctgcccccggaggagagggaagtggaagagacgccggaggagtggaacctctactacccgatgcagctggacctggagtatgtgaggagtggctgggacaactacgagtttgacatcaatgaagtggaggaaggccctgtcttcgccatgtgcatggcgggtgctcatgaccaggcgacgatggctaagtggatccagggcctgcaggagaccaacccaaccctggcccagatccccgtggtcttccgcctcgccgggtccacccgggagctccagacatcctctgcagggctggaggagccgcccctgcccgaggaccaccaggaagaagacgacaacctgcagcgacagcagcagggccagagctagtctgagccggcgcgagggcacgggctgtggcccgaggaggcggtggactgaaggcatgagatgccctttgagtgtacagcaaatcaatgttttcctgcttggggctctcttccctcatctctagcagtatggcatcccctccccaggatctcgggctgccagcgatgggcaggcgagacccctccagaatctgcaggcgcctctggttctccgaattcaaataaaaaggggcgggagcgctgttggttgtgcgcatgcgcagtttccaaaaaaaaaaaaa

SEQ ID NO: 129 - DNA, 854 bp, Homo sapiens
  eukaryotic translation elongation factor 1 beta 2 transcript variant 2 (EEF1B2), elongation factor 1-beta (EF1B, EEF1B, EF-1-beta), eukaryotic translation elongation factor 1 beta 1 (EEF1B1)
gccggaagtggccccagcctcgaggccgggcgtcttcggtcatctccggcgcttctagggctggttcccgtcatcttcgggagccgtggagctctcggatacagccgacaccatgggtttcggagacctgaaaagccctgccggcctccaggtgctcaacgattacctggcggacaagagctacatcgaggggtatgtgccatcacaagcagatgtggcagtatttgaagccgtgtccagcccaccgcctgccgacttgtgtcatgccctacgttggtataatcacatcaagtcttacgaaaaggaaaaggccagcctgccaggagtgaagaaagctttgggcaaatatggtcctgccgatgtggaagacactacaggaagtggagctacagatagtaaagatgatgatgacattgacctctttggatctgatgatgaggaggaaagtgaagaagcaaagaggctaagggaagaacgtcttgcacaatatgaatcaaagaaagccaaaaaacctgcacttgttgccaagtcttccatcttactagatgtgaaaccttgggatgatgagacagatatggcgaaattagaggagtgcgtcagaagcattcaagcagacggcttagtctggggctcatctaaactagttccagtgggatacggaattaagaaacttcaaatacagtgtgtagttgaagatgataaagttggaacagatatgctggaggagcagatcactgcttttgaggactatgtgcagtccatggatgtggctgctttcaacaagatctaaaatccatcctggatcatggcatttaaataaaagattgaaagattacaaaaaaaaaaaaaaaaaa

SEQ ID NO: 130 - DNA, 743 bp, Homo sapiens
  prefoldin subunit 5 transcript variant 1 (PFDN5, PFD5), c-myc binding protein, myc modulator-1 (MM-1, MM1)
gaggatcatagagctgtctggcgcagcgaggcctcccggcgccaccgagacgcgcagaggacggctagagcgttgctcgccgagagacttcctcttcgttaagtcggccttcccaacatggcgcagtctattaacatcacggagctgaatctgccgcagctagaaatgctcaagaaccagctggaccaggaagtggagttcttgtccacgtccattgctcagctcaaagtggtacagaccaagtatgtggaagccaaggactgtctgaacgtgctgaacaagagcaacgaggggaaagaattactcgtcccactgacgagttctatgtatgtccctgggaagctgcatgatgtggaacacgtgctcatcgatgtgggaactgggtactatgtagagaagacagctgaggatgccaaggacttcttcaagaggaagatagattttctaaccaagcagatggagaaaatccaaccagctcttcaggagaagcacgccatgaaacaggccgtcatggaaatgatgagtcagaagattcagcagctcacagccctgggggcagctcaggctactgctaaggcctgagagtttttgcagaaatggggcagagggacaccctttgggcgtggcttcctggtgatgggaagggtcttgtgttttaatgccaataaatgtgccagctgggcagaaaaaaaaaaaaaaaaaaaaaaaaaaaaaaaaaaaaaaaaaaaaaaaaaaaaaaaaaa

SEQ ID NO: 131 - DNA, 1721 bp, Homo sapiens
  polynucleotide kinase 3′-phosphatase (PNKP, PNK), bifunctional polynucleotide phosphatase/kinase, DNA 5′-kinase/3′-phosphatase, EIEE10, MCSZ
ccgaggaaccgaccgccgccggccgggttgcaggcggggcacctcgggcaggacctccctggtcggaagtggccgtgagcccaagccgcggtcccgggccggcacccaggatgggcgaggtggaggccccgggccgcttgtggctcgagagcccccctgggggagcgccccccatcttcctgccctcggacgggcaagccctggtcctgggcaggggacccctgacccaggttacggaccggaagtgctccagaactcaagtggagctggtcgcagatcctgagacccggacagtggcagtgaaacagctgggagttaacccctcaactaccgggacccaggagttgaagccggggttggagggctctctgggggtgggggacacactgtatttggtcaatggcctccacccactgaccctgcgctgggaagagacccgcacaccagaatcccagccagatactccgcctggcacccctctggtgtcccaagatgagaagagagatgctgagctgccgaagaagcgtatgcggaagtcaaaccccggctgggagaacttggagaagttgctagtgttcaccgcagctggggtgaaaccccagggcaaggtggctggctttgatctggacgggacgctcatcaccacacgctctgggaaggtctttcccactggccccagtgactggaggatcttgtacccagagattccccgtaagctccgagagctggaagccgagggctacaagctggtgatcttcaccaaccagatgagcatcgggcgcgggaagctgccagccgaggagttcaaggccaaggtggaggctgtggtggagaagctgggggtccccttccaggtgctggtggccacgcacgcaggcttgtaccggaagccggtgacgggcatgtgggaccatctgcaggagcaggccaacgacggcacgcccatatccatcggggacagcatctttgtgggagacgcagccggacgcccggccaactgggccccggggcggaagaagaaagacttctcctgcgccgatcgcctgtttgccctcaaccttggcctgcccttcgccacgcctgaggagttctttctcaagtggccagcagccggcttcgagctcccagcctttgatccgaggactgtctcccgctcagggcctctctgcctccccgagtccagggccctcctgagcgccagcccggaggtggttgtcgcagtgggattccctggggccgggaagtccacctttctcaagaagcacctcgtgtcggccggatatgtccacgtgaacagggacacgctaggctcctggcagcgctgtgtgaccacgtgtgagacagccctgaagcaagggaaacgggtcgccatcgacaacacaaacccagacgccgcgagccgcgccaggtacgtccagtgtgcccgagccgcgggcgtcccctgccgctgcttcctcttcaccgccactctggagcaggcgcgccacaacaaccggtttcgagagatgacggactcctctcatatccccgtgtcagacatggtcatgtatggctacaggaagcagttcgaggccccaacgctggctgaaggcttctctgccatcctggagatcccgttccggctatgggtggagccgaggctggggcggctgtactgccagttctccgagggctgagccccgcccagctcccctccacaataaacgctgtttctccttgag

SEQ ID NO: 132 - DNA, 2889 bp, Homo sapiens
  septin 8 transcript variant 1 (SEPT8), SEP2
gcggcggggctccggctgcgctcgtggccgggccgggcggggaggccggtcccgcgggcgggggcaggggcggctccgcggcttctcccgccgccgccgccaaggggagtttccaggaagtggccatattggatccattcagccgcagccgcccgggcggagcgcgtcccgcagccggctggtccctgtcgctgcccctgcgctcgtcccagcccacccgcccggtgcggagctcgccatggcggccaccgacctggagcgcttctcgaatgcagagccagagccccggagcctctccctgggcggccatgtgggtttcgacagcctccccgaccagctggtcagcaagtcggtcactcagggcttcagcttcaacatcctctgtgtgggggagaccggcattggcaaatccacactgatgaacacactcttcaacacgaccttcgagactgaggaagccagtcaccatgaggcatgcgtgcgcctgcggccccagacctatgacctccaggagagcaacgtgcagctcaagctgaccattgtggatgccgtgggctttggggatcagatcaataaggatgagagttacaggcccatagttgactacatcgatgcgcagtttgaaaattatctgcaggaggagctgaagatccgccgctcgctcttcgactaccatgacacaaggatccacgtttgcctctacttcatcacgcccacagggcactccctgaagtctctagatctagtgaccatgaagaaactagacagcaaggtgaacattattcccatcatcgccaaggctgacaccatctccaagagcgagctccacaagttcaagatcaagatcatgggcgagttggtcagcaacggggtccagatctaccagttccccacggatgatgaggctgttgcagagattaacgcagtcatgaatgcacatctgccctttgccgtggtgggcagcaccgaggaggtgaaggtggggaacaagctggtccgagcacggcagtacccctggggagtggtgcaggtggagaatgagaatcactgcgacttcgtgaagctgcgggagatgttgatccgggtgaacatggaagacctccgcgagcagacccacagccggcactacgagctctaccggcgctgcaagttggaggagatgggctttcaggacagcgatggtgacagccagcccttcagcctacaagagacatacgaggccaagaggaaggagttcctaagtgagctgcagaggaaggaggaagagatgaggcagatgtttgtcaacaaagtgaaggagacagagctggagctgaaggagaaggaaagggagctccatgagaagtttgagcacctgaagcgggtccaccaggaggagaagcgcaaggtggaggaaaagcgccgggaactggaggaggagaccaacgccttcaatcgccggaaggctgcggtggaggccctgcagtcgcaggccttgcacgccacctcgcagcagcccctgaggaaggacaaggacaagaagaacagatcagatataggagcacaccagccgggcatgagcctctccagctctaaggtgatgatgaccaaggccagtgtggagcccttgaactgcagcagctggtggcccgccatacagtgctgcagctgcctggtcagggatgcgacgtggagggaaggattcctctgaggcagcagctccaacacatggggccagctcaggaccaccagggcatggaactggagaccatggtttttaatgttagaacagaaaacgccatacttttcctatatcaatgatcaaaagtgcaaacaatttaaatttccatcagggaacatcaaatgttgcccaacccttttcattcctatccatggctccgtaaggggcttgaggcttaatgcccatcctgtggccaagctgagcttccactccgggaccaaaaaaaaaaaaaagtctgctttgtgacatcatcgttatgagcggaaagtacctagatgacaatgtttccattctgaaaaatagaaacatactattcaagaccaaggtagcagaaaagttacttgtatctgcttatcataagacgaaactctgcaacttggcaacggtggccagttttcgtaatgaaacagtctttagtaatttaatcttcatgcttcataacaaaccaaaaccccatgagatttccacattgcataattttgccttactaacagaatcatatccttaaggatgaccatcattcccccaactaaaacaaatacaaactaatgtatgatatttttttaagtgccagatcaatatggtctaaagcttcaataaggattgtgtgtaggtgaataaagacagctaagtgaatgtgtgtaaagtgtagcaaaagcagacagatatttatgtacagtattcatagaatggaaagttaaatatttttgcagtgtgtatttaaaagagaaactcaccataatagtgccgtctaaaaatctttgtaaagttaatttaatgtcctttagaagtgggagtctggtggaactgtgttggatttaagataccttttcactcttccgtatgtcatgagccttgtgcgtcacctcactgtggtgcatgtgcaagggcgtgtgcacgcctgtgctttgccatcccatgttgtaaacagctgttccaaaggcacaaacgagtttagggtagactctgtaaacacctccttactcactatagtcaagaagtccagcggcgtcccaatatagaggtcccagtgcagtctgtccagaatagccagctccatcctcagcagctcattcggggaatagtcagagccatagtgctttgtgaagtcttttacttgtggaataaactgtaaaaagaaaataaagaggccaaagccctacatcatg

SEQ ID NO: 133 - DNA, 1397 bp, Homo sapiens
  cold inducible RNA binding protein transcript variant 1 (CIRBP, CIRP), glycine-rich RNA binding protein, A18 hnRNP
aggatgtgtagggggcggggcccggcggaagcgtatataaggccgggctcggggacgcccccccctcactcgcgcgttaggaggctcgggtcgttgtggtgcgctgtcttcccgcttgcgtcagggacctgcccgactcagtggccgccatggcatcagatgaaggcaaactttttgttggagggctgagttttgacaccaatgagcagtcgctggagcaggtcttctcaaagtacggacagatctctgaagtggtggttgtgaaagacagggagacccagagatctcggggatttgggtttgtcacctttgagaacattgacgacgctaaggatgccatgatggccatgaatgggaagtctgtagatggacggcagatccgagtagaccaggcaggcaagtcgtcagacaaccgatcccgtgggtaccgtggtggctctgccgggggccggggcttcttccgtgggggccgaggacggggccgtgggttctctagaggaggaggggaccgaggctatggggggaaccggttcgagtccaggagtgggggctacggaggctccagagactactatagcagccggagtcagagtggtggctacagtgaccggagctcgggcgggtcctacagagacagttatgacagttacgctacacacaacgagtaaaaacccttcctgctcaagatcgtccttccaatggctgtgtgtttaaagattgtgggagcttcgctgaacgttaatgtgtagtaaatgcacctccttgtattcccactttcgtagtcatttcggttctgatcttgtcaaacccagcctgaccgcttctgacgccgggatggcctcgttactagacttttctttttaaggaagtgctgtttttttttgagggttttcaaaacattttgaaaagcatttacttttttgaccacgagccatgagttttcaaaaaaatcgggggttgtgtgggtttttggtttttgttttagtttttggttgcgttgcctttttttttttagtggggttggccccatgaagtgggtgccccactcacttctctgagatcgaacggactgtgaatccgctctttgtcggaagctgagcaagctgtggcttttttccaactccgtgtgacgtttctgagtgtagtgtggtaggaccccggcgggtgtggcagcaactgccctggagccccagcccctgcgtccatctgtgctgtgcgccccacagtagacgtgcagacgtccctgagaggttcttgaagatgtttatttatattgtccttttttactggaagacgtacgcatactccatcgatgttgtatttgcagtggctgaggaattcttgtacgcagttttctttggctttacgaagccgattaaaagaccgtgtgaaatgaa

SEQ ID NO: 134 - DNA, 2528 bp, Homo sapiens
  ATP-binding cassette, sub-family B (MDR/TAP), member 7, mitochondrial transcript variant 1 (ABCB7), ABC transporter 7 protein, ATP-binding cassette transporter 7, ASAT; Atm1p; EST140535
aggtagccgaattcagtccgccagtgtcccataatcctcttctctcggttcctctttcctcgctcaagatggcgctgctcgcgatgcattcttggcgctgggcggccgcggcggctgctttcgaaaagcgccggcactccgcgattctgatccggcctttagtctctgttagcggctcaggtccgcagtggaggccacatcaactcggcgccttgggaaccgctcgagcctaccagcagattccagagtcattaaaaagtatcacatggcagagattgggaaaaggcaattcaggacagttcttagatgctgcaaaggctctccaggtatggccactgatagaaaagaggacatgttggcatggtcatgcaggaggaggactccacacagacccaaaagaagggttaaaagatgttgatactcggaaaatcataaaagcaatgctttcttatgtgtggcccaaagacaggccagatctacgagctagagttgccatttcgctgggatttttgggtggtgcaaaggccatgaatattgtggttcccttcatgtttaaatatgctgtagacagcctcaaccagatgtcgggaaacatgctgaacctgagtgatgcaccaaatacagttgcaaccatggcaacagcagttctgattggctatggtgtatcaagagctggagctgctttttttaacgaagttcgaaatgcagtatttggcaaggtagcccagaattcaatccgaagaatagccaaaaatgtctttctccatcttcacaacctggatctgggttttcacctgagcagacagacgggagctttatctaaggctattgacagaggaacaaggggtatcagttttgtcctgagtgctttggtatttaatcttcttcccatcatgtttgaagtgatgcttgtcagtggtgttttgtattacaaatgcggtgcccagtttgctttggtaacccttggaacacttggtacatacacagcattcacagttgcagtcacacggtggagaactagatttagaatagaaatgaacaaagcagataatgatgcaggtaatgctgctatagactcactgctgaattatgaaactgtgaagtattttaataatgaaagatatgaagcacagagatatgatggatttttgaagacgtatgagactgcttcattgaaaagtacctctactctggctatgctgaactttggtcaaagtgctattttcagtgtcggtttaacagctataatggtgctcgccagtcagggaattgtggcaggtacccttactgttggagatctagtaatggtgaatggactgctttttcagctttcattacccctgaactttctgggaactgtatatagagagactagacaagcactcatagatatgaacaccttgtttactctactcaaggtagacacccaaattaaagacaaagtgatggcatctccccttcagatcacaccacagacagctaccgtggcctttgataatgtgcattttgaatacattgagggccagaaagtccttagtggaatatcctttgaagtccctgcaggaaagaaagtggccattgtaggaggtagtgggtcagggaaaagcacaatagtgaggctattatttcgcttctatgagcctcaaaagggtagcatttatcttgctggtcaaaatatacaagatgtgagcctggaaagccttcggagggcagtgggagtggtacctcaggatgctgtcctcttccataatactatttattacaacctcttatatggaaacatcagtgcttcacctgaggaagtgtatgcagtggcaaaattagctggacttcatgatgcaattcttcgaatgccacatggatatgacacccaagtaggggaacgaggactcaagctttcaggaggagaaaagcaaagagtagcaattgcaagagccattttgaaggaccccccagtcatactctatgatgaagctacttcatcgttagattcgattactgaagagactattcttggtgccatgaaggatgtggtcaaacacagaacttctattttcattgcacacagattgtcaacagtggttgatgcagatgaaatcattgtcttggatcagggtaaggtagccgaacgtggtacccaccatggtttgcttgctaaccctcatagtatctattcagaaatgtggcatacacagagcagccgtgtgcagaaccatgataaccccaaatgggaagcaaagaaagaaaatatatccaaagaggaggaaagaaagaaactacaagaagaaattgtcaatagtgtgaaaggctgtggaaactgttcgtgctaagtcacataagacattttctttttttgttgttttggactacatatttgcactgaagcagaattgttttattaaaaaaatcatacattcccattttctataatccttcttttagataagatttatttaaaaggggatttgagttttacatctttcatagtctatttaatgtggcatctgtatttatccccaaattatttt

SEQ ID NO: 135 - DNA, 1136 bp, Homo sapiens
  alpha-N-acetyltransferase 1A, N-terminal acetyltransferase complex ARD1 subunit homolog A transcript variant 1 (ARD1A, ARD1), N-alpha-acetyltransferase 10, NatA catalytic subunit, ARD1P, DXS707, MCOPS1, NATD, TE2
gaccggctccgcgcattggcccgccccgtgcgctggcctgtcccgcgcattggccaagcggccgggaggacgggcccggcggccgtgcacttccggccggtggccggcccggcgcgcaccgccccttccgccgtcgcccagcgagcccagctccggtcccagccccggccgtcccggcgtcgcttcggagcgcggcggcagctgactgcgccttcacgatccgctgggacccgcgagccccgccgccgttatgaacatccgcaatgcgaggccagaggacctaatgaacatgcagcactgcaacctcctctgcctgcccgagaactaccagatgaaatactacttctaccatggcctttcctggccccagctctcttacattgctgaggacgagaatgggaagattgtggggtatgtcctggccaaaatggaagaggacccagatgatgtgccccatggacatatcacctcattggctgtgaagcgttcccaccggcgcctcggtctggctcagaaactgatggaccaggcctctcgagccatgatagagaacttcaatgccaaatatgtctccctgcatgtcaggaagagtaaccgggccgccctgcacctctattccaacaccctcaactttcagatcagtgaagtggagcccaaatactatgcagatggggaggacgcctatgccatgaagcgggacctcactcagatggccgacgagctgaggcggcacctggagctgaaagagaagggcaggcacgtggtgctgggtgccatcgagaacaaggtggagagcaaaggcaattcacctccgagctcaggagaggcctgtcgcgaggagaagggcctggctgccgaggatagtggtggggacagcaaggacctcagcgaggtcagcgagaccacagagagcacagatgtcaaggacagctcagaggcctccgactcagcctcctagagcctgccccatcccctcctcaccccacgagctttcacaataaattcgctccgtggcactggggaactttgtgtgtgagcgcgcgcacatttagagggtgtgtttctccaggtcctctggtggggatgtgagccttggccttttgacccagagcatcctgaaaaaaaaaaaaaaaaaa

SEQ ID NO: 136 - DNA, 6587 bp, Homo sapiens
  envoplakin (EVPL, EVPK), 210 kDa paraneoplastic pemphigus antigen, 210 kDa cornified envelope precursor protein
agtgtggccgttgtgggtgcatgcgcgtcaggcctgggacccggccgcccgcccgctgcctcacctgcaaggaggggcctcccagaaactcccttccccagtgcccagccgccccacctcgccagacttagctgaccagccagtgaggacgcccgctgcctcccacctgccctcctgccgtctttcgccagccaagcccagcctgagccagcacttgcctttacgaccatgttcaaggggctgagcaaaggctcccaggggaaggggtcccccaagggctcccccgccaaggggtcccccaaaggctcccccagcaggcacagccgggctgccacccaggagctggcccttctcatctcccgcatgcaagccaacgccgaccaggtggagcgggacatcctggagacgcagaagaggctgcagcaggaccggctgaacagtgagcagagccaggccctgcagcaccagcaggagacgggccgcagcctgaaggaggctgaggtgctgctcaaggacctcttcctggacgtggacaaggcccggcggctcaagcacccgcaggctgaggagattgagaaggacatcaagcagctgcacgagcgggtgacccaggagtgtgcggagtaccgtgccctgtacgagaagatggtgctgccccccgacgtgggacccagggtcgactgggcacgcgtgctggagcagaaacagaagcaggtctgcgcaggccagtacgggccgggcatggcggagctggagcaacagatcgccgagcacaacatcctgcagaaggagatcgacgcctatgggcagcagctgcggagcctcgtggggccggatgcagccaccatccggagccaataccgagacctactgaaggcggcgtcgtggcgcgggcagagcctgggcagcctgtacacgcacctccagggctgcacgcggcagctgagcgccctggctgagcagcagcgccgcatcctgcagcaggactggagcgacctcatggccgaccctgcgggcgtgcggcgggagtacgagcacttcaagcagcacgagctgctgagccaggagcagagcgtgaaccagctggaggacgacggcgagcgcatggtggagctgcggcaccccgcggtggggcccatccaggcccaccaggaggccctgaagatggagtggcagaacttcctgaacctgtgtatctgccaggagacccagctgcagcacgtggaggactaccgccggttccaggaagaggccgactcagtcagccagaccctggcgaagctcaactccaacttggatgccaagtacagccctgcacctgggggcccccctggcgcccccacagagctgctgcaacagctggaggcagaggaaaaacggctggccgtcaccgagagggccactggggacctgcagcggcgaagccgggatgtggcccctctgccacagcgaagaaacccccctcagcagcccctgcacgtggacagcatctgcgactgggactcaggagaagtgcagctgctgcagggtgagcggtataagctggtagataacactgacccgcacgcctgggtcgtgcagggccctggcggggagaccaagcgtgctcccgccgcctgcttctgcatcccagcaccagaccctgatgctgtggccagggcctcccggctggcctcagagctgcaggccctgaagcagaaattggccacagtccagagccgcctgaaggccagtgctgtggagtctcttcggcccagccagcaggctccatctggctcagacctggccaacccacaggcccagaagctcctgacacagatgacccggctggatggagacctgggacagatagagaggcaggtgctggcctgggcgcgggccccgctgagccgccccacacccttggaggacttggagggccgcatccacagccatgagggcacagcccagcgcctgcagagcctgggaacggagaaggagacagcccagaaggagtgcgaggcgtttctgtccacgcggcccgtgggccccgctgccctgcagctgcccgtagccctcaacagcgtgaagaacaagttcagtgacgtgcaggttctgtgcagcctctacggggagaaagccaaggctgccctggatctggagcggcagatccaggatgcggacagggtcatccgaggcttcgaggccaccctggtgcaggaggcccccatccctgctgaaccgggggctctgcaggagagggtcagcgagctgcagcgccagcggagggagctgctggaacagcagacctgcgtgctgcggctacaccgcgcgctgaaggcctcggagcacgcatgcgctgccctgcagaacaacttccaggagttctgccaagacctgcctcgccagcagcgccaggtgcgagccctcaccgaccgctaccacgccgtaggggaccagctggacctgcgggagaaggtggtgcaggatgccgccctcacctaccagcagttcaagaactgcaaggataacctgagctcctggctggagcacctgccccgcagccaggtgcggcccagcgacggccccagccagatcgcctacaagctgcaggcgcagaagaggctgacgcaggagatccagagccgagagcgggacagggccacagcatcccacctctcccaggccctgcaggcagcgctccaggactatgagctccaggcagacacctaccgctgctctttggagcccaccctggcagtgtcagcccccaagagaccccgagtggctcccctgcaagagagcatccaagcccaggagaagaaccttgcaaaggcctatactgaggttgcagcagcacagcagcagctgctccagcagctggagtttgctagaaaaatgctggagaagaaggagctcagtgaggacatccgaaggacccatgatgcaaagcagggctccgagagccctgcccaagcagggagagagtcagaggccctgaaggcccagctggaagaggagaggaagcgggtggcccgggtgcagcatgagctggaggcgcagaggagccaactgctgcagctgaggacccagcggcccttggagaggctggaggagaaggaagtggtagagttctaccgggacccccagctggagggcagcctgtccagggtgaaggcccaggtggaggaggagggcaagcggcgggctggcctgcaggcagacctggaagtggcagcccagaaggtcgtgcagctggaaagcaagaggaagaccatgcagcctcatctgctgaccaaggaggtcacccaggtggagagggaccccggcctggacagccaggcggcccagctcaggatccagatccagcagctccgcggggaggatgccgtcatctcggcccggctggaagggctgaagaaggagctactggcccttgagaagagggaggtggacgtgaaggagaaggtcgtggtgaaagaggtagtcaaggtggagaagaatctggaaatggtcaaggcagcccaggctctgaggctgcagatggaggaggatgctgcgcggaggaagcaggcggaggaggctgtggccaagctacaggctcgcatcgaagacctggagcgggctatcagctcggtggagcccaaggtcatcgtgaaggaggtgaagaaggtggagcaggacccagggctcctccaggagtcctccaggctgaggagcctcctcgaggaggagaggaccaagaacgcgacgctggccagggagctgagcgacctgcacagcaagtacagcgtggtggagaagcagaggcccaaagtgcagctccaggagcgcgtccacgagatcttccaggtggatccggagacagagcaggagatcactcggctcaaggccaagctgcaggagatggcgggcaagaggagcggtgtggagaaggaggtggagaagctgctgcccgacctggaggtcctgcgggcccagaagcccacggtggagtacaaggaggtgacccaggaggtggtgaggcatgagaggagccccgaggtgctgcgtgagatcgaccgcctgaaggctcagctcaacgagctcgtcaacagccacgggcgctcccaggagcagctcatccgcctgcagggtgagcgcgacgagtggaggcgcgagcgggccaaggtggagaccaagacggtgagcaaggaggtggtgcgccacgagaaggacccggtgctggagaaagaagcagagcggctccgccaggaggtgcgggaggcggcccagaagaggcgggccgcggaggacgcggtgtacgagctgcagagcaagcgcctgctgctggagaggaggaagcccgaggagaaggtggtggtgcaggaggtggtggtcacccagaaggacccgaagctgcgcgaggagcacagccggctgagcgggagcctggatgaggaggtgggccggcggcgccagctagagcttgaggtgcagcagctgcgggccggcgtggaggagcaggagggcctgctcagcttccaggaggaccgcagcaagaagctggccgtggagagggagctgcggcagctgaccttgaggatccaggagctcgagaagcggcctcccacggtgcaggagaagatcatcatggaggaagtggtcaagctggagaaggacccggacctggagaagtccacggaagccctgcggtgggacctggaccaggagaagacccaggtaaccgagctgaatcgggagtgcaagaacctgcaggtccagattgacgtcctccagaaagccaaatcgcaggagaagaccatctacaaggaagtgatccgggtgcagaaggaccgcgtcctggaagatgagcgggcccgcgtgtgggagatgctcaacagggagcgcacggcccggcaggcccgggaggaggaggcacggcgcctgcgggagcgcattgaccgggccgagacgctggggagaacctggtcccgggaggagtccgagctgcagagggcccgggaccaggccgaccaggagtgtgggcggctgcagcaggagctgcgggctctggagaggcagaagcagcagcagacactgcagctgcaggaggagtcgaagctgctcagccagaagacggagagcgagcgacagaaggcggcccagcggggccaggagctctcgcggctggaggcggccatcctccgcgagaaggaccagatctacgagaaggagcggacgctccgggacctccacgccaaggtgagccgggaggagctcagccaggagacccagacgcgagagaccaacctttccaccaagatctccatcctggaacccgagacggggaaggacatgtccccatacgaggcctacaagaggggcatcatcgacaggggccagtacttgcagctgcaggagctcgagtgtgactgggaggaggtcaccacctcggggccctgtggggaggagtctgtgctcctggaccgcaagagcgggaagcagtactccatcgaggccgccctccgctgccggcgcatctctaaggaggagtaccatctgtacaaggacggccacctgcccatctccgagtttgcgctgcttgtagctggggagaccaagccaagctcctcactctccatcggctctatcatctccaagtccccgctcgcctccccggccccccagagcaccagtttcttctctcccagcttctctctcgggctcggtgatgacagcttccctatcgccgggatctatgacacaaccacagacaacaagtgcagcatcaagacggccgtggccaagaacatgctggaccccatcactgggcagaagctactggaggcccaggcggccacagggggcatcgtggacctgctcagccgtgagcgctactctgtgcacaaggcgatggagaggggcctgatcgagaacacctccacacagaggctgcttaacgcccagaaggccttcaccggcatcgaggaccccgtcaccaagaagaggctctcggtgggcgaggccgtccagaagggctggatgccccgggagagcgtgctcccacacctgcaggtgcagcacctgaccggggggctcatcgaccccaagaggacaggccgcatccccatccagcaggccctcctctccgggatgatcagtgaagagctggcccagctcctgcaggacgagtccagctacgagaaggatttgacagaccccatctccaaggaacggctgagctacaaggaggccatgggccgctgccgcaaagaccccctgagcggcctgctgctcctgccagcggcactggaggggtaccgctgctaccgctccgcctcccccaccgtcccgcgctcccttcgctgacacgggccaaggagccagtggggaagtgcgtgtgttgggccaggtaggatacgtacacctcttgcctcagagcagcctcatcccaggcagtgggtcttccctctgtccaaccactgttttattattttactaacatggtgatgggctccctcccctaaccttggtgcctgatccatccccagaccaggacagcagccactcagttcttcctccacctccacccagtgatcccaataaacgaattctgtctccccgtg

SEQ ID NO: 137 - DNA, 11426 bp, Homo sapiens
  laminin alpha-5 chain (LAMA5), laminin-10 subunit alpha, laminin-11 subunit alpha, laminin-15 subunit alpha
agacccgccgggctcccgccgcgcgcgctgtccctggagctcggggacgcggcccggagccgggaagatggcgaagcggctctgcgcggggagcgcactgtgtgttcgcggcccccggggccccgcgccgctgctgctggtcgggctggcgctgctgggcgcggcgcgggcgcgggaggaggcgggcggcggcttcagcctgcacccgccctacttcaacctggccgagggcgcccgcatcgccgcctccgcgacctgcggagaggaggccccggcgcgcggctccccgcgccccaccgaggacctttactgcaagctggtagggggccccgtggccggcggcgaccccaaccagaccatccggggccagtactgtgacatctgcacggctgccaacagcaacaaggcacaccccgcgagcaatgccatcgatggcacggagcgctggtggcagagtccaccgctgtcccgcggcctggagtacaacgaggtcaacgtcaccctggacctgggccaggtcttccacgtggcctacgtcctcatcaagtttgccaactcaccccggccggacctctgggtgctggagcggtccatggacttcggccgcacctaccagccctggcagttctttgcctcctccaagagggactgtctggagcggttcgggccacagacgctggagcgcatcacacgggacgacgcggccatctgcaccaccgagtactcacgcatcgtgcccctggagaacggagagatcgtggtgtccctggtgaacggacgtccgggcgccatgaatttctcctactcgccgctgctacgtgagttcaccaaggccaccaacgtccgcctgcgcttcctgcgtaccaacacgctgctgggccatctcatggggaaggcgctgcgggaccccacggtcacccgccggtattattacagcatcaaggatatcagcatcggaggccgctgtgtctgccacggccacgcggatgcctgcgatgccaaagaccccacggacccgttcaggctgcagtgcacctgccagcacaacacctgcgggggcacctgcgaccgctgctgccccggcttcaatcagcagccgtggaagcctgcgactgccaacagtgccaacgagtgccagtcctgtaactgctacggccatgccaccgactgttactacgaccctgaggtggaccggcgccgcgccagccagagcctggatggcacctatcagggtgggggtgtctgtatcgactgccagcaccacaccaccggcgtcaactgtgagcgctgcctgcccggcttctaccgctctcccaaccaccctctcgactcgccccacgtctgccgccgctgcaactgcgagtccgacttcacggatggcacctgcgaggacctgacgggtcgatgctactgccggcccaacttctctggggagcggtgtgacgtgtgtgccgagggcttcacgggcttcccaagctgctacccgacgccctcgtcctccaatgacaccagggagcaggtgctgccagccggccagattgtgaattgtgactgcagcgcggcagggacccagggcaacgcctgccggaaggacccaagggtgggacgctgtctgtgcaaacccaacttccaaggcacccattgtgagctctgcgcgccagggttctacggccccggctgccagccctgccagtgttccagccctggagtggccgatgaccgctgtgaccctgacacaggccagtgcaggtgccgagtgggcttcgagggggccacatgtgatcgctgtgcccccggctactttcacttccctctctgccagttgtgtggctgcagccctgcaggaaccttgcccgagggctgcgatgaggccggccgctgcctatgccagcctgagtttgctggacctcattgtgaccggtgccgccctggctaccatggtttccccaactgccaagcatgcacctgcgaccctcggggagccctggaccagctctgtggggcgggaggtttgtgccgctgccgccccggctacacaggcactgcctgccaggaatgcagccccggctttcacggcttccccagctgtgtcccctgccactgctctgctgaaggctccctgcacgcagcctgtgacccccggagtgggcagtgcagctgccggccccgtgtgacggggctgcggtgtgacacatgtgtgcccggtgcctacaacttcccctactgcgaagctggctcttgccaccctgccggtctggccccagtggatcctgcccttcctgaggcacaggttccctgtatgtgccgggctcacgtggaggggccgagctgtgaccgctgcaaacctgggttctggggactgagccccagcaaccccgagggctgtacccgctgcagctgcgacctcaggggcacactgggtggagttgctgagtgccagccgggcaccggccagtgcttctgcaagccccacgtgtgcggccaggcctgcgcgtcctgcaaggatggcttctttggactggatcaggctgactattttggctgccgcagctgccggtgtgacattggcggtgcactgggccagagctgtgaaccgaggacgggcgtctgccggtgccgccccaacacccagggccccacctgcagcgagcctgcgagggaccactacctcccggacctgcaccacctgcgcctggagctggaggaggctgccacacctgagggtcacgccgtgcgctttggcttcaaccccctcgagttcgagaacttcagctggaggggctacgcgcagatggcacctgtccagcccaggatcgtggccaggctgaacctgacctcccctgaccttttctggctcgtcttccgatacgtcaaccggggggccatgagtgtgagcgggcgggtctctgtgcgagaggagggcaggtcggccacctgcgccaactgcacagcacagagtcagcccgtggccttcccacccagcacggagcctgccttcatcaccgtgccccagaggggcttcggagagccctttgtgctgaaccctggcacctgggccctgcgtgtggaggccgaaggggtgctcctggactacgtggttctgctgcctagcgcatactacgaggcggcgctcctgcagctgcgggtgactgaggcctgcacataccgtccctctgcccagcagtctggcgacaactgcctcctctacacacacctccccctggatggcttcccctcggccgccgggctggaggccctgtgtcgccaggacaacagcctgccccggccctgccccacggagcagctcagcccgtcgcacccgccactgatcacctgcacgggcagtgatgtggacgtccagcttcaagtggcagtgccacagccaggccgctatgccctagtggtggagtacgccaatgaggatgcccgccaggaggtgggcgtggccgtgcacaccccacagcgggccccccagcaggggctgctctccctgcacccctgcctgtacagcaccctgtgccggggcactgcccgggatacccaggaccacctggctgtcttccacctggactcggaggccagcgtgaggctcacagccgaacaggcacgcttcttcctgcacggggtcactctggtgcccattgaggagttcagcccggagttcgtggagccccgggtcagctgcatcagcagccacggcgcctttggccccaacagtgccgcctgtctgccctcgcgcttcccaaagccgccccagcccatcatcctcagggactgccaggtgatcccgctgccgcccggcctcccgctgacccacgcgcaggatctcactccagccatgtccccagctggaccccgacctcggccccccaccgctgtggaccctgatgcagagcccaccctgctgcgtgagccccaggccaccgtggtcttcaccacccatgtgcccacgctgggccgctatgccttcctgctgcacggctaccagccagcccaccccaccttccccgtggaagtcctcatcaacgccggccgcgtgtggcagggccacgccaacgccagcttctgtccacatggctacggctgccgcaccctggtggtgtgtgagggccaggccctgctggacgtgacccacagcgagctcactgtgaccgtgcgtgtgcccaagggccggtggctctggctggattatgtactcgtggtccctgagaacgtctacagctttggctacctccgggaggagcccctggataaatcctatgacttcatcagccactgcgcagcccagggctaccacatcagccccagcagctcatccctgttctgccgaaacgctgctgcttccctctccctcttctataacaacggagcccgtccatgtggctgccacgaagtaggtgctacaggccccacgtgtgagcccttcgggggccagtgtccctgccatgcccatgtcattggccgtgactgctcccgctgtgccaccggatactggggcttccccaactgcaggccctgtgactgcggtgcccgcctctgtgacgagctcacgggccagtgcatctgcccgccacgcaccatcccgcccgactgcctgctgtgccagccccagacctttggctgccaccccctggtcggctgtgaggagtgtaactgctcagggcccggcatccaggagctcacagaccctacctgtgacacagacagcggccagtgcaagtgcagacccaacgtgactgggcgccgctgtgatacctgctctccgggcttccatggctacccccgctgccgcccctgtgactgtcacgaggcgggcactgcgcctggcgtgtgtgaccccctcacagggcagtgctactgtaaggagaacgtgcagggccccaaatgtgaccagtgcagccttgggaccttctcactggatgctgccaaccccaaaggttgcacccgctgcttctgctttggggccacggagcgctgccggagctcgtcctacacccgccaggagttcgtggatatggagggatgggtgctgctgagcactgaccggcaggtggtgccccacgagcggcagccagggacggagatgctccgtgcagacctgcggcacgtgcctgaggctgtgcccgaggctttccccgagctgtactggcaggccccaccctcctacctgggggaccgggtgtcatcctacggtgggaccctccgttatgaactgcactcagagacccagcggggagatgtctttgtccccatggagagcaggccggatgtggtgctgcagggcaaccagatgagcatcacattcctggagccggcataccccacgcctggccacgttcaccgtgggcagctgcagctggtggaggggaacttccggcatacggagacgcgcaacactgtgtcccgcgaggagctcatgatggtgctggccagcctggagcagctgcagatccgtgccctcttctcacagatctcctcggctgtcttcctgcgcagggtggcactggaggtggccagcccagcaggccagggggccctggccagcaatgtggagctgtgcctgtgccccgccagctaccggggggactcatgccaggaatgtgcccccggcttctatcgggacgtcaaaggtctcttcctgggccgatgtgtcccttgtcagtgccatggacactcagaccgctgcctccctggctctggcgtctgtgtggactgccagcacaacaccgaaggggcccactgtgagcgctgccaggctggcttcgtgagcagcagggacgaccccagcgccccctgtgtcagctgcccctgccccctctcagtgccttccaacaacttcgccgagggctgtgtcctgcgaggcggccgcacccagtgcctctgcaaacctggttatgcaggtgcctcctgcgagcggtgtgcgcccggattctttgggaacccactggtgctgggcagctcctgccagccatgcgactgcagcggcaacggtgaccccaacttgctcttcagcgactgcgaccccctgacgggcgcctgccgtggctgcctgcgccacaccactgggccccgctgcgagatctgtgcccccggcttctacggcaacgccctgctgcccggcaactgcacccggtgcgactgtaccccatgtgggacagaggcctgcgacccccacagcgggcactgcctgtgcaaggcgggcgtgactgggcggcgctgtgaccgctgccaggagggacattttggtttcgatggctgcgggggctgccgcccgtgtgcttgtggaccggccgccgagggctccgagtgccacccccagagcggacagtgccactgccgaccagggaccatgggaccccagtgccgcgagtgtgcccctggctactgggggctccctgagcagggctgcaggcgctgccagtgccctgggggccgctgtgaccctcacacgggccgctgcaactgccccccggggctcagcggggagcgctgcgacacctgcagccagcagcatcaggtgcctgttccaggcgggcctgtgggccacagcatccactgtgaagtgtgtgaccactgtgtggtcctgctcctggatgacctggaacgggccggcgccctcctccccgccattcacgagcaactgcgtggcatcaatgccagctccatggcctgggcccgtctgcacaggctgaacgcctccatcgctgacctgcagagccagctccggagccccctgggcccccgccatgagacggcacagcagctggaggtgctggagcagcagagcacaagcctcgggcaggacgcacggcggctaggcggccaggccgtggggacccgagaccaggcgagccaattgctggccggcaccgaggccacactgggccatgcgaagacgctgttggcggccatccgggctgtggaccgcaccctgagcgagctcatgtcccagacgggccacctggggctggccaatgcctcggctccatcaggtgagcagctgctccggacactggccgaggtggagcggctgctctgggagatgcgggcccgggacctgggggccccgcaggcagcagctgaggctgagttggctgcagcacagagattgctggcccgggtgcaggagcagctgagcagcctctgggaggagaaccaggcactggccacacaaacccgcgaccggctggcccagcacgaggccggcctcatggacctgcgagaggctttgaaccgggcagtggacgccacacgggaggcccaggagctcaacagccgcaaccaggagcgcctggaggaagccctgcaaaggaagcaggagctgtcccgggacaatgccaccctgcaggccactctgcatgcggctagggacaccctggccagcgtcttcagattgctgcacagcctggaccaggctaaggaggagctggagcgcctcgccgccagcctggatggggctcggaccccactgctgcagaggatgcagaccttctccccggcgggcagcaagctgcgtctagtggaggccgccgaggcccacgcacagcagctgggccagctggcactcaatctgtccagcatcatcctggacgtcaaccaggaccgcctcacccagagggccatcgaggcctccaacgcctacagccgcatcctgcaggccgtgcaggctgccgaggatgctgctggccaggccctgcagcaggcggaccacacgtgggcgacggtggtgcggcagggcctggtggaccgagcccagcagctcctggccaacagcactgcactagaagaggccatgctccaggaacagcagaggctgggccttgtgtgggctgccctccagggtgccaggacccagctccgagatgtccgggccaagaaggaccagctggaggcgcacatccaggcggcgcaggccatgcttgccatggacacagacgagacaagcaagaagatcgcacatgccaaggctgtggctgctgaagcccaggacaccgccacccgtgtgcagtcccagctgcaggccatgcaggagaatgtggagcggtggcagggccagtacgagggcctgcggggccaggacctgggccaggcagtgcttgacgcaggccactcagtgtccaccctggagaagacgctgccccagctgctggccaagctgagcatcctggagaaccgtggggtgcacaacgccagcctggccctgtccgccagcattggccgcgtgcgagagctcattgcccaggcccggggggctgccagtaaggtcaaggtgcccatgaagttcaacgggcgctcaggggtgcagctgcgcaccccacgggatcttgccgaccttgctgcctacactgccctcaagttctacctgcagggcccagagcctgagcctgggcagggtaccgaggatcgctttgtgatgtacatgggcagccgccaggccactggggactacatgggtgtgtctctgcgtgacaagaaggtgcactgggtgtatcagctgggtgaggcgggccctgcagtcctaagcatcgatgaggacattggggagcagttcgcagctgtcagcctggacaggactctccagtttggccacatgtccgtcacagtggagagacagatgatccaggaaaccaagggtgacacggtggcccctggggcagaggggctgctcaacctgcggccagacgacttcgtcttctacgtcggggggtaccccagtaccttcacgccccctcccctgcttcgcttccccggctaccggggctgcatcgagatggacacgctgaatgaggaggtggtcagcctctacaacttcgagaggaccttccagctggacacggctgtggacaggccttgtgcccgctccaagtcgaccggggacccgtggctcacggacggctcctacctggacggcaccggcttcgcccgcatcagcttcgacagtcagatcagcaccaccaagcgcttcgagcaggagctgcggctcgtgtcctacagcggggtgctcttcttcctgaagcagcagagccagttcctgtgcttggccgtgcaagaaggcagcctcgtgctgttgtatgactttggggctggcctgaaaaaggccgtcccactgcagcccccaccgcccctgacctcggccagcaaggcgatccaggtgttcctgctggggggcagccgcaagcgtgtgctggtgcgtgtggagcgggccacggtgtacagcgtggagcaggacaatgatctggagctggccgacgcctactacctggggggcgtgccgcccgaccagctgcccccgagcctgcgacggctcttccccaccggaggctcagtccgtggctgcgtcaaaggcatcaaggccctgggcaagtatgtggacctcaagcggctgaacacgacaggcgtgagcgccggctgcaccgccgacctgctggtggggcgcgccatgactttccatggccacggcttccttcgcctggcgctctcgaacgtggcaccgctcactggcaacgtctactccggcttcggcttccacagcgcccaggacagtgccctgctctactaccgggcgtccccggatgggctatgccaggtgtccctgcagcagggccgtgtgagcctacagctcctgaggactgaagtgaaaactcaagcgggcttcgccgatggtgccccccattacgtcgccttctacagcaatgccacgggagtctggctgtatgtcgatgaccagctccagcagatgaagccccaccggggaccaccccccgagctccagccgcagcctgaggggcccccgaggctcctcctgggaggcctgcctgagtctggcaccatttacaacttcagtggctgcatcagcaacgtcttcgtgcagcggctcctgggcccacagcgcgtatttgatctgcagcagaacctgggcagcgtcaatgtgagcacgggctgtgcacccgccctgcaagcccagaccccgggcctggggcctagaggactgcaggccaccgcccggaaggcctcccgccgcagccgtcagcccgcccggcatcctgcctgcatgctgcccccacacctcaggaccacccgagactcctaccagtttgggggttccctgtccagtcacctggagtttgtgggcatcctggcccgacataggaactggcccagtctctccatgcacgtcctcccgcgaagctcccgaggcctcctcctcttcactgcccgtctgaggcccggcagcccctccctggcgctcttcctgagcaatggccacttcgttgcacagatggaaggcctcgggactcggctccgcgcccagagccgccagcgctcccggcctggccgctggcacaaggtctccgtgcgctgggagaagaaccggatcctgctggtgacggacggggcccgggcctggagccaggaggggccgcaccggcagcaccagggggcagagcacccccagccccacaccctctttgtgggcggcctcccggccagcagccacagctccaaacttccggtgaccgtcgggttcagcggctgtgtgaagagactgaggctgcacgggaggcccctgggggcccccacacggatggcaggggtcacaccctgcatcttgggccccctggaggcgggcctgttcttcccaggcagcgggggagttatcactttagacctcccaggagctacactgcctgatgtgggcctggaactggaggtgcggcccctggcagtcaccggactgatcttccacttgggccaggcccggacgcccccctacttgcagttgcaggtgaccgagaagcaagtcctgctgcgggcggatgacggagcaggggagttctccacgtcagtgacccgcccctcagtgctgtgtgatggccagtggcaccggctagcggtgatgaaaagcgggaatgtgctccggctggaggtggacgcgcagagcaaccacaccgtgggccccttgctggcggctgcagctggtgccccagcccctctgtacctcgggggcctgcctgagcccatggccgtgcagccctggccccccgcctactgcggctgcatgaggaggctggcggtgaaccggtcccccgtcgccatgactcgctctgtggaggtccacggggcagtgggggccagtggctgcccagccgcctaggacacagccaaccccggcccctggtcaggcccctgcagctgcctcacaccgccccttgtgctcgcctcataggtgtctatttggactctaagctctacgggtgacagatcttgtttctgaagatggtttaagttatagcttcttaaacgaaagaataaaatactgcaaaatgtttttatatttggcccttccacccatttttaattgtgagagatttgtcaccaatcatcactggttcctccttaaaaattaaaaagtaacttctgtgtaa

SEQ ID NO: 138 - DNA, 6930 bp, Homo sapiens
  myosin, heavy chain 14, non-muscle transcript variant 3 (MYH14), nonmuscle myosin heavy chain II-C (NMHC II-C, NMHC-II-C), myosin-14, MYH14 variant protein, DFNA4, DFNA4A, FP17425, MHC16, MYH17, PNMHH
ctctttctccccaggccgaagcctcgggacggccctggaagccgaccatggcagccgtgaccatgtcggtgcccgggcggaaggcgccccccaggccgggcccagtgcccgaggcggcccagccgttcctgttcacgccccgcgggcccagcgcgggtggcgggcctggctcgggcacctccccgcaggtggagtggacggcccggcgtctcgtgtgggtgccttcggagcttcacgggttcgaggcggcggcgctgcgggacgaaggcgaggaggaggcggaggtggagctggcggagagcgggaggcggctgcgactgccgcgggaccagatccagcgcatgaacccgcccaagttcagcaaggccgaggacatggccgagctgacctgcctcaacgaggcctcggtcctgcacaacctccgggagcggtactactccggcctcatctacacgtactccggccttttctgtgtggtcatcaacccgtacaagcagcttcccatctacacagaagccattgtggagatgtaccggggcaagaagcgccacgaggtgccaccccacgtgtacgcagtgaccgagggggcctatcggagcatgctgcaggatcgtgaggaccagtccattctctgcactggagagtctggagctgggaagacggaaaacaccaagaaggtcatccagtacctcgcccacgtggcgtcgtctccaaagggcaggaaggagccgggtgtccccgcctccgtcagcaccgtgtcttatggtgagctggagcggcagctgcttcaggccaaccccatcctagaggcctttggcaatgccaagacagtgaagaatgacaactcctcccgattcggcaaattcatccgcatcaactttgatgttgccgggtacatcgtgggcgccaacattgagacctacctgctggagaagtcgcgggccatccgccaggccaaggacgagtgcagcttccacatcttctaccagctgctggggggcgctggagagcagctcaaagccgacctcctcctcgagccctgctcccactaccggttcctgaccaacgggccgtcatcctctcccggccaggagcgggaactcttccaggagacgctggagtcgctgcgggtcctgggattcagccacgaggaaatcatctccatgctgcggatggtctcagcagttctccagtttggcaacattgccttgaagagagaacggaacaccgatcaagccaccatgcctgacaacacagctgcacagaagctctgccgcctcttgggactgggggtgacggatttctcccgagccttgctcacccctcgcatcaaagttggccgagactatgtgcagaaagcccagactaaggaacaggctgacttcgcgctggaggccctggccaaggccacctacgagcgcctcttccgctggctggttctgcgcctcaaccgggccttggaccgcagcccccgccaaggcgcctccttcctgggcatcctggacatcgcgggctttgagatcttccagctgaactccttcgagcagctctgcatcaactacaccaacgagaagctgcagcagctcttcaaccacaccatgttcgtgctggagcaggaggagtaccagcgtgagggcatcccctggaccttcctcgactttggcctcgacctgcagccctgcatcgacctcatcgagcggccggccaacccccctggactcctggccctgctggatgaggagtgctggttcccgaaggccacagacaagtcgtttgtggagaaggtagcccaggagcagggcggccaccccaagttccagcggccgaggcacctgcgggatcaggccgacttcagtgttctccactacgcgggcaaggtcgactacaaggccaacgagtggctgatgaaaaacatggaccctctgaatgacaacgtcgcagccttgctccaccagagcacagaccggctgacggcagagatctggaaagacgaacatgggggcttccagcagttctctttccttggctccttcccaccgtcgcccccaggatctgcagagaggtgcagctctgctatttctccgccaggggtggagggcatcgtggggctggaacaggtgagcagcctgggcgacggcccaccaggtggccgcccccgtcggggtatgttccggacagtgggacagctctacaaggagtccctgagccgcctcatggccacactcagcaacaccaaccccagttttgtccgctgcattgtccccaaccacgagaagagggccgggaagctggagccacggctggtgctggaccagcttcgctgcaacggggtcctggagggcatccgcatctgtcgccagggcttccccaaccgcatcctcttccaggagttccggcagcgatacgagatcctgacacccaatgccatccccaagggcttcatggatgggaagcaggcctgtgaaaagatgatccaggcgctggaactggaccccaacctctaccgcgtgggacagagcaagatcttcttccgggctggggtcctggcccagctggaagaggagcgagacctgaaggtcaccgacatcatcgtctccttccaggcagctgcccggggatacctggctcgcagggccttccagaagcgccagcagcagcagagcgccctgagggtgatgcagcggaactgcgcggcctacctcaagctgagacactggcagtggtggcggctgtttaccaaggtgaagccactgctgcaggtgacgcggcaggatgaggtgctgcaggcacgggcccaggagctgcagaaagtgcaggagctacagcagcagagcgcccgcgaagttggggagctccagggccgagtggcacagctggaagaggagcgcgcccgcctggcagagcaattgcgagcagaggcagaactgtgtgcagaggccgaggagacgcgggggaggctggcagcccgcaagcaggagctggagctggtggtgtcagagctggaggctcgcgtgggcgaggaggaggagtgcagccgtcaaatgcaaaccgagaagaagaggctgcagcagcacatacaggagctagaggcccaccttgaggctgaggagggtgcgcggcagaagctgcagctggagaaggtgacgacagaggcaaaaatgaagaaatttgaagaggacctgctgctcctggaagaccagaattccaagctgagcaaggagcggaagctgctggaagatcgtctggccgagttctcatcccaggcagctgaggaggaggagaaggtcaagagcctcaataagctacggctcaaatatgaggccacaatcgcagacatggaggaccgcctacggaaggaggagaagggtcgccaggagctggagaagctgaagcggaggctggatggggagagctcagagctgcaggagcagatggtggagcagcaacagcgggcagaggagctgcgggcccagctgggccggaaggaggaggagctgcaggctgccctggccagggcagaagacgagggtggggcccgggcccagctgctgaaatccctgcgggaggctcaagcagccctggccgaggcccaggaggacctggagtctgagcgtgtggccaggaccaaggcggagaagcagcgccgggacctgggcgaggagctggaggcgctgcggggcgagctggaggacacgctggactccaccaacgcacagcaggagctccggtccaagagggaacaggaggtgacggagctgaagaagactctggaggaggagactcgcatccacgaggcggcagtgcaggagctgaggcagcgccacggccaggccctgggggagctggcggagcagctggagcaggcccggaggggcaaaggtgcatgggagaagacccggctggccctggaggccgaggtgtccgagctgcgggcagaactgagcagcctgcagactgcacgtcaggagggtgagcagcggaggcgccgcctggagttacagctgcaggaggtgcagggccgggctggtgatggggagagggcacgagcggaggctgctgagaagctgcagcgagcccaggctgaactggagaatgtgtctggggcgctgaacgaggctgagtccaaaaccatccgtcttagcaaggagctgagcagcacagaagcccagctgcacgatgcccaggagctgctgcaggaggagaccagggcgaaattggccttggggtcccgggtgcgagccatggaggctgaggcagccgggctgcgtgagcagctggaggaggaggcagctgccagggaacgggcgggccgtgaactgcagactgcccaggcccagctttccgagtggcggcggcgccaggaggaggaggcaggggcactggaggcaggggaggaggcacggcgccgggcagcccgggaggccgaggccctgacccagcgcctggcagaaaagacagagaccgtggatcggctggagcggggccgccgccggctgcagcaggagctggacgacgccaccatggacctggagcagcagcggcagcttgtgagcaccctggagaagaagcagcgcaagtttgaccagcttctggcagaggagaaggcagctgtacttcgggcagtggaggaacgtgagcgggccgaggcagagggccgggagcgtgaggctcgggccctgtcactgacacgggcactggaggaggagcaggaggcacgtgaggagctggagcggcagaaccgggccctgcgggctgagctggaggcactgctgagcagcaaggatgacgtcggcaagagcgtgcatgagctggaacgagcctgccgggtagcagaacaggcagccaatgatctgcgagcacaggtgacagaactggaggatgagctgacagcggccgaggatgccaagctgcgtctggaggtgactgtgcaggctctcaagactcagcatgagcgtgacctgcagggccgtgatgaggctggtgaagagaggcggaggcagctggccaagcagctgagagatgcagaggtggagcgggatgaggagcggaagcagcgcactctggccgtggctgcccgcaagaagctggagggagagctggaggagctgaaggctcagatggcctctgccggccagggcaaggaggaggcggtgaagcagcttcgcaagatgcaggcccagatgaaggagctatggcgggaggtggaggagacacgcacctcccgggaggagatcttctcccagaatcgggaaagtgaaaagcgcctcaagggcctggaggctgaggtgctgcggctgcaggaggaactggccgcctcggaccgtgctcggcggcaggcccagcaggaccgggatgagatggcagatgaggtggccaatggtaaccttagcaaggcagccattctggaggagaagcgtcagctggaggggcgcctggggcagttggaggaagagctggaggaggagcagagcaactcggagctgctcaatgaccgctaccgcaagctgctcctgcaggtagagtcactgaccacagagctgtcagctgagcgcagtttctcagccaaggcagagagcgggcggcagcagctggaacggcagatccaggagctacggggacgcctgggtgaggaggatgctggggcccgtgcccgccacaagatgaccattgctgcccttgagtctaagttggcccaggctgaggagcagctagagcaagagaccagagagcgcatcctctctggaaagctggtgcgcagagctgagaagcggcttaaagaggtggtgctccaggtggaggaggagcggagggtggctgaccagctccgggaccagctggagaagggaaaccttcgagtcaagcagctgaagcggcagctggaggaggccgaggaggaggcatcccgggctcaggccggccgccggaggctgcagcgtgagctggaagatgtcacagagtcggccgagtccatgaaccgtgaagtgaccacactgaggaaccggcttcgacgcggccccctcaccttcaccacccgcacggtgcgccaggtcttccgactagaggagggcgtggcatccgacgaggaggcagaggaagcacagcctgggtctgggccatccccggagcctgaggggtccccaccagcccacccccagtgaccctaccctgtccccagatgcactaacagatggggcccagcccccttcctccctggaccccacgggcccctgtcccaggaaccccgccctctgacttcttgccctttggaaatggtgcagcactctggcatttatcacccccacctgggtcccctgcaacctcccatcaaaggatgacccctaaacacagaggagcggggcaggcagggaggcaatgactggagctaccttgcttgttgggggactgggtacagttggcaagctgtgtttccatcagctccctgtcctcctttcttccctcgttattgatctatagacattaggaagggagtgagacggctcctccaccatcctcagccagtgcaacccattccctctgcttctctctctctctctctctctccctccctctccttccctaccctctcaccatctttcttggcctctctgagggtctctctgtgcatctttttaggaatctcgctctcactctctacgtagccactctccttcccccatttctgcgtccacccctgaactcctgagcgacagaagccccaggcctccaccagccttgaacccttgcaaaggggcaggacaaggggacccctctcactcctgctgctgcccatgctctgccctcccttctggttgctctgagggttcggagcttccctctgggactaaaggagtgtcctttaccctcccagcctccaggctctggcagaaataaactccaacccgactggaccataaaaaaaaaaaaaaaaaa

SEQ ID NO: 139 - DNA, 1531 bp, Homo sapiens
  Rab geranylgeranyltransferase, beta subunit (RABGGTB, GGTB), geranylgeranyl transferase type-2 subunit beta, type II protein geranyl-geranyltransferase subunit beta, GGTase- II-beta; rab GGTase beta; rab GG transferase beta
gagaggcgcatctgcgcaggcgcccggctcctaagtctacccaggaactgaccctgctctctcctttccctgttagacatgggcactccacagaaggatgttattatcaagtcagatgcaccggacactttgttattggagaaacatgcagattatatcgcatcctatggctcaaagaaagatgattatgaatactgtatgtctgagtatttgagaatgagtggcatctattggggtctgacagtaatggatctcatgggacaacttcatcgcatgaatagagaagagattctggcatttattaagtcttgccaacatgaatgtggtggaataagtgctagtatcggacatgatcctcatcttttatacactcttagtgctgtccagattcttacgctgtatgacagtattaatgttattgacgtaaataaagttgtggaatatgttaaaggtctacagaaagaagatggttcttttgctggagatatttggggagaaattgacacaagattctctttttgtgcggtggcaactttggctttgttggggaagcttgatgctattaatgtggaaaaggcaatcgaatttgttttatcctgtatgaactttgacggtggatttggttgcagaccaggttctgaatcccatgctgggcagatctattgttgcacaggatttctggcaattacaagtcagttgcatcaagtaaattctgatttacttggctggtggctttgtgaacgacaattaccctcaggcgggctcaatggaaggccggagaagttaccagatgtatgctactcatggtgggtcctggcttccctaaagataattggaagacttcattggattgatagagagaaactgcgtaatttcattttagcatgtcaagatgaagaaacggggggatttgcagacaggccaggagatatggtggatccttttcataccttatttggaattgctggattgtcacttttgggagaagaacagattaaacctgttaatcctgtcttttgcatgcctgaagaagtgcttcagagagtgaatgttcagcctgagctagtgagctagattcattgaattgaaagttgcatagtatagttttgccattttaacatttctgtatttgaagtgcttatcgaatctaaaagtgactactgttaatattttgtatattgtgttaaattaattttaataaattatataattatacatattgtaaaataaagaccggtattttattttctgctttttattctgaagtcctgttattctgactacagttctttgtgtatacttctgtgtctgttatgttcaataactgagctaacataaaataactctaggtttctacttgatttttcccccatgtatacctttcatctgttctatagcaagttgatgtaaattggtttgtcaacaagaatgttaactgatgaaagtggatagaacccatacatgaattaaatgatgcacaaaataaatggctgttgaaatttggaaatgattgaaaaaaaaaaaaaaaaaa

SEQ ID NO: 140 - DNA, 514 bp, Homo sapiens
  60S ribosomal protein L27, L27
tccttctttcctttttgctggtagggccgggtggttgctgccgaaatgggcaagttcatgaaacctgggaaggtggtgcttgtcctggctggacgctactccggacgcaaagctgtcatcgtgaagaacattgatgatggcacctcagatcgcccctacagccatgctctggtggctggaattgaccgctacccccgcaaagtgacagctgccatgggcaagaagaagatcgccaagagatcaaagataaaatcttttgtgaaagtgtataactacaatcacctaatgcccacaaggtactctgtggatatccccttggacaaaactgtcgtcaataaggatgtcttcagagatcctgctcttaaacgcaaggcccgacgggaggccaaggtcaagtttgaagagagatacaagacaggcaagaacaagtggttcttccagaaactgcggttttagatgctttgttttgatcattaaaaattataaagaaaaaaaaaaaaaaaaaaaaaaaaaa

SEQ ID NO: 141 - DNA, 531 bp, Homo sapiens
  40S ribosomal protein S15, S15, homolog of rat insulinoma (RIG), insulinoma protein
ggcagtctcgcgataactgcgcaggcgcggaccaaagcgatctcttctgaggatccggcaagatggcagaagtagagcagaagaagaagcggaccttccgcaagttcacctaccgcggcgtggacctcgaccagctgctggacatgtcctacgagcagctgatgcagctgtacagtgcgcgccagcggcggcggctgaaccggggcctgcggcggaagcagcactccctgctgaagcgcctgcgcaaggccaagaaggaggcgccgcccatggagaagccggaagtggtgaagacgcacctgcgggacatgatcatcctacccgagatggtgggcagcatggtgggcgtctacaacggcaagaccttcaaccaggtggagatcaagcccgagatgatcggccactacctgggcgagttctccatcacctacaagcccgtaaagcatggccggcccggcatcggggccacccactcctcccgcttcatccctctcaagtaatggctcagctaataaaggcgcacatgactcc

SEQ ID NO: 142 - DNA, 1712 bp, Homo sapiens
  inosine-5′-monophosphate dehydrogenase 2, inosine monophosphate dehydrogenase type II, IMP (inosine monophosphate) dehydrogenase 2, IMP (inosine monophosphate) dehydrogenase 2, IMP oxireductase 2 (IMPDH2, IMPD2, IMPDH-II, IMPD 2; IMPDH 2)
cgaaatcggctggtttatattggcgcggcccagacggcagaggtctctgcggcgcggtcctcggagacacgcggcggtgtcctgtgttggccatggccgactacctgattagtgggggcacgtcctacgtgccagacgacggactcacagcacagcagctcttcaactgcggagacggcctcacctacaatgactttctcattctccctgggtacatcgacttcactgcagaccaggtggacctgacttctgctctgaccaagaaaatcactcttaagaccccactggtttcctctcccatggacacagtcacagaggctgggatggccatagcaatggcgcttacaggcggtattggcttcatccaccacaactgtacacctgaattccaggccaatgaagttcggaaagtgaagaaatatgaacagggattcatcacagaccctgtggtcctcagccccaaggatcgcgtgcgggatgtttttgaggccaaggcccggcatggtttctgcggtatcccaatcacagacacaggccggatggggagccgcttggtgggcatcatctcctccagggacattgattttctcaaagaggaggaacatgactgtttcttggaagagataatgacaaagagggaagacttggtggtagcccctgcaggcatcacactgaaggaggcaaatgaaattctgcagcgcagcaagaagggaaagttgcccattgtaaatgaagatgatgagcttgtggccatcattgcccggacagacctgaagaagaatcgggactacccactagcctccaaagatgccaagaaacagctgctgtgtggggcagccattggcactcatgaggatgacaagtataggctggacttgctcgcccaggctggtgtggatgtagtggttttggactcttcccagggaaattccatcttccagatcaatatgatcaagtacatcaaagacaaataccctaatctccaagtcattggaggcaatgtggtcactgctgcccaggccaagaacctcattgatgcaggtgtggatgccctgcgggtgggcatgggaagtggctccatctgcattacgcaggaagtgctggcctgtgggcggccccaagcaacagcagtgtacaaggtgtcagagtatgcacggcgctttggtgttccggtcattgctgatggaggaatccaaaatgtgggtcatattgcgaaagccttggcccttggggcctccacagtcatgatgggctctctcctggctgccaccactgaggcccctggtgaatacttcttttccgatgggatccggctaaagaaatatcgcggtatgggttctctcgatgccatggacaagcacctcagcagccagaacagatatttcagtgaagctgacaaaatcaaagtggcccagggagtgtctggtgctgtgcaggacaaagggtcaatccacaaatttgtcccttacctgattgctggcatccaacactcatgccaggacattggtgccaagagcttgacccaagtccgagccatgatgtactctggggagcttaagtttgagaagagaacgtcctcagcccaggtggaaggtggcgtccatagcctccattcgtatgagaagcggcttttctgaaaagggatccagcacacctcctcggtttttttttcaataaaagtttagaaagaaaaaaaaaaaaaaaaaaaaaaa

SEQ ID NO: 143 - DNA, 1655 bp, Homo sapiens
  sigma non-opioid intracellular receptor 1 transcript variant 1, sigma 1-type opioid receptor (SIGMAR1, hSigmaR1, SIG-1R, sigma1R), SR31747 binding protein 1 (SR-BP1, SR-BP, SRBP), aging-associated gene 8 protein, ALS16
ctccgaggccgtgagcgcaaagcctcaggccccggctccctcctgagctgcgccgtgccaggccgcccgccgggatgcagtgggccgtgggccggcggtgggcgtgggccgcgctgctcctggctgtcgcagcggtgctgacccaggtcgtctggctctggctgggtacgcagagcttcgtcttccagcgcgaagagatagcgcagttggcgcggcagtacgctgggctggaccacgagctggccttctctcgtctgatcgtggagctgcggcggctgcacccaggccacgtgctgcccgacgaggagctgcagtgggtgttcgtgaatgcgggtggctggatgggcgccatgtgccttctgcacgcctcgctgtccgagtatgtgctgctcttcggcaccgccttgggctcccgcggccactcggggcgctactgggctgagatctcggataccatcatctctggcaccttccaccagtggagagagggcaccaccaaaagtgaggtcttctacccaggggagacggtagtacacgggcctggtgaggcaacagctgtggagtgggggccaaacacatggatggtggagtacggccggggcgtcatcccatccaccctggccttcgcgctggccgacactgtcttcagcacccaggacttcctcaccctcttctatactcttcgctcctatgctcggggcctccggcttgagctcaccacctacctctttggccaggacccttgaccagccaggcctgaaggaagacctgcggatggacaggagcgggcaggcccgcacatatccacttgctggagcccatgtttacagacagggacatacaccatgcagatcctgagttcctgctgtatgagcagggatatccatgcttatgtatccaaacacagagacccatgggaacaaatgagacacatatagatactgagacctgtgtgtacagtaggaccatgcactcacacccatctggagagggagcccccggtataccaagggagccagttgtgttcagacacacacatcacagcttgactcactaactgaggcctttccatagctccacagcttcccacctcctccccaccaaaccggggttctagagttaaggatgggggagggtattatactgcctcagtctgactcctcaacccagcagcaatttgaggggatgagggggaagaggagctgccttttggaggcccccttcacctgcagctatgatgcccttccccttctcccctgtcctcaccatatgccttatccccattctactcccctgctatgcaagtgcccctgtggcttgtccccaaccccctcagcaacaaagctcagctggggaacgagagtaatttgaagaatgcttgaagtcagcgtcttccattccagaaagacccccattcttcctttgggggtatgatgtggaagctggtttcagcccaggacccaccactgaggagaggatctagacaggtgggcctaattccaaggggcccttcctggcctggagaaggccttttacacacacacaacacatacacacacacacacacacacacatatcacagttttcacacagcccctgctgcattctctgtccatctgtctgtttctattaataaagatttgttgatctgttcc

SEQ ID NO: 144 - DNA, 898 bp, Homo sapiens
  ATP synthase, H+ transporting, mitochondrial F0 complex, subunit C2 (subunit 9) transcript variant 2, ATP synthase F(0) complex subunit C2, mitochondrial precursor (ATP5G2, ATP5A), ATP synthase lipid-binding protein, mitochondrial, ATP synthase proteolipid P2
ccacgttacggatcggcttactccgcggagttggcctcatttctgcagtcggcgctccctgtagtttctcctctcgaacgccaggtggagcaaccggccggataccgccacagccctggcaggcggcgctgtgatgcctgagctgatcctgtatgttgcaatcactctatccgtggctgagcgactcgttggcccgggtcacgcatgcgctgagccttcctttcgctcttcccgctgctccgcccctctctgtcttctctgcagtgggagcagctctcctgccacagctcctcaccccctgaaaatgttcgcctgctccaagtttgtctccactccctccttggtcaagagcacctcacagctgctgagccgtccgctatctgcagtggtgctgaaacgaccggagatactgacagatgagagcctcagcagcttggcagtctcatgtccccttacctcacttgtctctagccgcagcttccaaaccagcgccatttcaagggacatcgacacagcagccaagttcattggagctggggctgccacagttggggtggctggttctggggctgggattggaactgtgtttgggagcctcatcattggttatgccaggaacccttctctgaagcaacagctcttctcctacgccattctgggctttgccctctcggaggccatggggctcttttgtctgatggtagcctttctcatcctctttgccatgtgaaggagccgtctccacctcccatagttctcccgcgtctggttggccccgtgtgttccttttcctatacctccccaggcagcctggggaacgtggttggctcagggtttgacagagaaaagacaaataaatactgtattaataagatgtttcttgaaaaaaaaaaaaaaa

SEQ ID NO: 145 - RNA, 15 nt, Artificial Sequence
  synthetic 5′ untranslated region (5′UTR) consensus sequence pyrimidine rich translational element (PRTE)
cuuccuuucccugcu

SEQ ID NO: 146 - DNA, 26 bp, Artificial Sequence
  synthetic YB1 5′UTR cloning oligonucleotide deletion (20-34) forward
gctacaagcttgggcttatcccgcct

SEQ ID NO: 147 - DNA, 26 bp, Artificial Sequence
  synthetic YB1 5′UTR cloning  oligonucleotide deletion (20-34) reverse
tcgatccatggggttgcggtgatggt

SEQ ID NO: 148 - DNA, 40 bp, Artificial Sequence
  synthetic site-directed mutagenesis oligonucleotide deletion (20-34) forward
tgggcttatcccgcctgtccttcgatcggtagcgggagcg

SEQ ID NO: 149 - DNA, 40 bp, Artificial Sequence
  synthetic site-directed mutagenesis oligonucleotide deletion (20-34) reverse
cgctcccgctaccgatcgaaggacaggcgggataagccca

SEQ ID NO: 150 - DNA, 55 bp, Artificial Sequence
  synthetic site-directed mutagenesis oligonucleotide transversion (20-34) forward
tgggcttatcccgcctgtccgcggtaagagcgatcttcgatcggtagcgggagcg

SEQ ID NO: 151 - DNA, 55 bp, Artificial Sequence
  synthetic site-directed mutagenesis oligonucleotide transversion (20-34) reverse
cgctcccgctaccgatcgaagatcgctcttaccgcggacaggcgggataagccca

SEQ ID NO: 152 - DNA, 20 bp, Artificial Sequence
  synthetic human qPCR oligonucleotide beta-actin forward
gcaaagacctgtacgccaac

SEQ ID NO: 153 - DNA, 20 bp, Artificial Sequence
  synthetic human qPCR oligonucleotide beta-actin reverse
agtacttgcgctcaggagga

SEQ ID NO: 154 - DNA, 20 bp, Artificial Sequence
  synthetic human qPCR oligonucleotide CD44 forward
caacaacacaaatggctggt

SEQ ID NO: 155 - DNA, 24 bp, Artificial Sequence
  synthetic human qPCR oligonucleotide CD44 reverse
ctgaggtgtctgtctctttcatct

SEQ ID NO: 156 - DNA, 20 bp, Artificial Sequence
  synthetic human qPCR oligonucleotide vimentin forward
ggcccagctgtaagttggta

SEQ ID NO: 157 - DNA, 18 bp, Artificial Sequence
  synthetic human qPCR oligonucleotide vimentin reverse
ggagcgagagtggcagag

SEQ ID NO: 158 - DNA, 19 bp, Artificial Sequence
  synthetic human qPCR oligonucleotide Snail1 forward
cactatgccgcgctctttc

SEQ ID NO: 159 - DNA, 25 bp, Artificial Sequence
  synthetic human qPCR oligonucleotide Snail1 reverse
gctggaaggtaaactctggattaga

SEQ ID NO: 160 - DNA, 21 bp, Artificial Sequence
  synthetic human qPCR oligonucleotide YB1 forward
tcgccaaagacagcctagaga

SEQ ID NO: 161 - DNA, 22 bp, Artificial Sequence
  synthetic human qPCR oligonucleotide YB1 reverse
tctgcgtcggtaattgaagttg

SEQ ID NO: 162 - DNA, 21 bp, Artificial Sequence
  synthetic human qPCR oligonucleotide MTA1 forward
caaagtggtgtgcttctaccg

SEQ ID NO: 163 - DNA, 21 bp, Artificial Sequence
  synthetic human qPCR oligonucleotide MTA1 reverse
cggccttatagcagactgaca

SEQ ID NO: 164 - DNA, 20 bp, Artificial Sequence
  synthetic human qPCR oligonucleotide PLAU forward
ttgctcaccacaacgacatt

SEQ ID NO: 165 - DNA, 20 bp, Artificial Sequence
  synthetic human qPCR oligonucleotide PLAU reverse
ggcaggcagatggtctgtat

SEQ ID NO: 166 - DNA, 19 bp, Artificial Sequence
  synthetic human qPCR oligonucleotide FGFBP1 forward
actggatccgtgtgctcag

SEQ ID NO: 167 - DNA, 20 bp, Artificial Sequence
  synthetic human qPCR oligonucleotide FGFBP1 reverse
gagcagggtgaggctacaga

SEQ ID NO: 168 - DNA, 23 bp, Artificial Sequence
  synthetic human qPCR oligonucleotide ARID5B forward
tggactcaacttcaaagacgttc

SEQ ID NO: 169 - DNA, 20 bp, Artificial Sequence
  synthetic human qPCR oligonucleotide ARID5B reverse
acgttcgtttcttcctcgtc

SEQ ID NO: 170 - DNA, 20 bp, Artificial Sequence
  synthetic human qPCR oligonucleotide CTGF forward
ctcctgcaggctagagaagc

SEQ ID NO: 171 - DNA, 21 bp, Artificial Sequence
  synthetic human qPCR oligonucleotide CTGF reverse
gatgcactttttgcccttctt

SEQ ID NO: 172 - DNA, 19 bp, Artificial Sequence
  synthetic human qPCR oligonucleotide RND3 forward
aaaaactgcgctgctccat

SEQ ID NO: 173 - DNA, 21 bp, Artificial Sequence
  synthetic human qPCR oligonucleotide RND3 reverse
tcaaaactggccgtgtaattc

SEQ ID NO: 174 - DNA, 20 bp, Artificial Sequence
  synthetic human qPCR oligonucleotide KLF6 forward
aaagctcccacttgaaagca

SEQ ID NO: 175 - DNA, 21 bp, Artificial Sequence
  synthetic human qPCR oligonucleotide KLF6 reverse
ccttcccatgagcatctgtaa

SEQ ID NO: 176 - DNA, 18 bp, Artificial Sequence
  synthetic human qPCR oligonucleotide BCL6 forward
ttccgctacaagggcaac

SEQ ID NO: 177 - DNA, 20 bp, Artificial Sequence
  synthetic human qPCR oligonucleotide BCL6 reverse
tgcaacgatagggtttctca

SEQ ID NO: 178 - DNA, 20 bp, Artificial Sequence
  synthetic human qPCR oligonucleotide FOXA1 forward
agggctggatggttgtattg

SEQ ID NO: 179 - DNA, 18 bp, Artificial Sequence
  synthetic human qPCR oligonucleotide FOXA1 reverse
accgggacggaggagtag

SEQ ID NO: 180 - DNA, 18 bp, Artificial Sequence
  synthetic human qPCR oligonucleotide GDF15 forward
ccggatactcacgccaga

SEQ ID NO: 181 - DNA, 20 bp, Artificial Sequence
  synthetic human qPCR oligonucleotide GDF15 reverse
agagatacgcaggtgcaggt

SEQ ID NO: 182 - DNA, 18 bp, Artificial Sequence
  synthetic human qPCR oligonucleotide HBP1 forward
gctggtggtgttgtcgtg

SEQ ID NO: 183 - DNA, 22 bp, Artificial Sequence
  synthetic human qPCR oligonucleotide HBP1 reverse
catgttatggtgctctgactgc

SEQ ID NO: 184 - DNA, 20 bp, Artificial Sequence
  synthetic human qPCR oligonucleotide Twist1 forward
catcctcacacctctgcatt

SEQ ID NO: 185 - DNA, 20 bp, Artificial Sequence
  synthetic human qPCR oligonucleotide Twist1 reverse
ttcctttcagtggctgattg

SEQ ID NO: 186 - DNA, 23 bp, Artificial Sequence
  synthetic human qPCR oligonucleotide LEF1 forward
ccttggtgaacgagtctgaaatc

SEQ ID NO: 187 - DNA, 20 bp, Artificial Sequence
  synthetic human qPCR oligonucleotide LEF1 reverse
gaggtttgtgcttgtctggc

SEQ ID NO: 188 - DNA, 20 bp, Artificial Sequence
  synthetic human qPCR oligonucleotide rpS19 forward
gctggccaaacataaagagc

SEQ ID NO: 189 - DNA, 20 bp, Artificial Sequence
  synthetic human qPCR oligonucleotide rpS19 reverse
ctgggtctgacaccgtttct

SEQ ID NO: 190 - DNA, 19 bp, Artificial Sequence
  synthetic human qPCR oligonucleotide 5S rRNA forward
gcccgatctcgtctgatct

SEQ ID NO: 191 - DNA, 20 bp, Artificial Sequence
  synthetic human qPCR oligonucleotide 5S rRNA reverse
agcctacagcacccggtatt

SEQ ID NO: 192 - DNA, 20 bp, Artificial Sequence
  synthetic human qPCR oligonucleotide firefly luciferase forward
aatcaaagaggcgaactgtg

SEQ ID NO: 193 - DNA, 20 bp, Artificial Sequence
  synthetic human qPCR oligonucleotide firefly luciferase reverse
ttcgtcttcgtcccagtaag

SEQ ID NO: 194 - DNA, 20 bp, Artificial Sequence
  synthetic mouse qPCR oligonucleotide beta actin forward
ctaaggccaaccgtgaaaag

SEQ ID NO: 195 - DNA, 20 bp, Artificial Sequence
  synthetic mouse qPCR oligonucleotide beta actin reverse
accagaggcatacagggaca

SEQ ID NO: 196 - DNA, 20 bp, Artificial Sequence
  synthetic mouse qPCR oligonucleotide Yb1 forward
gggttacagaccacgattcc

SEQ ID NO: 197 - DNA, 18 bp, Artificial Sequence
  synthetic mouse qPCR oligonucleotide Yb1 reverse
ggcgataccgacgttgag

SEQ ID NO: 198 - DNA, 20 bp, Artificial Sequence
  synthetic mouse qPCR oligonucleotide vimentin forward
tccagcagcttcctgtaggt

SEQ ID NO: 199 - DNA, 20 bp, Artificial Sequence
  synthetic mouse qPCR oligonucleotide vimentin reverse
ccctcacctgtgaagtggat

SEQ ID NO: 200 - DNA, 20 bp, Artificial Sequence
  synthetic mouse qPCR oligonucleotide Cd44 forward
acagtaccttacccaccatg

SEQ ID NO: 201 - DNA, 20 bp, Artificial Sequence
  synthetic mouse qPCR oligonucleotide Cd44 reverse
ggatgaatcctcggaattac

SEQ ID NO: 202 - DNA, 20 bp, Artificial Sequence
  synthetic mouse qPCR oligonucleotide Mta1 forward
agtgcgcctaatccgtggtg

SEQ ID NO: 203 - DNA, 22 bp, Artificial Sequence
  synthetic mouse qPCR oligonucleotide Mta1 reverse
ctgaggatgagagcagctttcg

SEQ ID NO: 204 - RNA, 19 nt, Artificial Sequence
  synthetic siRNA/shRNA Control (D-001810-01) sequence
ugguuuacaugucgacuaa

SEQ ID NO: 205 - RNA, 19 nt, Artificial Sequence
  synthetic siRNA/shRNA vimentin (L-003551) sequence
ucacgaugaccuugaauaa

SEQ ID NO: 206 - RNA, 19 nt, Artificial Sequence
  synthetic siRNA/shRNA vimentin (L-003551) sequence
ggaaauggcucgucaccuu

SEQ ID NO: 207 - RNA, 19 nt, Artificial Sequence
  synthetic siRNA/shRNA vimentin (L-003551) sequence
gagggaaacuaaucuggau

SEQ ID NO: 208 - RNA, 19 nt, Artificial Sequence
  synthetic siRNA/shRNA vimentin (L-003551) sequence
uuaagacgguugaaacuag

SEQ ID NO: 209 - RNA, 19 nt, Artificial Sequence
  synthetic siRNA/shRNA YB1 (L-10213) sequence
cugaguaaaugccggcuua

SEQ ID NO: 210 - RNA, 19 nt, Artificial Sequence
  synthetic siRNA/shRNA YB1 (L-10213) sequence
cgacgcagacgcccagaaa

SEQ ID NO: 211 - RNA, 19 nt, Artificial Sequence
  synthetic siRNA/shRNA YB1 (L-10213) sequence
guaaggaacggauaugguu

SEQ ID NO: 212 - RNA, 19 nt, Artificial Sequence
  synthetic siRNA/shRNA YB1 (L-10213) sequence
gcggaggcagcaaauguua

SEQ ID NO: 213 - RNA, 19 nt, Artificial Sequence
  synthetic siRNA/shRNA MTA1 (L-004127) sequence
ucacggacauucagcaaga

SEQ ID NO: 214 - RNA, 19 nt, Artificial Sequence
  synthetic siRNA/shRNA MTA1 (L-004127) sequence
ggaccaaaccgcaguaaca

SEQ ID NO: 215 - RNA, 19 nt, Artificial Sequence
  synthetic siRNA/shRNA MTA1 (L-004127) sequence
gcaucuuguuggacauauu

SEQ ID NO: 216 - RNA, 19 nt, Artificial Sequence
  synthetic siRNA/shRNA MTA1 (L-004127) sequence
ccagcaucauugaguacua

SEQ ID NO: 217 - RNA, 19 nt, Artificial Sequence
  synthetic siRNA/shRNA CD44 (L-009999) sequence
gaauauaaccugccgcuuu

SEQ ID NO: 218 - RNA, 19 nt, Artificial Sequence
  synthetic siRNA/shRNA CD44 (L-009999) sequence
caaguggacucaacggaga

SEQ ID NO: 219 - RNA, 19 nt, Artificial Sequence
  synthetic siRNA/shRNA CD44 (L-009999) sequence
cgaagaaggugugggcaga

SEQ ID NO: 220 - RNA, 19 nt, Artificial Sequence
  synthetic siRNA/shRNA CD44 (L-009999) sequence
gaucaacaguggcaaugga

SEQ ID NO: 221 - RNA, 19 nt, Artificial Sequence
  synthetic siRNA/shRNA 4EBP1 (L-003005) sequence
cugauggagugucggaacu

SEQ ID NO: 222 - RNA, 19 nt, Artificial Sequence
  synthetic siRNA/shRNA 4EBP1 (L-003005) sequence
caucuaugaccggaaauuc

SEQ ID NO: 223 - RNA, 19 nt, Artificial Sequence
  synthetic siRNA/shRNA 4EBP1 (L-003005) sequence
gcaauagcccagaagauaa

SEQ ID NO: 224 - RNA, 19 nt, Artificial Sequence
  synthetic siRNA/shRNA 4EBP1 (L-003005) sequence
gagauggacauuuaaagca

SEQ ID NO: 225 - RNA, 19 nt, Artificial Sequence
  synthetic siRNA/shRNA 4EBP2 (L-018671) sequence
gcagcuaccucaugacuau

SEQ ID NO: 226 - RNA, 19 nt, Artificial Sequence
  synthetic siRNA/shRNA 4EBP2 (L-018671) sequence
ggaggaacucgaaucauuu

SEQ ID NO: 227 - RNA, 19 nt, Artificial Sequence
  synthetic siRNA/shRNA 4EBP2 (L-018671) sequence
gcaauucucccauggcuca

SEQ ID NO: 228 - RNA, 19 nt, Artificial Sequence
  synthetic siRNA/shRNA 4EBP2 (L-018671) sequence
uugaacaacuugaacaauc

SEQ ID NO: 229 - RNA, 19 nt, Artificial Sequence
  synthetic siRNA/shRNA rictor (LL-016984) sequence
gacacaagcacuucgauua

SEQ ID NO: 230 - RNA, 19 nt, Artificial Sequence
  synthetic siRNA/shRNA rictor (LL-016984) sequence
gaagauuuauugaguccua

SEQ ID NO: 231 - RNA, 19 nt, Artificial Sequence
  synthetic siRNA/shRNA rictor (LL-016984) sequence
gcgagcugauguagaauua

SEQ ID NO: 232 - RNA, 19 nt, Artificial Sequence
  synthetic siRNA/shRNA rictor (LL-016984) sequence
gggaauacaacuccaaaua

SEQ ID NO: 233 - DNA, 19 bp, Artificial Sequence
  synthetic siRNA/shRNA PTEN (SH-003023-01-10) sequence
gctaagagaggtttccgaa

SEQ ID NO: 234 - DNA, 19 bp, Artificial Sequence
  synthetic siRNA/shRNA PTEN (SH-003023-02-10) sequence
agactgatgtgtatacgta